(19) United States
(12) Patent Application Publication
(10) Pub. No.: US 2006/0089355 A1
(43) Pub. Date: Apr. 27, 2006

(54) Methods of treating alzheimer's disease using aromatically substituted w-amino-alkanoic acid amides and alkanoic acid diamides

(76) Inventors: Michel Maillard, Redwood Shores, CA (US); Varghese John, San Francisco, CA (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/517,981

(22) PCT Filed: Jun. 11, 2003
(86) PCT No.: PCT/US03/18283

§ 371 Date: Jul. 14, 2005

Related U.S. Application Data
(60) Provisional application No. 60/387,756, filed on Jun. 11, 2002.

(51) Int. Cl. A61K 31/5377; A61K 31/455; A61K 31/495; A61K 31/445; A61K 31/401; A61K 31/165

(52) U.S. Cl. 514/235.2; 514/355; 514/616; 514/317; 514/423; 514/255.01

(57) ABSTRACT

Disclosed are methods for treating Alzheimer's disease, and other diseases, and/or inhibiting beta-secretase enzyme, and/or inhibiting de-position of A beta peptide in a mammal, by use of compounds of formula (I) wherein the variables R₁, R₂, R₃, R₄, R₅, X₁, and X₂ are defined herein.

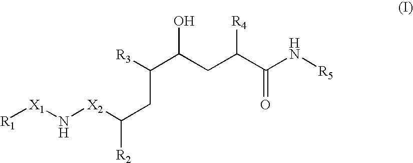

This application claims priority to Provisional U.S. Patent Application Ser. No. 60/387,756, filed Jun. 11, 2002.

### FIELD OF THE INVENTION

The present invention relates to the treatment of Alzheimer's disease and other similar diseases, and more specifically to the use of compounds that inhibit beta-secretase, an enzyme that cleaves amyloid precursor protein to produce A beta peptide, a major component of the amyloid plaques found in the brains of Alzheimer's sufferers, in such methods.

### BACKGROUND OF THE INVENTION

Alzheimer's disease (AD) is a progressive degenerative disease of the brain primarily associated with aging. Clinical presentation of AD is characterized by loss of memory, cognition, reasoning, judgment, and orientation. As the disease progresses, motor, sensory, and linguistic abilities are also affected until there is global impairment of multiple cognitive functions. These cognitive losses occur gradually, but typically lead to severe impairment and eventual death in the range of four to twelve years.

Alzheimer's disease is characterized by two major pathologic observations in the brain: neurofibrillary tangles and beta amyloid (or neuritic) plaques, comprised predominantly of an aggregate of a peptide fragment know as A beta. Individuals with AD exhibit characteristic beta-amyloid deposits in the brain (beta amyloid plaques) and in cerebral blood vessels (beta amyloid angiopathy) as well as neurofibrillary tangles. Neurofibrillary tangles occur not only in Alzheimer's disease but also in other dementia-inducing disorders. On autopsy, large numbers of these lesions are generally found in areas of the human brain important for memory and cognition.

Smaller numbers of these lesions in a more restricted anatomical distribution are found in the brains of most aged humans who do not have clinical AD. Amyloidogenic plaques and vascular amyloid angiopathy also characterize the brains of individuals with Trisomy 21 (Down's Syndrome), Hereditary Cerebral Hemorrhage with Amyloidosis of the Dutch-Type (HCHWA-D), and other neurodegenerative disorders. Beta-amyloid is a defining feature of AD, now believed to be a causative precursor or factor in the development of disease. Deposition of A beta in areas of the brain responsible for cognitive activities is a major factor in the development of AD. Beta-amyloid plaques are predominantly composed of amyloid beta peptide (A beta, also sometimes designated betaA4). A beta peptide is derived by proteolysis of the amyloid precursor protein (APP) and is comprised of 39-42 amino acids. Several proteases called secretases are involved in the processing of APP.

Cleavage of APP at the N-terminus of the A beta peptide by beta-secretase and at the C-terminus by one or more gamma-secretases constitutes the beta-amyloidogenic pathway, i.e. the pathway by which A beta is formed. Cleavage of APP by alpha-secretase produces alpha-sAPP, a secreted form of APP that does not result in beta-amyloid plaque formation. This alternate pathway precludes the formation of A beta peptide. A description of the proteolytic processing fragments of APP is found, for example, in U.S. Pat. Nos. 5,441,870; 5,721,130; and 5,942,400.

An aspartyl protease has been identified as the enzyme responsible for processing of APP at the beta-secretase cleavage site. The beta-secretase enzyme has been disclosed using varied nomenclature, including BACE, Asp, and Memapsin. See, for example, Sindha et al., 1999, Nature 402:537-554 (p501) and published PCT application WO00/17369.

Several lines of evidence indicate that progressive cerebral deposition of beta-amyloid peptide (A beta) plays a seminal role in the pathogenesis of AD and can precede cognitive symptoms by years or decades. See, for example, Selkoe, 1991, Neuron 6:487. Release of A beta from neuronal cells grown in culture and the presence of A beta in cerebrospinal fluid (CSF) of both normal individuals and AD subjects has been demonstrated. See, for example, Seubert et al., 1992, Nature 359:325-327.

It has been proposed that A beta peptide accumulates as a result of APP processing by beta-secretase, thus inhibition of this enzyme's activity is desirable for the treatment of AD. In vivo processing of APP at the beta-secretase cleavage site is thought to be a rate-limiting step in A beta production, and is thus a therapeutic target for the treatment of AD. See for example, Sabbagh, M., et al., 1997, Alz. Dis. Rev. 3, 1-19.

BACE1 knockout mice fail to produce A beta, and present a normal phenotype. When crossed with transgenic mice that over express APP, the progeny show reduced amounts of A beta in brain extracts as compared with control animals (Luo et al., 2001 Nature Neuroscience 4:231-232). This evidence further supports the proposal that inhibition of beta-secretase activity and reduction of A beta in the brain provides a therapeutic method for the treatment of AD and other beta amyloid disorders.

At present there are no effective treatments for halting, preventing, or reversing the progression of Alzheimer's disease. Therefore, there is an urgent need for pharmaceutical agents capable of slowing the progression of Alzheimer's disease and/or preventing it in the first place.

Compounds that are effective inhibitors of beta-secretase, that inhibit beta-secretase-mediated cleavage of APP, that are effective inhibitors of A beta production, and/or are effective to reduce amyloid beta deposits or plaques, are needed for the treatment and prevention of disease characterized by amyloid beta deposits or plaques, such as AD.

U.S. Pat. No. 5,641,778 discloses aromatically substituted ω-amino-alkanoic acid amides and alkanoic acid diamides of the formula 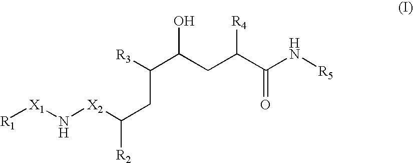
wherein R₁ is a 2-R_{A}-3-R_{B}-phenyl radical, a 2-R_{A}-4-R_{C}-phenyl radical, a 2-R_{A}-pyridin-3-yl radical a 3-R_{A}-pyridin-2-yl radical or a 1-R_{D}-indol-3-yl radical,

wherein one of the radicals R_{A} and R_{B} is an aliphatic or heterecycloaliphatic-aliphatic radical or free or aliphatically, araliphatically or heteroaraliphatically etherified hydroxy and the other is hydrogen, an aliphatic radical or free or esterified or amidated carboxy,

R_{C} is hydrogen, an aliphatic radical, free or aliphatically, araliphatically, heterearaliphatically or heterearylaliphatically etherified hydroxy or an unsubstituted or heteroaliphatically substituted amino group, and

R_{D} is an aliphatic, araliphatic or heteroaliphatic radical,

one of the radicals X₁ and X₂ is carbonyl and the other is methylene,

R₂ is an aliphatic radical,

R₃ is unsubstituted or aliphatically substituted amino,

R₄ is an aliphatic or araliphatic radical, and

R₅ is an aliphatic or cycloaliphatic-aliphatic radical or an optionally hydrogenated and/or oxo-substituted heteroaryl radical or an optionally hydrogenated and/or oxo-substituted heteroaryl or heteroaliphatyl radical bonded via a carbon atom,

and salts thereof.

U.S. Pat. No. 5,641,778 discloses how to make the above compounds and how to use them in inhibiting the natural enzyme, renin; the disclosure of U.S. Pat. No. 5,641,778 is incorporated herein by reference in its entirety.

### SUMMARY OF INVENTION

The present invention relates to methods of treating a subject who has, or in preventing a subject from developing, a disease or condition selected from the group consisting of Alzheimer's disease, for helping prevent or delay the onset of Alzheimer's disease, for helping to slow the progression of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which comprises administration of a therapeutically effective amount of a compound of formula (I): 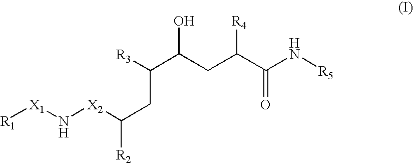
wherein R₁ is a 2-R_{A}-3-R_{B}-phenyl radical, a 2-R_{A}-4-R_{C}-phenyl radical, a 2-R_{A}-pyridin-3-yl radical a 3-R_{A}-pyridin-2-yl radical or a 1-R_{D}-indol-3-yl radical,

wherein one of the radicals R_{A} and R_{B} is an aliphatic or heterecycloaliphatic-aliphatic radical or free or aliphatically, araliphatically or heteroaraliphatically etherified hydroxy and the other is hydrogen, an aliphatic radical or free or esterified or amidated carboxy,

R_{C} is hydrogen, an aliphatic radical, free or aliphatically, araliphatically, heterearaliphatically or heterearylaliphatically etherified hydroxy or an unsubstituted or heteroaliphatically substituted amino group, and

R_{D} is an aliphatic, araliphatic or heteroaliphatic radical,

one of the radicals X₁ and X₂ is carbonyl and the other is methylene,

R₂ is an aliphatic radical,

R₃ is unsubstituted or aliphatically substituted amino,

R₄ is an aliphatic or araliphatic radical, and

R₅ is an aliphatic or cycloaliphatic-aliphatic radical or an optionally hydrogenated and/or oxo-substituted heteroaryl radical or an optionally hydrogenated and/or oxo-substituted heteroaryl or heteroaliphatyl radical bonded via a carbon atom; and salts of the mentioned compounds where salt-forming groups are present.

### DETAILED DESCRIPTION OF THE INVENTION

U.S. Pat. No. 5,641,778 discloses various compounds of the formula I:

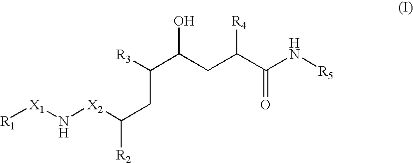

where R₁, R₂, R₃, R₄, R₅, X₁, and X₂ are as defined above, which are useful as inhibitors of the enzyme, renin. This patent does not have any disclosure with regard to Alzheimer's disease.

U.S. Pat. No. 5,641,778 discloses how to make the above compounds and how to use them for the treatment of hypertension related disorders. U.S. Pat. No. 5,641,778 is incorporated herein by reference, in its entirety.

In one aspect, the present invention relates to methods of treating a subject who has, or in preventing a subject from developing, a disease or condition selected from the group consisting of Alzheimer's disease, for helping prevent or delay the onset of Alzheimer's disease, for helping to slow the progression of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which comprises administration of a therapeutically effective amount of a compound of formula (I):

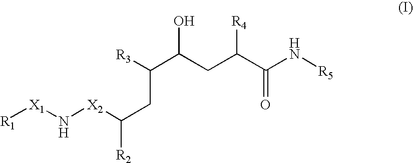

where R₁, R₂, R₃, R₄, R₅, X₁, and X₂ are as defined above.

Preferred compounds of formula (I) include the following:
(2S,4S,5S,7R)-N-(4-Amino-7-butylcarbamoyl-5-hydroxy-2-isopropyl-octyl)-2-(3-methoxypropoxy)-benzamide;
(2S,4S,5S,7R)-N-(4-Amino-7-butylcarbamoyl-5-hydroxy-2-isopropyl-octyl)-3-methoxy-2-(3-methoxypropoxy)-benzamide;
(2S,4S,5S,7R)-N-(4-Amino-7-butylcarbamoyl-5-hydroxy-2-isopropyl-octyl)-4-methoxy-2-(3-methoxypropoxy)-benzamide;
(2S,4S,5S,7R)-N-(4-Amino-7-butylcarbamoyl-5-hydroxy-2-isopropyl-octyl)-3-(3-methoxypropoxy)-benzamide;
(2S,4S,5S,7R)-N-(7-Butylcarbamoyl-4-formylamino-5-hydroxy-2-isopropyl-octyl)-3-methoxy-2-(3-methoxypropoxy)-benzamide;
(2R,4S,5S,7R)-1-Benzyl-1H-indole-3-carboxylic acid N-(4-amino-7-butylcarbamoyl-5-hydroxy-2-isopropyl-octyl)-amide;
(2R,4S,5S,7R)-1-(2-Methoxyethyl)-1H-indole-3-carboxylic acid N-(4-amino-7-butylcarbamoyl-5-hydroxy-2-isopropyl-octyl)-amide;
(2R,4S,5S,7R)-1-Pyridin-2-yl-1H-indole-3-carboxylic acid N-(4-amino-7-butylcarbamoyl-5-hydroxy-2-isopropyl-octyl)-amide;
(2R,4S,5S,7R)-1-(2-Methoxybenzyl)-1H-indole-3-carboxylic acid N-(4-amino-7-butylcarbamoyl-5-hydroxy-2-isopropyl-octyl)-amide;
(2R,4S,5S,7R)-N-(4-Amino-7-butylcarbamoyl-5-hydroxy-2-isopropyl-octyl)-2-(3-methoxypropoxy)-benzamide;
(2R,4S,5S,7R)-N-(4-Amino-7-butylcarbamoyl-5-hydroxy-2-methyl-octyl)-2-(3-methoxypropoxy)-benzamide;
(2R,4S,5S,7R)-N-(4-Amino-7-butylcarbamoyl-5-hydroxy-2-methyl-octyl)-2-(3-methoxypropoxy)-benzamide;
(2S,4S,5S,7S)-N-(4-Amino-7-butylcarbamoyl-5-hydroxy-2-isopropyl-8-methyl-nonyl)-2-(3-methoxypropoxy)-benzamide;
(2S,4S,5S,7S)-N-(4-Amino-7-butylcarbamoyl-5-hydroxy-2-isopropyl-8-methyl-nonyl)-2-(4-methoxybutoxy)-benzamide;
(2S,4S,5S,7S)-N-(4-Amino-7-butylcarbamoyl-5-hydroxy-2-isopropyl-8-methyl-nonyl)-2-propoxy-benzamide;
(2S,4S,5S,7S)-N-(4-amino-7-butylcarbamoyl-5-hydroxy-2-isopropyl-8-methyl-nonyl)-2-(2-methoxyethoxy)-benzamide;
(2S,4S,5S,7S)-N-(4-Amino-7-butylcarbamoyl-5-hydroxy-2-isopropyl-8-methyl-nonyl)-2-[2-(2-methoxyethoxy)-ethoxy]-benzamide;
(2S,4S,5S,7S)-N-(4-Amino-7-butylcarbamoyl-5-hydroxy-2-isopropyl-8-methyl-nonyl)-4-methoxy-2-(3-methoxypropoxy)-benzamide;
(2S,4S,5S,7S)-N-(4-Amino-7-butylcarbamoyl-5-hydroxy-2-isopropyl-8-methyl-nonyl)-4-methoxy-3-(3-methoxypropoxy)-benzamide;
4S,5S,7S)-N-(4-amino-7-butylcarbamoyl-5-hydroxy-2-isopropyl-8-methyl-nonyl)-2-(propoxymethyl)-benzamide;
4S,5S,7S)-N-(4-amino-7-butylcarbamoyl-5-hydroxy-2-isopropyl-8-methyl-nonyl)-2-acetamido-benzamide;
(2S,4S,5S,7S)-N-(4-Amino-7-butylcarbamoyl-5-hydroxy-2-isopropyl-8-methyl-nonyl)-2-[2-(acetamido)-ethoxy]-benzamide;
(2S,4S,5S,7S)-N-(4-Amino-7-butylcarbamoyl-5-hydroxy-2-isopropyl-8-methyl-nonyl)-2-(4-methoxybut-2-enoxy)-benzamide;
(2S,4S,5S,7S)-N-(4-Amino-7-butylcarbamoyl-5-hydroxy-2-isopropyl-8-methyl-nonyl)-2-(4-methoxybutoxy)-4-methyl-benzamide;
(2S,4S,5S,7S)-N-[4-Amino-7-butylcarbamoyl-5-hydroxy-2-isopropyl-8-methyl-nonyl]-2-(3-methoxypropoxy)-nicotinamide;
(2S,4S,5S,7S)-N-[4-Amino-7-butylcarbamoyl-5-hydroxy-2-isopropyl-8-methyl-nonyl]-3-(4-methoxybutoxy)-pyridine-2-carboxylic acid amide;
(2S,4S,5S,7S)-N-(4-Amino-7-butylcarbamoyl-5-hydroxy-2-isopropyl-8-methyl-nonyl)-2-hydroxy-benzamide;
(2S,4S,5S,7S)-N-(4-Amino-7-butylcarbamoyl-5-hydroxy-2-isopropyl-8-methyl-nonyl)-2-[2-(methoxymethoxy)-ethoxy]-benzamide;
(2S,4S,5S,7S)-N-[4-Amino-5-hydroxy-2-isopropyl-8-methyl-7-(2-morpholin-4-ylethylcarbamoyl)-nonyl]-2-(3-methoxypropoxy)-benzamide;
(2S,4S,5S,7S)-N-[4-Amino-5-hydroxy-2-isopropyl-8-methyl-7-(2-morpholin-4-ylethylcarbamoyl)-nonyl]-2-(4-methoxybutoxy)-benzamide;
(2S,4S,5S,7S)-N-[4-Amino-5-hydroxy-2-isopropyl-8-methyl-7-(2-morpholin-4-ylethylcarbamoyl)-nonyl]-2-(2-methoxyethoxy)-benzamide;
(2S,4S,5S,7S)-N-[4-Amino-5-hydroxy-2-isopropyl-8-methyl-7-(2-morpholin-4-ethylcarbamoyl)-nonyl]-2-(3-methoxypropoxy)-nicotinamide;
(2S,4S,5S,7S)-N-[4-Amino-5-hydroxy-2-isopropyl-8-methyl-7-(2-morpholin-4-ylethylcarbamoyl)-nonyl]-3-(4-methoxybutoxy)-pyridine-2-carboxylic acid amide;
(2S,4S,5S,7S)-N-[4-Amino-5-hydroxy-2-isopropyl-8-methyl-7-(2-morpholin-4-ylethylcarbamoyl)-nonyl]-2-(4-methoxybut-2-enoxy)-benzamide;
(2S,4S,5S,7S)-N-[4-Amino-5-hydroxy-2-isopropyl-8-methyl-7-(2-morpholin-4-ylethylcarbamoyl)-nonyl]-2-(4-methoxybutoxy)-4-methyl-benzamide;
(2S,4S,5S,7S)-N-[4-Amino-5-hydroxy-2-isopropyl-8-methyl-7-(2-morpholin-4-ylethylcarbamoyl)-methyl-nonyl]-2-(5-methoxypentyloxy)-benzamide;
(2S,4S,5S,7S)-N-[4-Amino-5-hydroxy-2-isopropyl-8-methyl-7-(3-morpholin-4-ylpropylcarbamoyl)-nonyl]-2-(4-methoxybutoxy)-benzamide;
(2S,4S,5S,7S)-N-(4-Amino-7-butylcarbamoyl-5-hydroxy-2-isopropyl-8-methyl-nonyl)-2-(4-methoxybutoxy)-4-(morpholin-4-ylmethyl)-benzamide;
(2S,4S,5S,7S)-N-(4-Amino-7-butylcarbamoyl hydroxy-2-isopropyl-8-methyl-nonyl-2-(4-methoxybutoxy)-4-[2-(morpholin-4-yl)-ethoxy]-benzamide;
(2S,4S,5S,7S)-N-(4-Amino-7-butylcarbamoyl-5-hydroxy-2-isopropyl-8-methyl-nonyl)-4-[3-(dimethylamino)-propoxy]-2-(4-methoxybutoxy)-benzamide;
(2S,4S,5S,7S)-N-(4-Amino-7-butylcarbamoyl-5-hydroxy-2-isopropyl-8-methyl-nonyl)-2-(4-methoxybutoxy)-4-(piperidin-1-yl)methyl-benzamide;
(2S,4S,5S,7S)-N-(4-Amino-7-butylcarbamoyl-5-hydroxy-2-isopropyl-8-methyl-nonyl)-2-(4-methoxybutoxy)-4-(pyrrolidin-1-yl)methyl-benzamide;
(2S,4S,5S,7S)-N-(4-Amino-7-butylcarbamoyl-5-hydroxy-2-isopropyl-8-methyl-nonyl)-2-(4-methoxybutoxy)-4-(2-piperidin-1-ylethoxy)-benzamide;
(2S,4S,5S,7S)-N-(4-Amino-7-butylcarbamoyl-5-hydroxy-2-isopropyl-8-methyl-nonyl)-4-dimethylaminomethyl-2-(4-methoxybutoxy)-benzamide;
(2S,4S,5S,7S)-N-(4-Amino-7-butylcarbamoyl-5-hydroxy-2-isopropyl-8-methyl-nonyl)-2-(4-methoxybutoxy)-4-(4-methylpiperazin-1-yl)methyl-benzamide;
(2S,4S,5S,7S)-N-(4-Amino-7-butylcarbamoyl-5-hydroxy-2-isopropyl-8-methyl-nonyl)-4-(4-acetylpiperazin-1-yl)methyl-2-(4-methoxybutoxy)-benzamide;
(2S,4S,5S,7S)-N-(4-Amino-7-butylcarbamoyl-5-hydroxy-2-isopropyl-8-methyl-nonyl)-2-(3-aminopropoxy)-benzamide;
(2S,4S,5S,7S)-N-(4-Amino-7-butylcarbamoyl-5-hydroxy-2-isopropyl-8-methyl-nonyl)-2-(2-aminoethoxy)-benzamide;
(2S,4S,5S,7S)-N-(4-Amino-7-butylcarbamoyl-5-hydroxy-2-isopropyl-8-methyl-nonyl)-2-[2-(4-acetylpiperazin-1-yl)-ethoxy]-benzamide;
(2S,4S,5S,7S)-N-(4-Amino-7-butylcarbamoyl-5-hydroxy-2-isopropyl-8-methyl-nonyl)-2-[2-(morpholin-4-yl)-ethyl]-benzamide;
(2S,4S,5S,7S)-N-(4-Amino-7-butylcarbamoyl-5-hydroxy-2-isopropyl-8-methyl-nonyl)-2-(3-dimethylaminopropoxy)-benzamide;
(2S,4S,5S,7S)-N-(4-Amino-7-butylcarbamoyl-5-hydroxy-2-isopropyl-8-methyl-nonyl)-2-[3-(morpholin-4-yl)-propoxy]-benzamide;
(2S,4S,5S,7S)-N-(4-Amino-7-butylcarbamoyl-5-hydroxy-2-isopropyl-8-methyl-nonyl)-2-[2-(morpholin-4-yl)-ethoxy]-benzamide;
(2S,4S,5S,7S)-N-(4-Amino-7-butylcarbamoyl-5-hydroxy-2-isopropyl-8-methyl-nonyl)-2-(2(4-methoxypiperidin-1-yl)-ethyl]-benzamide;
(2S,4S,5S,7S)-N-(4-Amino-7-butylcarbamoyl-5-hydroxy-2-isopropyl-8-methyl-nonyl)-2-[2(4-acetylpiperazin-1-yl)-ethyl]-benzamide;
(2S,4S,5S,7S)-4-Amino-5-hydroxy-2,7-diisopropyl-octanedioic acid 8-butylamide 1-[2-(3-methoxypropoxy)-benzyl]amide;
(2S,4S,5S,7S)-4-Amino-5-hydroxy-2,7-diisopropyl-octanedioic acid 8-butylamide 1-[3-(3-methoxypropoxy)-benzyl]amide;
(2S,4S,5S,7S)-4-Amino-5-hydroxy-2,7-diisopropyl-octandioic acid 8-butylamide 1-[2-(4-methoxybutoxy)-benzyl]amide;
(2S,4S,5S,7S)-4-Amino-5-hydroxy-2,7-diisopropyl-octandioic acid 8-butylamide 1-[2-(5-methoxypentyloxy)-benzyl]amide;
(2S,4S,5S,7S)-N1-(4-Amino-7-butylcarbamoyl-5-hydroxy-2-isopropyl-8-methyl-nonyl)-N-4-methyl-2-(4-methoxybutoxy)-terephthaldiamide;
(2S,4S,5S,7S)-N1-(4-Amino-7-butylcarbamoyl-5-hydroxy-2-isopropyl-8-methyl-nonyl)-N-4-[(2-morpholin-4-yl)-ethyl]-2-(4-methoxybutoxy)-terephthaldiamide;
(2S,4S,5S,7S)-N1-(4-Amino-7-butylcarbamoyl-5-hydroxy-2-isopropyl-8-methyl-nonyl)-2-(4-methoxybutoxy)-terephthaldiamide;
(2S,4S,5S,7S)-N-4-(4-Amino-7-butylcarbamoyl-5-hydroxy-2-isopropyl-8-methyl-nonyl)-3-(4-methoxybutoxy)-terephthalmic acid;
(2S,4S,5S,7S)-N-(4-Amino-7-butylcarbamoyl hydroxy-2-isopropyl-8-methyl-nonyl)-4-butylcarbamoylmethoxy-2-(4-methoxybutoxy)-benzamide;
(2S,4S,5S,7S)-[4-(4-Amino-7-butylcarbamoyl-5-hydroxy-2-isopropyl-8-methyl-nonylcarbamoyl)-3-(4-methoxybutoxy)-phenoxy]-acetic acid;
(2S,4S,5S,7S)-N-{4-Amino-5-hydroxy-2-isopropyl-8-methyl-7-[2-(morpholin-4-yl)-ethylcarbamoyl]-nonyl}-2-(4-methoxybutoxy)-4-[2-(morpholin-4-yl)-ethylcarbamoylmethoxy]-benzamide;
(2S,4S,5S,7S)-N-(4-Amino-7-butylcarbamoyl-5-hydroxy-2-isopropyl-8-methyl-nonyl)-2-(4-methoxybutoxy)-4-(1H-tetrazol-5-ylmethoxy)-benzamide;
(2S,4S,5S,7S,2R′)-N-[4-Amino-7-(2′-methylcarbamoyl-propylcarbamoyl)-5-hydroxy-2-isopropyl-8-methyl-nonyl]-2-(4-methoxybutoxy)-benzamide;
(2S,4S,5S,7S)-N-(4-Amino-7-[2-(dimethylaminocarbamoyl)-ethylcarbamoyl]-5-hydroxy-2-isopropyl-8-methyl-nonyl)-2-(4-methoxybutoxy)-benzamide;
(2S,4S,5S,7S)-N-[4-Amino-7-(3-carbamoylpropylcarbamoyl)-5-hydroxy-2-isopropyl-8-methyl-nonyl]-2-(4-methoxybutoxy)-benzamide;
(2S,4S,5S,7S)-N-[4-Amino-7-(2-carbamoyl-2-methylpropylcarbamoyl)-5-hydroxy-2-isopropyl-8-methyl-nonyl]-2-(4-methoxybutoxy)-benzamide;
(2S,4S,5S,7S)-N-{4-Amino-5-hydroxy-2-isopropyl-8-methyl-7-(3-(morpholin-4-yl)-3-oxopropylcarbamoyl]-nonyl}-2-(4-methoxybutoxy)-benzamide;
(2S,4S,5S,7S)-N-{7-[2-(4-Acetylpiperidin-1-yl)-ethylcarbamoyl]-4amino-5-hydroxy-2-isopropyl-8-methyl-nonyl}-2-(4-methoxybutoxy)-benzamide;
(2S,4S,5S,7S)-N-[4-Amino-5-hydroxy-2-isopropyl-8-methyl-7-(2-thiomorpholin-4-ylethylcarbamoyl)-methyl-nonyl]-2-(4-methoxybutoxy)-benzamide;
(2S,4S,5S,7S)-N-(4-Amino-7-(2-carbamoyl-2-methylpropylcarbamoyl)-5-hydroxy-2-isopropyl-8-methyl-nonyl)-2-(4-methoxybutoxy)-4-(2-morpholin-4-ylmethoxy)-benzamide;
(2S,4S,5S,7S)-N-(4-Amino-7-(2-carbamoyl-2-methylpropylcarbamoyl)-5-hydroxy-2-isopropyl-8-methy-nonyl)-2-(4-methoxybutoxy)-4-(morpholin-4-ylmethyl)-benzamide;
(2S,4S,5S,7S)-N-[4-Amino-7-(2-carbamoyl-2-methylpropylcarbamoyl)-5-hydroxy-2-isopropyl-8-methyl-nonyl]-2-(2-morpholin-4-ylethoxy)-benzamide;
(2S,4S,5S,7S)-N-{4-Amino-5-hydroxy-2-isopropyl-7-[2-(4-methoxycarbonylpiperidin-1-yl)-ethylcarbamoyl]-8-methyl-nonyl}-2-(4-methoxybutoxy)-benzamide;
(2S,4S,5S,7R)-N-{4-Amino-5-hydroxy-2-methyl-7-[(2-morpholin-4-ylethyl)-carbamoyl]-octyl}-2-(3-methoxypropoxy)-benzamide; and
(2S,4S,5S,7S)-N-{4-Amino-5-hydroxy-2-isopropyl-8-methyl-7-[2-(morpholin-4-yl)-ethyl-carbamoyl]-nonyl}-4-carbamoylmethoxy-2-(4-methoxybutoxy)-benzamide;
   or pharmaceutically acceptable salts thereof.

In one aspect, this method of treatment can be used where the disease is Alzheimer's disease.

In another aspect, this method of treatment can help prevent or delay the onset of Alzheimer's disease.

In another aspect, this method of treatment can help slow the progression of Alzheimer's disease.

In another aspect, this method of treatment can be used where the disease is mild cognitive impairment.

In another aspect, this method of treatment can be used where the disease is Down's syndrome.

In another aspect, this method of treatment can be used where the disease is Hereditary Cerebral Hemorrhage with Amyloidosis of the Dutch-Type.

In another aspect, this method of treatment can be used where the disease is cerebral amyloid angiopathy.

In another aspect, this method of treatment can be used where the disease is degenerative dementias.

In another aspect, this method of treatment can be used where the disease is diffuse Lewy body type of Alzheimer's disease.

In another aspect, this method of treatment can treat an existing disease, such as those listed above.

In another aspect, this method of treatment can prevent a disease, such as those listed above, from developing or progressing.

The methods of the invention employ therapeutically effective amounts: for oral administration from about 0.1 mg/day to about 1,000 mg/day; for parenteral, sublingual, intranasal, intrathecal administration from about 0.5 to about 100 mg/day; for depo administration and implants from about 0.5 mg/day to about 50 mg/day; for topical administration from about 0.5 mg/day to about 200 mg/day; for rectal administration from about 0.5 mg to about 500 mg.

In a preferred aspect, the therapeutically effective amounts for oral administration is from about 1 mg/day to about 100 mg/day; and for parenteral administration from about 5 to about 50 mg daily.

In a more preferred aspect, the therapeutically effective amounts for oral administration is from about 5 mg/day to about 50 mg/day.

The present invention also includes the use of a compound of formula (I), or a pharmaceutically acceptable salt thereof for the manufacture of a medicament for use in treating a subject who has, or in preventing a subject from developing, a disease or condition selected from the group consisting of Alzheimer's disease, for helping prevent or delay the onset of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, diffuse Lewy body type of Alzheimer's disease and who is in need of such treatment.

In one aspect, this use of a compound of formula (I) can be employed where the disease is Alzheimer's disease.

In another aspect, this use of a compound of formula (I) can help prevent or delay the onset of Alzheimer's disease.

In another aspect, this use of a compound of formula (I) can help slow the progression of Alzheimer's disease.

In another aspect, this use of a compound of formula (I) can be employed where the disease is mild cognitive impairment.

In another aspect, this use of a compound of formula (I) can be employed where the disease is Down's syndrome.

In another aspect, this use of a compound of formula (I) can be employed where the disease is Hereditary Cerebral Hemorrhage with Amyloidosis of the Dutch-Type.

In another aspect, this use of a compound of formula (I) can be employed where the disease is cerebral amyloid angiopathy.

In another aspect, this use of a compound of formula (I) can be employed where the disease is degenerative dementias.

In another aspect, this use of a compound of formula (I) can be employed where the disease is diffuse Lewy body type of Alzheimer's disease.

In a preferred aspect, the subject is a human subject or human patient.

In a preferred aspect, this use of a compound of formula (I) is a pharmaceutically acceptable salt of an acid selected from the group consisting of acids hydrochloric, hydrobromic, hydroiodic, nitric, sulfuric, phosphoric, citric, methanesulfonic, CH₃—(CH₂)ₙ—COOH where n is 0 thru 4, HOOC—(CH₂)ₙ—COOH where n is as defined above, HOOC—CH═CH—COOH, and phenyl-COOH.

The present invention also includes methods for inhibiting beta-secretase activity, for inhibiting cleavage of amyloid precursor protein (APP), in a reaction mixture, at a site between Met596 and Asp597, numbered for the APP-695 amino acid isotype, or at a corresponding site of an isotype or mutant thereof; for inhibiting production of amyloid beta peptide (A beta) in a cell; for inhibiting the production of beta-amyloid plaque in an animal; and for treating or preventing a disease characterized by beta-amyloid deposits in the brain. These methods each include administration of a therapeutically effective amount of a compound of formula (I), or a pharmaceutically acceptable salt thereof.

The present invention also includes a method for inhibiting beta-secretase activity, including exposing said beta-secretase to an effective inhibitory amount of a compound of formula (I), or a pharmaceutically acceptable salt thereof.

In one aspect, this method includes exposing said beta-secretase to said compound in vitro.

In another aspect, this method includes exposing said beta-secretase to said compound in a cell.

In another aspect, this method includes exposing said beta-secretase to said compound in a cell in an animal.

In another aspect, this method includes exposing said beta-secretase to said compound in a human.

The present invention also includes a method for inhibiting cleavage of amyloid precursor protein (APP), in a reaction mixture, at a site between Met596 and Asp597, numbered for the APP-695 amino acid isotype; or at a corresponding site of an isotype or mutant thereof, including exposing said reaction mixture to an effective inhibitory amount of a compound of formula (I), or a pharmaceutically acceptable salt thereof.

In one aspect, this method employs a cleavage site: between Met652 and Asp653, numbered for the APP-751 isotype; between Met 671 and Asp 672, numbered for the APP-770 isotype; between Leu596 and Asp597 of the APP-695 Swedish Mutation; between Leu652 and Asp653 of the APP-751 Swedish Mutation; or between Leu671 and Asp672 of the APP-770 Swedish Mutation.

In another aspect, this method exposes said reaction mixture in vitro.

In another aspect, this method exposes said reaction mixture in a cell.

In another aspect, this method exposes said reaction mixture in an animal cell.

In another aspect, this method exposes said reaction mixture in a human cell.

The present invention also includes a method for inhibiting production of amyloid beta peptide (A beta) in a cell, including administering to said cell an effective inhibitory amount of a compound of formula (I), or a pharmaceutically acceptable salt thereof.

In an embodiment, this method includes administering to an animal.

In an embodiment, this method includes administering to a human.

The present invention also includes a method for inhibiting the production of beta-amyloid plaque in an animal, including administering to said animal an effective inhibitory amount of a compound of formula (I), or a pharmaceutically acceptable salt thereof.

In one embodiment of this aspect, this method includes administering to a human.

The present invention also includes a method for treating or preventing a disease characterized by beta-amyloid deposits in the brain including administering to a subject an effective therapeutic amount of a compound of formula (I), or a pharmaceutically acceptable salt thereof.

In one aspect, this method employs a compound at a therapeutic amount in the range of from about 0.1 to about 1000 mg/day.

In another aspect, this method employs a compound at a therapeutic amount in the range of from about 15 to about 1500 mg/day.

In another aspect, this method employs a compound at a therapeutic amount in the range of from about 1 to about 100 mg/day.

In another aspect, this method employs a compound at a therapeutic amount in the range of from about 5 to about 50 mg/day.

In another aspect, this method can be used where said disease is Alzheimer's disease.

In another aspect, this method can be used where said disease is Mild Cognitive Impairment, Down's Syndrome, or Hereditary Cerebral Hemorrhage with Amyloidosis of the Dutch Type.

The present invention also includes a composition including beta-secretase complexed with a compound of formula (I), or a pharmaceutically acceptable salt thereof.

The present invention also includes a method for producing a beta-secretase complex including exposing beta-secretase to a compound of formula (I), or a pharmaceutically acceptable salt thereof, in a reaction mixture under conditions suitable for the production of said complex.

In an embodiment, this method employs exposing in vitro.

In an embodiment, this method employs a reaction mixture that is a cell.

The present invention also includes a component kit including component parts capable of being assembled, in which at least one component part includes a compound of formula (I) enclosed in a container.

In an embodiment, this component kit includes lyophilized compound, and at least one further component part includes a diluent.

The present invention also includes a container kit including a plurality of containers, each container including one or more unit dose of a compound of formula (I), or a pharmaceutically acceptable salt thereof.

In an embodiment, this container kit includes each container adapted for oral delivery and includes a tablet, gel, or capsule.

In an embodiment, this container kit includes each container adapted for parenteral delivery and includes a depot product, syringe, ampoule, or vial.

In an embodiment, this container kit includes each container adapted for topical delivery and includes a patch, medipad, ointment, or cream.

The present invention also includes an agent kit including a compound of formula (I), or a pharmaceutically acceptable salt thereof; and one or more therapeutic agents selected from the group consisting of an antioxidant, an anti-inflammatory, a gamma secretase inhibitor, a neurotrophic agent, an acetyl cholinesterase inhibitor, a statin, an A beta peptide, and an anti-A beta antibody.

The present invention provides compounds, compositions, kits, and methods for inhibiting beta-secretase-mediated cleavage of amyloid precursor protein (APP). More particularly, the compounds, compositions, and methods of the invention are effective to inhibit the production of A beta peptide and to treat or prevent any human or veterinary disease or condition associated with a pathological form of A beta peptide.

The compounds, compositions, and methods of the invention are useful for treating humans who have Alzheimer's Disease (AD), for helping prevent or delay the onset of AD, for treating subjects with mild cognitive impairment (MCI), and preventing or delaying the onset of AD in those subjects who would otherwise be expected to progress from MCI to AD, for treating Down's syndrome, for treating Hereditary Cerebral Hemorrhage with Amyloidosis of the Dutch Type, for treating cerebral beta-amyloid angiopathy and preventing its potential consequences such as single and recurrent lobar hemorrhages, for treating other degenerative dementias, including dementias of mixed vascular and degenerative origin, for treating dementia associated with Parkinson's disease, dementia associated with progressive supranuclear palsy, dementia associated with cortical basal degeneration, and diffuse Lewy body type AD.

The compounds of the invention possess beta-secretase inhibitory activity. The inhibitory activities of the compounds of the invention are readily demonstrated, for example, using one or more of the assays described herein or known in the art.

The compounds of formula (I) can form salts when reacted with acids. Pharmaceutically acceptable salts are preferred over the corresponding amines of formula (I) since they frequently produce compounds which are generally more water soluble, stable and/or more crystalline. Pharmaceutically acceptable salts are any salt which retains the activity of the parent compound and does not impart any deleterious or undesirable effect on the subject to whom it is administered and in the context in which it is administered. Pharmaceutically acceptable salts include acid addition salts of both inorganic and organic acids. The preferred pharmaceutically acceptable salts include salts of the following acids acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, succinic, sulfamic, sulfanilic, sulfonic, sulfuric, tannic, tartaric, teoclic and toluenesulfonic. For other acceptable salts, see Int. J. Pharm., 33, 201-217 (1986) and J. Pharm. Sci., 66(1), 1, (1977).

The present invention provides kits, and methods for inhibiting beta-secretase enzyme activity and A beta peptide production. Inhibition of beta-secretase enzyme activity halts or reduces the production of A beta from APP and reduces or eliminates the formation of beta-amyloid deposits in the brain.

Methods of the Invention

The compounds of the invention, and pharmaceutically acceptable salts thereof, are useful for treating humans or animals suffering from a condition characterized by a pathological form of beta-amyloid peptide, such as beta-amyloid plaques, and for helping to prevent or delay the onset of such a condition. For example, the compounds are useful for treating Alzheimer's disease, for helping prevent or delay the onset of Alzheimer's disease, for treating subjects with MCI (mild cognitive impairment) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobal hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type Alzheimer's disease. The compounds and compositions of the invention are particularly useful for treating, preventing, or slowing the progression of Alzheimer's disease. When treating or preventing these diseases, the compounds of the invention can either be used individually or in combination, as is best for the subject.

With regard to these diseases, the term “treating” means that compounds of the invention can be used in humans with existing disease. The compounds of the invention will not necessarily cure the subject who has the disease but will delay or slow the progression or prevent further progression of the disease thereby giving the individual a more useful life span.

The term “preventing” means that that if the compounds of the invention are administered to those who do not now have the disease but who would normally develop the disease or be at increased risk for the disease, they will not develop the disease. In addition, “preventing” also includes delaying the development of the disease in an individual who will ultimately develop the disease or would be at risk for the disease due to age, familial history, genetic or chromosomal abnormalities, and/or due to the presence of one or more biological markers for the disease, such as a known genetic mutation of APP or APP cleavage products in brain tissues or fluids. By delaying the onset of the disease, compounds of the invention have prevented the individual from getting the disease during the period in which the individual would normally have gotten the disease or reduce the rate of development of the disease or some of its effects but for the administration of compounds of the invention up to the time the individual ultimately gets the disease. Preventing also includes administration of the compounds of the invention to those individuals thought to be predisposed to the disease.

In a preferred aspect, the compounds of the invention are useful for slowing the progression of disease symptoms.

In another preferred aspect, the compounds of the invention are useful for preventing the further progression of disease symptoms.

In treating or preventing the above diseases, the compounds of the invention are administered in a therapeutically effective amount. The therapeutically effective amount will vary depending on the particular compound used and the route of administration, as is known to those skilled in the art.

In treating a subject displaying any of the diagnosed above conditions a physician may administer a compound of the invention immediately and continue administration indefinitely, as needed. In treating subjects who are not diagnosed as having Alzheimer's disease, but who are believed to be at substantial risk for Alzheimer's disease, the physician should preferably start treatment when the subject first experiences early pre-Alzheimer's symptoms such as, memory or cognitive problems associated with aging. In addition, there are some subjects who may be determined to be at risk for developing Alzheimer's through the detection of a genetic marker such as APOE4 or other biological indicators that are predictive for Alzheimer's disease. In these situations, even though the subject does not have symptoms of the disease, administration of the compounds of the invention may be started before symptoms appear, and treatment may be continued indefinitely to prevent or delay the onset of the disease.

Dosage Forms and Amounts

The compounds of the invention can be administered orally, parenterally, (IV, IM, depo-IM, SQ, and depo SQ), sublingually, intranasally (inhalation), intrathecally, topically, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

About 1 to 500 mg of a compound or mixture of compounds of the invention or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the subject treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, an that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds of the invention can be administered orally, parenterally (IV, IM, depo-IM, SQ, and depo-SQ), sublingually, intranasally (inhalation), intrathecally, topically, or rectally. Dosage forms known to those skilled in the art are suitable for delivery of the compounds of the invention.

Compounds of the invention may be administered enterally or parenterally. When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the subject 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to inhibit beta-secretase activity, to inhibit A beta production, to inhibit A beta deposition, or to treat or prevent AD is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a subject may be started at one dose, that dose may be varied over time as the subject's condition changes.

Compounds of the invention may also be advantageously delivered in a nano crystal dispersion formulation. Preparation of such formulations is described, for example, in U.S. Pat. No. 5,145,684. Nano crystalline dispersions of HIV protease inhibitors and their method of use are described in U.S. Pat. No. 6,045,829. The nano crystalline formulations typically afford greater bioavailability of drug compounds.

The compounds of the invention can be administered parenterally, for example, by IV, IM, depo-IM, SC, or depo-SC. When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg. In part because of the forgetfulness of the subjects with Alzheimer's disease, it is preferred that the parenteral dosage form be a depo formulation.

The compounds of the invention can be administered sublingually. When given sublingually, the compounds of the invention should be given one to four times daily in the amounts described above for IM administration.

The compounds of the invention can be administered intranasally. When given by this route, the appropriate dosage forms are a nasal spray or dry powder, as is known to those skilled in the art. The dosage of the compounds of the invention for in intranasal administration is the amount described above for IM administration.

The compounds of the invention can be administered intrathecally. When given by this route the appropriate dosage form can be a parenteral dosage form as is known to those skilled in the art. The dosage of the compounds of the invention for intrathecal administration is the amount described above for IM administration.

The compounds of the invention can be administered topically. When given by this route, the appropriate dosage form is a cream, ointment, or patch. Because of the amount of the compounds of the invention to be administered, the patch is preferred. When administered topically, the dosage is from about 0.5 mg/day to about 200 mg/day. Because the amount that can be delivered by a patch is limited, two or more patches may be used. The number and size of the patch is not important, what is important is that a therapeutically effective amount of the compounds of the invention be delivered as is known to those skilled in the art. The compounds of the invention can be administered rectally by suppository as is known to those skilled in the art. When administered by suppository, the therapeutically effective amount is from about 0.5 mg to about 500 mg.

The compounds of the invention can be administered by implants as is known to those skilled in the art. When administering a compound of the invention by implant, the therapeutically effective amount is the amount described above for depot administration.

The invention here is the new compounds of the invention and new methods of using the compounds of the invention. Given a particular compound of the invention and a desired dosage form, one skilled in the art would know how to prepare and administer the appropriate dosage form.

The compounds of the invention are used in the same manner, by the same routes of administration, using the same pharmaceutical dosage forms, and at the same dosing schedule as described above, for preventing disease or treating subjects with MCI (mild cognitive impairment) and preventing or delaying the onset of Alzheimer's disease in those who would progress from MCI to AD, for treating or preventing Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type of Alzheimer's disease.

The compounds of the invention can be used with each other or with other agents used to treat or prevent the conditions listed above. Such agents include gamma-secretase inhibitors, anti-amyloid vaccines and pharmaceutical agents such as donepezil hydrochloride (ARICEPT Tablets), tacrine hydrochloride (COGNEX Capsules) or other acetylcholine esterase inhibitors and with direct or indirectneurotropic agents of the future.

In addition, the compounds of the invention can also be used with inhibitors of P-glycoproten (P-gp). The use of P-gp inhibitors is known to those skilled in the art. See for example, Cancer Research, 53, 4595-4602 (1993), Clin. Cancer Res., 2, 7-12 (1996), Cancer Research, 56, 4171-4179 (1996), International Publications WO99/64001 and WO01/10387. The important thing is that the blood level of the P-gp inhibitor be such that it exerts its effect in inhibiting P-gp from decreasing brain blood levels of the compounds of the invention. To that end the P-gp inhibitor and the compounds of the invention can be administered at the same time, by the same or different route of administration, or at different times. The important thing is not the time of administration but having an effective blood level of the P-gp inhibitor.

Suitable P-gp inhibitors include cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979, PSC-833, GF-102,918 and other steroids. It is to be understood that additional agents will be found that do the same function and are also considered to be useful.

The P-gp inhibitors can be administered orally, parenterally, (IV, IM, IM-depo, SQ, SQ-depo), topically, sublingually, rectally, intranasally, intrathecally and by implant.

The therapeutically effective amount of the P-gp inhibitors is from about 0.1 to about 300 mg/kg/day, preferably about 0.1 to about 150 mg/kg daily. It is understood that while a subject may be started on one dose, that dose may have to be varied over time as the subject's condition changes.

When administered orally, the P-gp inhibitors can be administered in usual dosage forms for oral administration as is known to those skilled in the art. These dosage forms include the usual solid unit dosage forms of tablets and capsules as well as liquid dosage forms such as solutions, suspensions and elixirs. When the solid dosage forms are used, it is preferred that they be of the sustained release type so that the P-gp inhibitors need to be administered only once or twice daily. The oral dosage forms are administered to the subject one thru four times daily. It is preferred that the P-gp inhibitors be administered either three or fewer times a day, more preferably once or twice daily. Hence, it is preferred that the P-gp inhibitors be administered in solid dosage form and further it is preferred that the solid dosage form be a sustained release form which permits once or twice daily dosing. It is preferred that what ever dosage form is used, that it be designed so as to protect the P-gp inhibitors from the acidic environment of the stomach. Enteric coated tablets are well known to those skilled in the art. In addition, capsules filled with small spheres each coated to protect from the acidic stomach, are also well known to those skilled in the art.

In addition, the P-gp inhibitors can be administered parenterally. When administered parenterally they can be administered IV, IM, depo-IM, SQ or depo-SQ. The P-gp inhibitors can be given sublingually. When given sublingually, the P-gp inhibitors should be given one thru four times daily in the same amount as for IM administration.

The P-gp inhibitors can be given intranasally. When given by this route of administration, the appropriate dosage forms are a nasal spray or dry powder as is known to those skilled in the art. The dosage of the P-gp inhibitors for intranasal administration is the same as for IM administration.

The P-gp inhibitors can be given intrathecally. When given by this route of administration the appropriate dosage form can be a parenteral dosage form as is known to those skilled in the art.

The P-gp inhibitors can be given topically. When given by this route of administration, the appropriate dosage form is a cream, ointment or patch. Because of the amount of the P-gp inhibitors needed to be administered the path is preferred. However, the amount that can be delivered by a patch is limited. Therefore, two or more patches may be required. The number and size of the patch is not important, what is important is that a therapeutically effective amount of the P-gp inhibitors be delivered as is known to those skilled in the art. The P-gp inhibitors can be administered rectally by suppository as is known to those skilled in the art.

The P-gp inhibitors can be administered by implants as is known to those skilled in the art.

There is nothing novel about the route of administration nor the dosage forms for administering the P-gp inhibitors. Given a particular P-gp inhibitor, and a desired dosage form, one skilled in the art would know how to prepare the appropriate dosage form for the P-gp inhibitor.

The compounds employed in the methods of the invention can be used in combination, with each other or with other therapeutic agents or approaches used to treat or prevent the conditions listed above. Such agents or approaches include: acetylcholine esterase inhibitors such as tacrine (tetrahydroaminoacridine, marketed as COGNEX®, donepezil hydrochloride, (marketed as Aricept® and rivastigmine (marketed as Exelon®); gamma-secretase inhibitors; anti-inflammatory agents such as cyclooxygenase II inhibitors; anti-oxidants such as Vitamin E and ginkolides; immunological approaches, such as, for example, immunization with A beta peptide or administration of anti-A beta peptide antibodies; statins; and direct or indirect neurotropic agents such as Cerebrolysin®, AIT-082 (Emilieu, 2000, Arch. Neurol. 57:454), and other neurotropic agents of the future.

It should be apparent to one skilled in the art that the exact dosage and frequency of administration will depend on the particular compounds employed in the methods of the invention administered, the particular condition being treated, the severity of the condition being treated, the age, weight, general physical condition of the particular subject, and other medication the individual may be taking as is well known to administering physicians who are skilled in this art.

Inhibition of APP Cleavage

The compounds of the invention inhibit cleavage of APP between Met595 and Asp596 numbered for the APP695 isoform, or a mutant thereof, or at a corresponding site of a different isoform, such as APP751 or APP770, or a mutant thereof (sometimes referred to as the “beta secretase site”). While not wishing to be bound by a particular theory, inhibition of beta-secretase activity is thought to inhibit production of beta amyloid peptide (A beta). Inhibitory activity is demonstrated in one of a variety of inhibition assays, whereby cleavage of an APP substrate in the presence of a beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compound inhibitors of the invention are known. Representative assay systems are described, for example, in U.S. Pat. Nos. 5,942,400, 5,744,346, as well as in the Examples below.

The enzymatic activity of beta-secretase and the production of A beta can be analyzed in vitro or in vivo, using natural, mutated, and/or synthetic APP substrates, natural, mutated, and/or synthetic enzyme, and the test compound. The analysis may involve primary or secondary cells expressing native, mutant, and/or synthetic APP and enzyme, animal models expressing native APP and enzyme, or may utilize transgenic animal models expressing the substrate and enzyme. Detection of enzymatic activity can be by analysis of one or more of the cleavage products, for example, by immunoassay, fluorometric or chromogenic assay, HPLC, or other means of detection. Inhibitory compounds are determined as those having the ability to decrease the amount of beta-secretase cleavage product produced in comparison to a control, where beta-secretase mediated cleavage in the reaction system is observed and measured in the absence of inhibitory compounds.

Beta-Secretase

Various forms of beta-secretase enzyme are known, and are available and useful for assay of enzyme activity and inhibition of enzyme activity. These include native, recombinant, and synthetic forms of the enzyme. Human beta-secretase is known as Beta Site APP Cleaving Enzyme (BACE), Asp2, and memapsin 2, and has been characterized, for example, in U.S. Pat. No. 5,744,346 and published PCT patent applications WO98/22597, WO00/03819, WO01/23533, and WO00/17369, as well as in literature publications (Hussain et al., 1999, Mol. Cell. Neurosci. 14:419-427; Vassar et al., 1999, Science 286:735-741; Yan et al., 1999, Nature 402:533-537; Sinha et al., 1999, Nature 40:537-540; and Lin et al., 2000, PNAS USA 97:1456-1460). Synthetic forms of the enzyme have also been described (WO98/22597 and WO00/17369). Beta-secretase can be extracted and purified from human brain tissue and can be produced in cells, for example mammalian cells expressing recombinant enzyme.

Preferred methods employ compounds that are effective to inhibit 50% of beta-secretase enzymatic activity at a concentration of less than about 50 micromolar, preferably at a concentration of less than about 10 micromolar, more preferably less than about 1 micromolar, and most preferably less than about 10 nanomolar.

APP Substrate

Assays that demonstrate inhibition of beta-secretase-mediated cleavage of APP can utilize any of the known forms of APP, including the 695 amino acid “normal” isotype described by Kang et al., 1987, Nature 325:733-6, the 770 amino acid isotype described by Kitaguchi et. al., 1981, Nature 331:530-532, and variants such as the Swedish Mutation (KM670-1NL) (APP-SW), the London Mutation (V7176F), and others. See, for example, U.S. Pat. No. 5,766,846 and also Hardy, 1992, Nature Genet. 1:233-234, for a review of known variant mutations. Additional useful substrates include the dibasic amino acid modification, APP-KK disclosed, for example, in WO 00/17369, fragments of APP, and synthetic peptides containing the beta-secretase cleavage site, wild type (WT) or mutated form, e.g., SW, as described, for example, in U.S. Pat. No. 5,942,400 and WO00/03819.

The APP substrate contains the beta-secretase cleavage site of APP (KM-DA or NL-DA) for example, a complete APP peptide or variant, an APP fragment, a recombinant or synthetic APP, or a fusion peptide. Preferably, the fusion peptide includes the beta-secretase cleavage site fused to a peptide having a moiety useful for enzymatic assay, for example, having isolation and/or detection properties. A useful moiety may be an antigenic epitope for antibody binding, a label or other detection moiety, a binding substrate, and the like.

Antibodies

Products characteristic of APP cleavage can be measured by immunoassay using various antibodies, as described, for example, in Pirttila et al., 1999, Neuro. Lett. 249:21-4, and in U.S. Pat. No. 5,612,486. Useful antibodies to detect A beta include, for example, the monoclonal antibody 6E10 (Senetek, St. Louis, Mo.) that specifically recognizes an epitope on amino acids 1-16 of the A beta peptide; antibodies 162 and 164 (New York State Institute for Basic Research, Staten Island, N.Y.) that are specific for human A beta 1-40 and 1-42, respectively; and antibodies that recognize the junction region of beta-amyloid peptide, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products, as described in U.S. Pat. Nos. 5,604,102 and 5,721,130.

Assay Systems

Assays for determining APP cleavage at the beta-secretase cleavage site are well known in the art. Exemplary assays, are described, for example, in U.S. Pat. Nos. 5,744,346 and 5,942,400, and described in the Examples below.

Cell Free Assays

Exemplary assays that can be used to demonstrate the inhibitory activity of the compounds of the invention are described, for example, in WO00/17369, WO 00/03819, and U.S. Pat. Nos. 5,942,400 and 5,744,346. Such assays can be performed in cell-free incubations or in cellular incubations using cells expressing a beta-secretase and an APP substrate having a beta-secretase cleavage site.

An APP substrate containing the beta-secretase cleavage site of APP, for example, a complete APP or variant, an APP fragment, or a recombinant or synthetic APP substrate containing the amino acid sequence: KM-DA or NL-DA, is incubated in the presence of beta-secretase enzyme, a fragment thereof, or a synthetic or recombinant polypeptide variant having beta-secretase activity and effective to cleave the beta-secretase cleavage site of APP, under incubation conditions suitable for the cleavage activity of the enzyme. Suitable substrates optionally include derivatives that may be fusion proteins or peptides that contain the substrate peptide and a modification useful to facilitate the purification or detection of the peptide or its beta-secretase cleavage products. Useful modifications include the insertion of a known antigenic epitope for antibody binding; the linking of a label or detectable moiety, the linking of a binding substrate, and the like.

Suitable incubation conditions for a cell-free in vitro assay include, for example: approximately 200 nanomolar to 10 micromolar substrate, approximately 10 to 200 picomolar enzyme, and approximately 0.1 nanomolar to 10 micromolar inhibitor compound, in aqueous solution, at an approximate pH of 4-7, at approximately 37 degrees C., for a time period of approximately 10 minutes to 3 hours. These incubation conditions are exemplary only, and can be varied as required for the particular assay components and/or desired measurement system. Optimization of the incubation conditions for the particular assay components should account for the specific beta-secretase enzyme used and its pH optimum, any additional enzymes and/or markers that might be used in the assay, and the like. Such optimization is routine and will not require undue experimentation.

One useful assay utilizes a fusion peptide having maltose binding protein (MBP) fused to the C-terminal 125 amino acids of APP-SW. The MBP portion is captured on an assay substrate by anti-MBP capture antibody. Incubation of the captured fusion protein in the presence of beta-secretase results in cleavage of the substrate at the beta-secretase cleavage site. Analysis of the cleavage activity can be, for example, by immunoassay of cleavage products. One such immunoassay detects a unique epitope exposed at the carboxy terminus of the cleaved fusion protein, for example, using the antibody SW192. This assay is described, for example, in U.S. Pat. No. 5,942,400.

Cellular Assay

Numerous cell-based assays can be used to analyze beta-secretase activity and/or processing of APP to release A beta. Contact of an APP substrate with a beta-secretase enzyme within the cell and in the presence or absence of a compound inhibitor of the invention can be used to demonstrate beta-secretase inhibitory activity of the compound. Preferably, assay in the presence of a useful inhibitory compound provides at least about 30%, most preferably at least about 50% inhibition of the enzymatic activity, as compared with a non-inhibited control.

In one embodiment, cells that naturally express beta-secretase are used. Alternatively, cells are modified to express a recombinant beta-secretase or synthetic variant enzyme as discussed above. The APP substrate may be added to the culture medium and is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the beta-secretase APP cleavage site can be used, provided that the expressed APP is permitted to contact the enzyme and enzymatic cleavage activity can be analyzed.

Human cell lines that normally process A beta from APP provide a useful means to assay inhibitory activities of the compounds of the invention. Production and release of A beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a compound inhibitor to demonstrate inhibition of enzymatic activity as compared with a control. Activity of beta-secretase can be measured by analysis of one or more cleavage products of the APP substrate. For example, inhibition of beta-secretase activity against the substrate APP would be expected to decrease release of specific beta-secretase induced APP cleavage products such as A beta.

Although both neural and non-neural cells process and release A beta, levels of endogenous beta-secretase activity are low and often difficult to detect by EIA. The use of cell types known to have enhanced beta-secretase activity, enhanced processing of APP to A beta, and/or enhanced production of A beta are therefore preferred. For example, transfection of cells with the Swedish Mutant form of APP (APP-SW); with APP-KK; or with APP-SW-KK provides cells having enhanced beta-secretase activity and producing amounts of A beta that can be readily measured.

In such assays, for example, the cells expressing APP and beta-secretase are incubated in a culture medium under conditions suitable for beta-secretase enzymatic activity at its cleavage site on the APP substrate. On exposure of the cells to the compound inhibitor, the amount of A beta released into the medium and/or the amount of CTF99 fragments of APP in the cell lysates is reduced as compared with the control. The cleavage products of APP can be analyzed, for example, by immune reactions with specific antibodies, as discussed above.

Preferred cells for analysis of beta-secretase activity include primary human neuronal cells, primary transgenic animal neuronal cells where the transgene is APP, and other cells such as those of a stable 293 cell line expressing APP, for example, APP-SW.

In Vivo Assays: Animal Models

Various animal models can be used to analyze beta-secretase activity and/or processing of APP to release A beta, as described above. For example, transgenic animals expressing APP substrate and beta-secretase enzyme can be used to demonstrate inhibitory activity of the compounds of the invention. Certain transgenic animal models have been described, for example, in U.S. Pat. Nos. 5,877,399; 5,612,486; 5,387,742; 5,720,936; 5,850,003; 5,877,015, and 5,811,633, and in Ganes et al., 1995, Nature 373:523. Preferred are animals that exhibit characteristics associated with the pathophysiology of AD. Administration of the compound inhibitors of the invention to the transgenic mice described herein provides an alternative method for demonstrating the inhibitory activity of the compounds. Administration of the compounds in a pharmaceutically effective carrier and via an administrative route that reaches the target tissue in an appropriate therapeutic amount is also preferred.

Inhibition of beta-secretase mediated cleavage of APP at the beta-secretase cleavage site and of A beta release can be analyzed in these animals by measure of cleavage fragments in the animal's body fluids such as cerebral fluid or tissues. Analysis of brain tissues for A beta deposits or plaques is preferred.

On contacting an APP substrate with a beta-secretase enzyme in the presence of an inhibitory compound of the invention and under conditions sufficient to permit enzymatic mediated cleavage of APP and/or release of A beta from the substrate, the compounds of the invention are effective to reduce beta-secretase-mediated cleavage of APP at the beta-secretase cleavage site and/or effective to reduce released amounts of A beta. Where such contacting is the administration of the inhibitory compounds of the invention to an animal model, for example, as described above, the compounds are effective to reduce A beta deposition in brain tissues of the animal, and to reduce the number and/or size of beta amyloid plaques. Where such administration is to a human subject, the compounds are effective to inhibit or slow the progression of disease characterized by enhanced amounts of A beta, to slow the progression of AD in the, and/or to prevent onset or development of AD in a subject at risk for the disease.

Unless defined otherwise, all scientific and technical terms used herein have the same meaning as commonly understood by one of skill in the art to which this invention belongs. All patents and publications referred to herein are hereby incorporated by reference for all purposes.

Definitions

Unless defined otherwise, all scientific and technical terms used herein have the same meaning as commonly understood by one of skill in the art to which this invention belongs.

All patents and publications referred to herein are hereby incorporated by reference for all purposes.

APP, amyloid precursor protein, is defined as any APP polypeptide, including APP variants, mutations, and isoforms, for example, as disclosed in U.S. Pat. No. 5,766,846.

A beta, amyloid beta peptide, is defined as any peptide resulting from beta-secretase mediated cleavage of APP, including peptides of 39, 40, 41, 42, and 43 amino acids, and extending from the beta-secretase cleavage site to amino acids 39, 40, 41, 42, or 43.

Beta-secretase (BACE1, Asp2, Memapsin 2) is an aspartyl protease that mediates cleavage of APP at the amino-terminal edge of A beta. Human beta-secretase is described, for example, in WO00/17369.

Pharmaceutically acceptable refers to those properties and/or substances that are acceptable to the subject from a pharmacological/toxicological point of view and to the manufacturing pharmaceutical chemist from a physical/chemical point of view regarding composition, formulation, stability, subject acceptance and bioavailability.

A therapeutically effective amount is defined as an amount effective to reduce or lessen at least one symptom of the disease being treated or to reduce or delay onset of one or more clinical markers or symptoms of the disease.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Aliphatic radicals are, for example, lower alkyl, hydroxy-lower alkyl, lower alkanoyloxy-lower alkyl, lower alkoxy-lower alkyl, lower alkoxy-lower alkoxy-lower alkyl, lower alkoxy-lower alkoxy-lower alkyl, a free or amidated carboxy or carboxy-lower alkyl group, free or esterified or amidated dicarboxy-lower alkyl, free or esterified or amidated carboxy-(hydroxy)-lower alkyl, lower alkanesulfonyl-lower alkyl or unsubstituted or N-mono- or N,N-di-lower alkylated sulfamoyl-lower alkyl.

Free or aliphatically, araliphatically, heterocycloaliphatically-aliphatically or heteroarylaliphatically etherified hydroxy is, for example, hydroxy, lower alkoxy, hydroxy-lower alkoxy, lower alkanoyloxy-lower alkoxy, lower alkoxy-lower alkoxy, lower alkoxy-lower alkoxy-lower alkoxy, polyhalo-lower alkoxy or cyano-lower alkoxy; an amino-lower alkoxy radical that is unsubstituted or N-lower alkanoylated or N-mono- or N,N-di-lower alkylated or N,N-disubstituted by lower alkylene, hydroxy-, lower alkoxy- or lower alkoxy-lower alkoxy-lower alkylene, by unsubstituted or N′-lower alkanoylated, lower alkoxycarbonyl- or lower alkoxy-lower alkyl-N′-substituted or N′-lower alkylated aza-lower alkylene, by oxa-lower alkylene, or by optionally S-oxidised thia-lower alkylene; or is unsubstituted or substituted phenyl- or pyridyl-lower alkoxy, or free or amidated carboxy or carboxy-lower alkoxy or tetrazolyl-lower alkoxy.

Heteroaliphatic radicals are, for example, amino-lower alkyl radicals that are unsubstituted or N-lower alkanoylated or N-mono- or N,N-di-lower alkylated or N,N-disubstituted by lower alkylene, hydroxy-, lower alkoxy- or lower alkoxy-lower alkoxy-lower alkylene, by unsubstituted or N′-lower alkanoylated, lower alkoxycarbonyl- or lower alkoxy-lower alkyl-N′-substituted or N′-lower alkylated aza-lower alkylene, by oxa-lower alkylene or by optionally S-oxidised thia-lower alkylene; or N-mono- or N,N-di-lower alkylated thiocarbamoyl-lower alkyl radicals. Araliphatic or heteroaliphatic radicals are, for example, unsubstituted or substituted phenyl- or pyridyl-lower alkyl groups.

Cycloaliphatic-aliphatic radicals are, for example, cycloalkyl-lower alkyl or free or esterified or amidated carboxycycloalkyl-lower alkyl.

Unsubstituted or aliphatically substituted amino is, for example, unsubstituted or N-lower alkanoylated or N-mono- or N,N-di-lower alkylated amino.

Unsubstituted or heteroaliphatically substituted amino is, for example, amino that is unsubstituted or N-lower alkanoylated or N-mono- or N,N-di-lower alkylated or N,N-di-substituted by lower alkylene, hydroxy-, lower alkoxy-, lower alkoxycarbonyl- or lower alkoxy-lower alkoxy-lower alkylene, by unsubstituted or N′-lower alkanoylated, lower alkoxycarbonyl- or lower alkoxy-lower alkyl-N′-substituted or N′-lower alkylated aza-lower alkylene, by oxa-lower alkylene or by optionally S-oxidised thia-lower alkylene.

Free or esterified or amidated carboxy is, for example, free or aliphatically or araliphatically etherified carboxy or aliphatically substituted carbamoyl.

Suitable substituents of phenyl, phenyl-lower alkoxy, pyridyl-lower alkyl, pyridyl-lower alkoxy and optionally hydrogenated and/or oxo-substituted heteroaryl are, for example, lower alkyl, lower alkoxy, hydroxy, nitro, amino, lower alkylamino, di-lower alkylamino, halogen and trifluoromethyl, it being possible for up to 3, especially 1 or 2, of those substituents to be present, which may be identical or different.

Amino that is unsubstituted or N-lower alkanoylated or N-mono- or N,N-di-lower alkylated or N,N-disubstituted by lower alkylene, hydroxy-, lower alkoxy-, lower alkoxycarbonyl- or lower alkoxy-lower alkoxy-lower alkylene, by unsubstituted or N-lower alkylated, N-lower alkanoylated or lower alkoxycarbonyl- or lower alkoxy-lower alkyl-N′-substituted aza-lower alkylene, by oxa-lower alkylene or by optionally S-oxidised thia-lower alkylene is, for example, amino, lower alkanoylamino, lower alkylamino, di-lower alkylamino, an unsubstituted or hydroxy-, lower alkoxy- or lower alkoxy-lower alkyl-substituted piperidino or pyrrolidino group, such as piperidino, hydroxypiperidino, lower alkoxypiperidino, lower alkoxy-lower alkoxypiperidino, lower alkoxycarbonylpiperidino, pyrrolidino, hydroxypyrrolidino, lower alkoxypyrrolidino, lower alkoxy-lower alkoxypyrrolidino, unsubstituted or N′-lower alkylated, N′-lower alkanoylated or lower alkoxycarbonyl- or lower alkoxy-lower alkyl-N′-substituted piperazino, such as piperazino, N′-lower alkylpiperazino, N′-lower alkanoylpiperazino, N′-lower alkoxycarbonylpiperazino or N′-lower alkoxy-lower alkylpiperazino, unsubstituted or lower alkylated morpholino, such as morpholino or lower alkylmorpholino, or optionally S-oxidised thiomorpholino, such as thiomorpholino, S-oxythiomorpholino or S,S-dioxythiomorpholino.

Amino-lower alkyl that is unsubstituted or N-lower alkanoylated or N-mono- or N,N-di-lower alkylated or N,N-disubstituted by lower alkylene, hydroxy-, lower alkoxy-, lower alkoxycarbonyl- or lower alkoxy-lower alkoxy-lower alkylene, by unsubstituted or N-lower alkylated, N-lower alkanoylated or lower alkoxycarbonyl- or lower alkoxy-lower alkyl-N′-substituted aza-lower alkylene; by oxa-lower alkylene or by optionally S-oxidised thia-lower alkylene is, for example, amino-lower alkyl, lower alkanoylamino-lower alkyl, lower alkylamino-lower alkyl, di-lower alkylamino-lower alkyl, unsubstituted or hydroxy-, lower alkoxy- or lower alkoxy-lower alkyl-substituted piperidino- or pyrrolidino-lower alkyl, such as piperidino-lower alkyl, hydroxypiperidino-lower alkyl, lower alkoxypiperidino-lower alkyl, lower alkoxy-lower alkoxypiperidino-lower alkyl, lower alkoxycarbonylpiperidino-lower alkyl, pyrrolidino-lower alkyl, hydroxypyrrolidino-lower alkyl, lower alkoxypyrrolidino-lower alkyl, lower alkoxy-lower alkoxypyrrolidino-lower alkyl, unsubstituted or N′-lower alkylated, N′-lower alkanoylated or lower alkoxycarbonyl- or lower alkoxy-lower alkyl-N′-substituted piperazino-lower alkyl, such as piperazino-lower alkyl, N′-lower alkylpiperazino-lower alkyl, N′-lower alkanoylpiperazino-lower alkyl, N′-lower alkoxycarbonylpiperazino-lower alkyl or N′-lower alkoxy-lower alkylpiperazino-lower alkyl, unsubstituted or lower alkylated morpholino-lower alkyl, such as morpholino-lower alkyl or lower alkylmorpholino-lower alkyl, or optionally S-oxidised thiomorpholino-lower alkyl, such as thiomorpholino-lower alkyl, S-oxythiomorpholino-lower alkyl or S,S-dioxythiomorpholino-lower alkyl.

Amino-lower alkoxy that is unsubstituted or N-lower alkanoylated or N-mono- or N,N-di-lower alkylated, N,N-disubstituted by lower alkylene, hydroxy-, lower alkoxy- or lower alkoxy-lower alkoxy-lower alkylene, by unsubstituted or N-lower alkylated, N-lower alkanoylated or lower alkoxycarbonyl- or lower alkoxy-lower alkyl-N′-substituted aza-lower alkylene, by oxa-lower alkylene or by optionally S-oxidised thia-lower alkylene is, for example, amino-lower alkoxy, lower alkanoylamino-lower alkoxy, lower alkylamino-lower alkoxy, di-lower alkylamino-lower alkoxy, unsubstituted or hydroxy-, lower alkoxy- or lower alkoxy-lower alkyl-substituted piperidino- or pyrrolidino-lower alkoxy, such as piperidino-lower alkoxy, hydroxypiperidino-lower alkoxy, lower alkoxypiperidino-lower alkoxy, lower alkoxy-lower alkoxypiperidino-lower alkoxy, pyrrolidino-lower alkoxy, hydroxypyrrolidino-lower alkoxy, lower alkoxypyrrolidino-lower alkoxy, lower alkoxy-lower alkoxypyrrolidino-lower alkoxy, unsubstituted or N′-lower alkylated, N′-lower alkanoylated or lower alkoxycarbonyl- or lower alkoxy-lower alkyl-N′-substituted piperazino-lower alkoxy, such as piperazino-lower alkoxy, N′-lower alkylpiperazino-lower alkoxy, N′-lower alkanoylpiperazino-lower alkoxy, N′-lower alkoxycarbonylpiperazino-lower alkoxy or N′-lower alkoxy-lower alkylpiperazino-lower alkoxy, unsubstituted or lower alkylated morpholino-lower alkoxy, such as morpholino-lower alkoxy or lower alkylmorpholino-lower alkoxy, or optionally S-oxidised thiomorpholino-lower alkoxy, such as thiomorpholino-lower alkoxy, S-oxythiomorpholino-lower alkoxy or S,S-dioxythiomorpholino-lower alkoxy.

Optionally S-oxidised lower alkylthio-lower alkoxy is, for example, lower alkylthio-lower alkoxy, lower alkanesulfinyl-lower alkoxy or lower alkanesulfonyl-lower alkoxy.

Free or amidated carboxy is, for example, carboxy, carbamoyl, lower alkyl carbamoyl, di-lower alkylcarbamoyl, unsubstituted or hydroxy-, lower alkoxy- or lower alkoxy-lower alkyl-substituted piperidino- or pyrrolidino-carbonyl, such as piperidinocarbonyl, hydroxy piperidinocarbonyl, lower alkoxypiperidinocarbonyl, lower alkoxy-lower alkoxypiperidino carbonyl, pyrrolidinocarbonyl, hydroxypyrrolidinocarbonyl, lower alkoxypyrrolidinocarbonyl, lower alkoxy-lower alkoxypyrrolidinocarbonyl, unsubstituted or N′-lower alkylated, N′-lower alkanoylated or lower alkoxycarbonyl- or lower alkoxy-lower alkyl-N′-substituted piperazinocarbonyl, such as piperazinocarbonyl, N′-lower alkylpiperazinocarbonyl, N′-lower alkanoylpiperazinocarbonyl, N′-lower alkoxycarbonylpiperazinocarbonyl or N′-lower alkoxy-lower alkylpiperazinocarbonyl, unsubstituted or lower alkylated morpholinocarbonyl, such as morpholinocarbonyl or lower alkylmorpholinocarbonyl, or optionally S-oxidised thiomorpholinocarbonyl, such as thiomorpholinocarbonyl, S-oxythiothiomorpholinocarbonyl or S,S-dioxythiomorpholinocarbonyl.

Free or esterified carboxy is, for example, carboxy or lower alkoxycarbonyl.

Free or amidated carboxy-lower alkoxy is, for example, carboxy-lower alkoxy, carbamoyl-lower alkoxy, lower alkylcarbamoyl-lower alkoxy, di-lower alkylcarbamoyl-lower alkoxy, unsubstituted or hydroxy-, lower alkoxy- or lower alkoxy-lower alkyl-substituted piperidino- or pyrrolidino-carbonyl-lower alkoxy, such as piperidinocarbonyl-lower alkoxy, hydroxypiperidinocarbonyl-lower alkoxy, lower alkoxypiperidinocarbonyl-lower alkoxy, lower alkoxy-lower alkoxypiperidinocarbonyl-lower alkoxy, pyrrolidinocarbonyl-lower alkoxy, hydroxy pyrrolidinocarbonyl-lower alkoxy, lower alkoxypyrrolidinocarbonyl-lower alkoxy, lower alkoxy-lower alkoxypyrrolidinocarbonyl-lower alkoxy, unsubstituted or N′-lower alkylated, N′-lower alkanoylated or lower alkoxycarbonyl- or lower alkoxy-lower alkyl-N′-substituted piperazinocarbonyl-lower alkoxy, such as piperazinocarbonyl-lower alkoxy, N′-lower alkylpiperazinocarbonyl-lower alkoxy, N′-lower alkanoylpiperazinocarbonyl-lower alkoxy, N′-lower alkoxycarbonylpiperazinocarbonyl or N′-lower alkoxy-lower alkylpiperazinocarbonyl-lower alkoxy, unsubstituted or lower alkylated morpholinocarbonyl-lower alkoxy, such as morpholinocarbonyl-lower alkoxy or lower alkylmorpholinocarbonyl-lower alkoxy, or optionally S-oxidised thiomorpholinocarbonyl-lower alkoxy, such as thiomorpholinocarbonyl-lower alkoxy, S-oxythiomorpholinocarbonyl or S,S-dioxythiomorpholinocarbonyl-lower alkoxy.

Free or amidated carboxy-lower alkyl is, for example, carboxy-lower alkyl, carbamoyl-lower alkyl, lower alkylcarbamoyl-lower alkyl, di-lower alkylcarbamoyl-lower alkyl, unsubstituted or hydroxy-, lower alkoxy- or lower alkoxy-lower alkyl-substituted piperidino- or pyrrolidinocarbonyl-lower alkyl, such as piperidinocarbonyl-lower alkyl, hydroxypiperidinocarbonyl-lower alkyl, lower alkoxypiperidinocarbonyl-lower alkyl, lower alkoxy-lower alkoxy piperidinocarbonyl-lower alkyl, pyrrolidinocarbonyl-lower alkyl, hydroxypyrrolidinocarbonyl-lower alkyl, lower alkoxypyrrolidinocarbonyl-lower alkyl, lower alkoxy-lower alkoxypyrrolidinocarbonyl-lower alkyl, unsubstituted or N′-lower alkylated, N′-lower alkanoylated or lower alkoxycarbonyl- or lower alkoxy-lower alkyl-N′-substituted piperazinocarbonyl-lower alkyl, such as piperazinocarbonyl-lower alkyl, N′-lower alkylpiperazinocarbonyl-lower alkyl, N′-lower alkanoylpiperazinocarbonyl-lower alkyl, N′-lower alkoxycarbonylpiperazinocarbonyl or N′-lower alkoxy-lower alkylpiperazinocarbonyl-lower alkyl, unsubstituted or lower alkylated morpholinocarbonyl-lower alkyl, such as morpholinocarbonyl-lower alkyl or lower alkylmorpholinocarbonyl-lower alkyl, or optionally S-oxidised thiomorpholinocarbonyl-lower alkyl, such as thiomorpholinocarbonyl-lower alkyl, S-oxythiomorpholinocarbonyl-lower alkyl or S,S-dioxythiomorpholinocarbonyl-lower alkyl.

Free or esterified carboxy-lower alkyl is, for example, carboxy-lower alkyl or lower alkoxycarbonyl-lower alkyl.

Unsubstituted or N-lower alkanoylated or N-mono- or N,N-di-lower alkylated amino is, for example, amino, lower alkanoylamino, lower alkylamino or di-lower alkylamino.

Free or esterified or amidated dicarboxy-lower alkyl is, for example, dicarboxy-lower alkyl, lower alkoxycarbonyl(carboxy)-lower alkyl, di-lower alkoxycarbonyl-lower alkyl, dicarbamoyl-lower lower alkyl, carbamoyl (carboxy)-lower alkyl, di(lower alkylcarbamoyl)-lower alkyl or di(di-lower alkylcarbamoyl)-lower alkyl.

Free or esterified or amidated carboxy(hydroxy)-lower alkyl is for example, carboxy(hydroxy)-lower alkyl, lower alkoxycarbonyl(hydroxy)-lower alkyl, carbamoyl(hydroxy)-lower alkyl, lower alkylcarbamoyl(hydroxy)-lower alkyl or di-lower alkycarbamoyl(hydroxy)-lower alkyl.

Free or esterified or amidated carboxycycloalkyl-lower alkyl is, for example, carboxycycloalkyl-lower alkyl, lower alkoxycarbonylcycloalkyl-lower alkyl, carbamoylcycloalkyl-lower alkyl, lower alkylcarbamoylcycloalkyl-lower alkyl or di-lower alkylcarbamoylcycloalkyl-lower alkyl.

Unsubstituted or N-mono- or N,N-di-lower alkylated thiocarbamoyl-lower alkyl is, for example, thiocarbamoyl-lower alkyl, N-lower alkylthiocarbamoyl-lower alkyl or N,N-di-lower alkylthiocarbamoyl-lower alkyl.

Unsubstituted or N-mono- or N,N-di-lower alkylated sulfamoyl-lower alkyl is, for example, sulfamoyl-lower alkyl, lower alkylsulfamoyl-lower alkyl or di-lower alkylsulfamoyl-lower alkyl.

Optionally hydrogenated and/or oxo-substituted heteroaryl radicals are, for example, optionally partially hydrogenated and/or benzofused 5-membered aza-, diaza-, triaza-, oxadiaza- or tetraaza-aryl or 6-membered aza- or diaza-aryl radicals, such as unsubstituted or oxo-substituted pyrrolidinyl, e.g. pyrrolidinyl or oxopyrrolidinyl, imidazolyl, e.g. imidazol-4-yl, benzimidazolyl, e.g. benzimidazol-2-yl, oxadiazolyl, e.g. 1,2,4-oxadiazol-5-yl, pyridyl, e.g. pyridin-2-yl, oxopiperidinyl, dioxopipeddinyl, oxothiazolyl, oxo-oxazolinyl or quinolinyl, e.g. quinolin-2-yl, or unsubstituted or N-lower alkanoylated piperidyl, such as 1-lower alkanoylpiperidinyl.

Lower alkyl substituted by an optionally hydrogenated and/or oxo-substituted heteroaryl radical that is bonded via a carbon atom contains as optionally hydrogenated heteroaryl radical, for example, an optionally partially hydrogenated and/or benzofused 5-membered aza-, diaza-, triaza-, oxadiaza- or tetraaza-aryl radical or 6-membered aza- or diaza-aryl radical and is, for example, unsubstituted or oxo-substituted pyrrolidinyl-lower alkyl, e.g. pyrrolidinyl-lower alkyl or oxopyrrolidinyl-lower alkyl, imidazolyl-lower alkyl, benzimidazolyl-lower alkyl, oxadiazolyl-lower alkyl, pyridyl-lower alkyl, oxopiperidinyl-lower alkyl, dioxopiperidinyl-lower alkyl, oxothiazolyl-lower alkyl, oxo-oxazonlinyl-lower alkyl or quinolinyl-lower piperidyl-lower alkyl, oxothiazolyl-lower alkyl, oxo-oxazolinyl-lower alkyl or quinolinyl-lower alkyl, also morpholinocarbonyl-lower alkyl or unsubstituted or N-lower alkanoylated piperidyl-lower alkyl, such as 1-lower alkanoyl piperidinyl-lower alkyl.

Amino-lower alkoxy is, for example, amino-C₁-C₇ alkoxy, such as 2-aminoethoxy, 3-aminopropyloxy, 4-aminobutyloxy or 5-aminopentyloxy.

Amino-lower alkyl is, for example, amino-C₁-C₄ alkyl, such as 2-aminoethyl, 3-aminopropyl or 4-aminobutyl.

Benzimidazolyl-lower alkyl is, for example, benzimidazolyl-C₁-C₄ alkyl, such as benzimidazolylmethyl, 2-benzimidazolylethyl, 3-benzimidazolylpropyl or 4-benzimidazolyl butyl.

Carbamoyl(carboxy)-lower alkyl is, for example, carbamoyl(carboxy)-C₁-C₇ alkyl, especially carbamoyl(carboxy)-C₂-C₇ alkyl, such as 2-carbamoyl-1-carboxyethyl, 1-carbamoyl-2-carboxyethyl, 3-carbamoyl-2-carboxypropyl or 2-carbamoyl-3-carboxypropyl.

Carbamoyl(hydroxy)-lower alkyl is, for example, carbamoyl-C₁-C₄ (hydroxy)alkyl, such as 1-carbamoyl-2-hydroxyethyl.

Carbamoylcycloalkyl-lower alkyl has, for example, from 3 to 8, especially from 5 to 7, ring members and is, for example, carbamoylcyclopentyl-, carbamoylcyclohexyl- or carbamoylcycloheptyl-methyl.

Carbamoyl-lower alkoxy is, for example, carbamoyl-C₁-C₇ alkoxy, such as carbamoylmethoxy, 2-carbamoylethoxy, 3carbamoylpropyloxy, 2-(3-carbamoyl)propyloxy, 2-carbamoylpropyloxy, 3-(1-carbamoyl)propyloxy, 2-(2-carbamoyl)propyloxy, 2-(carbamoyl-2-methyl)propyloxy, 4-carbamoylbutyloxy, 1-carbamoylbutyloxy, 1-(1-carbamoyl-2-methyl)butyloxy, 3-(4-carbamoyl-2-methyl)butyloxy, especially 3-carbamoylpropyloxy or 2-carbamoyl-2-methyl-ethoxy.

Carbamoyl-lower alkyl is, for example, carbamoyl-C₁-C₇ alkyl, such as carbamoylmethyl, 2-carbamoylethyl, 3-carbamoylpropyl, 2-(3-carbamoyl)propyl, 2-carbamoylpropyl, 3-(1-carbamoyl)propyl, 2-(2-carbamoyl)propyl, 2-(carbamoyl-2-methyl)propyl, 4-carbamoylbutyl, 1-carbamoylbutyl, 1-(1-carbamoyl-2-methyl)butyl, 3-(4-carbamoyl-2-methyl)butyl, especially 3-carbamoylpropyl or 2-carbamoyl-2-methyl-ethyl.

Carboxycycloalkyl-lower alkyl has, for example, from 3 to 8, especially from 5 to 7, ring members and is, for example, carboxycyclopentyl-, carboxycyclohexyl- or carboxycycloheptyl-methyl.

Carboxy(hydroxy)-lower alkyl is, for example, carboxy-C₁-C₇ (hydroxy)alkyl, such as 1-carboxy-2-hydroxyethyl.

Carboxy-lower alkoxy is, for example, carboxy-C₁-C₄ alkoxy, such as carboxymethoxy, 2-carboxyethoxy, 2- or 3-carboxypropyloxy, 2-carboxy-2-methyl-propyloxy, 2-carboxy-2-ethyl-butyl or 4-carboxybutyloxy, especially carboxymethoxy.

Carboxy-lower alkyl is, for example, carboxy-C₁-C₄ alkyl, such as carboxymethyl, 2-carboxy-ethyl, 2- or 3-carboxpropyl, 2-carboxy-2-methyl-propyl, 2-carboxy-2-ethyl-butyl or 4-carboxybutyl, especially carboxymethyl.

Quinolinyl-lower alkyl is, for example, quinolinyl-C₁-C₄ alkyl, such as quinolinylmethyl, 2-quinolinylethyl or 3-quinolinylpropyl, especially quinolinylmethyl.

Cyano-lower alkoxy is, for example, cyano-C₁-C₄ alkoxy, such as cyanomethoxy, 2-cyanoethoxy, 2- or 3-cyanopropyloxy, 2-cyano-2-methyl-propyloxy, 2-cyano-2-ethyl-butyloxy or 4-cyanobutyloxy, especially cyanomethoxy.

Cyano-lower alkyl is, for example, cyano-C₁-C₄ alkyl, such as cyanomethyl, 2-cyanoethyl, 2- or 3-cyanopropyl, 2-cyano-2-methyl-propyl, 2-cyano-2-ethyl-butyl or 4-cyanobutyl, especially cyanomethyl.

Cycloalkyl-lower alkyl has, for example, from 3 to 8, especially from 5 to 7, ring members and is, for example, cyclopentyl, cyclohexyl or cycloheptyl, also cyclopropyl, cyclobutyl or cyclooctyl.

Di(di-lower alkylcarbamoyl)-lower alkyl is, for example, di-(di-C₁-C₄ alkylcarbamoyl)-C₁-C₄ alkyl, such as 1,2-di(di-C₁-C₄ alkylcarbamoyl)ethyl or 1,3-di(di-C₁-C₄ alkylcarbamoyl)propyl, wherein C₁-C₄ alkyl is, for example, methyl, ethyl or propyl.

Di(lower alkylcarbamoyl)-lower alkyl is, for example, di(C₁-C₄ alkylcarbamoyl)-C₁-C₄ alkyl, such as 1,2-di(C₁-C₄ alkylcarbamoyl)ethyl or 1,3-di(C₁-C₄ alkylcarbamoyl)propyl, wherein C₁-C₄ alkyl is, for example, methyl, ethyl or propyl.

Dicarbamoyl-lower alkyl is, for example, dicarbamoyl-C₁-C₄ alkyl, such as 1,2-dicarbamoyl-ethyl or 1,3-dicarbamoylpropyl.

Dicarboxy-lower alkyl is, for example, dicarboxy-C₁-C₄ alkyl, such as 1,2-dicarboxyethyl or 1,3-dicarboxypropyl.

Di-lower alkyl-Di-lower alkylamino-lower alkoxy is, for example, N,N-di-C₁-C₄ alkylamino-C₁-C₄ alk as 2-dimethylaminoethoxy, 3-dimethylaminopropyloxy, 4-dimethylaminobutyloxy, 2-diethylaminoethyoxy, 2-(N-methyl-N-ethyl-amino)ethoxy or 2-(N-butyl-N-methyl-amino)ethoxy, especially 3-dimethylaminopropyloxy.

Di-lower alkylamino-lower alkyl is, for example, N,N-di-C₁-C₄ alkylamino-C₁-C₄ alkyl, such as 2-dimethylaminoethyl, 3-dimethylaminopropyl, 4-dimethylaminobutyl, 2-diethylaminoethyl, 2-(N-methyl-N-ethyl-amino)ethyl or 2-(N-butyl-N-methyl-amino)ethyl, especially dimethylaminomethyl.

Di-lower alkoxycarbonyl-lower alkyl is, for example, di-lower alkoxycarbonyl-C₁-C₄ alkyl, such as 1,2-dimethoxycarbonylethyl, 1,3-dimethoxycarbonylpropyl, 1,2-dimethoxycarbonylethyl or 1,3-diethoxycarbonylpropyl.

Di-lower alkylamino is, for example, di-C₁-C₄ alkylamino, such as dimethylamino, N-methyl-N-ethylamino, diethylamino, N-methyl-N-propylamino or N-butyl-N-methyl-amino.

Di-lower alkylamino-lower alkoxy is, for example, N,N-di-C₁-C₄ alkylamino-C₁-C₄ alkoxy, such as 2-dimethylaminoethoxy, 3-dimethylaminopropyloxy, 2-dimethylaminopropyloxy, 2-(dimethylamino-2-methyl)propyloxy or 2-(1-dimethylamino-3-methyl)butyloxy, especially 3-dimethylaminopropyloxy.

Di-lower alkylcarbamoyl is, for example, di-C₁-C₄ alkylcarbamoyl, such as dimethylcarbamoyl, N-methyl-N-ethylcarbamoyl, diethylcarbamoyl, N-methyl-N-propylcarbamoyl or N-butyl-N-methyl-carbamoyl.

Di-lower alkylcarbamoyl(hydroxy)-lower alkyl is, for example, di-C₁-C₄ alkylcarbamoyl-C₁-C₇ (hydroxy)alkyl, such as 1-dimethylcarbamoyl- or 1-diethylcarbamoyl-2-hydroxy-ethyl.

Di-lower alkylcarbamoylcycloalkyl-lower alkyl has, for example, from 3 to 8, especially from 5 to 7, ring members and is, for example, di-C₁-C₄ alkylcarbamoyl-C₅-C₇ cycloalkyl-C₁-C₄ alkyl, such as dimethylcarbamoylcyclopentyl-, dimethylcarbamoylcyclohexyl- or dimethylcarbamoylcycloheptyl-methyl.

Di-lower alkylcarbamoyl-lower alkoxy is, for example, N,N-di-C₁-C₄ alkylcarbamoyl-C₁-C₄ alkoxy, such as 2-dimethylcarbamoylethoxy, 3-dimethylcarbamoylpropyloxy, 2-dimethylcarbamoylpropyloxy, 2-(dimethylcarbamoyl-2-methyl) propyloxy or 2-(1-dimethylcarbamoyl-3-methyl)butyloxy, especially 2-dimethylcarbamoylethoxy.

Di-lower alkylcarbamoyl-lower alkyl is, for example, N,N-di-C₁-C₄ alkylcarbamoyl-C₁-C₄ alkyl, such as 2-dimethylcarbamoylethyl, 3-dimethylcarbamoylpropyl, 2-dimethylcarbamoyl propyl, 2-(dimethylcarbamoyl-2-methyl)propyl or 2-(1-dimethylcarbamoyl-3-methyl)butyl, especially 2-dimethylcarbamoylethyl.

Di-lower alkylsulfamoyl-lower alkyl is, for example, N,N-di-C₁-C₄ alkylsulfamoyl-C₁-C₄ alkyl, N,N-dimethylsulfamoyl-C₁-C₄ alkyl, such as N,N-dimethylsulfamoylmethyl, (N,N-dimethylcarbamoyl)ethyl, 3-(N,N-dimethylcarbamoyl)propyl or 4-(N,N-dimethylcarbamoyl)butyl, especially N,N-dimethylcarbamoylmethyl.

Dioxopiperidinyl-lower alkyl is, for example, dioxopiperidino-C₁-C₄ alkyl, such as 2,6-dioxopiperidin-1-ylmethyl, such as 2-(2,6-dioxopiperidin-1-yl)ethyl or 2,6-dioxopiperidin-4-yl-methyl.

S,S-Dioxothiomorpholinocarbonyl-lower alkoxy is, for example, S,S-dioxothiomorpholinocarbonyl-C₁-C₄ alkoxy, such as S,S-dioxothiomorpholinocarbonylmethoxy,2-(S,S-dioxo)thiomorpholinocarbonyl ethoxy, 3-(S,S-dioxo)thiomorpholinocarbonylpropyloxy or 1- or 2-[4-(S,S-dioxo)thiomorpholinocarbonyl]butyloxy.

S,S-Dioxothiomorpholinocarbonyl-lower alkyl is, for example, S,S-dioxothiomorpholinocarbonyl-C₁-C₄ alkyl, such as S,S-dioxothiomorpholinocarbonylmethyl, 2-(S,S-dioxo)thiomorpholinocarbonylethyl, 3-(S,S-dioxo)thiomorpholinocarbonylpropyl or 1- or 2-[4-(S,S-dioxo)thiomorpholinocarbonyl]butyl.

S,S-Dioxothiomorpholino-lower alkoxy is, for example, S,S-dioxothiomorpholino-C₁-C₄ alkoxy, such as S,S-dioxothiomorpholinomethoxy, 2-(S,S-dioxo)thiomorpholinoethoxy, 3-(S,S-dioxo)thiomorpholinopropyloxy or 1- or 2-[4-(S,S-dioxo)thiomorpholino]butyloxy.

S,S-Dioxothiomorpholino-lower alkyl is, for example, S,S-dioxothiomorpholino-C₁-C₄ alkyl, such as S,S-dioxothiomorpholinomethyl, 2-(S,S-dioxo)thiomorpholinoethyl, 3-(S,S-dioxo)thiomorpholinopropyl or 1- or 2-[4-(S,S-dioxo)thiomorpholino]butyl.

Hydroxy-lower alkoxy is, for example, hydroxy-C₂-C₇ alkoxy, especially hydroxy-C₂-C₄ alkoxy, such as 2-hydroxyethoxy, 3-hydroxypropyloxy or 4-hydroxybutyloxy.

Hydroxy-lower alkoxy-lower alkyl is, for example, hydroxy-C₁-C₄ alkoxy-C₁-C₄ alkyl, such as 2-hydroxyethoxymethyl, 2-(2hydroxyethoxy)ethyl, 3-(3-hydroxypropyloxy)propyl or 4-(2-hydroxybutyloxy)butyl, especially 2-(3-hydroxypropyloxy)ethyl or 2-(4-hydroxybutyloxy)ethyl.

Hydroxy-lower alkyl is, for example, hydroxy-C₂-C₇ alkyl, especially hydroxy-C₂-C₄ alkyl, such as 2-hydroxyethyl, 3-hydroxypropyl or 4-hydroxybutyl.

Hydroxy-lower alkylene, together with the carbon atom that binds its free valencies, is, for example, 3-hydroxypyrrolidino or 3- or 4-hydroxypiperidino.

Hydroxypiperidinocarbonyl is, for example, 3- or 4-hydroxypiperidinocarbonyl.

Hydroxypiperidinocarbonyl-lower alkoxy is, for example, hydroxypiperidinocarbonyl-C₁-C₄ alkoxy, such as 3- or 4-hydroxypiperidinocarbonylmethoxy.

Hydroxypiperidinocarbonyl-lower alkyl is, for example, hydroxypiperidinocarbonyl-C₁-C₄ alkyl, such as 3- or 4-hydroxypiperidinocarbonylmethyl.

Hydroxypiperidino-lower alkoxy is, for example, 3- or 4-hydroxypiperidino-C₁-C₄ alkoxy, such as 3- or 4-hydroxypiperidino-4-ylmethoxy, 2-(3- or 4-hydroxypiperidino)ethoxy, 3-(3- or 4-hydroxypiperidino)propyloxy or 4-(3- or 4-hydroxypiperidino)butyloxy.

Hydroxypiperidino-lower alkyl is, for example, 3- or 4-hydroxypiperidino-C₁-C₄ alkyl, such as 3- or 4-hydroxypiperidino-4-ylmethyl, 2-(3- or 4-hydroxypiperidino)ethyl, 3-(3- or 4-hydroxypiperidino)propyl or 4-(3- or 4-hydroxypiperidino)butyl.

Hydroxypyrrolidinocarbonyl-lower alkoxy is, for example, hydroxypyrrolidinocarbonyl-C₁-C₄ alkoxy, such as 3-hydroxypyrrolidinocarbonylmethoxy.

Hydroxypyrrolidinocarbonyl-lower alkyl is, for example, hydroxypyrrolidinocarbonyl-C₁-C₄ alkyl, such as 3-hydroxypyrrolidinocarbonylmethyl

Hydroxypyrrolidino-lower alkoxy is, for example, 3-hydroxypyrrolidino-C₁-C₄ alkoxy, such as 3-hydroxypiperidinopyrrolidinomethoxy.

Hydroxypyrrolidino-lower alkyl is, for example, 3- or 4-hydroxypyrrolidino-C₁-C₄ alkyl, such as 3-hydroxypyrrolidinomethyl.

Imidazolyl-lower alkyl is, for example, imidazolyl-C₁-C₄ alkyl, such as imidazolylmethyl, 2-imidazolylethyl, 3-imidazolylpropyl or 4-imidazolylbutyl.

Morpholinocarbonyl-lower alkoxy is, for example, morpholinocarbonyl-C₁-C₄ alkoxy, such as morpholinocarbonylmethoxy, 2-morpholinocarbonylethoxy, 3-morpholinocarbonylpropyloxy or 4-morpholinocarbonylbutyloxy.

Morpholinocarbonyl-lower alkyl is, for example, morpholinocarbonyl-C₁-C₄ alkyl, such as morpholinocarbonylmethyl, 2-morpholinocarbonylethyl, 3-morpholinocarbonylpropyl or 4-morpholinocarbonylbutyl, especially 2-morpholinocarbonylethyl.

Morpholino-lower alkoxy is, for example, morpholino-C₃-C₄ alkoxy, such as morpholinomethoxy, 2-morpholinoethoxy, 3-morpholinopropyloxy or 4-morpholinobutyloxy, especially 2-morpholinoethoxy or 3-morpholinopropyloxy.

Morpholino-lower alkyl is, for example, morpholino-C₁-C₄ alkyl, such as morpholinomethyl, 2-morpholinocarbonylethyl, 3-morpholinopropyl methyl, 2-morpholinobutyloxy, especially morpholinomethyl, 2-morpholinoethyl or 3-morpholinopropyl.

Morpholino-lower alkylcarbamoyl-lower alkoxy is, for example, N-(morpholino-C₁-C₄ alkylcarbamoyl)-C₄-C₄ alkoxy, such as especially 2-morpholinoethylcarbamoylmethoxy.

Lower alkanoylamino-lower alkyl is, for example, N—C₁-C₄ alkanoylamino-C₁-C₄ alkyl, such as 2-acetoxyaminoethyl.

Lower alkanoylamino is, for example, N—C₁-C₇ alkanoylamino, such as formylamino, acetylamino or pivaloylamino.

Lower alkanoylamino-lower alkoxy preferably carries the lower alkanoylamino group in a position higher than the co-position and is, for example, N—C₁-C₇ alkanoylamino-C₁-C₄ alkoxy, such as 2-formylaminoethoxy, 2-acetylaminoethoxy or 2-pivaloylaminoethoxy, especially 2-acetylaminoethoxy.

Lower alkanoyloxy-lower alkoxy preferably carries the lower alkanoyloxy group in a position higher than the .alpha.-position and is, for example, C₁-C₇ alkanoyloxy-C₁-C₄ alkoxy, such as 4-acetyloxybutyloxy.

Lower alkanoyloxy-lower alkyl preferably carries the lower alkanoyloxy group in a position higher than the .alpha.-position and is, for example, C₁-C₇ alkanoyloxy-C₁-C₄ alkyl, such as 4-acetoxybutyl.

Lower alkanoylpiperazinocarbonyl is, for example, N—C₂-C₇ alkanoylpiperazinocarbonyl, such as 4-acetylpiperazinocarbonyl.

Lower alkanoylpiperazinocarbonyl-lower alkoxy is, for example, N′-C₂-C₇ alkanoylpiperazinocarbonyl-C₁-C₄ alkoxy, such as 4-acetylpiperazinocarbonylmethoxy.

Lower alkanoylpiperazinocarbonyl-lower alkyl is, for example, N′-C₂-C₇ alkanoylpiperazinocarbonyl-C₁-C₄ alkyl, such as especially N′-acetylpiperazinomethyl.

Lower alkanoylpiperazino-lower alkoxy is, for example, N′-C₂-C₇ alkanoylpiperazino-C₁-C₄-alkoxy, such as 4-acetylpiperazinomethoxy.

Lower alkanoylpiperazino-lower alkyl is, for example, N′-C₂-C₇ alkanoylpiperazino-C₁-C₄ alkyl, such as 4-acetylpiperazinomethyl.

Lower alkanoylpiperidinyl is, for example, N′-C₂-C₇ alkanoylpiperidin-4-yl, such as 1-acetylpiperidin-4-ylmethyl. Lower alkanoylpiperidinyl-lower alkyl is, for example N′-C₂-C₇ alkxanoylpiperidin-4-yl-C₁-C₄-)alkyl, such as especially 2-(1-acetylpiperidin-4-yl)ethyl.

Lower alkanesulfinyl-lower alkoxy is, for example, C₂-C₇ alkanesulfinyl-C₁-C₄ alkoxy, such as methanesulfinylmethoxy or 3-methanesulfinyl-2-hydroxy-propyloxy.

Lower alkanesulfonyl-lower alkoxy is, for example, C₁-C₇ alkanesulfonyl-C₁-C₄ alkoxy, such as methanesulfonylmethoxy or 3-methanesulfonyl-2-hydroxy-propyloxy.

Lower alkanesulfonyl-lower alkyl is, for example, C₁-C₇ alkanesulfonyl-C₁-C₄ alkyl, such as ethanesulfonylmethyl, 2ethanesulfonylethyl, 3-ethanesulfonylpropyl or 3-(1,1-dimethylethanesulfonyl)propyl.

Lower alkoxy is, for example, C₁-C₇ alkoxy, preferably C₁-C₄ alkoxy, such as methoxy, ethoxy, propyloxy, isopropyloxy, butyloxy, isobutyloxy, sec-butyloxy, tert-butyloxy, pentyloxy or a hexyloxy or heptyloxy group.

Lower alkoxycarbonyl is, for example, C₂-C₄ alkoxycarbonyl, such as methoxycarbonyl or methoxycarbonyl, ethoxycarbonyl, propyloxycarbonyl, isopropyloxycarbonyl, butyloxycarbonyl, isobutyloxycarbonyl, sec-butyloxycarbonyl, tert-butyloxycarbonyl, pentyloxycarbonyl or a hexyloxycarbonyl or heptyloxycarbonyl group.

Lower alkoxycarbonyl(carboxy)-lower alkyl is, for example, C₁-C₄ alkoxycarbonyl (carboxy)-C₁-C₇ alkyl, especially C₁-C₄ alkoxycarbonyl(carboxy)-C₂-C₇ alkyl, such as 2-methoxycarbonyl-1-carboxyethyl,1-methoxycarbonyl-2-carboxyethyl, 3-methoxycarbonyl-2-carboxy-propyl or 2-methoxycarbonyl-3-carboxy-propyl.

Lower alkoxycarbonyl(hydroxy)-lower alkyl is, for example, C₁-C₄ alkoxycarbonyl-C₁-C₇-(hydroxy)alkyl, such as 1-methoxycarbonyl- or 1-ethoxycarbonyl-2-hydroxy-ethyl.

Lower alkoxycarbonylcycloalkyl-lower alkyl has, for example, from 3 to 8, especially from 5 to 7, ring members and is, for example, C₁-C₄ alkoxycarbonylcyclopentyl-, C₁-C₄ alkoxycarbonylcyclohexyl- or C₁-C₄ alkoxycarbonylcycloheptyl-methyl.

Lower alkoxycarbonyl-lower alkyl is, for example, C₁-C₄ alkoxycarbonyl-C₁-C₄ alkyl, such as methoxycarbonyl- or ethoxycarbonyl-methoxy, 2-methoxycarbonyl- or 2-ethoxycarbonylethoxy, 3-methoxycarbonyl- or 3-ethoxycarbonyl-propyloxy or 4-ethoxycarbonylbutyloxy.

Lower alkoxycarbonylpiperazinocarbonyl is, for example, N′-C₁-C₄ alkoxycarbonylpiperazinocarbonyl, such as 4-methoxycarbonylpiperazinocarbonyl.

Lower alkoxycarbonylpiperazino-lower alkoxy is, for example, N′-C₁-C₄ alkoxycarbonylpiperazinocarbonyl-C₁-C₄ alkoxy, such as 2-(4-methoxycarbonylpiperazinocarbonyl)ethoxy.

Lower alkoxycarbonylpiperazino-lower alkyl is, for example, N′-C₁-C₄ alkoxycarbonylpiperazinocarbonyl-C₁-C₄ alkyl, such as 2-(4-methoxycarbonylpiperazioncarbonyl)ethyl.

Lower alkoxycarbonylpiperidinyl-lower alkyl is, for example, N′-C₁-C₄ alkoxycarbonylpiperazinocarbonyl-C₁-C₄ alkyl, such as 2-(2-methoxycarbonylpiperazinocarbonyl)ethyl.

Lower alkoxy-lower alkenyloxy is, for example, C₁-C₄ alkoxy-C₂-C₄ alkenyloxy, such as 4-methoxybut-2-enyloxy.

Lower alkoxy-lower alkoxy is, for example, C₁-C₄ alkoxy-C₂-C₄ alkoxy, such as 2-methoxy-, 2-ethoxy- or 2-propyloxy-ethoxy, 3-methoxy- or 3-ethoxy-propyloxy or 4-methoxybutyloxy, especially 2-methoxyethoxy, 3-methoxypropyloxy, 4-methoxybutyloxy, 5-methoxypentyloxy.

Lower alkoxy-lower alkoxy-lower alkoxy is, for example, C₁-C₄ alkoxy-C₁-C₄ alkoxy-C₁-C₄ alkoxy, such as 2-methoxy-, 2-ethoxy- or 2-propyloxy-ethoxymethoxy, 2-(2-methoxy-, 2-ethoxy- or 2-propyloxy-ethoxy)ethoxy, 3-(3-methoxy- or 3-ethoxy-propyloxy)propyloxy or 4-(2-methoxybutyloxy)butyloxy, especially 2-(methoxymethoxy)ethoxy or 2-(2-methoxyethoxy)ethoxy.

Lower alkoxy-lower alkoxy-lower alkyl is, for example, C₁-C₄ alkoxy-C₁-C₄ alkoxy-C₁-C₄ alkyl, such as 2-methoxy-, 2-ethoxy- or 2-propyloxy-ethoxymethyl, 2-(2-methoxy-, 2-ethoxy- or 2-propyloxy-ethoxy)ethyl, 3-(3-methoxy- or 3-ethoxy-propyloxy)propyl or 4-(2-methoxybutyloxy)butyl, especially 2-(3-methoxypropyloxy)ethyl or 2-(4-methoxybutyloxy)ethyl.

Lower alkoxy-lower alkoxy-lower alkylene, together with the carbon atom that binds its free valencies, is, for example, C₁-C₄ alkoxy-C₁-C₄ alkoxy-C₁-C₄ alkylene, such as 3-(3-methoxypropyloxy)pyrrolidino, 3-(3-methoxypropyloxy)piperidino or 4-(3-methoxypropyloxy)piperidino.

Lower alkoxy-lower alkoxypiperidinocarbonyl is, for example, C₁-C₄ alkoxy-C₁-C₄ alkoxypiperidinocarbonyl, such as 3(3-methoxypropyloxy)- or 4-(3-methoxypropyloxy)-piperidinocarbonyl.

Lower alkoxy-lower alkoxypiperidinocarbonyl-lower alkoxy is, for example, C₁-C₄ alkoxy-C₁-C₄ alkoxy-piperidinocarbonyl-C₁-C₄ alkoxy, such as 3-(3-methoxypropyloxy)- or 4-(3-methoxypropyl)-piperidinocarbonylmethoxy.

Lower alkoxy-lower alkoxypiperidinocarbonyl-lower alkyl is, for example, C₁-C₄ alkoxy-C₁-C₄ alkoxy-piperidinocarbonyl-C₁-C₄ alkyl, such as 3-(3-methoxypropyloxy)- or 4-(3-methoxypropropyloxy)-piperidinocarbonylmethyl.

Lower alkoxy-lower alkoxypiperidino-lower alkoxy is, for example, 3- or 4-C₁-C₄ alkoxy-C₁-C₄ alkoxy-piperidino-C₁-C₄ alkoxy, such as 3-(3-methoxypropyloxy)- or 4-(3-methoxypropyloxy)piperidinomethoxy, 2-[3-(3-methoxypropyloxy)- or 2-[4-methoxypropyloxy)-piperidino]ethoxy, 3-(3- or 4-hydroxypiperidino)propyloxy or 4-(3- or 4-hydroxypiperidino)butyloxy.

Lower alkoxy-lower alkoxypiperidino-lower alkyl is, for example, 3- or 4-C₁-C₄ alkoxy-C₁-C₄ alkoxy-piperidino-C₁-C₄ alkyl, such as 3-(3-methoxypropyloxy)- or 4-(3-methoxypropyloxy)piperidino-4-ylmethyl, 2-(3- or 4-hydroxypiperidino)ethyl, 3-(3- or 4-hydroxypiperidino)propyl or 4-(3- or 4-hydroxypiperidino)butyl.

Lower alkoxy-lower alkoxypyrrolidinocarbonyl-lower alkoxy is, for example, C₁-C₄ alkoxy-C₁-C₄ alkoxy-pyrrolidinocarbonyl- or hydroxypyrrolidinocarbonyl-C₁-C₄ alkoxy, such as 3-(3-methoxypropyloxy)pyrrolidinocarbonylmethoxy.

Lower alkoxy-lower alkoxypyrrolidinocarbonyl-lower alkyl is, for example, C₁-C₄ alkoxy-pyrrolidinocarbonyl)- or hydroxypyrrolidinocarbonyl-C₁-C₄ alkyl, such as 3-(3-methoxypropyloxy)pyrrolidinocarbonylmethyl.

Lower alkoxy-lower alkoxypyrrolidino-lower alkoxy is, for example, 3- or 4-C₁-C₄ alkoxy-C₁-C₄ alkoxy-pyrrolidino-C₁-C₄ alkoxy, such as 3-(3-methoxypropyloxy)pyrrolidin-1-ylmethoxy.

Lower alkoxy-lower alkoxypyrrolidino-lower alkyl is, for example, 3- or 4-C₁-C₄ alkoxy-C₁-C₄ alkoxy-pyrrolidino-C₁-C₄ alkyl, such as 3-(3-methoxypropyloxy)pyrrolidin-1-ylmethyl.

Lower alkoxy-lower alkyl is, for example, C₁-C₄ alkoxy-C₁-C₄ alkyl, such as ethoxymethyl, propyloxymethyl, butyloxymethyl, 2-methoxy-, 2-ethoxy- or 2-propyloxy-ethyl, 3-methoxy- or 3-ethoxy-propyl or 4-methoxybutyl, especially 3-methoxypropyl or 4-methoxybutyl, especially propyloxymethoxy.

Lower alkoxy-lower alkylene, together with the carbon atom that binds its free valencies, is, for example, C₁-C₄ alkoxy-C₁-C₄ alkylene, such as 3-methoxypyrrolidino, 3-methoxypiperidino or 4-methoxypiperidino.

Lower alkoxy-lower alkylpiperazinocarbonyl is, for example, N′-C₁-C₄ alkoxy-C₁-C₄ alkoxypiperazinocarbonyl, such as N′-(3-methoxypropyl)piperazinocarbonyl, N′-(4-methoxybutyl)piperazinocarbonyl or N′-(3-ethoxypropyl)piperazinocarbonyl.

Lower alkoxy-lower alkylpiperazinocarbonyl-lower alkoxy is, for example, N′-C₁-C₄ alkoxy-C₁-C₄ alkylpiperazinocarbonyl-C₁-C₄ alkoxy, such as N′-(3-methoxypropyl)piperazinocarbonylmethoxy, 2-[N′-(3-methoxypropyl)piperazinocarbonyl]ethoxy, 3-[N′-(3-methoxypropyl)piperazinocarbonyl]propyloxyor 4-[N′-(3-methoxypropyl)piperazinocarbonyl]butyloxy.

Lower alkoxy-lower alkylpiperazinocarbonyl-lower alkyl is, for example, N′-C₁-C₄ alkoxy-C₁-C₄ alkylpiperazinocarbonyl-C₁-C₄ alkyl, such as N′-(3-methoxypropyl)piperazinocarbonylmethyl, 2-[N′-(3-methoxypropyl)piperazinocarbonyl]ethyl, 3-[N′-(3-methoxypropyl)piperazinocarbonyl]propyl or 4-[N′-(3-methoxypropyl)piperazinocarbonyl]butyl.

Lower alkylpiperazino-lower alkoxy is, for example, N′-C₁-C₄ alkylpiperazino-C₁-C₄ alkoxy, such as N′-(3-methoxypropyl)piperazinomethoxy, 2-[N′-(3-methoxypropyl)piperazinolethoxy, 3-[N′-(3-methoxypropyl)piperazinolpropyloxy or 4-[N′-(3-methoxypropyl)piperazino]butyloxy.

Lower alkoxy-lower alkylpiperazino-lower alkyl is, for example, N′-C₁-C₄ alkoxy-C₁-C₄ alkylpiperazino-C₁-C₄ alkyl, such as N′-(3-methoxypropyl)piperazinomethyl, 2-[N′-(3-methoxypropyl)piperazino]ethyl, 3-[N′-(3-methoxypropyl)piperazino]propyl or 4-[N′-(3-methoxypropyl)piperazino]butyl.

Lower alkoxypiperidinocarbonyl is, for example, C₁-C₄ alkoxypiperidinocarbonyl, such as 3- or 4-methoxypiperidinocarabonyl, 3- or 4-ethoxypiperidinocarbonyl, 3- or 4-propyloxypiperidinocarbonyl or 3- or 4-butyloxypiperidinocarbonyl.

Lower alkoxypiperidinocarbonyl-lower alkoxy is, for example, C₁-C₄ alkoxypiperidinocarbonyl-C₁-C₄ alkoxy such as 3- or 4-methoxypiperidinocarbonylmethoxy, 2-(3- or 4-methoxypiperidinocarbonyl)ethoxy, 3-(3- or 4-methoxypiperidinocarbonyl)propyloxy or 4-(3- or 4-methoxypiperidinocarbonyl)butyloxy.

Lower alkoxypiperidinocarbonyl-lower alkyl is, for example, C₁-C₄ alkoxypiperidinocarbonyl-C₁-C₄ alkyl, such as 3- or 4-methoxypiperidinocarbonylmethyl, 2-(3- or 4-methoxypiperidinocarbonyl)ethyl, 3-(3- or 4-methoxypiperidinocarbonyl)propyl or 4-methoxypiperidinocarbonyl)butyl.

Lower alkoxypiperidino-lower alkoxy is, for example, C₁-C₄ alkoxypiperidino-C₁-C₄ alkoxy, such as 2-(3- or 4-methoxypiperidino)piperidinoethoxy, 3-(3- or 4-metholxypiperidino)piperidinopropyloxy or 4-(3- or 4-methoxypiperidino)piperidinobutyloxy.

Lower alkoxypiperidino-lower alkyl is, for example, C₁-C₄ alkoxypiperidino-C₁-C₄ alkyl, such as 3- or 4-methoxypiperidinomethyl, 2-(3- or 4-methoxypiperidino)piperidinoethyl, 3-(3- or 4-methoxypiperidino)piperidinopropyl or 4-(3- or 4-methoxypiperidino)piperidinobutyl.

Lower alkoxypyrrolidinocarbonyl-lower alkoxy is, for example, C₁-C₄ alkoxy-pyrrolidinocarbonyl-C₁-C₄ alkoxy, such as 3-methoxypyrrolidinocarbonylmethoxy, 2-(3-methoxypyrrolidinocarbonyl)ethoxy, 3-(3-methoxypyrrolidinocarbonyl)propyloxy or 4-(3-methoxypyrrolidinocarbonyl)butyloxy.

Lower alkoxypyrrolidinocarbonyl-lower alkyl is, for example, C₁-C₄ alkoxy-pyrrolidinocarbonyl-C₁-C₄ alkyl, such as 3-methoxypyrrolidinocarbonylmethyl, 2-(3-methoxypyrrolidinocarbonyl)ethyl, 3-(3-methoxypyrrolidinocarbonyl)propyl or 4-(3-methoxypyrrolidinocarbonyl)butyl.

Lower alkoxypyrrolidino-lower alkoxy is, for example, C₁-C₄ alkoxy-pyrrolidino-C₁-C₄ alkoxy, such as 3-methoxypyrrolidinomethoxy, 2-(3-methoxypyrrolidino)ethoxy, 3-(3-methoxypyrrolidino)propyloxy or 4-(3-methoxypyrrolidino)butyloxy.

Lower alkoxypyrrolidino-lower alkyl is, for example, C₁-C₄ alkoxy-pyrrolidino-C₁-C₄ alkyl, such as 3-methoxypyrrolidinomethyl, 2-(3-methoxypyrrolidino)ethyl, 3-(3-methoxypyrrolidino)propyl or 4-(3-methoxypyrrolidino)butyl.

Lower alkanoylamino-lower alkoxy is, for example, N—C₁-C₄ alkanoylamino-C₁-C₄ alkoxy, such as 2-acetyloxyaminoethoxy.

Lower alkyl may be straight-chained or branched and/or bridged and is, for example, corresponding C₁-C₇ alkyl, such as methyl, ethyl, propyl, isopropyl, butyl, isobutyl, sec-butyl, tert-butyl, or a pentyl, hexyl or heptyl group.

Lower alkylamino is, for example, C₁-C₄ alkylamino, such as methylamino, ethylamino, propylamino, butylamino, isobutylamino, sec-butylamino or tert-butylamino.

Lower alkylamino-lower alkoxy is, for example, C₁-C₄ alkylamino-C₁-C₄ alkoxy, such as propylaminomethoxy, 2-methylamino-, 2-ethylamino-, 2-propylamino- or 2-butylaminoethoxy, 3-ethylamino- or 3-propylamino-propyloxy or 4-methylaminobutoxy.

Lower alkylamino-lower alkoxy is, for example, C₁-C₄ alkylamino-C₁-C₄ alkoxy, such as propylaminomethoxy, 2-methylamino-, 2-ethylamino-, 2-propylamino- or 2-butylaminoethoxy, 3-ethylamino- or 3-propylamino-propyloxy or 4-methylaminobutoxy.

Lower alkylcarbamoyl is, for example, C₁-C₄ alkylcarbamoyl, such as methylcarbamoyl, ethylcarbamoyl, propylcarbamoyl, butylcarbamoyl, isobutylcarbamoyl, sec-butylcarbamoyl or tertbutylcarbamoyl, especially methylcarbamoyl.

Lower alkylcarbamoyl(hydroxy)-lower alkyl is, for example, C₁-C₄ alkylcarbamoyl-C₁-C₄ alkylcarbamoyl-C₁-C₇ (hydroxy)alkyl, such as 1-methylcarbamoyl- or 1-ethylcarbamoyl-2-hydroxy-ethyl.

Lower alkylcarbamoylcycloalkyl-lower alkyl has, for example, from 3 to 8, especially from 5 to 7, ring members and is, for example, C₁-C₄ alkylcarbamoyl-C₅-C₇ cycloalkyl-C₁-C₄ alkyl, such as methylcarbamoylcyclopentyl-, methylcarbamoylcyclohexyl- or methylcarbamoylcycloheptyl-methyl.

Lower alkylcarbamoyl-lower alkoxy is, for example, N—C₁-C₄ alkylcarbamoyl-C₁-C₄ alkoxy, such as 2-propylcarbamoylethoxy, 3-ethylcarbamoylpropyloxy, 2-ethylcarbamoylpropyloxy, 2-(methylcarbamoyl-2-methyl)propyloxy, 2-(1-methylcarbamoyl-3-methyl)butyloxy or especially butylcarbamoylmethoxy.

Lower alkylcarbamoyl-lower alkyl is, for example, N—C₁-C₄ alkylcarbamoyl-C₁-C₄ alkyl, such as 2-methylcarbamoylethyl, 3-methylcarbamoylpropyl, 2-methylcarbamoylpropyl, 2-(methylcarbamoyl-2-methyl)propylor 2-(1-methylcarbamoyl-3-methyl)butyl, especially 2-methylcarbamoylethyl.

Lower alkylcarbamoyl-lower alkyl is, for example, N—C₁-C₇ alkylcarbamoyl-C₁-C₄ alkyl, such as methyl- or dimethyl-carbamoyl-C₁-C₄ alkyl, e.g. methylcarbamoylmethyl, 2-methylcarbamoylethyl, 3-methylcarbamoylpropyl or especially 2-methylcarbamoyl-2-methyl-propyl.

Lower alkylene, together with the carbon atom that binds its free valencies, is, for example, pyrrolidino or piperidino.

Lower alkylmorpholinocarbonyl is, for example, 4-(C₁-C₄ alkyl)morpholinocarbonyl, such as 4-methylmorpholinocarbonyl, 4-ethylmorpholinocarbonyl, 4-propylmorpholinocarbonyl or 4-butylmorpholinocarbonyl.

Lower alkylmorpholinocarbonyl-lower alkoxy is, for example, C₁-C₄ alkylmorpholinocarbonyl-C₁-C₄ alkoxy, such as methylmorpholinocarbonylmethoxy, 2-methylmorpholinocarbonylethoxy, 3-methylmorpholinocarbonylpropyloxy or 4-methylmorpholinocarbonylbutyloxy.

Lower alkylmorpholinocarbonyl-lower alkyl is, for example, C₁-C₄ alkylmorpholinocarabonyl-C₁-C₄ alkyl, such as methylmorpholinocarbonylmethyl, 2-methylmorpholinocarbonylethyl, 3-methylmorpholinocarbonylpropyl or 4-methylmorpholinocarbonylbutyl, especially 2-methylmorpholinocarbonylethyl.

Lower alkylmorpholino-lower alkoxy is, for example, C₁-C₄ alkylmorpholino-C₁-C₄ alkoxy, such as methylmorpholinomethoxy, 2-methylmorpholinoethoxy, 3-methylmorpholinopropyloxy or 4-methylmorpholinobutyloxy, especially 2-methylmorpholinoethoxy or 3-methylmorpholinopropyloxy.

Lower alkylmorpholino-lower alkyl is, for example, C₁-C₄ alkylmorpholino-C₁-C₄ alkyl, such as methylmorpholinomethyl, 2-methylmorpholinocarbonylethyl, 3-methylmorpholinopropyl or 4-methylmorpholinobutyl.

Lower alkylpiperazinocarbonyl is, for example, N′-C₁-C₄ alkylpiperazinocarbonyl, such as N′-methylpiperazinocarbonyl, N′-ethylpiperazinocarbonyl, N′-propylpiperazinocarbonyl or N′-butylpiperazinocarbonyl.

Lower alkylpiperazinocarbonyl-lower alkoxy is, for example, N′-C₁-C₄ alkylpiperazinocarbonyl-C₁-C₄ alkoxy, such as N′-methylpiperazinocarbonylmethoxy, 2-(N′-methylpiperazinocarbonyl)ethoxy, 3-(N′-methylpiperazinocarbonyl)propyloxy or 4-(N′-methylpiperazinocarbonyl)butyloxy.

Lower alkylpiperazinocarbonyl-lower alkyl is, for example, N′-C₁-C₄ alkylpiperazinocarbonyl-C₁-C₄ alkyl, such as N′-methylpiperazinocarbonylmethyl, 2-(N′-methylpiperazinocarabonyl)ethyl, 3-(N′-methylpiperazinocarbonyl)propyl or 4-(N′-methylpiperazinocarbonyl)butyl, especially N′-methylpiperazinocarbonylmethyl.

Lower alkylpiperazino-lower alkoxy is, for example, N′-C₁-C₄ alkylpiperazino-C₁-C₄ alkoxy, such as N′-methylpiperazinomethoxy, 2-(N′-methylpiperazino)ethoxy, 3-(N′-methylpiperazino)propyloxy or 4-(N′-methylpiperazino)butyloxy.

Lower alkylpiperazino-lower alkyl is, for example, N′-C₁-C₄ alkylpiperazino-C₁-C₄ alkyl, such as N′-methylpiperazinomethyl, 2-(N′-methylpiperazino)ethyl, 3-(N′-methylpiperazino)propyl or 4-(N′-methylpiperazino)butyl, especially N′-methylpiperazinomethyl.

Lower alkylsulfamoyl-lower alkyl is, for example, N—C₁-C₇ alkylsulfamoyl-C₁-C₄ alkyl, such as N-methyl-, N-ethyl-, N-propyl- or N-butyl-sulfamoyl-C₁-C₄ alkyl, such as N-methyl-, N-ethyl-, N-propyl- or N-butyl-sulfamoylmethyl, 2-(N-methylsulfamoyl) ethyl, 2-(N-butylsulfamoyl)ethyl, 3-(N-methylsulfamoyl)propyl, 3-(N-butylsulfamoyl)propyl, or 4-(N-methylsulfamoyl)butyl, 4-(N-butylsulfamoyl)butyl or 4-(N,N-dimethylsulfamoyl)butyl, especially N-methyl-, N-butyl- or N,N-dimethyl-sulfamoylmethyl.

Lower alkylthio-lower alkoxy is, for example, N—C₁-C₄ alkylthio-C₁-C₄ alkoxy, such as methylthio-C₁-C₄ alkoxy, e.g. methylthiomethoxy, 2-methylthioethyoxy or 3-methylthiopropyloxy.

Oxadiazolyl-lower alkyl is, for example, 1,2,4-oxadiazol-5-yl-C₁-C₄ alkyl, such as 1,2,4-oxadiazol-5-ylmethyl.

Oxo-oxazolinyl-lower alkyl is, for example, oxo-oxazolinyl-C₁-C₄ alkyl, such as 5-oxo-oxazolin-3-ylmethyl.

Oxopiperidinyl-lower alkyl is, for example, oxopiperidinyl-C₁-C₄ alkyl, such as 2-oxopiperidin-1-ylmethyl or 2-oxopiperidin-4-ylmethyl.

Oxopyrrolidinyl-lower alkyl is, for example, oxopyrrolidinyl-C₁-C₄ alkyl, such as 2-oxopyrrolidin-1-ylmethy 2-oxo-pyrrolidin-4-ylmethyl or 2-oxo-pyrrolidin-5-ylmethyl.

Oxothiazolyl-lower alkyl is, for example, oxothiazolyl-C₁-C₄ alkyl, such as 2-oxothiazol-4-ylmethyl or 2-oxothiazol-5-ylmethyl.

S-Oxothiomorpholinocarbonyl-lower alkoxy is, for example, S-oxothiomorpholinocarbonyl-C₁-C₄ alkoxy, such as S-oxothiomorpholinocarbonylmethoxy, 2-(S-oxo)thiomorpholinocarbonylethoxy, 3-(S-oxo)thiomorpholinocarbonylpropyloxy or 1- or 2-[4-(S-oxo)thiomorpholinocarbonyl]butyloxy.

S-Oxothiomorpholino-lower alkyl is, for example, S-oxothiomorpholino-C₁-C₄ alkyl, such as S-oxothiomorpholinomethyl, 2-(S-oxo)thiomorpholinoethyl, 3-(S-oxo)thiomorpholinopropyl or 1- or 2-[4-(S-oxo)thiomorpholino]butyl.

Phenyl-lower alkoxy is, for example, phenyl-C₁-C₄ alkoxy, such as benzyloxy, 2-phenylethoxy, 3-phenylpropyloxy or 4-phenylbutyloxy.

Phenyl-lower alkyl is, for example, C₁-C₄ alkylmorpholino-C₁-C₄ alkyl, such as methylmorpholinomethyl, 2-methylmorpholinocarbonylethyl, 3-methylmorpholinopropyl or 4-methylmorpholinobutyl.

piperazinocarbonyl-lower alkoxy is, for example, piperazinocarbonyl-C₁-C₄ alkoxy, such as piperazinocarbonylmethoxy, 2-piperazinocarbonylethoxy, 3-piperazinocarbonylpropyloxy or 4-piperazinocarbonylbutyloxy.

piperazinocarbonyl-lower alkyl is, for example, piperazinocarbonyl-C₁-C₄ alkyl, such as piperazinocarbonylmethyl, 2-piperazinocarbonylethyl, 3-piperazinocarbonylpropyl or 4-piperazinocarbonylbutyl, especially piperazinocarbonylmethyl.

Piperidinocarbonyl-lower alkoxy is, for example, piperidinocarbonyl-C₁-C₄ alkoxy, such as piperidinocarbonylmethoxy, 2-piperidinocarbonylethoxy, 3-piperidinocarbonylpropyloxy or 4-piperidinocarbonylbutyloxy.

Piperidinocarbonyl-lower alkyl is, for example, piperidinocarbonyl-C₁-C₄ alkyl, such as piperidinocarbonylmethyl, 2-piperidinocarabonylethyl, 3-piperidinocarbonylpropyl or 4-piperidinocarbonylbutyl.

Piperidino-lower alkoxy is, for example, piperidino-C₁-C₄ alkoxy, such as 2-piperidinoethoxy, 3-piperidinopropyloxy or 4-piperidinobutyloxy, especially 2-piperidinoethoxy.

Piperidino-lower alkyl is, for example, piperidino-C₁-C₄ alkyl, such as piperidinomethyl, 2-piperidinoethyl, 3-piperidinopropyl or 4-piperidinobutyl, especially piperidinomethyl.

Polyhalo-lower alkoxy is, for example, di-, tri- or tetra-halo-C₁-C₄ alkoxy, such as trifluoromethoxy.

Pyridyl-lower alkoxy is, for example, pyridyl-C₁-C₄ alkoxy, such as pyridylmethoxy, 2-pyridylethoxy, 3-pyridylpropyloxy or 4-pyridylbutyloxy.

Pyridyl-lower alkyl is, for example, pyridyl-C₁-C₄ alkyl, such as pyridylmethyl, 2-pyridylethyl, 3-pyridylpropyl or 4-pyridylbutyl, especially pyridylmethyl.

Pyrrolidinocarbonyl-lower alkoxy is, for example, pyrrolidinocarbonyl-C₁-C₄ alkoxy, such as pyrrolidinocarbonylmethoxy, 2-pyrrolidinocarbonylethoxy, 3-pyrrolidinocarbonylpropyloxy or 4-pyrrolidinocarbonylbutyloxy.

Pyrrolidinocarbonyl-lower alkyl is, for example, pyrrolidinocarbonyl-C₁-C₄ alkyl, such as pyrrolidinocarbonylmethyl, 2-pyrrolidinocarbonylethyl, 3-pyrrolidinocarbonylpropyl or 4-pyrrolidinocarbonylbutyl.

Pyrrolidino-lower alkyl is, for example, pyrrolidino-C₁-C₄ alkyl, such as pyrrolidinomethyl, 2-pyrrolidinoethyl, 3-pyrrolidinopropyl or 4-pyrrolidinobutyl, especially pyrrolidinomethyl.

Pyrrolidinyl-lower alkyl is, for example, pyrrolidinyl-C₁-C₄ alkyl, such as pyrrolidin-2-ylmethyl, pyrrolidin-3-ylmethyl, 2-pyrrolidin-2-ylethyl, 2-pyrrolidin-3-ylethyl, 3-pyrrolidin-2-ylpropyl or 4-pyrrolidin-2-ylbutyl.

Sulfamoyl-lower alkyl is, for example, sulfamoyl-C₁-C₄ alkyl, such as sulfamoyl-C₁-C₄ alkyl, such as sulfamoylmethyl, 2-sulfamoylethyl, 3-sulfamoylpropyl or 4-sulfamoylbutyl.

Tetrazolyl-lower alkoxy is, for example, tetrazolyl-C₁-C₄ alkoxy, such as tetrazol-5-ylmethoxy, 2-(tetrazol-5-yl)ethoxy, 3-(tetrazol-5-yl)propyloxy or 4-(tetrazol-4-yl)butyloxy, especially tetrazol-5-yl methoxy.

Thiocarbamoyl-lower alkyl is, for example, thiocarbamoyl-C₁-C₄ alkyl, such as thiocarbamoylmethyl, 2-thiocarbamoylethyl, 3-thiocarbamoylpropyl or 4-thiocarbamoylbutyl.

Thiomorpholinocarbonyl-lower alkyl is, for example, thiomorpholinocarbonyl-C₁-C₄ alkyl, such as thiomorpholinocarbonylmethyl, 2-thiomorpholinocarbonylethyl, 3-thiomorpholinocarbonylpropyl or 1- or 2-(4-thiomorpholinocarbonyl)butyl.

Thiomorpholino-lower alkoxy is, for example, thiomorpholino-C₁-C₁-C₄ alkoxy, such as thiomorpholinomethoxy, 2-thiomorpholinomethoxy, 3-thiomorpholinopropyloxy or 1- or 2-(4-thiomorpholino)butyloxy.

Thiomorpholino-lower alkyl is, for example, thiomorpholino-C₁-C₄ alkyl, such as thiomorpholinomethyl, 2-thiomorpholinoethyl, 3-thiomorpholinopropyl or 1- or 2-(4-thiomorpholino)butyl, especially 2-thiomorpholinoethyl.

Depending on whether asymmetric carbon atoms are present, the compounds of the invention can be present as mixtures of isomers, especially as racemates, or in the form of pure isomers, especially optical antipodes.

Salts of compounds having salt-forming groups are especially acid addition salts, salts with bases or, where several salt-forming groups are present, can also be mixed salts or internal salts.

Salts are especially the pharmaceutically acceptable or non-toxic salts of compounds of formula I.

Such salts are formed, for example, by compounds of formula I having an acid group, for example a carboxy group or a sulfo group, and are, for example, salts thereof with suitable bases, such as non-toxic metal salts derived from metals of groups Ia, Ib, IIa and IIb of the Periodic Table of the Elements, for example alkali metal salts, especially lithium, sodium or potassium salts, or alkaline earth metal salts, for example magnesium or calcium salts, also zinc salts or ammonium salts, as well as salts formed with organic amines, such as unsubstituted or hydroxy-substituted mono-, di- or tri-alkylamines, especially mono-, di- or tri-lower alkylamines, or with quaternary ammonium bases, for example with methyl-, ethyl-diethyl- or triethyl-amine, mono-, bis- or tris-(2-hydroxy-lower alkyl)-amines, such as ethanol-, diethanol- or triethanol-amine, triS(hydroxymethyl)methylamine or 2-hydroxy-tertbutylamine, N,N-di-lower alkyl-N-(hydroxy-lower alkyl)-amines, such as N,N-dimethyl-N-(2-hydroxyethyl)-amine, or N-methyl-D-glucamine, or quaternary ammonium hydroxides, such as tetrabutylammonium hydroxide. The compounds of formula I having a basic group, for example an amino group, can form acid addition salts, for example with suitable inorganic acids, for example hydrohalic acids, such as hydrochloric acid or hydrobromic acid, or sulfuric acid with replacement of one or both protons, phosphoric acid with replacement of one or more protons, e.g. orthophosphoric acid or metaphosphoric acid, or pyrophosphoric acid with replacement of one or more protons, or with organic carboxylic, sulfonic, sulfo or phosphonic acids or N-substituted sulfamic acids, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, as well as with amino acids, such as the .alpha.-amino acids mentioned hereinbefore, and with methanesulfonic acid, ethanesulfonic acid, 2hydroxyethanesulfonic acid, ethane-1,2-disulfonic acid, benzenesulfonic acid, 4-methylbenzenenesulfonic acid, naphthalene-2-sulfonic acid, 2- or 3-phosphoglycerate, glucose-6-phosphate, or N-cyclohexylsulfamic acid (forming cyclamates) or with other acidic organic compounds, such as ascorbic acid. Compounds of formula I having acid and basic groups can also form internal salts.

For isolation and purification purposes it is also possible to use pharmaceutically unacceptable salts.

The groups of compounds mentioned below are not to be regarded as exclusive; rather, as appropriate, for example in order to replace general definitions with more specific definitions, parts of those groups of compounds can be interchanged or exchanged for the definitions given above, or omitted.

The invention relates especially to methods comprising compounds of formula I

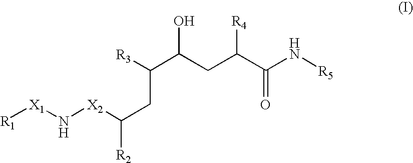

wherein

R₁ is a 2-R_{A}-3-R_{B}-phenyl radical, a 2-R_{A}-4R_{C}-phenyl radical, a 2-R_{A}-pyridin-3-yl radical, a 3-R_{A}-pyridin-2-yl radical or a 1-R_{D}-indol-3-yl radical, wherein one of the radicals R_{A} and R_{B} is lower alkyl, hydroxy-lower alkyl, lower alkanoyloxy-lower alkyl, lower alkoxy-lower alkyl, lower alkoxy-lower alkoxy-lower alkyl; an amino-lower alkyl or amino-lower alkoxy radical that is unsubstituted or N-lower alkanoylated or N-mono- or N,N-di-lower alkylated or N,N-disubstituted by lower alkylene, hydroxy-, lower alkoxy- or lower alkoxy-lower alkoxy-lower alkylene, by unsubstituted or N′-lower alkanoylated, lower alkoxy-carbonyl- or lower alkoxy-lower alkyl-N′-substituted or N′-lower alkylated aza-lower alkylene, by oxa-lower alkylene or by optionally S-oxidised thia-lower alkylene; hydroxy, lower alkoxy, hydroxy-lower alkoxy, lower alkanoyloxy-lower alkoxy, lower alkoxy-lower alkoxy, lower alkoxy-lower alkoxy-lower alkoxy, polyhalo-lower alkoxy, cyano-lower alkoxy, unsubstituted or substituted phenyl- or pyridyl-lower alkoxy, lower alkoxy-lower alkenyloxy, optionally S-oxidised lower alkylthio-lower alkoxy, or amino-lower alkoxy that is unsubstituted or N-lower alkanoylated or N-mono- or N,N-di-lower alkylated or N,N-disubstituted by lower alkylene, hydroxy-, lower alkoxy- or lower alkoxy-lower alkoxy-lower alkylene, by unsubstituted or N′-lower alkanoylated, lower alkoxycarbonyl- or lower alkoxy-lower alkyl-N′-substituted or N′-lower alkylated aza-lower alkylene, by oxa-lower alkylene or by optionally S-oxidised thia-lower alkylene; and the other is hydrogen, lower alkyl, carbamoyl, hydroxy, lower alkoxy or polyhalo-lower alkoxy,

R_{C} is hydrogen, lower alkyl, hydroxy, lower alkoxy, hydroxy-lower alkoxy, lower alkoxy-lower alkoxy, morpholino-lower alkylcarbamoyl-lower alkoxy, lower alkoxy-lower alkoxy-lower alkyl; an amino, amino-lower alkyl or amino-lower alkoxy group that is unsubstituted or N-lower alkanoylated or N-mono- or N,N-di-lower alkylated or N,N-disubstituted by lower alkylene, hydroxy-, lower alkoxy-, lower alkoxycarbonyl- or lower alkoxy-lower alkoxy-lower alkylene, by unsubstituted or N′-lower alkanoylated, lower alkoxycarbonyl- or lower alkoxy-lower alkyl-N′-substituted or N′-lower alkylated aza-lower alkylene, by oxa-lower alkylene or by optionally S-oxidised thia-lower alkylene; or a free or amidated carboxy- or carboxy-lower alkoxy group or tetrazolyl-lower alkoxy, and

R_{D} is lower alkyl, hydroxy-lower alkyl, lower alkoxy-lower alkyl, lower alkoxy-lower alkoxy-lower alkyl, hydroxy-lower alkoxy-lower alkyl, a free or amidated carboxy or carboxy-lower alkyl group, cyano-lower alkyl, or an unsubstituted or substituted phenyl- or pyridyl-lower alkyl group,

one of the radicals X₁ and X₂ is carbonyl and the other is methylene,

R₂ is lower alkyl,

R₃ is unsubstituted or N-lower alkanoylated or N-mono- or N,N-di-lower alkylated amino,

R₄ is lower alkyl or phenyl-lower alkyl, and

R₅ is lower alkyl, cycloalkyl-lower alkyl, hydroxy-lower alkyl, lower alkoxy-lower alkyl, lower alkanoyloxy-lower alkyl; amino-lower alkyl that is unsubstituted or N-lower alkanoylated or N-mono- or N,N-di-lower alkylated or N,N-disubstituted by lower alkylene, hydroxy-, lower alkoxy-, lower alkoxy-lower alkyl- or lower alkanoyloxy-lower alkylene, by unsubstituted or N′-lower alkanoylated, lower alkoxycarbonyl- or lower alkoxy-lower alkyl-N′-substituted or N′-lower alkylated aza-lower alkylene, by oxa-lower alkylene or by optionally S-oxidised thia-lower alkylene; free or esterified or amidated carboxy-lower alkyl, cyano-lower alkyl, free or esterified or amidated dicarboxy-lower alkyl, free or esterified or amidated carboxy(hydroxy)-lower alkyl, free or esterified or amidated carboxycycloalkyl-lower alkyl, lower alkanesulfonyl-lower alkyl, unsubstituted or N-mono- or N,N-di-lower alkylated thio-lower carbamoyl-lower alkyl, unsubstituted or N-mono- or N,N-di-lower alkylated sulfamoyl-lower alkyl or an optionally hydrogenated and/or oxo-substituted heteroaryl radical or lower alkyl substituted by an optionally hydrogenated and/or oxo-substituted heteroaryl radical that is bonded via a carbon atom,

and to the salts thereof.

The invention relates especially to methods comprising compounds of formula I

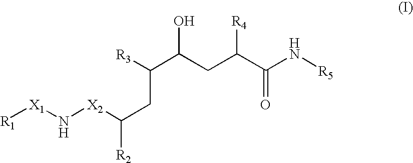

wherein

R₁ is a 2-R_{A}-3-R_{B}-phenyl radical, a 2-R_{A}-4-R_{C}-phenyl radical, a 2-R_{A}-pyridin-3-yl radical, a 3-R_{A}-pyridin-2-yl radical or a 1-R_{D}-indol-3-yl radical, wherein one of the radicals R_{A} a R_{B} is lower alkyl, hydroxy-lower alkyl, lower alkanoyloxy-lower alkyl, lower alkoxy-lower alkyl, lower alkoxy-lower alkoxy-lower alkyl, amino-lower alkyl, lower alkanoylamino-lower alkyl, lower alkylamino-lower alkyl, di-lower alkylamino-lower alkyl; piperidino- or pyrrolidino-lower alkyl that is unsubstituted or substituted by hydroxy, lower alkoxy or by lower alkoxy-lower alkyl; piperazino-lower alkyl that is unsubstituted or N′-lower alkylated, N′-lower alkanoylated or N′-substituted by lower alkoxycarbonyl or by lower alkoxy-lower alkyl; unsubstituted or lower alkylated morpholino-lower alkyl, optionally S-oxidised thiomorpholino-lower alkyl, amino-lower alkoxy, lower alkanoylamino-lower alkoxy, lower alkylamino-lower alkoxy, di-lower alkylamino-lower alkoxy; piperidino- or pyrrolidino-lower alkoxy that is unsubstituted or substituted by hydroxy, lower alkoxy or by lower alkoxy-lower alkyl; piperazino-lower alkoxy that is unsubstituted or N′-lower alkylated, N′-lower alkanoylated or N′-substituted by lower alkoxycarbonyl or by lower alkoxy-lower alkyl; unsubstituted or lower alkylated morpholino-lower alkoxy, optionally S-oxidised thiomorpholino-lower alkoxy, hydroxy, lower alkoxy, hydroxy-lower alkoxy, lower alkanoyloxy-lower alkoxy, lower alkoxy-lower alkoxy, lower alkoxy-lower alkoxy-lower alkoxy, polyhalo-lower alkoxy, cyano-lower alkoxy; phenyl- or pyridyl-lower alkoxy that is unsubstituted or substituted by lower alkyl, lower alkoxy, hydroxy, nitro, amino, lower alkylamino, di-lower alkylamino, halogen and/or by trifluoromethyl; lower alkoxy-lower alkenyloxy, lower alkylthio-lower alkoxy, lower alkanoylamino-lower alkoxy, lower alkanesulfonyl-lower alkoxy, amino-lower alkoxy, lower alkanoylamino-lower alkoxy, lower alkylamino-lower alkoxy, di-lower alkylamino-lower alkoxy; piperidino- or pyrrolidino-lower alkoxy that is unsubstituted or substituted by hydroxy, lower alkoxy or by lower alkoxy-lower alkyl; piperazino-lower alkoxy that is unsubstituted or N′-lower alkylated, N′-lower alkanoylated or N′-substituted by lower alkoxycarbonyl or by lower alkoxy-lower alkyl; unsubstituted or lower alkylated morpholino-lower alkoxy or optionally S-oxidised thiomorpholino-lower alkoxy, and the other is hydrogen, carbamoyl, hydroxy, lower alkoxy or polyhalo-lower alkoxy,

R_{C} is hydrogen, lower alkyl, lower alkoxy-lower alkoxy-lower alkyl, amino-lower alkyl, lower alkanoylamino-lower alkyl, lower alkylamino-lower alkyl, di-lower alkylamino-lower alkyl; piperidino- or pyrrolidino-lower alkyl that is unsubstituted or substituted by hydroxy, lower alkoxy or by lower alkoxy-lower alkyl; piperazino-lower alkyl that is unsubstituted or N′-lower alkylated, N′-lower alkanoylated or N′-substituted by lower alkoxycarbonyl or by lower alkoxy-lower alkyl; unsubstituted or lower alkylated morpholino-lower alkyl, optionally S-oxidised thiomorpholino-lower alkyl, di-lower alkylamino; a piperidino or pyrrolidino group that is unsubstituted or substituted by hydroxy, lower alkoxy or by lower alkoxy-lower alkyl; piperazino that is unsubstituted or N′-lower alkylated, N′-lower alkanoylated or N′-substituted by lower alkoxycarbonyl or by lower alkoxy-lower alkyl; unsubstituted or lower alkylated morpholino, optionally S-oxidised thiomorpholino, hydroxy, lower alkoxy, hydroxy-lower alkoxy, lower alkoxy-lower alkoxy, morpholino-lower alkylcarbamoyl-lower alkoxy, amino-lower alkoxy, lower alkanoylamino-lower alkoxy, lower alkylamino-lower alkoxy, di-lower alkylamino-lower alkoxy; piperidino- or pyrrolidino-lower alkoxy that is unsubstituted or substituted by hydroxy, lower alkoxy or by lower alkoxy-lower alkyl; piperazino-lower alkoxy that is unsubstituted or N′-lower alkylated, N′-lower alkanoylated or N′-substituted by lower alkoxycarbonyl or by lower alkoxy-lower alkyl; unsubstituted or lower alkylated morpholino-lower alkoxy, optionally S-oxidised thiomorpholino-lower alkoxy, carboxy-lower alkoxy, carbamoyl-lower alkoxy, lower alkylcarbamoyl-lower alkoxy, di-lower alkylcarbamoyl-lower alkoxy; piperidino- or pyrrolidino-carbonyl-lower alkoxy that is unsubstituted or substituted by hydroxy, lower alkoxy or by lower alkoxy-lower alkyl; piperazinocarbonyl-lower alkoxy that is unsubstituted or N′-lower alkylated, N′-lower alkanoylated or N′-substituted by lower alkoxycarbonyl or by lower alkoxy-lower alkyl; unsubstituted or lower alkylated morpholinocarbonyl-lower alkoxy, optionally S-oxidised thiomorpholinocarbonyl-lower alkoxy, tetrazolyl-lower alkoxy, carboxy, carbamoyl, lower alkylcarbamoyl or di-lower alkylcarbamoyl, and RD is lower alkyl, hydroxy-lower alkyl, lower alkoxy-lower alkyl, lower alkoxy-lower alkoxy-lower alkyl, hydroxy-lower alkoxy-lower alkyl, carboxy-lower alkyl, lower alkoxycarbonyl-lower alkyl, carbamoyl-lower alkyl, lower alkylcarbamoyl-lower alkyl, di-lower alkylcarbamoyl-lower alkyl; piperidino- or pyrrolidino-carbonyl-lower alkyl that is unsubstituted or substituted by hydroxy, lower alkoxy or by lower alkoxy-lower alkyl; piperazinocarbonyl-lower alkyl that is unsubstituted or N′-lower alkylated, N′-lower alkanoylated or N′-substituted by lower alkoxycarbonyl or by lower alkoxy-lower alkyl; unsubstituted or lower alkylated morpholinocarbonyl-lower alkyl, optionally S-oxidised thiomorpholinocarbonyl-lower alkyl, carboxy-lower alkyl, lower alkoxycarbonyl-lower alkyl or a phenyl- or pyridyl-lower alkyl group that is unsubstituted or substituted by lower alkyl, lower alkoxy, hydroxy, nitro, amino, lower alkylamino, di-lower alkylamino, halogen and/or by trifluoromethyl,

one of the radicals X₁ and X₂ is carbonyl and the other is methylene,

R₂ is lower alkyl,

R₃ is amino, lower alkanoylamino, lower alkylamino or di-lower alkylamino,

R₄ is lower alkyl or phenyl-lower alkyl and

R₅ is lower alkyl, cycloalkyl-lower alkyl, hydroxy-lower alkyl, lower alkoxy-lower alkyl, lower alkanoyloxy-lower alkyl; piperidino- or pyrrolidino-carbonyl-lower alkyl that is unsubstituted or substituted by hydroxy, lower alkoxy or by lower alkoxy-lower alkyl; piperazinocarbonyl-lower alkyl that is unsubstituted or N′-lower alkylated, N′-lower alkanoylated or N′-substituted by lower alkoxycarbonyl or by lower amorpholinocarbonyl-lower alkylated or lower alkylated morpholinocarbonyl-lower alkyl, optionally S-oxidised thiomorpholinocarbonyl-lower alkyl, carboxy-lower alkyl, lower alkoxycarbonyl-lower alkyl, carbamoyl-lower alkyl, lower alkylcarbamoyl-lower alkyl, di-lower alkylcarbamoyl-lower alkyl; piperidino- or pyrrolidino-carbonyl-lower alkyl that is unsubstituted or substituted by hydroxy, lower alkoxy or by lower alkoxy-lower alkyl; piperazinocarbonyl-lower alkyl that is unsubstituted or N′-lower alkylated, N′-lower alkanoylated or N′-substituted by lower alkoxycarbonyl or by lower alkoxy-lower alkyl; unsubstituted or lower alkylated morpholinocarbonyl-lower alkyl, optionally S-oxidised thiomorpholinocarbonyl-lower alkyl, cyano-lower alkyl, dicarboxy-lower alkyl, lower alkoxycarbonyl(carboxy)-lower alkyl, di-lower alkoxycarbonyl-lower alkyl, dicarbamoyl-lower alkyl, carbamoyl(carboxy)-lower alkyl, di-(lower alkylcarbamoyl)-lower alkyl, di-(di-lower alkylcarbamoyl)-lower alkyl, carboxy(hydroxy)-lower alkyl, lower alkoxycarbonyl(hydroxy)-lower alkyl, carbamoyl(hydroxy)-lower alkyl, lower alkylcarbamoyl(hydroxy)-lower alkyl or di-lower alkylcarbamoyl(hydroxy)-lower alkyl, carboxycycloalkyl-lower alkyl, lower alkoxycarbonylcycloalkyl-lower alkyl, carbamoylcycloalkyl-lower alkyl, lower alkylcarbamoylcycloalkyl-lower alkyl, di-lower alkylcarbamoylcycloalkyl-lower alkyl, lower alkanesulfonyl-lower alkyl, thiocarbamoyl-lower alkyl, N-lower alkylthiocarbamoyl-low alkyl, N-lower alkylthiocarbamoyl-lower alkyl or N,N-di-lower alkylthiocarbamoyl-lower alkyl, sulfamoyl-lower alkyl, lower alkylsulfamoyl-lower alkyl or di-lower alkylsulfamoyl-lower alkyl, unsubstituted or oxo-substituted pyrrolidinyl, imidazolyl, benzimidazolyl, oxadiazolyl, pyridyl, oxopiperidinyl, dioxopiperidinyl, oxothiazolyl, oxo-oxazolinyl or quinolinyl, unsubstituted or oxo-substituted pyrrolidinyl-lower alkyl, imidazolyl-lower alkyl, benzimidazolyl-lower alkyl, oxadiazolyl-lower alkyl, pyridyl-lower alkyl, oxopiperidinyl-lower alkyl, dioxopiperidinyl-lower alkyl, oxothiazolyl-lower alkyl, oxo-oxazolinyl-lower alkyl or quinolinyl-lower alkyl, morpholinocarbonyl-lower alkyl or unsubstituted or N-lower alkanoylated piperidyl-lower alkyl or unsubstituted or N-lower alkanoylated piperidyl, and the salts thereof.

The invention relates especially to methods comprising compounds of formula I wherein

R₁ is a 2-R_{A}-3-R_{B}-phenyl radical, a 2-R_{A}-4-R_{C}-phenyl radical, a 2-R_{A}-pyridin-3-yl radical, a 3-R_{A}-pyridin-2-yl radical or a 1-R_{D}-indol-3-yl radical,

wherein one of the radicals R_{A} and R_{B} is C₁-C₄ alkyl, hydroxy-C₁-C₄ alkyl, C₁-C₄ alkanoyloxy-C₁-C₄ alkyl, C₁-C₄ alkoxy-C₁-C₄ alkyl, C₁-C₄ alkoxy-C₁-C₄ alkoxy-C₁-C₄ alkyl, amino-C₁-C₄ alkyl, C₁-C₄ alkanoylamino-C₁-C₄ alkyl, C₁-C₄ alkylamino-C₁-C₄ alkyl, di-C₁-C₄ alkylamino-C₁-C₄ alkyl, piperidino-C₁-C₄-alkyl, hydroxypiperidino-C₁-C₄ alkyl, C₁-C₄ alkoxypiperidino-C₁-C₄ alkyl, C₁-C₄ alkoxy-C₁-C₄-alkoxypiperidino-C₁-C₄ alkyl, C₁-C₄ alkoxycarbonylpiperidino-C₁-C₄ alkyl, pyrrolidino-C₁-C₄ alkyl, hydroxypyrrolidino-C₁-C₄ alkyl, C₁-C₄ alkoxypyrrolidino-C₁-C₄ alkyl, C₁-C₄-alkoxy-C₁-C₄ alkoxypyrrolidino-C₁-C₄ alkyl, piperazino-C₁-C₄ alkyl, N′-C₁-C₄ alkylpiperazino-C₁-C₄ alkyl, N′-C₁-C₄ alkanoylpiperazino-C₁-C₄ alkyl, N′-C₁-C₄ alkoxycarbonylpiperazino-C₁-C₄ alkyl, N′-C₁-C₄ alkoxy-C₁-C₄ alkylpiperazino-C₁-C₄ alkyl, morpholino-C₁-C₄ alkyl, C₁-C₄ alkylmorpholino-C₁-C₄ alkyl, thiomorpholino-C₁-C₄ alkyl, S-oxythiomorpholino-C₁-C₄ alkyl, S, S-dioxythiomorpholino-C₁-C₄ alkyl, C₁-C₇ alkoxy, such as propyloxy, amino-C₁-C₇ alkoxy, C₁-C₄ alkanoylamino-C₁-C₄ alkoxy, C₁-C₄ alkylamino-C₁-C₄ alkoxy, di-C₁-C₄ alkylamino-C₁-C₄ alkoxy, piperidino-C₁-C₄ alkoxy, hydroxypiperidino-C₁-C₄ alkoxy, C₁-C₄ alkoxypiperidino-C₁-C₄ alkoxy, C₁-C₄ alkoxy-C₁-C₄ alkoxypiperidino-C₁-C₄ alkoxy, pyrrolidino-C₁-C₄ alkoxy, hydroxypyrrolidino-C₁-C₁-C₄ alkoxy, C₁-C₄ alkoxypyrrolidino-C₁-C₄ alkoxy, C₁-C₄ alkoxy-C₁-C₄ alkoxypyrrolidino-C₁-C₄ alkoxy, piperazino-C₁-C₄ alkoxy, N′-C₁-C₄ alkylpiperazino-C₁-C₄ alkoxy, N′-C₁-C₄ alkanoylpiperazino-C₁-C₄-C₄ alkoxy, N′-C₁-C₄ alkoxycarbonylpiperazino-C₁-C₄ alkoxy, N′-C₁-C₄ alkoxy-C₁-C₄ alkylpiperazino-C₁-C₄ alkoxy, morpholino-C₁-C₄ alkoxy or C₁-C₄ alkylmorpholino-C₁-C₄ alkoxy, thiomorpholino-C₁-C₄ alkoxy, S-oxythiomorpholino-C₁-C₄ alkoxy, S, S-dioxythiomorpholino-C₁-C₄ alkoxy, hydroxy, hydroxy-C₁-C₄ alkoxy, C₁-C₄ alkanoyloxy-C₁-C₄ alkoxy, C₁-C₄ alkoxy-C₁-C₄ alkoxy, C₁-C₄ alkoxy-C₁-C₄ alkoxy-C₁-C₄ alkoxy, polyhalo-C₁-C₄ alkoxy, cyano-C₁-C₄ alkoxy, carbamoyl-C₁-C₄ alkoxy, such as 2-carbamoylethoxy; phenyl- or pyridyl-C₁-C,alkoxy that is unsubstituted or substituted by C₁-C₄ alkyl, C₁-C₄ C₄ alkoxy, hydroxy, nitro, amino, C₁-C₄ alkylamino, di-C₁-C₄ alkylamino, halogen and/or by trifluoromethyl; C₁-C₄ alkoxy-C₁-C₄ alkenyloxy, C₁-C₄ alkylthio-C₁-C₄ alkoxy, C₁-C₄ alkanesulfinyl-C₁-C₄ alkoxy, C₁-C₄ alkanesulfonyl-C₁-C₄ alkoxy, amino-C₁-C₇ alkoxy, C₁-C₄ alkanoylamino-C₁-C₄ alkoxy, C₁-C₄ alkylamino-C₁-C₄ alkoxy, di-C₁-C₄ alkylamino-C₁-C₄ alkoxy, piperidino-C₁-C₄ alkoxy, hydroxypiperidino-C₁-C₄ alkoxy, C₁-C₄ alkoxypiperidino-C₁-C₄ alkoxy, C₁-C₄ alkoxy-C₁-C₄ alkoxypiperidino-C₁-C₄ alkoxy, pyrrolidino-C₁-C₄ alkoxy, hydroxypyrrolidino-C₁-C₄ alkoxy, C₁-C₄ alkoxypyrrolidino-C₁-C₄ alkoxy, C₁-C₄ alkoxy-C₁₋₄ alkoxypyrrolidino-C₁-C₄ alkoxy, piperazino-C₁-C₄ alkoxy, N′-C₁-C₄ alkylpiperazino-C₁-C₄ alkoxy, N′-C₁-C₄ alkanoylpiperazino-C₁-C₄ alkoxy, N′-C₁-C₄ alkoxycarbonylpiperazino-C₁-C₄-alkoxy, N′-C₁-C₄ alkoxy-C₁-C₄ alkylpiperazino-C₁-C₄ alkoxy, morpholino-C₁-C₄ alkoxy or C₁-C₄ alkylmorpholino-C₁-C₄ alkoxy or thiomorpholino-C₁-C₄ alkoxy, and the other is hydrogen, carbamoyl, C₁-C₄ alkyl, hydroxy, C₁-C₄ alkoxy or trihalo-C₁-C₄ alkoxy,

R_{C} is hydrogen, hydroxy, di-C₂-C₄ alkylamino, piperidino, pyrrolidino, morpholino, thiomorpholino, S-oxythiomorpholino, S,S-dioxythiomorpholino, C₁-C₄ alkoxy, hydroxy-C₁-C₄ alkoxy, C₁-C₄ alkoxy-C₁-C₄ alkoxy, morpholino-C₁-C₄ alkylcarbamoyl-C₁-C₄ alkoxy, C₁-C₄ alkoxy-C₁-C₄ alkoxy-C₁-C₄ alkyl, amino-C₁-C₄ alkyl, C₁-C₄ alkyl, C₁-C₄ alkylamino-C₁-C₄ alkyl, di-C₁-C₄ alkylamino-C₁-C₄ alkyl; piperidino- or pyrrolidino-C₁-C₄ alkyl that is unsubstituted or substituted by hydroxy, C₁-C₄ alkoxy or by C₁-C₄ alkoxy-C₁-C₄ alkyl; amino-C₁-C₄ alkyl, C₁-C₄ alkanoylamino-C₁-C₄ alkyl, C₁-C₄ alkylamino-C₁-C₄ alkylamino-C₁-C₄ alkyl, di-C₁-C₄ alkylamino-C₁-C₄ alkyl, piperidino-C₃-C₄ alkyl, hydroxypiperidino-C₁-C₄ alkyl, C₁-C₄ alkoxypiperidino-C₁-C₄ alkyl, C₁-C₄ alkoxy-C₁-C₄ alkoxypiperidino-C₁-C₄ alkyl, C₁-C₄ alkoxycarbonylpiperidino-C₁-C₄ alkyl, pyrrolidino-C₁-C₄ alkyl, hydroxypyrrolidino-C₁-C₄ alkyl, C₁-C₄ alkoxypyrrolidino-C₁-C₄ alkyl, C₁-C₄ alkoxypyrrolidino-C₁-C₄ alkyl, piperazino-C₁-C₄ alkyl, N′-C₁-C₄ alkanoylpiperazino-C₁-C₄ alkyl, N′-C₁-C₄ alkanoylpiperazino-C₁-C₄ alkyl, N′-C₁-C₄ alkoxycarbonylpiperazino-C₁-C₄ alkyl, N′-C₁-C₄ alkoxy-C₁-C₄ alkylpiperazino-C₁-C₄ alkyl, morpholino-C₁-C₄ alkyl, C₁-C₄ alkylmorpholino-C₁-C₄ alkyl, thiomorpholino-C₁-C₄ alkyl, S-oxythiomorpholino-C₁-C₄ alkyl, S, S-dioxythiomorpholino-C₁-C₄ alkyl, amino-C₁-C₇ alkoxy, C₁-C₄ alkanoylamino-C₁-C₄ alkoxy, C₁-C₄ alkylamino-C₁-C₄ alkoxy, di-C₁-C₄ alkylamino-C₁-C₄ alkoxy, piperidino-C₁-C₄ alkoxy, hydroxypiperidino-C₁-C₄ alkoxy, C₁-C₄ alkoxypiperdino-C₁-C₄ alkoxy, C₁-C₄ alkoxy-C₁-C₄ alkoxypiperidino-C₁-C₄ alkoxy, pyrrolidino-C₁-C₄ alkoxy, hydroxypyrrolidino-C₁-C₄ alkoxy, C₁-C₄ alkoxypyrrolidino-C₁-C₄ alkoxy, C₁-C₄ alkoxy-C₁-C₄ alkoxypyrrolidino-C₁-C₄ alkoxy, piperazino-C₁-C₄ alkoxy, N′-C₁-C₄ alkylpiperazino-C₁-C₄ alkoxy, N′-C₁-C₄ alkanoylpiperazino-C₁-C₄ alkoxy, N′-C₁-C₄ alkoxycarbonylpiperazino-C₁-C₄ alkoxy, N′-C₁-C₄ alkoxy-C₁-C₄ alkylpiperazino-C₁-C₄ alkoxy, morpholino-C₁-C₄ alkoxy or C₁-C₄ alkylmorpholino-C₁-C₄ alkoxy, thiomorpholino-C₁-C₄ alkoxy, S-oxythiomorpholino-C₁-C₄ alkoxy, S,S-dioxythiomorpholino-C₁-C₄ alkoxy, carboxy-C₃-C₄ alkoxy, carbamoyl-C₁-C₄ alkoxy, C₁-C₄ alkylcarbamoyl-C₁-C₄ alkoxy, di-C₁-C₄ alkylcarbamoyl-C₁-C₄ alkoxy, di-C₁-C₄ alkoxy, such as 3-dimethylaminopropyloxy, piperidinocarbonyl-C₁-C₄ alkoxy, hydroxypiperidinocarbonyl-C₁-C₄ alkoxy, C₁-C₄ alkoxypiperidinocarbonyl-C₁-C₄ alkoxy, C₁-C₄ alkoxy-C₁-C₄ alkoxypiperidinocarbonyl-C₁-C₄ alkoxy, pyrrolidinocarbonyl-C₁-C₄ alkoxy, hydroxypyrrolidinocarbonyl-C₁-C₄ alkoxy, C₁-C₄ alkoxypyrrolidinocarbonyl-C₁-C₄ alkoxy, C₁-C₄ alkoxy-C₁-C₄ alkoxypyrrolidinocarbonyl-C₁-C₄ alkoxy, piperazinocarbonyl-C₁-C₄ alkoxy, N′-C₁-C₄ alkylpiperazinocarbonyl-C₁-C₄ alkoxy, N′-C₁-C₄ alkoxycarabonylpiperazinocarbonyl or N′-C₁-C₄ alkoxy-C₁-C₄ alkylpiperazinocarbonyl-C₁-C₄ alkoxy, morpholinocarbonyl-C₁-C₄ alkoxy, C₁-C₁ alkylmorpholinocarbonyl-C₁-C₁ alkoxy, thiomorpholinocarbonyl-C₁-C₄ alkoxy, S-oxythio-morpholinocarbonyl, S,S-dioxythiomorpholinocarbonyl-C₁-C₄ alkoxy, tetrazolyl-C₁-C₁ alkoxy, carboxy, carbamoyl or C₁-C₄ alkylcarbamoyl, such as methylcarbamoyl, and

R_{D} is C₁-C₄ alkyl, hydroxy-C₁-C₄ alkyl, C₁-C₄ alkoxy-C₁-C₄ alkyl, C₄ alkoxy-C₁-C₄ alkoxy-C₁-C₄-alkyl, hydroxy-C₁-C₄ alkoxy-C₁-C₄ alkyl, carboxy-C₁-C₄ alkyl, C₁-C₄ alkoxycarbonyl-C₁-C₄ alkyl, carbamoyl-C₁-C₄ alkyl, C₁-C₄ alkylcarbamoyl-C₁-C₄ alkyl, di-C₁-C₄ alkylcarbamoyl-C₁-C₄ alkyl, piperidino-C₁-C₄ alkyl, hydroxypiperidino-C₁-C₄ alkyl, C₁-C₄ alkoxypiperidino-C₁-C₄ alkyl, C₁-C₄ alkoxy-C₁-C₄ alkoxypiperidino-C₁-C₄ alkyl, C₁-C₄ alkoxycarbonylpiperidino-C₁-C₄ alkyl, pyrrolidino-C₁-C₄ alkyl, hydroxypyrrolidino-C₁-C₄ alkyl, C₁-C₄ alkoxypyrrolidino-C₁-C₄ alkyl, C₁-C₄ alkoxy-C₁-C₄ alkoxypyrrolidino-C₁-C₄ alkyl, piperazino-C₁-C₄ alkyl, N′-C₁-C₄ alkylpiperazino-C₁-C₄ alkyl, N′-C₁-C₄ alkanoylpiperazino-C₁-C₄ alkyl, N′-C₁-C₄ alkoxycarbonylpiperazino-C₁-C₄ alkyl, N′—C₁-C₄ alkoxy-C₁-C₄ alkylpiperzino-C₁-C₄ alkyl, morpholino-C₁-C₄ alkyl, C₁-C₄ alkylmorpholino-C₁-C₄ alkyl, thiomorpholino-C₁-C₄ alkyl, S-oxythiomorpholino-C₁-C₄ alkyl, S, S-dioxythiomorpholino-C₁-C₄ alkyl, carboxy-C₁-C₄ alkyl, C₁-C₄ alkoxycarbonyl-C₁-C₄ alkyl, or is phenyl-C₁-C₄ alkyl or pyridyl-C₁-C₄ alkyl that is unsubstituted or substituted by C₁-C₄ alkyl, C₁-C₄ alkoxy, hydroxy, nitro, amino, C₁-C₄ alkylamino, di-C₁-C₄ alkylamino, halogen and/or by trifluoromethyl,

one of the radicals X₁ and X₂ is carbonyl and the other is methylene,

R₂ is C₁-C₄ alkyl,

R.sub.₃ is amino, C₁-C₄ alkanoylamino, C₁-C₄ alkylamino or di-C₁-C₄ alkylamino,

R₄ is C₁-C₄ alkyl or phenyl-C₁-C₄ alkyl, and

R₅ is C₁-C₄ alkyl, cycloalkyl-C₁-C₄ alkyl, hydroxy-C₁-C₄ alkyl, C₁-C₄ alkoxy-C₁-C₄ alkyl, C₁-C₄ alkanoyloxy-C₁-C₄ alkyl, piperidino-C₁-C₄ alkyl, hydroxypiperidino-C₁-C₄ alkyl, C₄ alkoxypiperidino-C₁-C₄ alkyl, C₁-C₄ alkoxy-C₁-C₄ alkoxypiperidino-C₁-C₄ alkyl, C₁-C₄ alkoxycarbonylpiperidino-C₁-C₄ alkyl, pyrrolidino-C₁-C₄ alkyl, hydroxypyrrolidino-C₁-C₄ alkyl, C₁-C₄ alkoxypyrrolidino-C₁-C₄ alkyl, C₁-C₄ alkoxy-C₁-C₄ alkoxypyrrolidino-C₁-C₄ alkyl, piperazino-C₁-C₄ alkyl, N′-C₁-C₄ alkylpiperazino-C₁-C₄ alkyl, N′-C₁-C₄ alkanoylpiperazino-C₁-C₄ alkyl, N₁—C₁-C₄ alkoxycarbonylpiperazino-C₁-C₄ alkyl, N′-C₁-C₄ alkoxy-C₁-C₄ alkylpiperazino-C₁-C₄ alkyl, morpholino-C₁-C₄ alkyl, C₁-C₄ alkylmorpholino-C₁-C₄ alkyl, thiomorpholino-C₁-C₁-alkyl, S-oxythiomorpholino-C₁-C₄ alkyl, S, S-dioxythiomorpholino-C₂-C₄ alkyl, carboxy-C₁-C₄ alkyl, C₁-C₄ alkoxycarbonyl-C₁-C₄ alkyl, carbamoyl-C₁-C₄ alkyl, C₁-C₄ alkylcarbamoyl-C₁-C₄ alkyl, di-C₁-C₄ alkylcarbamoyl-C₁-C₄ alkyl, piperidinocarbonyl-C₁-C₄ alkyl, hydroxypiperidinocarbonyl —C₁-C₄ alkyl, C₁-C₄ alkoxypiperidinocarbonyl-C₁-C₄ alkyl, C₁-C₄ alkyl, C₁-C₄ alkoxy-C₁-C₄ alkoxypiperidinocarbonyl-C₁-C₄ alkyl, pyrrolindinocarbonyl-C₁-C₄ alkyl, hydrolxypyrrolidinocarbonyl-C₁-C₄ alkyl, C₁-C₄ alkoxypyrrolidinocarbonyl-C₁-C₄ alkyl, C₁-C₄ alkoxy-C₁-C₄ alkoxypyrrolidinocarbonyl-C₁-C₄ alkyl, piperazinocarbonyl-C₁-C₄ alkyl, N′-C₁-C₄ alkylpiperazinocarbonyl-C₁-C₄ alkyl, N′-C₁-C₄ alkanoylpiperazinocarbonyl-C₁-C₄ alkyl, N′-C₁-C₄ alkoxycarbonylpiperazinocarbonyl, N′-C₁-C₄ alkoxy-C₁-C₄ alkylpiperazinocarbonyl-C₁-C₄ alkyl, morpholinocarbonyl-C₁-C₄ alkyl, C₁-C₄ alkylmorpholinocarbonyl-C₁-C₄ alkyl, thiomorpholinocarbonyl-C₁-C₄ alkyl, S-oxythiomorpholinocarbonyl-C₁-C₄ alkyl, S,S-dioxythiomorpholinocarbonyl-C₁-C₄ alkyl, carbamoyl-C₁-C₄ alkyl, C₁-C₄ alkylcarbamoyl-C₁-C₄ alkyl, di-C₁-C₄ alkylcarbamoyl-C₁-C₄ alkyl, cyano-C₁-C₄ alkyl, dicarboxy-C₁-C₄ alkyl, C₁-C₄ alkoxycarbonyl(carboxy)-C₁-C₄ alkyl, di-C₁-C₄ alkoxycarbonyl-C₁-C₄ alkyl, dicarbamoyl-C₁-C₄ alkyl, carbamoyl(carboxy) (carboxy)-C₁-C₄ alkyl, di-(C₁-C₄ alkylcarbamoyl)-C₁-C₄ alkyl, di-(di-C₁-C₄ alkylcarbamoyl)-C₁-C₄ alkyl, carbamoyl(hydroxy)-C₁-C₄ alkyl, C₁-C₄ alkylcarbamoyl(hydroxy)-C₁-C₄ alkyl or di-C₁-C₄ alkylcarbamoyl(hydroxy)-C₁-C₄ alkyl, carboxycycloalkyl-C₁-C₄ alkyl, C₁-C₄ alkoxycarbonylcycloalkyl-C₁-C₄ alkyl, carbamoylcycloalkyl-C₁-C₄ alkyl, C₁-C₄ alkylcarbamoylcycloalkyl-C₁-C₄ alkyl, di-C₁-C₄ alkylcarbamoylcycloalkyl-C₁-C₄ alkyl, amoylcycloalkyl-C₁-C₄ alkyl, C₁-C₄ alkanesulfonyl-C₁-C₄ alkyl, thiocarbamoyl-C₁-C₄ alkyl, N—C₁-C₄ alkylthiocarbamoyl-C₁-C₄ alkyl or N,N-di-C₁-C₄ alkylthiocarbamoyl-C₁-C₄ alkyl, sulfamoyl-C₁-C₄ alkyl, C₁-C₄ alkylsulfamoyl-C₁-C₄ alkyl or di-C₁-C₄ alkylsulfamoyl-C₁-C₄ alkyl, unsubstituted or oxo-substituted pyrrolidinyl, imidazolyl, benzimidazolyl, oxadiazolyl, pyridyl, oxopiperidinyl, dioxopiperidinyl, oxothiazolyl, oxo-oxazolinyl or quinolinyl, unsubstituted or oxo-substituted pyrrolidinyl-C₁-C₄ alkyl, imidazolyl-C₁-C₄ alkyl, benzimidazolyl-C₁-C₄ alkyl, oxadiazolyl-C₁-C₄ alkyl, pyridyl-C₁-C₄ alkyl, oxopiperidinyl-C₁-C₄ alkyl, dioxopiperidinyl-C₁-C₄ alkyl, oxothiazolyl-C₁-C₄ alkyl, oxo-oxazolinyl-C₁-C₄ alkyl or quinolinyl-C₁-C₄ alkyl, morpholinocarbonyl-C₁-C₄ alkyl or unsubstituted or N—C₁-C₄ alkanoylated piperidyl-C₁-C₄ alkyl or unsubstituted or N—C₁-C₄ alkanoylated piperidyl, and the salts thereof.

The invention relates especially to methods comprising compounds of formula I wherein

R₁ is a 2-R_{A}-3-R_{B}-phenyl radical, a 2-R_{A}-4-R_{C}-phenyl radical, a 2-R_{A}-pyridin-3-yl radical, a 3-R_{A}-pyridin-2-yl radical or a 1-R_{D}-indol-3-yl radical, wherein one of the radicals R_{A} and R_{B} is C₁-C₄ alkyl, C₁-C₄ alkoxy-C₁-C₄ alkyl, such as propyloxymethyl, di-C₁-C₄ alkylamino-C₁-C₄ alkyl, such as dimethylaminomethyl, piperidino-C₁-C₄ alkyl, such as piperidinomethyl, C₁-C₄ alkanoylpiperidinyl-C₁-C₄ alkyl, such as 2-methoxycarbonylpiperidin-4-yl)ethyl, pyrrolidino-C₁-C₄ alkyl, such as pyrrolidinomethyl, piperazino-C,-C₁-alkyl, N′-C₁-C₄ alkylpiperazino-C₁-C₄ alkyl, such as N′-methylpiperazinomethyl, N′-C₁-C₄ alkanoylpiperazino-C₁-C₄ alkyl, such as N′-acetylpiperazinomethyl, morpholino-C₁-C₄ alkyl, such as morpholinomethyl, 2-morpholinoethyl or 3-morpholinopropyl, C₁-C₄ alkylmorpholino-C₁-C₄ alkyl, thiomorpholino-C₁-C₄ alkyl, such as 2-thiomorpholinoethyl, amino-C₁-C₇ alkoxy, such as 2-aminoethoxy, 3-aminopropyloxy, C₁-C₄ alkanoylamino-C₁-C₄ alkoxy, such as 2-acetylaminoethoxy, di-C₁-C₄-alkylamino-C₁-C₄-alkoxy, such as 3-dimethylaminopropyloxy, piperidino-C₁-C₄ alkoxy, such as 2-piperidinoethoxy, morpholino-C₁-C₄ alkoxy, such as 2-morpholinoethoxy or 3-morpholinopropyloxy, hydroxy, C₁-C₇ alkoxy, such as propyloxy, C₁-C₄ alkoxy-C₁-C₄ alkoxy, such as 2-methoxyethoxy, 3-methoxypropyloxy, 4-methoxybutyloxy or 5-methoxypentyloxy, C₁-C₄ alkoxy-C₁-C₄ alkoxy, such as 2-(methoxymethoxy)ethoxy or 2-(2-methoxyethoxy)ethoxy, C₁ C₄ alkoxy-C₁-C₄ alkenyloxy, such as 4-methoxy-but-2-enyloxy, amino-C₂-C₁-C₇ alkoxy, such as 2-aminoethoxy or 3-aminopropyloxy, C₃-C₄ alkanoylamino-C₁-C₄ alkoxy, such as 2-acetylaminoethoxy, di-C₁-C₄ alkylamino-C₁-C₄ alkoxy, such as 3-dimethylaminopropyloxy, piperidino-C₁-C₄ alkoxy, such as 2-piperidinoethoxy, morpholino-C₁-C₄ alkoxy, such as 2-morpholinoethoxy or 3-morpholinopropyloxy, carbamoyl, carbamoyl-C₁-C₄ alkoxy, such as 2-carbamoylethoxy, and the other is hydrogen, C₁-C₄ alkyl, such as methyl, hydroxy, or C₁-C₄ alkoxy, such as methoxy.

R_{C} is hydrogen, hydroxy, C₁-C₄ alkoxy, such as methoxy, C₁-C₄ alkoxy, such as 2-methoxyethoxy, 3-methoxypropyloxy, 4-methoxybutyloxy or 5-metholxypentyloxy, morpholino-C₁-C₄ alkylcarbamoyl-C₁-C₄ alkoxy, such as 2-morpholinoethylcarbamoylmethoxy, di-C₁-C₄ alkylamino-C₁-C₄ alkyl, such as dimethylaminomethyl, piperidino-C₁-C₄ alkyl, such as piperidinomethyl, C₁-C₄ alkoxycarbonylpiperidino-C₁-C₄ alkyl, such as 2-(1-methoxycarbonylpiperidin-4-yl)ethyl, pyrrolidino-C₁-C₄ alkyl, such as pyrrolidinomethyl, piperazinocarbonyl-C₁-C₄ alkyl, N′-C₁-C₄ alkylpiperazinocarbonyl-C₁-C₄ alkyl, such as N′acetylpiperazinocarbonyl-C₁-C₄ alkyl, such as N′-acetylpiperazinocarbonylmethyl, morpholino, morpholino-C₁-C₄ alkyl, such as morpholinomethyl, 2-morpholinoethyl or 3-morpholinopropyl, thiomorpholino-C₁-C₄ alkyl, such as 2-thiomorpholinoethyl, C₁-C₄ alkoxy, such as methoxy, amino-C₁-C₇ alkoxy, such as 2-aminoethoxy or 3-aminopropyloxy, C₁-C₄ C₄ alkanoylamino-C₁-C₄ alkoxy, such as 2-acetylaminoethoxy, di-C₁-C₄ alkylamino-C₁-C₄ alkoxy, such as 3-dimethylaminopropyloxy, piperidino-C₁-C₄ alkoxy, such as 2-piperidinoethoxy, morpholino-C₁-C₄ alkoxy, such as 2-morpholinoethoxy or 3-morpholinopropyloxy, morpholino-C₁-C₄ alkylcarbamoyl-C₁-C₄ alkoxy, such as 2-morpholinoethylcarbamoylmethoxy, carboxy, carbamoyl, C₁-C₄ alkylcarbamoyl, such as methylcarbamoyl, carboxy-C₁-C₄ alkoxy, such as carboxymethoxy, carbamoyl-C₁-C₄ alkoxy, such as 2-carbamoylethoxy, C₁-C₄ alkylcarbamoyl-C₁-C₄ alkoxy, such as butylcarbamoylmethoxy, di-C₁-C₄ alkylamino-C₁-C₄ alkoxy, such as a 3-dimethylaminopropyloxy, or tetrazolyl-C₁-C₄ alkoxy, such as tetrazol-5-ylmethoxy, and

R_{D} is C₁-C₄ alkyl, such as methyl, C₁-C₄ alkoxy-C₁-C₄ alkyl, such as propyloxymethyl, carbamoyl-C₁-C₄ alkyl, such as 3-carbamoylpropyl or 2-carbamoyl-2-methyl-ethyl, C₁-C₄ alkylcarbamoyl-C₁-C₄ alkyl, such as 2-methylcarbamoyl-2-methyl-propyl, di-C₁-C₄ alkylcarbamoyl-C₁-C₄ alkyl, such as 2-dimethylcarbamoylethyl, piperidino-C₁-C₄ alkyl, such as pyrrolidinomethyl, or C₁-C₄ alkoxycarbonylpiperidino-C₁-C₄ alkyl, such as 2-(1-methoxycarbonylpiperidin-4-yl)ethyl,

one of the radicals X₁ and X₂ is carbonyl and the other is methylene,

R₂ is C₁-C₄ alkyl, such as methyl or isopropyl,

R₃ is amino or C₁-C₄ alkanoylamino, such as acetylamino,

R₄ is C₁-C₄ alkyl, such as methyl or isopropyl, and

R₅ is C₁-C₄ alkyl, such as butyl, C₁-C₄ alkoxy-C₁-C₄ alkyl, such as propyloxymethyl, C₁-C₄ alkoxycarbonylpiperidino-C₁-C₄ alkyl, such as 2-(1-methoxycarbonylpiperidin-4-yl)ethyl, pyrrolidino-C₁-C₄ alkyl, such as pyrrolidinomethyl, N′-C₁-C₄ alkylpiperazino-C₁-C₄ alkyl, such as N′-methylpiperazinomethyl, N′-C₁-C₄ alkoxycarbonylpiperazino-C₁-C₄ alkyl, such as N′-methoxycarbonylpiperazinomethyl, or N′-C₁-C₇ alkanoylpiperazino-C₁-C₄ alkyl, such as N′-acetylpiperazinomethyl, morpholino-C₁-C₄ alkyl, such as 2-morpholinoethyl or 3-morpholinopropyl, thiomorpholino-C₁-C₄ alkyl, such as 2-thiomorpholinoethyl, morpholinocarbonyl-C₁-C₄ alkyl, such as 2-morpholinocarbonylethyl, carbamoyl-C₁-C₄ alkyl, such as 3-carbamoylpropyl or 2-carbamoyl-2-methyl-ethyl, C₁-C₄ alkylcarbamoyl-C₁-C₄ alkyl, such as 2-methylcarbamoyl-2-methyl-ethyl, di-C₁-C₄ alkylcarbamoyl-C₁-C₄ alkyl, such as 2-dimethylcarbamoylethyl, piperidinocarbonyl-C₁-C₄ alkyl, such as piperidinocarbonylmethyl, piperazinocarbonyl-C₁-C₄ alkyl, N′-C₁-C₄ alkylpiperazinocarbonyl-C₁-C₄ alkyl, N′-C₁-C₄ alkanoylpiperazinocarbonyl-C₁-C₄ alkyl, such as N′-acetylpiperazinocarbonylmethyl, N′-C₁-C₄ alkylpiperazinocarbonyl-C₁-C₄ alkyl, such as N′-methylpiperazinocarbonylmethyl, or morpholinocarbonyl-C₁-C₄ alkyl, such as 2-morpholinocarbonylethyl,

and the salts thereof.

The invention relates above all to methods comprising compounds of formula I, especially of formula Ia

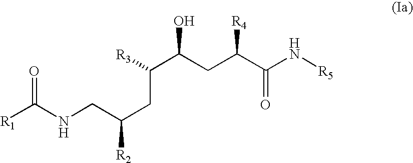

wherein R₁ is a 2-R_{A}-4-R_{C}-phenyl radical, a 2-R_{A}-pyridin-3-yl radical or a 3-R_{A}-pyridin-2-yl radical, wherein

R_{A}, is C₁-C₄ alkoxy-C₁-C₄ alkyl, such as propyloxymethyl, morpholino-C₁-C₄ alkyl, such as 2-morpholinoethyl or 3-morpholinopropyl, C₁-C₇ alkanoylpiperazino-C₁-C₄ alkyl, such as N′-acetylpiperazinomethyl, C₁-C₇ alkoxy, such as propyloxy, C₁-C₄ alkoxy-C₁-C₄ alkoxy, such as 2-methoxyethoxy, 3-methoxypropyloxy, 4-methoxybutyloxy or 5-methoxypentyloxy, C₁-C₄ alkoxy-C₁-C₄ alkenyloxy, such as 4-methoxy-but-2-enyloxy, C₁-C₄ alkoxy-C₁ C₄ alkoxy, such as 2-(methoxymethoxy)ethoxy or 2-(2-methoxyethoxy)ethoxy, amino-C₁-C₄ alkoxy, such as 2-aminoethoxy or 3-aminopropyloxy, di-C₁-C₄ alkylamino-C₁-C₄ alkoxy, such as 3-dimethylaminopropyloxy, carbamoyl-C₁-C₄ alkoxy, such as 2-carbamoylethoxy, or carbamoyl, and

R_{C} is hydrogen, di-C₁-C₄ alkylamino-C₁-C₄ alkyl, such as dimethylaminomethyl, piperidino-C₁-C₄ alkyl, such as piperidinomethyl, pyrrolidino-C₁-C₄ alkyl, such as pyrrolidinomethyl, morpholino-C₁-C₄ alkyl, such as morpholinomethyl, C₁-C₇ alkanoylpiperazino-C₁-C₄ alkyl, such as N′-acetylpiperazinomethyl, or C₁-C₄ alkylpiperazino-C₁-C₄ alkyl, such as N′-methylpiperazinomethyl, morpholino, C₁-C₄ alkoxy, such as methoxy, morpholino-C₁-C₄ alkoxy, such as 2-morpholinoethoxy or 3-morpholinopropyloxy, morpholino-C₁-C₄ alkylcarbamoyl-C₁-C₄ alkoxy, such as 2-morpholinoethylcarbamoylmethoxy, piperidino-C₁-C₄ alkoxy, such as 2-piperidinoethoxy, carboxy, carbamoyl, C₁-C₄ alkylcarbamoyl, such as methylcarbamoyl, carboxy-C₁-C₄ alkoxy, such as carboxymethoxy, di-C₁-C₄ alkylamino-C₁-C₄ alkoxy, such as 3-dimethylaminopropyloxy, C₁-C₇ alkylcarbamoyl-C₁-C₄ alkoxy, such as butylcarbamoylmethoxy, or tetrazolyl-C₁-C₄ alkoxy, such as tetrazol-5-ylmethoxy,

X₁ is carbonyl and X₂ is methylene,

R₂ and R₄ are each independently of the other C₁-C₄ alkyl, such as methyl or isopropyl,

R₃ is amino and

R₅ is C₁-C₄ alkyl, such as butyl, morpholino-C₁-C₄ alkyl, such as 2-morpholinoethyl or 3-morpholinopropyl, thiomorpholino-C₁-C₄ alkyl, such as 2-thiomorpholinoethyl, morpholinocarbonyl-C₁-C₄ alkyl, such as 2-morpholinocarbonylethyl, carbamoyl-C₁-C₄ alkyl, such as 3-carbamoylpropyl or 2-carbamoyl-2-methyl-ethyl, C₁-C₄ alkylcarbamoyl-C₁-C₄ alkyl, such as 2-methylcarbamoyl-2-methyl-ethyl, di-C₁-C₄ alkylcarbamoyl-C₁-C₄ alkyl, such as 2-dimethylcarbamoylethyl, N′-C₁-C₄ alkylpiperazino-C₁-C₄ alkyl, such as N′-methylpiperazinomethyl, N′-C₁-C₄ alkoxycarbonylpiperazino-C₁-C₄ alkyl, such as N′-methoxycarbonylpiperazinomethyl, or N′-C₁-C₇ alkanoylpiperazino-C₁-C₄ alkyl, such as N′-acetylpiperazinomethyl, and the salts thereof, especially the pharmaceutically acceptable salts thereof.

The invention relates specifically to methods comprising the compounds of formula I or formula I-A mentioned in the Examples and to the salts thereof, especially the pharmaceutically acceptable salts thereof.

Synthesis of Compounds

The process according to the invention for the preparation of compounds of formula I is as follows:

a) a compound of formula II

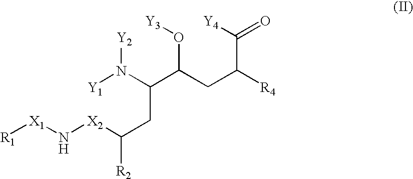

wherein Y₁ is lower alkyl, lower alkanoyl or an amino-protecting group, Y₂ is hydrogen or together with Y₃ is a bivalent protecting group, Y₃ is hydrogen, a hydroxy-protecting group or together with Y₂ is a bivalent protecting group or together with Y₄ is a direct bond, Y₄ is free or reactively etherified or esterified hydroxy or together with Y₃ is a direct bond and R₁, R₂, R₃, R₄, R₅, X₂ and X₂ are as defined for formula I, is reacted with an amine of formula III
H₂N—R₅ (III),

wherein R₅ is as defined for formula I, with the formation of an amide bond and any protecting groups present are removed, or

b) compounds of formulae IV and V

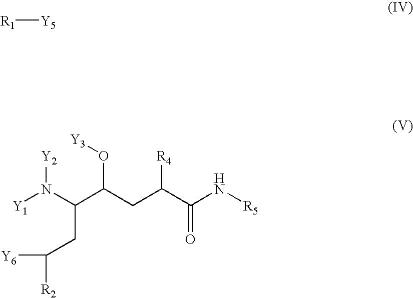

wherein Y₁ is lower alkyl, lower alkanoyl or an amino-protecting group, Y₂ is hydrogen or together with Y₃ is a bivalent protecting group, Y₃ is hydrogen, a hydroxy-protecting group or together with Y₂ is a bivalent protecting group, one of the radicals Y₅ and Y₆ is an aminomethyl group and the other is a free or functionally modified carboxy group and R₁, R₂, R₃, R₄ and R₅ are as defined for formula I, are condensed with one another and any protecting groups present are removed, or

c) for the preparation of compounds of formula I wherein R₃ is amino, in a compound of formula VI

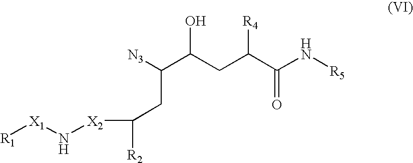

wherein R₁, R₂, R₄, R₅, X₁ and X₂ are as defined for formula I and Y₃ is hydrogen or a hydroxy-protecting group, the azido group is reduce to amino and condensed an any protecting groups present are removed,

and in each case, if desired, a compound of formula I having at least one salt-forming group obtainable by one of the above-mentioned processes is converted into its salt, or an obtainable salt is converted into the free compound or into a different salt and/or mixtures of isomers that may be obtainable are separated and/or a compound of formula I according to the invention is converted into a different compound of formula I according to the invention. Functional groups in starting materials the reaction of which is to be avoided, especially carboxy, amino and hydroxy groups, can be protected by suitable protecting groups (conventional protecting groups) which are customarily used in the synthesis of peptide compounds, and also in the synthesis of cephalosporins and penicillins as well as nucleic acid derivatives and sugars. Those protecting groups may already be present in the precursors and are intended to protect the functional groups in question against undesired secondary reactions, such as acylation, etherification, esterification, oxidation, solvolysis, etc. In certain cases the protecting groups can additionally cause the reactions to proceed selectively, for example stereoselectively. It is characteristic of protecting groups that they can be removed easily, i.e. without undesired secondary reactions taking place, for example by solvolysis, reduction, photolysis, and also enzymatically, for example under physiological conditions. Protecting groups may also be present in the end products, however. Compounds of formula I having protected functional groups may have greater metabolic stability or pharmacodynamic properties that are better in some other way than the corresponding compounds having free functional groups.

The protection of functional groups by such protecting groups, the protecting groups themselves and the reactions for their removal are described, for example, in standard works such as J. F. W. McOmie, “Protective Groups in Organic Chemistry”, Plenum Press, London and New York 1973, in Th. W. Greene, “Protective Groups in organic Synthesis”, Wiley, New York 1981, in “The Peptides”, Volume 3 (E. Gross and J. Meienhofer, eds.), Academic Press, London and New York 1981, in “Methoden der organischen Chemie”, Houben-Weyl, 4th edition, Volume 15/1, Georg Thieme Velag, Stuttgart 1974, in H.-D. Jakubke and H. Jescheit, “Aminosauren, Peptide, Proteine” (“Amino acids, peptides, proteins”), Velag Chemie, Weinheim, Deerfield Beach and Basle 1982, and in Jochen Lehmann, “Chemie der Kohlenhydrate: Monosacchadn und Derivatel” (“The Chemistry of carbohydrates: monosaccharin and derivatives”), Georg Thieme Verlag, Stuttgart 1974.

Amino-protecting groups Y₁ are, for example, acyl groups other than lower alkanoyl, also arylmethyl, lower alkylthio, 2-acyl-lower alk-1-enyl or silyl. The group Y₁ —N(Y₂)— can also be in the form of an azido group.

Acyl groups other than lower alkanoyl are, for example, halo-lower alkanoyl, for example 2-haloacetyl, such as 2-chloro-, 2-bromo-, 2-iodo-, 2,2,2-trifluoro- or 2,2,2-trichloro-acetyl, unsubstituted or substituted, for example halo-, lower alkoxy- or nitro-substituted, benzoyl, for example benzoyl, 4-chlorobenzoyl, 4-methoxybenzoyl or 4-nitrobenzoyl, or lower alkoxycarbonyl that is branched in the 1-position of the lower alkyl radical or suitably substituted in 1- or 2-position, for example tertiary lower alkoxycarbonyl, such as tert-butyloxycarbonyl, arylmethoxycarbonyl having one or two aryl radicals which are phenyl that is unsubstituted or mono- or poly-substituted, for example, by lower alkyl, for example tertiary lower alkyl, such as tertiary butyl, lower alkoxy, such as methoxy, hydroxy, halogen, such as chlorine, and/or by nitro, for example benzyloxycarbonyl, unsubstituted or substituted benzyloxycarbonyl, such as 4-nitrobenzyloxycarbonyl, diphenylmethoxycarbonyl, fluorenylmethoxycarbonyl or substituted diphenylmethoxycarbonyl, such as di(4-methoxyphenyl)methoxycarbonyl, aroylmethoxycarbonyl wherein the aroyl group is preferably benzoyl that is unbstituted or substituted, for example, by halogen, such as bromine, for example phenacyloxycarbonyl, 2-halo-lower alkoxycarbonyl, for example 2,2,2-trichloroethoxycarbonyl, 2-bromoethoxycarbonyl or 2-iodoethoxycarbonyl, 2-(tri-substituted silyl)alkoxycarbonyl, for example 2-tri-lower alkylsily-lower alkoxycarbonyl, for example 2-trimethylsilylethoxycarabonyl or 2-(di-n-butyl-methyl-silyl)-ethoxycarbonyl, or triarylsilyl-lower alkoxycarbonyl, for example 2-triphenylsilylethoxycarbonyl.

In a 2-acyl-lower alk-1-enyl radical that can be used as an amino-protecting group, acyl is, for example, the corresponding radical of a lower alkanecarboxylic acid, of a benzoic acid that is unsubstituted or substituted, for example, by lower alkyl, such as methyl or tertiary butyl, lower alkoxy, such as methoxy, halogen, such as chlorine, and/or by nitro, or especially of a carbonic acid semiester, such as a carbonic acid lower alkyl semiester. Corresponding protecting groups are especially 1-lower alkanoyl-prop-1-en-2-yl, for example 1-acetyl-prop-1-en-2-yl, or lower alkoxycarbonyl-prop-1-en-2-yl, for example 1-ethoxycarbonyl-prop-1-en-2-yl.

A silylamino group is, for example, a tri-lower alkylsilylamino group, for example trimethyl-silylamino. The silicon atom of the silylamino group can also be substituted by only two lower alkyl groups, for example methyl groups, and by the amino group or carboxy group of a second molecule of formula I. Compounds having such protecting groups can be prepared, for example, using dimethylchlorosilane as silylating agent.

An amino group can also be protected by conversion into the protonated form; suitable corresponding anions are especially those of strong inorganic acids, such as sulfuric acid, phosphoric acid or hydrohalic acids, for example the chlorine or bromine anion, or of organic sulfonic acids, such as p-toluenesulfonic acid.

Preferred amino-protecting groups Y₁ are acyl radicals of carbonic acid semiesters, such as lower alkoxycarbonyl, especially tert-butyloxycarbonyl or fluorenylmethoxycarbonyl, unsubstituted or lower alkyl-, lower alkoxy-, nitro- and/or halo-substituted α-phenyl- or α,α-diphenyl-lower alkoxycarbonyl, such as benzyloxycarbonyl, p-nitrobenzyloxycarbonyl or diphenylmethoxycarbonyl, or 2-halo-lower alkoxycarbonyl, e.g. 2,2,2-trichloroethoxycarbonyl, yl, also trityl.

Hydroxy-protecting groups Y₃ are, for example, acyl groups, for example lower alkanoyl that is substituted by halogen, such as chlorine, for example 2,2-dichloroacetyl, or especially acyl radicals of a carbonic acid semiester mentioned for protected amino groups. A preferred hydroxy-protecting group is, for example, 2,2,2-trichloroethoxycarbonyl, 4-nitrobenzyloxycarbonyl, diphenylmethoxycarbonyl or trityl. A further suitable hydroxy-protecting group Y₃ is tri-lower alkylsilyl, for example trimethylsilyl, triisopropylsilyl or dimethyl-tertbutylsilyl, a readily removable esterifying group, for example an alkyl group, such as tertiary lower alkyl, for example tertiary butyl, an oxa- or a thia-aliphatic or -cycloaliphatic, especially 2-oxa- or 2-thia-aliphatic or -cycloaliphatic, hydrocarbon radical, for example 1-lower alkoxy-lower alkyl or 1-lower alkylthio-lower alkyl, for example methoxymethyl, 1-methoxyethyl, 1-ethoxyethyl, methylthiomethyl, 1-methylthioethyl or 1-ethylthioethyl, or 2-oxa- or 2-thiacycloalkyl having from 5 to 7 ring atoms, for example 2-tetrahydrofuryl or 2-tetrahydropyranyl, or a corresponding thia analogue, and also 1-phenyl-lower alkyl, for example benzyl, diphenylmethyl or trityl, wherein the phenyl radicals can be substituted, for example, by halogen, for example chlorine, lower alkoxy, for example methoxy, and/or by nitro.

Bivalent protecting groups formed by Y₂ and Y₃ together are, for example, methylene groups substituted by one or two alkyl radicals or by an alkylene radical and are accordingly unsubstituted or substituted alkylidene, such as lower alkylidene, for example isopropylidenene, cycloalkylidene, for example cyclohexylidene, also carbonyl or benzylidene.

Process variant a): If Y₄ in starting materials of formula II is reactively etherified or esterified hydroxy, the terminal group —(═O)—Y₄, is a reactively functionally modified carboxylic acid function and is, for example, in the form of an activated ester or anhydride. The reactive acid derivatives can also be formed in situ.

Such activated esters of compounds of formula II are especially internal esters, for example γ-lactones, also esters unsaturated at the linking carbon atom of the esterifying radical, for example of the vinyl ester type, such as vinyl esters (obtainable, for example, by transesterification of a corresponding ester with vinyl acetate; activated vinyl ester method), carbamoyl esters (obtainable, for example, by treatment of the corresponding acid with an isoxazolium reagent; 1,2-oxazolium or Woodward method), or 1-lower alkoxyvinyl esters (obtainable, for example, by treatment of the corresponding acid with a lower alkoxyacetylene; ethoxyacetylene method), or esters of the amidino type, such as N,N′-disubstituted amidino esters (obtainable, for example, by treatment of the corresponding acid with a suitable N,N′-disubstituted carbodiimide, for example N, N′-dicyclohexylcarbodiimide; carbodiimide method), or N,N-disubstituted amidino esters (obtainable, for example, by treatment of the corresponding acid with an N,N-disubstituted cyanamide; cyanamide method), suitable aryl esters, especially phenyl esters suitably substituted by electron-attracting substituents (obtainable, for example, by treatment of the corresponding acid with a suitably substituted phenol, for example 4-nitrophenol, 4-methylsulfonylphenol, 2,4,5-trichlorophenol, 2,3,4,5,6-pentachlorophenol or 4-phenyldiazophenol, in the presence of a condensation agent, such as N,N′-dicyclohexylcarbodiimide; activated aryl esters method), cyanomethyl esters (obtainable, for example, by treatment of the corresponding acid with chloroacetonitrile in the presence of a base; cyanomethyl esters method), thioesters, especially unsubstituted or substituted, for example nitro-substituted, phenylthio esters (obtainable, for example, by treatment of the corresponding acid with unsubstituted or substituted, for example nitro-substituted thiophenols, inter alia by the anhydride or carbodiimide method; activated thiol esters method), or especially amino or amido esters (obtainable, for example, by treatment of the corresponding acid with an N-hydroxyamino or N-hydroxyimido compound, for example N-hydroxysuccinimide, N-hydroxypiperidine, N-hydroxyphthalimide, N-hydroxy-5-norbornene-2,3-dicarboxylic acid imide, 1-hydroxybenzotriazole or 3-hydroxy-3,4-dihydro-1,2,3-benzotriazin-4one, for example by the anhydride or carbodiimide method; activated N-hydroxy esters method).

The condensation of internal esters, especially γ-lactones, i.e. compounds of formula II wherein Y₃ and Y₄ together form a direct bond, is advantageously carried out in the presence of a basic condensation agent, preferably 2-hydroxypyridine at elevated temperature. This process variant is especially excellently suitable for the reaction with sterically hindered amines.

Anhydrides of acids of formula II may be symmetric or preferably mixed anhydrides of those acids, for example anhydrides with inorganic acids, such as acid halides, especially acid chlorides (obtainable, for example, by treatment of the corresponding acid with thionyl chloride, phosphorus pentachloride or oxalyl chloride; acid chloride method), azides (obtainable, for example, from a corresponding acid ester via the corresponding hydrazide and treatment thereof with nitrous acid; azide method), anhydrides with carbonic acid semiesters, for example carbonic acid lower alkyl semiesters (obtainable, for example, by treatment of the corresponding acid with chloroformic acid lower alkyl esters or with a 1-lower alkoxycarbonyl-2-lower alkoxy-1,2-dihydroquinoline; mixed O-alkylcarbonic acid anhydrides method), or anhydrides with dihalogenated, especially dichlorinated, phosphoric acid (obtainable, for example, by treatment of the corresponding acid with phosphorus oxychloride; phosphorus oxychloride method), anhydrides with other phosphoric acid derivatives (for example those obtainable with phenyl-N-phenylphosphoramidochloridate) or with phosphorous acid derivatives, or anhydrides with organic acids, such as mixed anhydrides with organic carboxylic acids (obtainable, for example, by treatment of the corresponding acid with an unsubstituted or substituted lower alkane- or phenyl-lower alkane-carboxylic acid halide, for example phenylacetic acid chloride, pivalic acid chloride or trifluoroacetic acid chloride; mixed carboxylic acid anhydrides method) or with organic sulfonic acids (obtainable, for example, by treatment of a salt, such as an alkali metal salt, of the corresponding acid with a suitable organic sulfonic acid halide, such as a lower alkane- or aryl-, for example methane- or p-toluene-sulfonic acid chloride; mixed sulfonic acid anhydrides method) and symmetric anhydrides (obtainable, for example, by condensation of the corresponding acid in the presence of a carbodiimide or 1-diethylaminopropyne; symmetric anhydrides method).

Several methods can be used to prepare the starting materials of formula II. For example, a compound of formula IIa

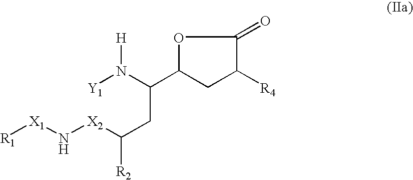

wherein X₁ is methylene, X₂ is carbonyl and Y₁ is an amino-protecting group, especially tertbutyloxycarbonyl, is obtained, for example, by reacting E-1,4-dibromobut-2-ene first with a compound of formula VII

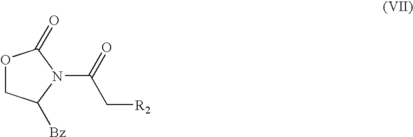

and then with a compound of formula VIII

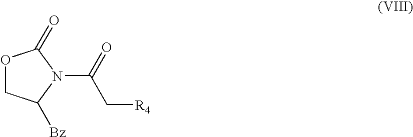

to form the corresponding compound of formula IX

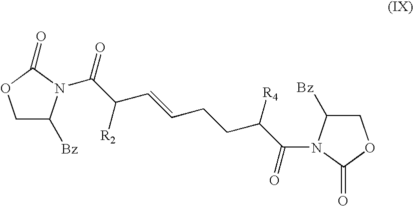

converting that compound, for example by treatment with a customary halogenating agent, such as elemental halogen, especially bromine or iodine, or preferably with an N-halo-succinimide, especially N-bromosuccinimide in 1,2-dimethoxyethane (DME), into the corresponding compound of formula X

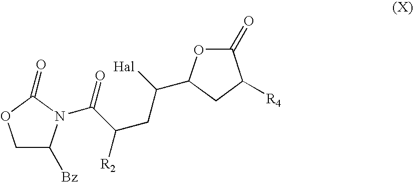

wherein Hal is halogen, separating the desired isomer in respect of R₂ and R₄ and in that isomer replacing the halogen atom by azido, for example by treatment with tetrabenzyl ammonium azide in toluene, and in the resulting compound of formula XI

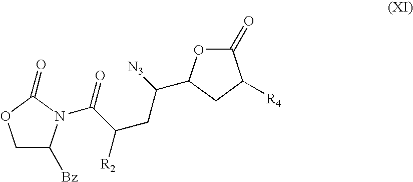

wherein R₂ and R₄ are as defined above and Bz is benzyl, hydrolysing the 4-benzyl-2-oxo-oxazolidin-1-ylcarbonyl group selectively to carboxy, reclosing, using a acid catalyst, a lactone ring which may have been opened; in the resulting compound of formula XII

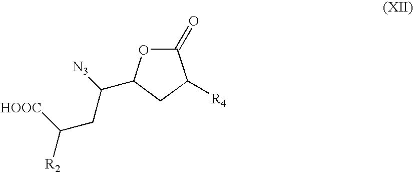

reducing the azido group to amino in customary manner, for example using hydrogen on palladium on carbon, temporarily protecting the amino group formed with an amino-protecting group Y₄, for example tert-butyloxycarbonyl, for example by reaction with di-tertbutyl dicarbonate, and condensing the resulting compound of formula XIII

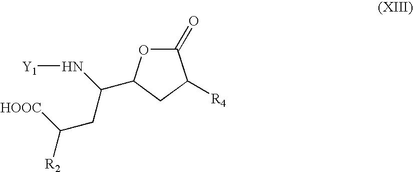

in customary manner, for example as described below under Process variant c), with a compound of formula IV
R₁—Y₅ (IV)

wherein Y₅ is aminomethyl.

Intermediates of formula IIa, wherein X₁ is carbonyl, X₂ is methylene and Y₁ is an amino-protecting group, for example tert-butyloxycarbonyl, can be obtained from compounds of formula XII obtainable as described above, by first reducing the carboxy group to hydroxymethyl, for example by reaction with a chloroformic acid ester and subsequent treatment with sodium borohydride, and then reducing the azido group to amino, for example using hydrogen in the presence of palladium on carbon, protecting the amino group formed with an amino-protecting group Y₄, for example with tert-butyloxycarbonyl, for example by reaction with di-tert-butyl dicarbonate, and in the resulting compound of formula XIV

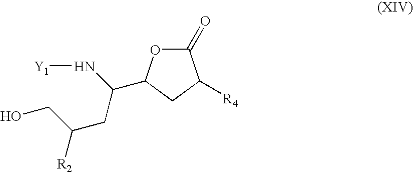

replacing the terminal hydroxy group by azido in customary manner, for example by treatment first with methanesulfonyl chloride and then with sodium azide; in the resulting compound of formula XV

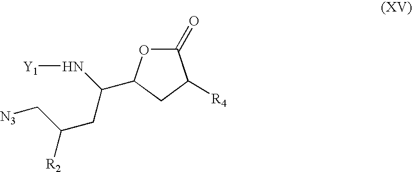

the azido group is reduced to amino in customary manner, for example as described above, and then substituted by the desired radical R₁ by reaction with a carboxylic acid of formula IV wherein Y₅ is carboxy. Starting materials of formula IIb

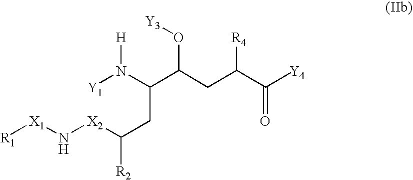

wherein Y₁ is an amino-protecting group, especially tert-butyloxycarbonyl, Y₃ is a hydroxy-protecting group, such as tri-lower alkylsilyl, Y₄ is hydroxy, X₁ is carbonyl and X₂ is methylene, can be prepared from azides of formula XV, for example by treatment with an alkali metal hydroxide, such as lithium hydroxide, subsequent reaction with tert-butyl-(dimethyl)silyl chloride, followed by customary reduction of the azido group to amino and, finally, reaction with a compound of formula IV
R₁—Y₅ (IV)

wherein Y₅ is free or reactively functionally modified carboxy.

Process variant b): Free or functionally modified carboxy Y₅ and Y₆, in starting materials of formulae IV and V, respectively, is, for example, free carboxy or carboxy present in the form of an ester or an anhydride. The reactive acid derivatives can also be formed in situ.

Esters of acids of formulae IV and V wherein Y₅ and Y₆ respectively, are carboxy are, for example, the aliphatic, araliphatic or aromatic esters thereof, such as a lower alkyl ester or a phenyl-lower alkyl ester that is unsubstituted or substituted in the phenyl moiety, for example by lower alkyl, lower alkoxy, halogen and/or by nitro, or a phenyl ester that is unsubstituted or substituted, for example, by lower alkyl, lower alkoxy, halogen and/or by nitro. Also suitable are activated esters. Suitable activated esters are especially esters unsaturated at the linking carbon atom of the esterifying radical, for example of the vinyl ester type, such as vinyl esters (obtainable, for example, by transesterification of a corresponding ester with vinyl acetate; activated vinyl ester method), carbamoyl esters (obtainable, for example, by treatment of the corresponding acid with an isoxazolium reagent; 1,2-oxazolium or Woodward method), or 1-lower alkoxyvinyl esters (obtainable, for example, by treatment of the corresponding acid with a lower alkoxyacetylene; ethoxyacetylene method), or esters of the amidino type, such as N,N′-disubstituted amidino esters (obtainable, for example, by treatment of the corresponding acid with a suitable N,N′-disubstituted carbodiimide, for example N,N′-dicyclohexylcarbodiimide; carbodiimide method), or N,N-disubstituted amidino esters (obtainable, for example, by treatment of the corresponding acid with an N,N-disubstituted cyanamide; cyanamide method), suitable aryl esters, especially phenyl esters suitably substituted by electron-attracting substituents (obtainable, for example, by treatment of the corresponding acid with a suitably substituted phenol, for example 4-nitrophenol, 4-methylsulfonylphenol, 2,4,5-trichlorophenol, 2,3,4,5,6-pentachlorophenol or 4-phenyldiazophenol, in the presence of a condensation agent, such as N,N′-dicyclohexylcarbodiimide; activated aryl esters method), cyanomethyl esters (obtainable, for example, by treatment of the corresponding acid with chloroacetonitrile in the presence of a base; cyanomethyl esters method), thioesters, especially unsubstituted or substituted, for example nitro-substituted, phenylthio esters (obtainable, for example, by treatment of the corresponding acid with unsubstituted or substituted, for example nitrosubstituted, thiophenols, inter alia by the anhydride or carbodiimide method; activated thiol esters method), or especially amino or amido esters (obtainable, for example, by treatment of the corresponding acid with an N-hydroxyamino or N-hydroxyamido compound, for example N-hydroxysuccinimide, N-hydroxypiperidine, N-hydroxyphthalimide, N-hydroxy-5-norbornene-2,3-dicarboxylic acid imide, 1-hydroxybenzotriazole or 3-hydroxy-3,4-dihydro-1,2,3-benzotriazin-4-one, for example by the anhydride or carbodiimide method; activated N-hydroxy esters method).

Anhydrides of acids of formulae IV and V wherein Y₅ and Y₆, respectively, are carboxy may be symmetric or preferably mixed anhydrides of those acids, for example anhydrides with inorganic acids, such as acid halides, especially acid chlorides (obtainable, for example, by treatment of the corresponding acid with thionyl chloride, phosphorus pentachloride or oxalyl chloride; acid chloride method), azides (obtainable, for example, from a corresponding acid ester via the corresponding hydrazide and treatment thereof with nitrous acid; azide method), anhydrides with carbonic acid semiesters, for example carbonic acid lower alkyl semiesters (obtainable, for example, by treatment of the corresponding acid with chloroformic acid lower alkyl esters or with a 1-lower alkoxycarbonyl-2-lower alkoxy-1,2-dihydroquinoline; 1-R_{D}-indol-3yl radical, mixed O-alkylcarbonic acid anhydrides method), or anhydrides with dihalogenated, especially dichlorinated, phosphoric acid (obtainable, for example, by treatment of the corresponding acid with phosphorus oxychloride; phosphorus oxychloride method), anhydrides with other phosphoric acid derivatives (for example those obtainable with phenyl-N-phenylphosphoramidochloridate) or with phosphorous acid derivatives, or anhydrides with organic acids, such as mixed anhydrides with organic carboxylic acids (obtainable, for example, by treatment of the corresponding acid with an unsubstituted or substituted lower alkane- or phenyl-lower alkane-carboxylic acid halide, for example phenylacetic acid chloride, pivalic acid chloride or trifluoroacetic acid chloride; mixed carboxylic acid anhydrides method) or with organic sulfonic acids (obtainable, for example, by treatment of a salt, such as an alkali metal salt, of the corresponding acid with a suitable organic sulfonic acid halide, such as a lower alkane- or aryl-, for example methane- or p-toluene-sulfonic acid chloride; mixed sulfonic acid anhydrides method) and symmetric anhydrides (obtainable, for example, by condensation of the corresponding acid in the presence of a carbodiimide or 1-diethylaminopropyne; symmetric anhydrides method).

The condensation of compounds of formulae IV and V can be carded out in a manner known per se, for example as described in standard works, such as Houben-Weyl, “Methoden der organischen Chemie”, 4th edition, Volume 15/11 (1974), Volume IX (1955), Volume E 11 (1985), Georg Thieme Verlag, Stuttgart, “The Peptides” (E. Gross and J. Meienhofer, eds.), Volumes 1 and 2, Academic Press, London and New York, 1979/1980, or M. Bodansky, “Principles of Peptide Synthesis”, Springer-Verlag, Berlin 1984.

The condensation of a free carboxylic acid with the corresponding amine can be carried out preferably in the presence of one of the customary condensation agents. Customary condensation agents are, for example, carbodiimides, for example diethyl-, dipropyl-, N-ethyl-N′-(3-dimethylaminopropyl)-carbodiimide or especially dicyclohexylcarbodiimide, also suitable carbonyl compounds, for example carbonyldiimidazole, 1,2-oxazolium compounds, for example 2-ethyl-5-phenyl-1,2-oxazolium-3′-sulfonate and 2-tert-butyl-5-methylisoxazolium perchlorate, or a suitable acylamino compound, for example 2-ethoxy-1-ethoxycarbonyl-1,2-dihydroquinoline, also activated phosphoric acid derivatives, for example diphenylphosphoryl azide, diethylphosphoryl cyanide, phenyl-N-phenylphosphoroamidochloridate, biS(2-oxo-3-oxazolidinyl)phosphinic acid chloride or 1-benzotdazolyloxytris (dimethylamino)phosphonium-hexafluorophosphate.

If desired, an organic base is added, for example a tri-lower alkylamine having bulky radicals, for example ethyldiisopropylamine, and/or a heterocyclic base, for example pyridine, N-methylmorpholine or preferably 4-dimethylaminopyridine.

The condensation of activated esters, reactive anhydrides or reactive cyclic amides with the corresponding amines is customarily carried out in the presence of an organic base, for example simple tri-lower alkylamines, for example triethylamine or tributylamine, or one of the above-mentioned organic bases. If desired, a condensation agent may additionally be used as described for free carboxylic acids.

The condensation of acid anhydrides with amines can be effected, for example, in the presence of inorganic carbonates, for example ammonium or alkali metal carbonates or hydrogen carbonates, such as sodium or potassium carbonate or hydrogen carbonate (usually together with a sulfate).

Carboxylic acid chlorides, for example the chlorocarbonic acid derivatives derived from the acid of formula II, are condensed with the corresponding amines preferably in the presence of an organic amine, for example the above-mentioned tri-lower alkylamines or heterocyclic bases, where appropriate in the presence of a hydrogen sulfate.

The condensation is preferably carried out in an inert, aprotic, preferably anhydrous, solvent or solvent mixture, for example in a carboxylic acid amide, for example formamide or dimethylformamide, a halogenated hydrocarbon, for example methylene chloride, carbon tetrachloride or chlorobenzene, a ketone, for example acetone, a cyclic ether, for example tetrahydrofuran, an ester, for example ethyl acetate, or a nitrile, for example acetonitrile, or in a mixture thereof, as appropriate at reduced or elevated temperature, for example in a temperature range of from approximately −40° C. to approximately +100° C., preferably from approximately −10° C. to approximately +50° C., and in the case where arylsulfonyl esters are used also at approximately +100° C. to +200° C., and if necessary under an inert gas atmosphere, for example a nitrogen or argon atmosphere.

Aqueous, for example alcoholic, solvents, for example ethanol, or aromatic solvents, for example benzene or toluene, may also be used When alkali metal hydroxides are present as bases, acetone can also be added where appropriate.

The condensation can also be carried out in accordance with the technique known as solid-phase synthesis which originates from R. Merrifield and is described, for example, in Angew. Chem. 97, 801-812 (1985), Naturwissenschaften 71,252-258 (1984) or in R. A. Houghten, Proc. Natl. Acad. Sci. U.S.A. 82, 5131-5135 (1985).

In a preferred variant of that process, which is suitable especially for the preparation of compounds of formula I wherein X₁ is carbonyl, X₂ is methylene and R₁ is, for example, a 1-R_{D}-indol-3-yl radical, the starting material used is a carboxylic acid of formula IV which is reacted with the amine component of formula V in the presence of a cyanophosphonic acid diester, for example cyanophosphonic acid diethyl ester, or a benzotriazolyloxy-triS(di-lower alkylamino)phosphonium salt, for example 1-benzotriazolyloxy-triS(dimethylamino)phosphonium-hexafluoro-phosphate or -chloride, and a tertiary organic amine, such as a tri-lower alkylamine, for example trimethylamine, and in a polar solvent, for example a nitrile, such as acetonitrile, an amide, such as dimethylformamide, N,N-dimethylacetamide, N-methylpyrrolidone or hexamethylphosphoric acid triamide, a urea, for example N,N′-dimethyl-N,N′-propylenylurea, lower alkoxy-lower alkanol, for example diethylene glycol monomethyl ether, in dimethyl sulfoxide or in a mixture of the mentioned solvents or in a mixture of one or more of the mentioned solvents with water, at temperatures of from −30° C. to 100° C., preferably from 20° C. to 80° C., the comments made above applying in respect of the protecting groups.

Starting materials of formula IV are known or can be prepared analogously to the method of formation of known compounds of formula IV.

Starting materials of formula V wherein Y₆ is amino, Y₁ is, for example, tert-butyloxycarbonyl and Y₂ and Y₃ together are, for example, isopropylidene, can be prepared, for example, in accordance with methods known per se, by condensing a compound of formula XVI

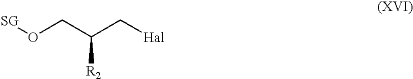

wherein SG is a hydroxy-protecting group, such as □-phenyl-lower alkyl, especially benzyl, Hal is halogen and R₂ is as defined, with a compound of formula XVII

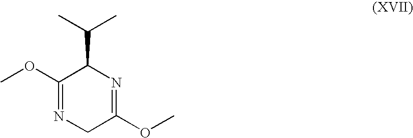

first hydrolysing the resulting compound of formula XVIII

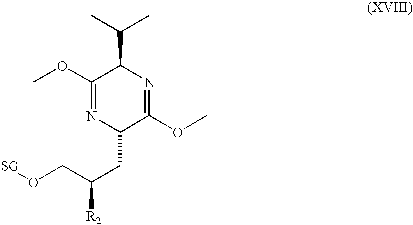

in customary manner, for example in the presence of dilute hydrochloric acid, and then reacting the product with di-tert-butyl dicarbonate, reacting the resulting compound of formula XIX

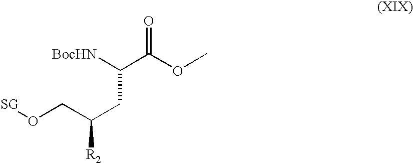

wherein Boc is tert-butyloxycarbonyl, in succession with dibutylaluminium hydride, with an N—R₅-methacrylamide, butyllithium and triisopropyloxytitanium chloride and, after separation of the resulting stereoisomeric mixture, with hydrogen in the presence of [Ru₂Cl₄-(S)-(BINAP)₂]NEt₃ and with dimethoxypropene and with p-toluenesulfonic acid, and in the resulting compound of formula XX

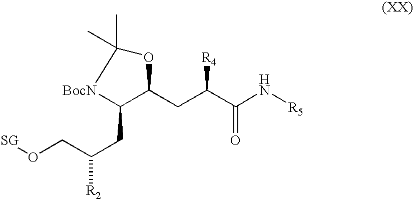

converting the protected hydroxy group SG—O—into amino in customary manner, for example by hydrogenolytic debenzylation, for example with hydrogen in the presence of palladium on carbon, reaction with a sulfonyl halide, such as methanesulfonyl chloride, further reaction with an alkali metal azide, such as sodium azide, and hydrogenation again, for example with hydrogen in the presence of palladium on carbon.

Process variant c): (Reduction of the azido group):

In starting materials of formula VI, functional groups that are not intended to participate in the reaction are protected by one of the protecting groups mentioned under Process a).

Reducing agents suitable for the reduction of the azido group are those which under the reaction conditions of the process reduce an optionally functionalised hydroxy group or azido group selectively or more rapidly than the amide groups present in compounds of formula I.

The reduction is preferably carried out with hydrogen in the presence of suitable heavy metal catalysts, for example Raney nickel or platinum or palladium catalysts, for example platinum or palladium on active carbon.

Intermediates of formula VI can be prepared, for example, by reacting E-1,4-dibromobut-2-ene first with a compound of formula VII

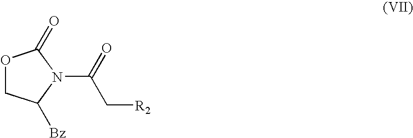

and then with a compound of formula VIII

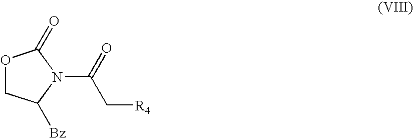

to form the corresponding compound of formula IX

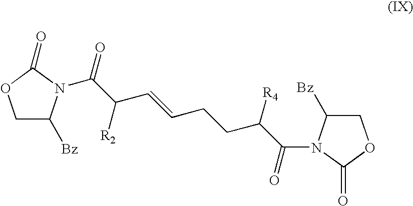

converting that compound, for example by treatment with a customary halogenating agent, such as elemental halogen, especially bromine or iodine, or preferably with an N-halosuccinimide, especially N-bromosuccinimide in 1,2-dimethoxyethane (DME), into the corresponding compound of formula X

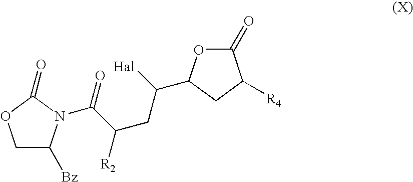

wherein Hal is halogen; separating the desired isomer in respect of R₂ and R₄ and in that isomer replacing the halogen atom by azido, for example by treatment with tetra benzylammonium azide in toluene, and in the resulting compound of formula XI

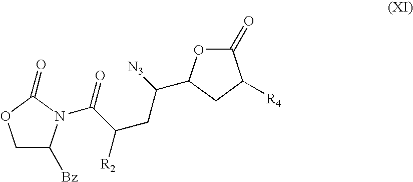

wherein R₂ and R₄ are as defined above and Bz is benzyl, hydrolysing the 4-benzyl-2-oxo-oxazolidin-1-ylcarbonyl group selectively to carboxy; reclosing, using an acid catalyst, a lactone ring which may have been opened; condensing the resulting compound of formula XII

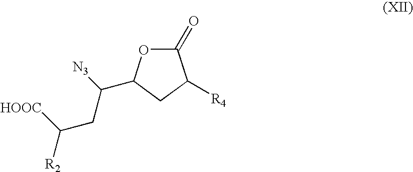

or a reactive functional carboxy derivative thereof, with a compound of formula IV
R₁—Y₅ (IV),

wherein Y₅ is aminomethyl, and condensing the resulting compound of formula XXI

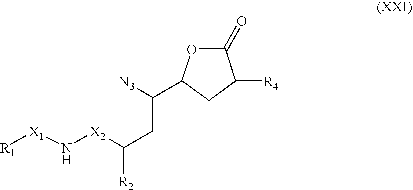

wherein X₁ is methylene and X₂ is carbonyl, in customary manner, for example as described under Process variant a), with an amine of formula III
H₂N—R₅ (III)

wherein R₅ is as defined for formula I.

Intermediates of formula VI wherein X₁ is carbonyl and X₂ is methylene can be prepared, for example, by converting the carboxy group into aminomethyl, especially in a manner analogous to that described for the preparation of compounds of formula IIb, at the stage of the compound of formula XII.

The removal of protecting groups that are not constituents of the desired end product of formula I, for example carboxy-, amino-, hydroxy- and/or mercapto-protecting groups, which may be carried out subsequent to the process variants described above, is effected in a manner known per se, for example by means of solvolysis, especially hydrolysis, alcoholysis or acidolysis, or by means of reduction, especially hydrogenolysis or chemical reduction, as well as photolysis, as appropriate stepwise or simultaneously, it being possible also to use enzymatic methods. The removal of the protecting groups is described, for example, in the standard works mentioned hereinabove in the section relating to protecting groups.

For example, protected carboxy, for example tertiary lower alkoxycarbonyl, lower alkoxycarbonyl substituted in the 2-position by a trisubstituted silyl group or in the 1-position by lower alkoxy or by lower alkylthio, or unsubstituted or substituted diphenylmethoxycarbonyl, can be converted into free carboxy by treatment with a suitable acid, such as formic acid or trifluoroacetic acid, where appropriate with the addition of a nucleophilic compound, such as phenol or anisole. Unsubstituted or substituted benzyloxycarbonyl can be cleaved, for example, by means of hydrogenolysis, i.e. by treatment with hydrogen in the presence of a metal hydrogenation catalyst, such as a palladium catalyst. In addition, suitably substituted benzyloxycarbonyl, such as 4-nitrobenzyloxycarbonyl, can be converted into free carboxy also by reduction, for example by treatment with an alkali metal dithionite, such as sodium dithionite, or with a reducing metal, for example zinc, or a reducing metal salt, such as a chromium(II) salt, for example chromium(II) chloride, customarily in the presence of a hydrogen-yielding agent that, together with the metal, is capable of producing nascent hydrogen, such as an acid, especially a suitable carboxylic acid, such as an unsubstituted or substituted, for example hydroxy-substituted, lower alkanecarboxylic acid, for example acetic acid, formic acid, glycolic acid, diphenylglycolic acid, lactic acid, mandelic acid, 4-chloromandelic acid or tartaric acid, or in the presence of an alcohol or thiol, water preferably being added. By treatment with a reducing metal or metal salt, as described above, 2-halo-lower alkoxycarbonyl (where appropriate after conversion of a 2-bromo-lower alkoxycarbonyl group into a corresponding 2-iodo-lower alkoxycarbonyl group) or aroylmethoxycarbonyl can also be converted into free carboxy. Aroylmethoxycarbonyl can be cleaved also by treatment with a nucleophilic, preferably salt-forming, reagent, such as sodium thiophenolate or sodium iodide. 2-(Tri-substituted silyl)-lower alkoxycarbonyl, such as 2-tri-lower alkylsilyl-lower alkoxycarbonyl, can be converted into free carboxy also by treatment with a salt of hydrofluoric acid that yields the fluoride anion, such as an alkali metal fluoride, for example sodium or potassium fluoride, where appropriate in the presence of a macrocyclic polyether (“crown ether”), or with a fluoride of an organic quaternary base, such as tetra-lower alkylammonium fluoride or tri-lower alkylarylammonium fluoride, for example tetraethylammonium fluoride or tetrabutylammonium fluoride, in the presence of an aprotic, polar solvent, such as dimethyl sulfoxide or N,N-dimethylacetamide. Carboxy protected in the form of organ silyloxycarbonyl, such as tri-lower alkylsilyloxycarbonyl, for example trimethylsilyloxycarbonyl, can be freed in customary manner by solvolysis, for example by treatment with water, an alcohol or an acid, or, furthermore, a fluoride, as described above. Esterified carboxy can also be freed enzymatically, for example by means of esterases or suitable peptidases.

A protected amino group is freed in a manner known per se and, according to the nature of the protecting groups, in various ways, preferably by solvolysis or reduction. 2-Halo-lower alkoxycarbonylamino (where appropriate after conversion of a 2-bromo-lower alkoxycarbonylamino group into a 2-iodo-lower alkoxycarbonylamino group), aroylmethoxycarbonylamino or 4-nitrobenzyloxycarbonylamino can be cleaved, for example, by treatment with a suitable reducing agent, such as zinc in the presence of a suitable carboxylic acid, such as aqueous acetic acid. Aroylmethoxycarbonylamino can be cleaved also by treatment with a nucleophitic, preferably salt-forming, reagent, such as sodium thiophenolate, and 4-nitrobenzyloxycarbonylamino also by treatment with an alkali metal dithionite, for example sodium dithionite. Unsubstituted or substituted diphenylmethoxycarbonylamino, tert-lower alkoxycarbonylamino or 2-(tri-substituted silyl)-lower alkoxycarbonylamino, such as 2-tri-lower alkylsilyl-lower alkoxycarbonylamino, can be cleaved by treatment with a suitable acid, for example formic or trifluoroacetic acid; unsubstituted or substituted benzyloxycarbonylamino can be cleaved, for example, by means of hydrogenolysis, i.e. by treatment with hydrogen in the presence of a suitable hydrogenation catalyst, such as a palladium catalyst; unsubstituted or substituted triarylmethylamino or formylamino can be cleaved, for example, by treatment with an acid, such as a mineral acid, for example hydrochloric acid, or an organic acid, for example formic, acetic or trifluoroacetic acid, where appropriate in the presence of water; and an amino group protected in the form of silylamino can be freed, for example, by means of hydrolysis or alcoholysis. An amino group protected by 2-haloacetyl, for example 2-chloroacetyl, can be freed by treatment with thiourea in the presence of a base, or with a thiolate salt, such as an alkali metal thiolate of thiourea, and subsequent solvolysis, such as alcoholysis or hydrolysis, of the resulting condensation product. An amino group protected by 2-(tri-substituted silyl)-lower alkoxycarbonyl, such as 2-tri-lower alkylsilyl-lower alkoxycarbonyl, can be converted into the free amino group also by treatment with a salt of hydrofluoric acid that yields fluoride anions, as indicated above in connection with the freeing of a correspondingly protected carboxy group. Likewise, silyl, such as trimethylsilyl, bonded directly to a hetero atom, such as nitrogen, can be removed using fluoride ions.

Amino protected in the form of an azido group is converted into free amino, for example, by reduction, for example by catalytic hydrogenation with hydrogen in the presence of a hydrogenation catalyst, such as platinum oxide, palladium or Raney nickel, by reduction using mercapto compounds, such as dithiothreitol or mercaptoethanol, or by treatment with zinc in the presence of an acid, such as acetic acid. The catalytic hydrogenation is preferably carried out in an inert solvent, such as a halogenated hydrocarbon, for example methylene chloride, or in water or in a mixture of water and an organic solvent, such as an alcohol or dioxane, at approximately from 20° to 25° C., or with cooling or heating.

A hydroxy or mercapto group protected by a suitable acyl group, by a tri-lower alkylsilyl group or by unsubstituted or substituted 1-phenyl-lower alkyl is freed analogously to a correspondingly protected amino group. A hydroxy or mercapto group protected by 2,2-dichloroacetyl is freed, for example, by basic hydrolysis, and a hydroxy or mercapto group protected by tertiary lower alkyl or by a 2-oxa- or 2-thia-aliphatic or -cycloaliphatic hydrocarbon radical is freed by acidolysis, for example by treatment with a mineral acid or a strong carboxylic acid, for example trifluoroacetic acid. Mercapto protected by pyridyldiphenylmethyl can be freed, for example, using mercury(II) salts at pH 2-6 or by zinc/acetic acid or by electrolytic reduction; acetamidomethyl and isobutyrylamidomethyl can be removed, for example, by reaction with mercury(II) salts at pH 2-6; 2-chloroacetamido methyl can be removed, for example, using 1-piperidinothiocarboxamide; and S-ethylthio, S-tert-butylthio and S-sulfo can be cleaved, for example, by thiolysis with thiophenol, thio glycolic acid, sodium thiophenolate or 1,4-dithiothreitol. Two hydroxy groups or an adjacent amino and hydroxy group which are protected together by means of a bivalent protecting group, preferably, for example, by a methylene group mono- or di-substituted by alkyl, such as lower alkylidene, for example isopropyldene, cycloalkyldene, for example cyclohexyldene, or benzyldene, can be freed by acid solvolysis, especially in the presence of a mineral acid or a strong organic acid. 2-Halo-lower alkoxycarbonyl is also removed using the above-mentioned reducing agents, for example a reducing metal, such as zinc, reducing metal salts, such as chromium(II)salts, or using sulfur compounds, for example sodium dithionite or preferably sodium sulfide and carbon disulfide.

When several protected functional groups are present, if desired the protecting groups may be so selected that more than one such group can be removed simultaneously, for example by acidolysis, such as by treatment with trifluoroacetic acid, or with hydrogen and a hydrogenation catalyst, such as a palladium on carbon catalyst. Conversely, the groups may also be so selected that they are not all removed simultaneously, but rather they are removed in a desired sequence or only some of them are removed.

In each of the processes mentioned above, the starting compounds may also be used in the form of salts, provided that the reaction conditions allow it.

Compounds of formula I obtainable in accordance with the process can be converted into different compounds of formula I in customary manner.

For example, in a compound of formula I obtainable in accordance with the process, a carboxy group in free or reactive form may be estefified or amidated or an esterified or amidated carboxy group may be converted into a free carboxy group.

For the esterification or amidation of a carboxy group in a compound of formula I, if desired the free acid can be used or the free acid can be converted into one of the above-mentioned reactive derivatives and reacted with an alcohol, with ammonia, or with a primary or secondary amine, or, in the case of esterification, the free acid or a reactive salt, for example the caesium salt, can be reacted with a reactive derivative of an alcohol. For example, the caesium salt of a carboxylic acid can be reacted with a halide or sulfonic acid ester corresponding to the alcohol. The esterification of the carboxy group can also be carried out with other customary alkylating agents, for example with diazomethane, Meerwein salts or 1-substituted 3-aryltriazenes.

For the conversion of an esterified or amidated carboxy group into the free carboxy group it is possible to use one of the methods described above for the removal of carboxy-protecting groups or, if desired, alkaline hydrolysis in accordance with the reaction conditions mentioned in Organikum, 17th edition, VEB Deutscher Verlag der Wissenschaften, Berlin 1988.

In a compound of formula I obtainable in accordance with the process, an esterified carboxy group can be converted into an unsubstituted or substituted carboxamide group by aminolysis with ammonia or with a primary or secondary amine, optionally in the presence of a suitable condensation agent or catalyst. The aminolysis can be carried out in accordance with the reaction conditions mentioned for such reactions in organikum, 15th edition, VEB Deutscher Verlag der Wissenschaften, Berlin (East) 1976.

A free amino group present in a compound of formula I obtainable in accordance with the process can be acylated or alkylated, for example to introduce a radical R₆ other than hydrogen. The acylation and the alkylation can be carried out in accordance with one of the methods mentioned for protecting groups or according to one of the procedures mentioned in Organikum, 17th edition, VEB Deutscher Verlag der Wissenschaften, Berlin (East) 1988.

Furthermore, a free hydroxy group present in a compound of formula I obtainable in accordance with the process, for example as a constituent of the radical R₅, can be acylated. The acylation can be carried out with acylating reagents in accordance with one of the methods mentioned for protecting groups or according to one of the procedures mentioned in organikum, 17th edition, VEB Deutscher Verlag der Wissenschaften, Berlin (East) 1988.

In a compound of formula I obtainable in accordance with the process it is also possible to obtain from a sulfide the corresponding sulfoxide or sulfone, that is to say to oxidise a thio group to a sulfinyl or sulfonyl group or a sulfinyl group to sulfonyl, and also to oxidise thiomorpholino to S-oxy- or S,S-dioxy-thiomorpholino.

The oxidation to the sulfone can be carried out with most of the customary oxidising agents. It is especially preferable to use oxidising agents that oxidise the thio group or the sulfide sulfur selectively in the presence of other functional groups, for example amino or hydroxy groups, of the compound of formula I in question, for example aromatic or aliphatic peroxycarboxylic acids, for example peroxybenzoic acid, monoperphthalic acid, m-chloroperbenzoic acid, peracetic acid, performic acid or trifluoroperacetic acid. The oxidation with peroxycarboxylic acids is carried out in the customary solvents suitable for that purpose, for example chlorinated hydrocarbons, for example methylene chloride or chloroform, ethers, such as diethyl ether, esters, such as ethyl acetate or the like, at temperatures of from −78° C. to room temperature, for example from −20° C. to +10° C., preferably about 0° C. The peroxycarboxylic acid can also be formed in situ, for example with hydrogen peroxide in acetic acid or formic acid that optionally contains acetic anhydride, for example with 30% or 90% hydrogen peroxide in acetic acidacetic anhydride. Other peroxo compounds are also suitable, for example potassium peroxomonosulfate in lower alkanol/water mixtures, for example methanol/water or ethanol/water, or in aqueous acetic acid at temperatures of from −70° C. to +30° C., for example from −20° C. to room temperature, also sodium metaperiodate in methanol or methanol/water mixtures at temperatures of from 0° C. to 50° C., for example about room temperature. If stoichiometric amounts of the mentioned oxidising agents are used it is also possible to obtain the corresponding sulfoxides.

If desired, it is possible by reduction of a sulfonyl group or a sulfone radical in an obtainable compound of formula I to obtain the corresponding thio compound or the corresponding sulfide, for example with diisobutylaluminium hydride in ether or tetrahydrofuran.

In compounds of formula I it is also possible to reduce a free or esterified carboxy group to hydroxymethyl in customary manner, for example using a di-light metal hydride, such as lithium aluminium hydride or sodium boranate, in an inert solvent, such as an ether, for example in tetrahydrofuran.

In compounds of formula I it is also possible to replace hydroxy R_{A}, R_{B} and/or R_{C} by one of the etherified hydroxy groups mentioned under formula I by reacting the corresponding compound of formula I wherein R_{A}, R_{B} and/or R_{C} is hydroxy in customary manner, for example in the presence of a basic condensation agent, with a compound of the formula(e) R_{A}—Y, R_{B}—Y and/or R_{C}—Y wherein one of the radicals R_{A} and R_{B} is an aliphatic, araliphatic or heteroaraliphatic radical, for example an amino-lower alkoxy radical that is unsubstituted or N-lower alkanoylated or N-mono- or N,N-di-lower alkylated or N,N-disubstituted by lower alkylene, hydroxy-, lower alkoxy- or lower alkoxy-lower alkoxy-lower alkylene, by unsubstituted or N′-lower alkanoylated, lower alkoxycarbonyl- or lower alkoxy-lower alkyl-N′-substituted or N′-lower alkylated aza-lower alkylene, by oxa-lower alkylene or by optionally S-oxidised thia-lower alkylene; lower alkoxy, hydroxy-lower alkoxy, lower alkanoyloxy-lower alkoxy, lower alkoxy-lower alkoxy, lower alkoxy-lower alkoxy-lower alkoxy, polyhalo-lower alkoxy, cyano-lower alkoxy, unsubstituted or substituted phenyl- or pyridyl-lower alkoxy, lower alkoxy-lower alkenyloxy, optionally S-oxidised lower alkylthio-lower alkoxy, or amino-lower alkoxy that is unsubstituted or N-lower alkanoylated or N-mono- or N,N-di-lower alkylated or N,N-disubstituted by lower alkylene, hydroxy-, lower alkoxy- or lower alkoxy-lower alkoxy-lower alkylene, by unsubstituted or N′-lower alkanoylated, lower alkoxycarbonyl- or lower alkoxy-lower alkyl-N′substituted or N′-lower alkylated aza-lower alkylene; by oxa-lower alkylene or by optionally S-oxidised thia-lower alkylene; and the other is hydrogen, lower alkyl, carbamoyl, hydroxy, lower alkoxy or polyhalo-lower alkoxy,

R_{C} is an aliphatic, araliphatic, heteroaraliphatic or heteroarylaliphatic radical, for example hydroxy, lower alkoxy, hydroxy-lower alkoxy, lower alkoxy-lower alkoxy, morpholino-lower alkylcarbamoyl-lower alkoxy; an amino-lower alkoxy group that is unsubstituted or N-lower alkanoylated or N-mono- or N,N-di-lower alkylated or N,N-disubstituted by lower alkylene, hydroxy-, lower alkoxy-, lower alkoxycarbonyl- or lower alkoxy-lower alkoxy-lower alkylene, by unsubstituted or N′-lower alkanoylated or lower alkoxycarbonyl- or lower alkoxy-lower alkyl-N′-substituted or N′-lower alkylated aza-lower alkylene, by oxa-lower alkylene or by optionally S-oxidised thia-lower alkylene; or a free or amidated carboxy or carboxy-lower alkoxy group or tetrazolyl-lower alkoxy, and

Y is reactive esterified hydroxy, especially hydroxy esterified by a mineral acid, by sulfuric acid or by an organic sulfonic acid, such as halogen, preferably chlorine, bromine or iodine, a group of the formula—O—SO₂—O—R_{A}, —O—SO₂—O—R_{B} or —O—SO₂—R_{A} or lower alkanesulfonyloxy or unsubstituted or substituted benzenesulfonyloxy, especially methane-, ethane-, benzene-, p-toluene- or p-bromobenzene-sulfonyl.

The reaction is, as mentioned, preferably carried out in the presence of a basic condensation agent, such as an alkali metal carbonate, for example potassium carbonate, in an inert solvent, such as a lower alkanol, such as methanol, ethanol, butanol, tert-butanol or especially amyl alcohol, advantageously at elevated temperature, for example in a temperature range of approximately from 40° C. to 140° C., if necessary with removal of the resulting water of reaction by distillation, for example by azeotropic distillation.

It is also possible for salts of compounds of formula I obtainable in accordance with the process to be converted in a manner known per se into the free compounds, for example by treatment with a base, such as an alkali metal hydroxide, a metal carbonate or metal hydrogen carbonate, or ammonia, or another of the salt-forming bases mentioned at the beginning, or with an acid, such as a mineral acid, for example with hydrochloric acid, or another of the salt-forming acids mentioned at the beginning.

Resulting salts can be converted into different salts in a manner known per se: acid addition salts, for example, by treatment with a suitable metal salt, such as a sodium, barium or silver salt, of a different acid in a suitable solvent in which an inorganic salt being formed is insoluble and is therefore eliminated from the reaction equilibrium, and basic salts by freeing of the free acid and conversion into a salt again.

The compounds of formula I, including their salts, may also be obtained in the form of hydrates or may include the solvent used for crystallisation.

As a result of the close relationship between the compounds in free form and in the form of their salts, hereinabove and hereinbelow any reference to the free compounds and their salts is to be understood as including also the corresponding salts and free compounds, respectively, as appropriate and expedient.

Stereoisomeric mixtures, that is to say mixtures of diastereoisomers and/or enantiomers, such as, for example, racemic mixtures, can be separated into the corresponding isomers in a manner known per se by suitable separating processes. For example, mixtures of diastereoisomers can be separated into the individual diastereoisomers by fractional crystallisation, chromatography, solvent partition etc. Racemates can be separated from one another, after conversion of the optical antipodes into diastereoisomers, for example by reaction with optically active compounds, for example optically active acids or bases, by chromatography on column materials charged with optically active compounds or by enzymatic methods, for example by selective reaction of only one of the two enantiomers. This separation can be carried out either at the stage of one of the starting materials or with the compounds of formula I themselves.

In a compound of formula I the configuration at individual chirality centres can be selectively reversed. For example, the configuration of asymmetric carbon atoms that carry nucleophilic substituents, such as amino or hydroxy, can be reversed by second order nucleophilic substitution, optionally after conversion of the bonded nucleophilic substituent into a suitable nucleofugal leaving group and reaction with a reagent introducing the original substituent, or the configuration at carbon atoms having hydroxy groups can be reversed by oxidation and reduction, analogously to the procedure in European Patent Application EP-A-0 236 734.

Also advantageous is the reactive functional modification of the hydroxy group and the subsequent replacement thereof by hydroxy with the configuration being reversed. For that purpose, the amino and hydroxy groups shown in formula I are bridged by a bivalent group, especially carbonyl, there being obtained a compound of formula XXII ##STR30## which can be cleaved again by treatment with thionyl chloride with the configuration being reversed.

The invention relates also to methods employing pharmaceutical compositions comprising compounds of formula I.

The above processes are described in detail in U.S. Pat. No. 5,641,778, herein incorporated by reference in its entirety.

The preparation of compounds that can be used as starting materials for the synthesis of the compounds of the invention is described in detail in U.S. Pat. No. 5,641,778, incorporated herein by reference.

The present invention may be better understood with reference to the following examples. These examples are intended to be representative of specific embodiments of the invention, and are not intended as limiting the scope of the invention.

### EXAMPLES

### Example A

Enzyme Inhibition Assay

The compounds of the invention are analyzed for inhibitory activity by use of the MBP-C125 assay. This assay determines the relative inhibition of beta-secretase cleavage of a model APP substrate, MBP-C125SW, by the compounds assayed as compared with an untreated control. A detailed description of the assay parameters can be found, for example, in U.S. Pat. No. 5,942,400. Briefly, the substrate is a fusion peptide formed of maltose binding protein (MBP) and the carboxy terminal 125 amino acids of APP-SW, the Swedish mutation. The beta-secretase enzyme is derived from human brain tissue as described in Sinha et al, 1999, Nature 40:537-540) or recombinantly produced as the full-length enzyme (amino acids 1-501), and can be prepared, for example, from 293 cells expressing the recombinant cDNA, as described in WO00/47618.

Inhibition of the enzyme is analyzed, for example, by immunoassay of the enzyme's cleavage products. One exemplary ELISA uses an anti-MBP capture antibody that is deposited on precoated and blocked 96-well high binding plates, followed by incubation with diluted enzyme reaction supernatant, incubation with a specific reporter antibody, for example, biotinylated anti-SW192 reporter antibody, and further incubation with streptavidin/alkaline phosphatase. In the assay, cleavage of the intact MBP-C125SW fusion protein results in the generation of a truncated amino-terminal fragment, exposing a new SW-192 antibody-positive epitope at the carboxy terminus. Detection is effected by a fluorescent substrate signal on cleavage by the phosphatase. ELISA only detects cleavage following Leu 596 at the substrate's APP-SW 751 mutation site.

Specific Assay Procedure:

Compounds are diluted in a 1:1 dilution series to a six-point concentration curve (two wells per concentration) in one 96-plate row per compound tested. Each of the test compounds is prepared in DMSO to make up a 10 millimolar stock solution. The stock solution is serially diluted in DMSO to obtain a final compound concentration of 200 micromolar at the high point of a 6-point dilution curve. Ten (10) microliters of each dilution is added to each of two wells on row C of a corresponding V-bottom plate to which 190 microliters of 52 millimolar NaOAc, 7.9% DMSO, pH 4.5 are pre-added. The NaOAc diluted compound plate is spun down to pellet precipitant and 20 microliters/well is transferred to a corresponding flat-bottom plate to which 30 microliters of ice-cold enzyme-substrate mixture (2.5 microliters MBP-C125SW substrate, 0.03 microliters enzyme and 24.5 microliters ice cold 0.09% TX100 per 30 microliters) is added. The final reaction mixture of 200 micromolar compound at the highest curve point is in 5% DMSO, 20 millimolar NaOAc, 0.06% TX100, at pH 4.5.

Warming the plates to 37 degrees C. starts the enzyme reaction. After 90 minutes at 37 degrees C., 200 microliters/well cold specimen diluent is added to stop the reaction and 20 microliters/well was transferred to a corresponding anti-MBP antibody coated ELISA plate for capture, containing 80 microliters/well specimen diluent. This reaction is incubated overnight at 4 degrees C. and the ELISA is developed the next day after a 2 hour incubation with anti-192SW antibody, followed by Streptavidin-AP conjugate and fluorescent substrate. The signal is read on a fluorescent plate reader.

Relative compound inhibition potency is determined by calculating the concentration of compound that showed a fifty percent reduction in detected signal (IC₅₀) compared to the enzyme reaction signal in the control wells with no added compound.

### Example B

Cell Free Inhibition Assay Utilizing a Synthetic APP Substrate

A synthetic APP substrate that can be cleaved by beta-secretase and having N-terminal biotin and made fluorescent by the covalent attachment of Oregon green at the Cys residue is used to assay beta-secretase activity in the presence or absence of the inhibitory compounds of the invention. Useful substrates include the following:

[SEQ ID NO: 1]
Biotin-SEVNLDAEFRC[Oregon green]KK
[SEQ ID NO: 2]
Biotin-SEVKMDAEFRC[Oregon green]KK
[SEQ ID NO: 3]
Biotin-GLNIKTEEISEISYEVEFRC[Oregon green]KK
[SEQ ID NO: 4]
Biotin-ADRGLTTRPGSGLTNIKTEEISEVNLDAEFC[Oregon green]KK
[SEQ ID NO: 5]
Biotin-FVNQHLC_{OX}GSHLVEALY-LVC_{OX}GERGFFYTPKAC[Oregon green]KK

The enzyme (0.1 nanomolar) and test compounds (0.001-100 micromolar) are incubated in pre-blocked, low affinity, black plates (384 well) at 37 degrees for 30 minutes. The reaction is initiated by addition of 150 millimolar substrate to a final volume of 30 microliter per well. The final assay conditions are: 0.001-100 micromolar compound inhibitor; 0.1 molar sodium acetate (pH 4.5); 150 nanomolar substrate; 0.1 nanomolar soluble beta-secretase; 0.001% Tween 20, and 2% DMSO. The assay mixture is incubated for 3 hours at 37 degrees C., and the reaction is terminated by the addition of a saturating concentration of immunopure streptavidin. After incubation with streptavidin at room temperature for 15 minutes, fluorescence polarization is measured, for example, using a LJL Acqurest (Ex485 nm/Em530 nm). The activity of the beta-secretase enzyme is detected by changes in the fluorescence polarization that occur when the substrate is cleaved by the enzyme. Incubation in the presence or absence of compound inhibitor demonstrates specific inhibition of beta-secretase enzymatic cleavage of its synthetic APP substrate.

### Example C

Beta-Secretase Inhibition: P26-P4′SW Assay

Synthetic substrates containing the beta-secretase cleavage site of APP are used to assay beta-secretase activity, using the methods described, for example, in published PCT application WO00/47618.

| | |
|---|---|
| The P26-P4′SW substrate is a peptide of | |
| the sequence: | |
| (biotin) CGGADRGLTTRPGSGLTNIKTEEISEVNLDAEF | [SEQ ID NO: 6] |
| The P26-P1 standard has the sequence: | |
| (biotin) CGGADRGLTTRPGSGLTNIKTEEISEVNL. | [SEQ ID NO: 7] |

Briefly, the biotin-coupled synthetic substrates are incubated at a concentration of from about 0 to about 200 micromolar in this assay. When testing inhibitory compounds, a substrate concentration of about 1.0 micromolar is preferred. Test compounds diluted in DMSO are added to the reaction mixture, with a final DMSO concentration of 5%. Controls also contain a final DMSO concentration of 5%. The concentration of beta secretase enzyme in the reaction is varied, to give product concentrations with the linear range of the ELISA assay, about 125 to 2000 picomolar, after dilution.

The reaction mixture also includes 20 millimolar sodium acetate, pH 4.5, 0.06% Triton X100, and is incubated at 37 degrees C. for about 1 to 3 hours. Samples are then diluted in assay buffer (for example, 145.4 nanomolar sodium chloride, 9.51 millimolar sodium phosphate, 7.7 millimolar sodium azide, 0.05% Triton X405, 6 g/liter bovine serum albumin, pH 7.4) to quench the reaction, then diluted further for immunoassay of the cleavage products.

Cleavage products can be assayed by ELISA. Diluted samples and standards are incubated in assay plates coated with capture antibody, for example, SW192, for about 24 hours at 4 degrees C. After washing in TTBS buffer (150 millimolar sodium chloride, 25 millimolar Tris, 0.05% Tween 20, pH 7.5), the samples are incubated with streptavidin-AP according to the manufacturer's instructions. After a one hour incubation at room temperature, the samples are washed in TTBS and incubated with fluorescent substrate solution A (31.2 g/liter 2-amino-2-methyl-1-propanol, 30 mg/liter, pH 9.5). Reaction with streptavidin-alkaline phosphate permits detection by fluorescence. Compounds that are effective inhibitors of beta-secretase activity demonstrate reduced cleavage of the substrate as compared to a control.

### Example D

Assays using Synthetic Oligopeptide-Substrates

Synthetic oligopeptides are prepared that incorporate the known cleavage site of beta-secretase, and optionally detectable tags, such as fluorescent or chromogenic moieties. Examples of such peptides, as well as their production and detection methods are described in U.S. Pat. No. 5,942,400, herein incorporated by reference. Cleavage products can be detected using high performance liquid chromatography, or fluorescent or chromogenic detection methods appropriate to the peptide to be detected, according to methods well known in the art.

By way of example, one such peptide has the sequence (biotin)-SEVNLDAEF [SEQ ID NO: 8], and the cleavage site is between residues 5 and 6. Another preferred substrate has the sequence ADRGLTTRPGSGLTNIKTEEISEVNLDAEF [SEQ ID NO: 9], and the cleavage site is between residues 26 and 27.

These synthetic APP substrates are incubated in the presence of beta-secretase under conditions sufficient to result in beta-secretase mediated cleavage of the substrate. Comparison of the cleavage results in the presence of the compound inhibitor to control results provides a measure of the compound's inhibitory activity.

### Example E

Inhibition of Beta-Secretase Activity—Cellular Assay

An exemplary assay for the analysis of inhibition of beta-secretase activity utilizes the human embryonic kidney cell line HEKp293 (ATCC Accession No. CRL-1573) transfected with APP751 containing the naturally occurring double mutation Lys651Met52 to Asn651Leu652 (numbered for APP751), commonly called the Swedish mutation and shown to overproduce A beta (Citron et al., 1992, Nature 360:672-674), as described in U.S. Pat. No. 5,604,102.

The cells are incubated in the presence/absence of the inhibitory compound (diluted in DMSO) at the desired concentration, generally up to 10 micrograms/ml. At the end of the treatment period, conditioned media is analyzed for beta-secretase activity, for example, by analysis of cleavage fragments. A beta can be analyzed by immunoassay, using specific detection antibodies. The enzymatic activity is measured in the presence and absence of the compound inhibitors to demonstrate specific inhibition of beta-secretase mediated cleavage of APP substrate.

### Example F

Inhibition of Beta-Secretase in Animal Models of AD

Various animal models can be used to screen for inhibition of beta-secretase activity. Examples of animal models useful in the invention include, but are not limited to, mouse, guinea pig, dog, and the like. The animals used can be wild type, transgenic, or knockout models. In addition, mammalian models can express mutations in APP, such as APP695-SW and the like described herein. Examples of transgenic non-human mammalian models are described in U.S. Pat. Nos. 5,604,102, 5,912,410 and 5,811,633.

PDAPP mice, prepared as described in Games et al., 1995, Nature 373:523-527 are useful to analyze in vivo suppression of A beta release in the presence of putative inhibitory compounds. As described in U.S. Pat. No. 6,191,166, 4 month old PDAPP mice are administered compound formulated in vehicle, such as corn oil. The mice are dosed with compound (1-30 mg/ml; preferably 1-10 mg/ml). After time, e.g., 3-10 hours, the animals are sacrificed, and brains removed for analysis.

Transgenic animals are administered an amount of the compound inhibitor formulated in a carrier suitable for the chosen mode of administration. Control animals are untreated, treated with vehicle, or treated with an inactive compound. Administration can be acute, i.e., single dose or multiple doses in one day, or can be chronic, i.e., dosing is repeated daily for a period of days. Beginning at time 0, brain tissue or cerebral fluid is obtained from selected animals and analyzed for the presence of APP cleavage peptides, including A beta, for example, by immunoassay using specific antibodies for A beta detection. At the end of the test period, animals are sacrificed and brain tissue or cerebral fluid is analyzed for the presence of A beta and/or beta-amyloid plaques. The tissue is also analyzed for necrosis.

Animals administered the compound inhibitors of the invention are expected to demonstrate reduced A beta in brain tissues or cerebral fluids and reduced beta amyloid plaques in brain tissue, as compared with non-treated controls.

### Example G

Inhibition of A Beta Production in Human Subjects

Subjects suffering from Alzheimer's Disease (AD) demonstrate an increased amount of A beta in the brain. AD subjects are administered an amount of the compound inhibitor formulated in a carrier suitable for the chosen mode of administration. Administration is repeated daily for the duration of the test period. Beginning on day 0, cognitive and memory tests are performed, for example, once per month.

Subjects administered the compound inhibitors are expected to demonstrate slowing or stabilization of disease progression as analyzed by changes in one or more of the following disease parameters: A beta present in CSF or plasma; brain or hippocampal volume; A beta deposits in the brain; amyloid plaque in the brain; and scores for cognitive and memory function, as compared with control, non-treated subjects.

### Example H

Prevention of A Beta Production in Subjects at Risk for AD

Subjects predisposed or at risk for developing AD are identified either by recognition of a familial inheritance pattern, for example, presence of the Swedish Mutation, and/or by monitoring diagnostic parameters. Subjects identified as predisposed or at risk for developing AD are administered an amount of the compound inhibitor formulated in a carrier suitable for the chosen mode of administration. Administration is repeated daily for the duration of the test period. Beginning on day 0, cognitive and memory tests are performed, for example, once per month.

Subjects administered the compound inhibitors are expected to demonstrate slowing or stabilization of disease progression as analyzed by changes in one or more of the following disease parameters: A beta present in CSF or plasma; brain or hippocampal volume; amyloid plaque in the brain; and scores for cognitive and memory function, as compared with control, non-treated subjects.

### EXAMPLES OF COMPOUNDS

All temperatures are in degrees Celsius, and all pressures are given in units of mbar

TLC Eluant Systems:

A hexane-ethyl acetate (9:1)

B hexane-ethyl acetate (4:1)

C hexane-ethyl acetate (3:1)

D hexane-ethyl acetate (2:1)

E hexane-ethyl acetate (1:1)

F hexane-ethyl acetate (1:2)

G hexane-ethyl acetate-glacial acetic acid (50:50:1)

H ethyl acetate-methanol-conc. ammonia (98:1:1)

I ethyl acetate-methanol-conc. ammonia (90:10:1)

J ethyl acetate-methanol-conc. ammonia (80:15:5)

K ethyl acetate-methanol-conc. ammonia (40:45:5)

L dichloromethane-methanol (90:10)

M dichloromethane-methanol (92:8)

N dichloromethane-methanol (95:5)

O dichloromethane-methanol (96:4)

P dichloromethane-methanol (97:3)

Q dichloromethane-methanol (98:2)

R dichloromethane-methanol (99:1)

S dichloromethane-methanol-conc. ammonia (99:1:1)

T dichloromethane-methanol-conc. ammonia (98:2:1)

U dichloromethane-methanol-conc. ammonia (96:4:1)

V dichloromethane-methanol-conc. ammonia (97:3:1)

W dichloromethane-methanol-conc. ammonia (90:10:1)

X dichloromethane-methanol-acetic acid (90:10:1)

Y dichloromethane-methanol-acetic acid (95:5:1)

HPLC eluent gradients on C18-Nucleosil® (5 μM), column length 25 cm: 20% acetonitrile/80% water/0.1% trifluoroacetic acid to 100% acetonitrile/0% water/0.1% trifluoroacetic acid for 20 minutes, then 100% acetonitrile/0.1% trifluoroacetic acid for 8 minutes.

The abbreviation “R_{f}(A)” means, for example, that the R_{f} value is determined in solvent system A. The ratio of solvents to one another is always given in parts by volume.

For the designation of the eluant systems, the same abbreviations are used in the case of flash chromatography and medium-pressure chromatography.

The short names and abbreviations used have the following meanings:

| | |
|---|---|
| bar | pressure in bar |
| C18-Nucleosil ® | trade name for HPLC reverse phase column |
| | material charged with octadecyl radicals |
| FAB-MS | fast atom bombardment mass spectroscopy |
| HRMS(FAB) | high resolution fast atom bombardment mass |
| | spectroscopy |
| TLC | thin-layer chromatography |
| FC | flash column chromatography |
| HPLC | high-performance liquid chromatography |
| Hyflo ® | trade name for filter aids (Fluka, Buchs, |
| | Switzerland) |
| Min | minute(s) |
| b.p. | boiling point at the pressure given in torr |
| ml | milliliters |
| R_{f} | ratio of the migration of a substance to the |
| | distance of the eluant front from the |
| | starting point in TLC |
| Rₜ | retention time of a substance in HPLC |
| m.p. | melting point |

### Example 1

A mixture of 2-(3-methoxypropoxy)-benzoic acid (0.105 g), biS(2-oxo-3-oxazolidinyl)phosphinic acid chloride (0.127 g) and triethylamine (0.140 ml) in dichloromethane (2 ml) is stirred at room temperature for one hour. Then a solution of (2R,4′S,5′S,2″S)-3-[4′-(2″-aminomethyl-3″-methylbutyl)-3′-(tert-butoxycarbonyl)-2′,2′-dimethyl-1,3-oxazolidin-5′-yl]-2-methylpropionic acid N-(butyl)amide (0.111 g) in dichloromethane (2 ml) and 4-dimethylaminopyridine (0.024 g) are added, and the reaction mixture is stirred overnight. After removal of the solvent by evaporation, saturated sodium hydrogen carbonate solution (30 ml) is added to the residue and extraction is then carried out with ethyl acetate (3×30 ml). The organic phases are dried over magnesium sulfate, concentrated by evaporation and purified by FC (20 g of silica gel, eluant F). (2S,4′S,5′S,2″R)—N-{2-[5′-(2″-butylcarbamoylpropyl)-3′-(tert-butoxycarbonyl)-2′,2′-dimethyl-1,3-oxazolidin-4′-ylmethyl]-3-methylbutyl}-2-(3-methoxypropoxy)-benzamide (0.112 g) is obtained in the form of a colourless oil: R_{f} (F)=0.28. HPLC Rₜ=21.0 min.

The (2R,4′S,5′S,2″S)-3-[4′-(2″-aminomethyl-3″-methybutyl)-3′-(tert-butoxycarbonyl)-2′,2′-dimethyl-1,3-oxazolidin-5′-yl]-2methylpropionic acid N-(butyl)amide used as starting material is prepared as follows:

a) (2R,4′S,5′S,2″S)-3-[4′-(2″-Aminomethyl-3″-methylbutyl)-3′-(tert-butoxycarbonyl)-2′,2^{o}-dimethyl-1,3-oxazolidin-5′-yl]-2-methylpropionic acid N-(butyl)amide: (2R,4′S,5′S,2″S)-3-[4-(2″-Azidomethyl-3″-methylbutyl)-3′-(tert-butoxycarbonyl)-2′,2′-dimethyl-1,3-oxazolidin-5′-yl]-2-methylpropionic acid N-(butyl)amide (0.435 g) dissolved in ethyl acetate (25 ml) is hydrogenated for 2 hours at room temperature and under normal pressure in the presence of 10% Pd/C (0.100 g). Filtration over Hyflo® and removal of the solvent yield 0.41 g of the crude title compound in the form of a pale-yellow oil: R_{f} (dichloromethane-methanol-conc. ammonia 350:50:1)=0.19. HPLC Rₜ=13.6 min. MS(FAB) m/e 442 (M⁺+1).

b) (2R,4′S,5′S,2″S)-3-[4′-(2″-Azidomethyl-3″-methylbutyl)-3′-(tert-butoxycarbonyl)-2′,2′-dimethyl-1,3-oxazolidin-5′-yl]-2-methylpropionic acid N-(butyl)amide: A mixture of (2S,4′S,5′-S,2″R)-methanesulfonic acid 2-[5′-(2″-butylcarbamoylpropyl)-3′-(tert-butoxycarbonyl)-2′,2′-dimethyl-1,3-oxazolidin-4′-ylmethyl]-3-methylbutyl ester (0.52 g) and sodium azide (0.65 g) in 1,3-dimethyl-3,4,5,6-tetrahydro-2(1H)-pydmidinone (5 ml) is stirred at 50° C. for 5 hours. The cooled reaction mixture is poured onto water (100 ml) and extracted with diethyl ether (3×100 ml). The organic phases are washed with water (2×100 ml) and brine (100 ml), dried over magnesium sulfate and concentrated by evaporation. FC (35 g of silica gel, eluant D) of the evaporation residue yields the title compound (0.438 g) in the form of a pale-yellow oil: R_{f} (E)=0.49. HPLC Rₜ=21.0 min. MS(FAB) m/e 468 (M⁺+1).

c) (2S,4′S,5′S,2″R)-Methanesulfonic acid 2-[5′-(2″-butylcarbamoylpropyl)-3′-(tert-butoxycarbonyl)-2′,2′-dimethyl-1,3-oxazolidin-4′-ylmethyl]-3-methylbutyl ester: To a stirred solution of (2R,4′S,5′S,2″S)-3-[4′-(2″-hydroxymethyl-3″-methyl-butyl)-3′-(tert-butoxycarbonyl)-2′,2′-dimethyl-1,3-oxazolidin-5′-yl]-2-methylpropionic acid N-(butyl)amide (0.442 g) in dichloromethane 5 ml) there are added at 0° C. first triethylamine (0.418 ml) and then methanesulfonic acid chloride (0.117 ml). The reaction mixture is stirred at 0° C. for one hour and then the solvent is concentrated to half. FC (25 g of silica gel, eluant E) yields the title compound (0.51 g) in the form of a colourless oil: R_{f} (E)=0.27. HPLC Rₜ=18.5 min. MS(FAB) m/e 521 (M⁺+1).

d) (2R,4′S,5′S,2″S)-3-[4′-(2″-Hydroxymethyl-3″-methyl-butyl)-3′-(tert-butoxycarbonyl)-2′,2′-dimethyl-1,3-oxazolidin-5′-yl]-2-methylpropionic acid N-(butyl)amide: (2R,4′S,5′S,2″S)-3-[4′-(2″-Benzyloxymethyl-3″-methyl-butyl)-3′-(tert-butoxycarbonyl)-2′,2′-dimethyl-1,3-oxazolidin-5′-yl]-2-methylpropionic acid N-(butyl)amide (2.20 g), dissolved in tetrahydrofuran (60 ml), is hydrogenated for 0.5 hour at room temperature and under normal pressure in the presence of 10% Pd/C Degussa E 101N (0.220 g). Filtration over Hyflo® and removal of the solvent yield the title compound (1.79 g) in the form of a colourless oil: R_{f} (E)=0.23. HPLC Rₜ=18.3 min. MS(FAB) m/e 443 (M⁺+1).

e) (2R,4′S,5′S,2″S)-3-[4′-(2″-Benzyloxymethyl-3″-methyl-butyl)-3′-(tert-butoxycarbonyl)-2′,2′-dimethyl-1,3-oxazolidin-5′-yl]-2-methylpropionic acid N-(butyl)amide: 2′,2′-dimethoxypropane (5 ml) and p-toluenesulfonic acid (0.01 g) are added in succession, with stirring, to a solution of (2RS,4S,5S,7S)-7-benzyloxymethyl-5-(tert-butoxycarbonyl)amino-4-hydroxy-2,8-dimethyl-nonanoic acid N(butyl)amide (3.0 g) in dichloremethane (30 ml), and the reaction mixture is then left to stand for 20 hours. The solvent is concentrated and the crude product, which consists of the two (2S,4′S,5′S,2″S)- and (2R,4′S,5′S,2″S)-diastereoisomers in the ratio 2:8 (HPLC Rₜ=23.3/23.5 min), is chromatographed (FC on 160 g of silica gel, eluant C). The stereoisomerically pure title compound (2.22 g) is obtained in the form of a colourless oil: R_{f} (E)=0.47. HPLC Rₜ=23.5 min.

f) (2RS,4S,5S,7S)-7-Benzyloxymethyl-5-(tert-butoxycarbonyl) amino-4-hydroxy-2,8-dimethyl-nonanoic acid N-(butyl) amide: A solution of (2S,3S,5S)-2-[3-(tert-butoxycarbonyl)amino-5-(benzyloxymethyl)-2-hydroxy-6-methylheptyl]-N-(butyl)acrylamide (A) (3.0 g) in anhydrous methanol (25 ml) is hydrogenated for 22 hours at room temperature and under a pressure of 25 bar in the presence of [Ru₂Cl₄ [(S)-BlNAP]₂]NEt₃ (39.5 mg). The reaction mixture is concentrated by evaporation and then purified by FC (100 g of silica gel, eluant E). The title compound (3.0 g) is obtained in the form of a pale-yellow oil: R_{f} (E)=0.15. HPLC Rₜ=19.4 min.

g) (2S,3S,5S)-2-[3-(tert-Butoxycarbonyl)amino-5-(benzyloxymethyl)-2-hydroxy-6-methylheptyl]-N-(butyl)acrylamide (A) and (2R,3S,5S)-2-[3-(tert-butoxycarbonyl)amino-5-(benzyloxymethyl)-2-hydroxy-6-methylheptyl]-N-(butyl)acrylamide (B): A 1.6M n-butyllithium solution in hexane (73.3 ml) is added at −75° C. over a period of 15 minutes, with stirring, to a solution of methacrylic acid N-(butyl)amide (7.92 g) in tetrahydrofuran (125 ml). When the addition is complete, the reaction mixture is stirred at 0° C. for 30 minutes and is cooled to −75° C., and a 1M chlorotitanium triisopropoxide solution in hexane (89.3 ml) is added dropwise over a period of 40 minutes. The mixture is stirred at −75° C. for a further 15 minutes and then a solution of (2S,4S)-2-(tert-butoxycarbonyl)amino-4-(benzyloxymethyl)-5-methyl-hexanal (9.10 g) in tetrahydrofuran (90 ml) is added dropwise at the same temperature over a period of 15 minutes. The reaction mixture is stirred at −75° C. for a further 75 minutes, and saturated ammonium chloride solution (150 ml) is then added at −20° C. The aqueous phase is extracted with diethyl ether (3×600 ml), and the combined organic phases are washed in succession with water (600 ml) and saturated sodium chloride solution (600 ml), dried over magnesium sulfate and concentrated by evaporation. The crude product is chromatographed on 1.3 kg of silica gel (eluant C) with separation of the mixture of diastereoisomers. Title compound A (3.01 g) is obtained in the form of a pale-yellow oil: R_{f} (D)=0.22. HPLC Rₜ=20.1 min. In addition, title compound B (5.70 g) is obtained in the form of a pale-yellow oil: R_{f} (D)=0.17. HPLC Rₜ=19.87 min.

h) (2S,4S)-2-(tert-Butoxycarbonyl)amino-4-(benzyloxymethyl) -5-methyl-hexanal: A 1.2M diisobutylaluminium hydride solution in toluene (51 ml) is slowly added at −75° C., with stirring, to a solution of (2S,4S)-2-(tert-butoxycarbonyl)amino-4-(benzyloxymethyl)-5-methyl-hexanoic acid methyl ester (9.70 g) in toluene (100 ml). The reaction mixture is stirred at the same temperature for a further 45 minutes, and then methanol (20 ml) is added carefully. The resulting mixture is poured onto 1N hydrochloric acid/ice (500 ml) and is extracted with ethyl acetate (3×500 ml). The organic phases are washed in succession with water (2×500 ml) and saturated sodium chloride solution (500 ml), are clarified by filtration over Hyflo® and are dried over magnesium sulfate. The solvent is removed by evaporation and the residue is dried under a high vacuum. The crude title compound (8.91 g) is obtained in the form of a colourless oil: R_{f} (B)=0.25. HPLC Rₜ=19.2 min.

i) (2S,4S)-2-(tert-butoxycarbonyl)amino-4-(benzylloxymethyl)-5-methyl-hexanoic acid methyl ester: ethyl diisopropylamine (17.4 ml) and a solution of di-tert-butyl dicarbonate (18.8 g) in dichloromethane (0.1 liter) are added in succession at 0° C., with stirring, to a solution of (2S,4S)-2-amino-4-(benzyloxymethyl)-5-methyl-hexanoic acid methyl ester (21.9 g) in dichloromethane (0.5 liter). The reaction mixture is stirred for a further 16 hours at room temperature and is then concentrated by evaporation. FC (2.4 kg of silica gel, ethyl acetate-hexane 1:6) of the evaporation residue yields the title compound (27.0 g) in the form of a slightly yellowish oil: R_{f} (B)=0.32. HPLC Rₜ=27.2 min.

j) (2S,4S)-2-Amino-4-(benzyloxymethyl)-5-methyl-hexanoic acid methyl ester: A 1N hydrochloric acid solution (400 ml) is added at room temperature to a solution of (2S,2′S,5R)-2-[2′(benzyloxymethyl)-3′-methylbutyl]-2,5-dihydro-5-isopropyl-3,6-dimethoxypyrazine (36.2 g) in acetonitrile (400 ml), and the mixture is stirred for 2 hours. The reaction mixture is then poured onto a mixture of saturated sodium hydrogen carbonate solution and ice (1 liter), and extraction washed out with dichloromethane (3×0.8 liter). The organic phases are washed with water (1 liter), dried over magnesium sulfate and concentrated by evaporation. The evaporation residue is purified by FC (2.4 kg of silica gel, dichloromethane-methanol-conc. ammonia 95:5:0.1). The title compound (21.9 g) is obtained in the form of a colourless oil: R_{f} (dichloromethaneomethanol-conc. ammonia 700:50:1)=0.34. HPLC Rₜ=13.6 min.

k) (2S,2′S,5R)-2-[2′-(Benzyloxymethyl)-3′-methylbutyl]-2,5-dihydro-5-isopropyl-3,6-dimethoxypyrazine: A 1.6M n-butyllithium solution in hexane (100 ml) is added dropwise at −75° C. to a solution of (2R)-2,5-dihydro-2-isopropyl-3,6-dimethoxypyrazine (29.5 g) in absolute tetrahydrofuran (530 ml). The reaction mixture is then stirred at −75° C. for 30 minutes, and then a solution of 2(S)-(benzyloxymethyl)-3-methyl-butyl bromide (29 g) in tetrahydrofuran (130 ml) is added over a period of 20 minutes. The reaction solution is stirred at −75° C. for a further 2 hours and is then left to stand at −18° C. for 64 hours. The reaction mixture is concentrated by evaporation, water (500 ml) is added, and extraction is carried out with diethyl ether (3×500 ml). The organic phases are washed with saturated sodium chloride solution (500 ml), dried over magnesium sulfate and concentrated by evaporation. The evaporation residue is purified by FC (2.4 kg of silica gel, ethyl acetate-hexane 1:15), and the title compound (36.2 g) is obtained in the form of a yellowish oil: R_{f} (B)=0.58. HPLC Rₜ=25.8 min.

The 2-(3-methoxypropoxy)-benzoic acid used as starting material is prepared as follows:

a) 1N sodium hydroxide solution (11.1 ml) is added to a solution of 2-(3-methoxypropoxy)-benzoic acid ethyl ester (2.4 g) in ethanol (20 ml) and water (10 ml), and the reaction mixture is stirred at 50° C. for 7 hours. The mixture is concentrated and the acidified aqueous phase is extracted with dichloremethane (3×40 ml). The organic phase is washed with saturated sodium chloride solution, dried over magnesium sulfate and concentrated. 2-(3-methoxypropoxy)-benzoic acid, R_{f} (hexane-ethyl acetate-glacial acetic acid 1:2:0.1)=0.43, is obtained in the form of a yellowish oil.

b) 2-(3-Methoxypropoxy)-benzoic acid ethyl ester: Dried potassium carbonate powder (3.49 g) is added, with stirring, to a solution of salicylic acid ethyl ester (3.5 g) in anhydrous acetone (50 ml), and then a solution of 3-methoxypropyl bromide (4.83 g) in anhydrous acetone (15 ml) is quickly added dropwise at room temperature. The suspension is heated under reflux for 38 hours. After cooling, filtration is carried out, the filtrate is concentrated and the residue is purified by FC (200 g of silica gel, eluant A). The title compound, R_{f} (hexane-ethyl acetate-glacial acetic acid 1:1:0.1)=0.39, is obtained in the form of a colouress oil.

### Example 2

In a manner analogous to that described in Example 1), the following compounds are prepared:

a) From 128 mg of (2R,4′S,5′S,2″S)-3-[4′-(2″-aminomethyl-3″-methylbutyl)-3′-(tert-butoxycarbonyl)-2′,2′-dimethyl-1,3-oxazolidin-5′-yl]-2-methylpropionic acid N-(butyl)amide and 139 mg of 3-methoxy-2-(3-methoxypropoxy)-benzoic acid, (2S,4′S,5′S,2″R)-N-{2-[5′-(2″-butylcarbamoylpropyl)-3′-(tert-butoxycarbonyl)-2′,2′-dimethyl-1,3-oxazolidin-4′-ylmethyl]-3-methylbutyl}-3-methoxy-2-(3-methoxypropoxy)-benzamide, R_{f} (L)=0.65; HPLC Rₜ=20.9 min; MS(FAB) m/e 664 (M⁺+1), in the form of a colorless oil.

b) From 128 mg of (2R,4′S,5′S,2″S)-3-[4′-(2″-aminomethyl-3″-methylbutyl)-3′-(tert-butoxycarbonyl)-2′,2′-dimethyl-1,3-oxazolidin-5′-yl]-2-methylpropionic acid N-(butyl)amide and 139 mg of 4-methoxy-2-(3-methoxypropoxy)-benzoic acid, (2S,4′S,5′S,2″R)-N-{2-[5.dbd.-(2″-butylcarbamoylpropyl)-3′-(tert-butoxycarbonyl)-2′,2′-dimethyl-1,3-oxazolidin-4′-ylmethyl]-3-methylbutyl}-4-methoxy-2-(3-methoxypropoxy)-benzamide, R_{f} (N)=0.17; HPLC Rₜ=21.4 min; MS(FAB) m/e 664 (M⁺+1), in the form of a colorless oil.

c) From 111 mg of (2R,4′S,5′S,2″S)-3-[4′-(2″-aminomethyl-3″-methylbutyl)-3′-(tert-butoxycarbonyl)-2′,2′-dimethyl-1,3-oxazolidin-5′-yl]-2-methylpropionic acid N-(butyl)amide and 105 mg of 3-(3-methoxypropoxy)-benzoic acid, (2S,4′S,5′S,2″R)-N-{2-[5′-(2″butylcarbamoylpropyl)-3′-(tert-butoxycarbonyl)-2′,2′-dimethyl-1,3-oxazolidin-4′-ylmethyl]-3-methylbutyl}-3-(3-methoxypropoxy)-benzamide, R_{f} (N)=0.26; HPLC Rₜ=20.7 min; MS(FAB) m/e 634 (M⁺+1), in the form of a white foam.

The 3-(3-methoxypropoxy)-benzoic acid used as starting material is prepared as follows:

a) Alkaline hydrolysis of 3-(3-methoxypropoxy)-benzoic acid methyl ester in a manner analogous to that described in Example 1) yields the title compound, R_{f} (hexane-ethyl acetate-glacial acetic acid 3:1:0.01)=0.18; m.p. 82°-84° C., in the form of a solid.

b) 3-(3-methoxypropoxy)-benzoic acid methyl ester: Sodium hydride in the form of an 80% dispersion in oil (0.39 g) is added at 0° C., with stirring, to a solution of 3-hydroxybenzoic acid methyl ester (2.04 g) in tetrahydrofuran (50 ml). After stirring for 30 minutes, a solution of 3-methoxypropyl bromide (3.08 g) in tetrahydrofuran (15 ml) is added dropwise at 0° C. The mixture is heated slowly to 50° C. and the white suspension is stirred for a further 30 hours. The mixture is poured onto ice-water (40 ml) and the aqueous phase is extracted with dichloromethane (3×40 ml). The combined organic phases are washed with saturated sodium chloride solution, dried over magnesium sulfate and concentrated. Purification of the crude product by FC (100 g of silica gel, eluant A) yields the title compound, R_{f} (C)=0.36, in the form of a yellowish oil.

The 4-methoxy-2-(3-methoxypropoxy)-benzoic acid and 3-methoxy-2-(3-methoxypropoxy)-benzoic acid used above as starting materials are prepared in a manner analogous to that described in Example 1).

### Example 3

p-Toluenesulfonic acid (2 mg) is added, with stirring, to a solution of (2S,4′S,5′S,2″R)-N-{2-[5′-(2″-butylcarbamoylpropyl)-3′-(tert-butoxycarbonyl)-2′,2′-dimethyl-1,3-oxazolidin-4′-ylmethyl]-3-methylbutyl}-2-(3-methoxypropoxy)-benzamide (105 mg) in methanol (3 ml), and the mixture is stirred for a further 24 hours at room temperature. The solvent is removed by evaporation at room temperature and the residue is purified by FC (40 g of silica gel, eluant N). (2S,4S,5S,7R)-N-[4-(tert-butoxycarbonyl) amino-7-butylcarbamoyl-5-hydroxy-2-isopropyl-octyl]-2-(3-methoxypropoxy)-benzamide (86 mg) is obtained in the form of a white foam: R_{f} (F)=0.09. HPLC Rₜ=17.2 min.

### Example 4

In a manner analogous to that described in Example 3), the following compounds are prepared:

a) From 150 mg of (2S,4′S,5′S,2″R)-N-{2-[5′-(2′-butylcarbamoylpropyl)-3′-(tert-butoxycarbonyl)-2′,2′-dimethyl-1,3-oxazolidin-4′-ylmethyl]-3-methylbutyl}-3-methoxy-2-(3-methoxypropoxy)-benzamide, (2S,4S,5S,7R)-N-[4-(tert-butoxycarbonyl)amino-7-butylcarbamoyl-5-hydroxy-2-isopropyl-octyl]-3-methoxy-2-(3-methoxypropoxy)-benzamide, R_{f} (F)=0.05; HPLC Rₜ=17.2 min; MS(FAB) m/e 624 (M⁺+1), in the form of a colorless oil.

b) From 172 mg of (2S,4′S,5′S,2″R)-N-{2-[5′-(2″-butylcarbamoylpropyl)-3′-(tert-butoxycarbonyl)-2′,2′-dimethyl-1,3-oxazolidin-4-ylmethyl]-3-methylbutyl}-4-methoxy-2-(3-methoxypropoxy)-benzamide, (2S,4S,5S,7R)-N-[4-(tert-butoxycarbonyl)amino-7-butylcarbamoyl-5-hydroxy-2-isopropyl-octyl]-4-methoxy-2-(3-methoxypropoxy)-benzamide, R_{f} (N)=0.30; HPLC Rₜ=17.6 min; MS(FAB) m/e 624 (M⁺+1), in the form of a colorless oil.

c) From 105 mg of (2S,4′S,5′S,2″R)-N-{2-[5′-(2″-butylcarbamoylpropyl)-3′-(tert-butoxycarbonyl)-2′,2′-dimethyl-1,3-oxazolidin-4′-ylmethyl]-3-methylbutyl}-3-(3-methoxypropoxy)-benzamide, (2S,4S,5S,7R)-N-[4-(tert-butoxycarbonyl)amino-7-butylcarbamoyl-5-hydroxy-2-isopropyl-octyl]-3-(3-methoxypropoxy)-benzamide, R_{f} (N)=0.18; HPLC Rₜ=17.0 min; MS(FAB) m/e 594 (M⁺+1), in the form of a white foam.

### Example 5

(2S,4S,5S,7R)-N-[4-(tert-Butoxycarbonyl)amino-7-butylcarbamoyl-5-hydroxy-2-isopropyl-octyl]-2-(3-methoxypropoxy)-benzamide (82 mg) is dissolved at 0° C. in 3 ml of a 4N hydrochloric acid solution in dioxane, and the solution is stirred at 0° C. for 2 hours. The reaction mixture is lyophilised and (2S,4S,5S,7R)-N-(4-amino-7-butylcarbamoyl-5-hydroxy-2-isopropyl-octyl)-2-(3-methoxypropoxy)-benzamide hydrochloride is obtained in the form of a white foam: R_{f} (L)=0.12. HPLC Rₜ=11.6 min. MS(FAB) m/e 494 (M⁺+1).

### Example 6

In a manner analogous to that described in Example 5), the following compounds are prepared by de-Bocylation:

a) From 117 mg of (2S,4S,5S,7R)-N-[4-(tert-butoxycarbonyl)amino-7-butylcarbamoyl-5-hydroxy-2-isopropyl-octyl]-3-methoxy-2-(3-methoxypropoxy)-benzamide, (2S,4S,5S,7R)-N-(4-amino-7-butylcarbamoyl-5-hydroxy-2-isopropyl-octyl)-3-methoxy-2-(3-methoxypropoxy)-benzamide hydrochloride: R_{f} (L)=0.15. HPLC Rₜ=11.7 min. MS(FAB) m/e 524 (M⁺+1).

b) From 119 mg of (2S,4S,5S,7R)-N-[4-(tert-butoxycarbonyl)amino-7-butylcarbamoyl-5-hydroxy-2-isopropyl-octyl]-4-methoxy-2-(3-methoxypropoxy)-benzamide, (2S,4S,5S,7R)-N-(4-amino-7-butylcarbamoyl-5-hydroxy-2-isopropyl-octyl)-4-methoxy-2-(3-methoxypropoxy)-benzamide hydrochloride: R_{f} (L)=0.13. HPLC Rₜ=12.3 min. MS(FAB) m/e 524 (M⁺+1).

c) From 82 mg of (2S,4S,5S,7R)-N-[4-(tert-butoxycarbonyl)amino-7-butylcarbamoyl-5-hydroxy-2-isopropyl-octyl]-3-(3-methoxypropoxy)-benzamide, (2S,4S,5S,7R)-N-(4-amino-7-butylcarbamoyl-5-hydroxy-2-isopropyl-octyl)-3-(3-methoxypropoxy)-benzamide hydrochloride: R_{f} (L)=0.20. HPLC Rₜ=11.4 min. MS(FAB) m/e 494 (M⁺+1).

### Example 7

Triethylamine (0.034 ml) and formic acid 4-nitrophenyl ester (28 mg) are added at room temperature, with stirring, to a solution of (2S,4S,5S,7R)-N-(4-amino-7-butylcarbamoyl-5-hydroxy-2-isopropyl-octyl)-3-methoxy-2-(3-methoxypropoxy)-benzamide hydrochloride (Example 6a) (67 mg) in dichloromethane (4 ml). The resulting reaction solution is stirred for a further 30 minutes and is then concentrated by evaporation. The residue is purified by FC (18 g of silica gel; eluant E, then ethyl acetate-hexane-methanol 5:5:1). (2S,4S,5S,7R)-N-(7-Butylcarbamoyl-4-formylamino-5-hydroxy-2-isopropyl-octyl)-3-methoxy-2-(3-methoxypropoxy)-benzamide is obtained in the form of a white foam: R_{f} (L)=0.58. HPLC Rₜ=13.2 min. MS(FAB) m/e 552 (M⁺+1)

### Example 8

(2R,4′S,5′S,2″R)-3-[4-(2″-Aminomethyl-3″-methylbutyl)-3′-(tert-butoxycarbonyl)-2′,2′-dimethyl-1,3-oxazolidin-5′-yl]-2-methylpropionic acid N-(butyl)amide (100 mg) is reacted in dichloromethane in accordance with the process described in Example 1) with 1-benzyl-1H-indole-3-carboxylic acid (114 mg) in the presence of biS(2-oxo-3-oxazolidinyl)phosphinic acid chloride (115 mg), triethylamine (0.13 ml) and a catalytic amount of 4-dimethylaminopyridine. When the reaction is complete, the solvent is removed by evaporation and the residue is immediately purified by FC (30 g of silica gel, eluant W). (2R,4′S,5′S,2″R)-1-benzyl-1H-indole-3-carboxylic acid N-{2-[5′-(2″-butylcarbamoylpropyl)-2′,2′-dimethyl-1,3-oxazolidin-4′-ylmethyl]-3-methylbutyl}-amide, R_{f} (L)=0.61, is obtained in the form of a yellowish oil.

The (2R,4′S,5′S,2″R)-3-[4′-(2″-aminomethyl-3″-methybutyl)-3′-(tert-butoxycarbonyl)-2′,2′-dimethyl-1,3-oxazolidin-5′-yl]-2-methylpropionic acid N-(butyl)amide used as starting material is prepared as follows:

a) (2R,4′S,5′S,2″R)-3-[4′-(2″-aminomethyl-3″-methylbutyl)-3′-(tert-butoxycarbonyl)-2′,2^{o}-dimethyl-1,3-oxazolidin-5′-yl]-2-methylpropionic acid N-(butyl)amide: The title compound is obtained in the form of a pale-yellow oil, R_{f} (W)=0.32, from (2R,4′S,5′S,2″R)-3-[4′-(2″-azidomethyl-3″-methylbutyl)-3′-(tert-butoxycarbonyl)-2′,2′-dimethyl-1,3-oxazolidin-5′-yl]-2-methylpropionic acid N-(butyl)amide (0.6 g) by hydrogenolysis analogously to Example 1a) and then purification by FC (100 g of silica gel, eluant V).

b) (2R,4′S,5′S,2″R)-3-[4′-(2″-Azidomethyl-3″-methylbutyl)-3′-(tert-butoxycarbonyl)-2′,2′-dimethyl-1,3-oxazolidin-5′-yl]-2-methylpropionic acid N-(butyl)amide: Reaction of (2R,4S,5S,2′R)-methanesulfonic acid 2-[5′-(2″-butylcarbamoylpropyl)-3′-(tert-butoxycarbonyl)-2′,2′-dimethyl-1,3-oxazolidin-4′-ylmethyl]-3-methylbutyl ester (1.30 g) with sodium azide (1.92 g) in a manner analogous to that described in Example 1b) and purification by FC (200 g of silica gel, eluant D) yield the title compound, R_{f} (E)=0.61, in the form of a pale-yellow oil.

c) (2R,4′S,5′S,2″R)-methanesulfonic acid 2-[5′-(2″-butylcarbamoylpropyl)-3′-(tert-butoxycarbonyl)-2′,2′-dimethyl-1, 3-oxazolidin-4′-ylmethyl]-3-methylbutyl ester: The title compound is obtained in a manner analogous to that described in Example 1c) in the form of a colourless oil (1.30 g), R_{f} (E)=0.33, starting from (2R,4′S,5′S,2″R)-3-[4′-(2″-hydroxymethyl-3″-methylbutyl)-3′-(tert-butoxycarbonyl)-2′,2′-dimethyl-1,3-oxazolidin-5′-yl]-2-methylpropionic acid N-(butyl)amide (1.10 g).

d) (2R,4′S,5′S,2″R)-3-[4′-(2″-Hydroxymethyl-3″-methylbutyl)-3′-(tert-butoxycarbonyl)-2′,2′-dimethyl-1,3-oxazolidin-5′-yl]-2-methylpropionic acid N-(butyl)amide: The title compound is obtained in the form of a white solid (1.16 g), R_{f} (hexane-ethyl acetate 1:3)=0.44; m.p. 110°-112° C., from (2R,4′S,5′S,2″R)-3-[4′-(2″-benzyloxymethyl-3″-methylbutyl)-3′-(tert-butoxycarbonyl)-2′,2′-dimethyl-1,3-oxazolidin-5′-yl]-2-methylpropionic acid N-(butyl)amide (1.64 g) by hydrogenation in a manner analogous to that described in Example 1d) and then purification by FC (50 g of silica gel, eluant gradient from E to ethyl acetate).

e) (2R,4′S,5′S,2″R)-3-[4′-(2″-Benzyloxymethyl-3″-methylbutyl)-3′-(tert-butoxycarbonyl)-2′,2′-dimethyl-1,3-oxazolidin-5′-yl]-2-methylpropionic acid N-(butyl)amide: A mixture of (2RS,4S,5S,7R)-7-benzyloxymethyl-5-(tert-butoxycarbonyl)amino-4-hydroxy-2,8-dimethyl-nonanoic acid N-(butyl)amide (1.9 g) and para-toluenesulfonic acid hydrate (0.037 g) in 2′,2′-dimethoxyprepane (35 ml) and dichloromethane (35 ml) is stirred at room temperature for one hour. Pyridine (16 .mu.l) and hexane (35 ml) are then added, the solvent is concentrated in vacuo, and the oily residue is chromatographed on 100 g of silica gel (eluant gradient from hexane-ethyl acetate 6:1 to 4:1). Fractions containing pure (2R,4′S,5′S,2″R)-diastereoisomer are combined, while mixed fractions are chromatographed again under the same conditions. Combination of the corresponding fractions, concentration of the solvent and drying of the residue under a high vacuum yield the title compound (1.64 g; R_{f} (E)=0.72; HPLC, Rₜ=22.8 min; MS(FAB) m/e 533 (M⁺+1)) together with a 6:4 mixture of the two (2R,4′S,5′S,2″R)- and (2R,4′S,5′S,2″S)-diastereoisomers (0.28 g; R_{f} (E)=0.72/0.66; HPLC, Rₜ=22.8 and 23.0 min).

f) (2RS,4S,5S,7R)-7-Benzyloxymethyl-5-(tert-butoxycarbonyl)amino-4-hydroxy-2,8-dimethyl-nonanoic acid N-(butyl)amide: In a manner analogous to that described in Example 1f), hydrogenation of the stereoisomerically pure (2S,3S,5R)-2-[3-(tert-butoxycarbonyl)amino-5-(benzyloxymethyl)-2-hydroxy-6-methylheptyl]-N-(butyl)acrylamide (A) (2.5 g, 5.10 mmol) in the presence of catalytic amounts of [Ru₂Cl₄[(S)-BlNAP]₂]NEt₃ (30 mg) yields the title compound in the form of a mixture of diastereoisomers (1.91 g) that cannot be separated on silica gel, the (2R,4S,5S,2′R)-isomer preferentially being formed: yellowish oil, R_{f} (E)=0.25, MS(FAB) m/e 493 (M⁺+1).

g) (2S,3S,5R)-2-[3-(tert-Butoxycarbonyl)amino-5-(benzyloxymethyl)-2-hydroxy-6-methylheptyl]-N-(butyl)acrylamide (A) and (2R,3S,5R)-2-[3-(tert-butoxycarbonyl)amino-5-(benzyloxymethyl)-2-hydroxy-6-methylheptyl]-N-(butyl)acrylamide (B): In a manner analogous to that described in Example 1g), there is obtained first, by reduction of (2S,4R)-2-(tert-butoxycarbonyl)amino-4-(benzyloxymethyl)-5-methyl-hexanoic acid methyl ester (8.0 g), dissolved in anhydrous toluene (120 ml), with a 1.2M diisobutylaluminium hydride solution in toluene (34.9 ml), (2S,4R)-2-(tert-butoxycarbonyl)amino-4-(benzyloxymethyl)-5-methyl-hexanal in the form of a pale-yellow oil (R_{f} (N)=0.6). The crude aldehyde is reacted in a manner analogous to that described in Example 1g) without being purified further. The crude product (14.8 g) obtained after aqueous working-up is chromatographed over 1.0 kg of silica gel using an eluant gradient from B to E, with separation of the two (2S,3S,5R)- and (2R,3S,5R)-diastereoisomers. There are obtained title compound A (2.52 g, 24%) in the form of a light-yellow oil, R_{f} (E)=0.55, HPLC Rₜ=19.8 min, MS(FAB) m/e 491 (M⁺+1), and title compound B (3.76 g, 35%) in the form of a light-yellow oil, R_{f} (E)=0.42, HPLC Rₜ=19.6 min. MS(FAB) m/e 491 (M⁺+1).

h) (2S,4R)-2-(tert-Butoxycarbonyl)amino-4-(benzyloxymethyl)-5-methyl-hexanoic acid methyl ester: In a manner analogous to that described in Examples 1i and 1j), (2S,2′R,5R)-2-[2′-(benzyloxymethyl)-3′-methylbutyl]-2,5-dihydro-5-isopropyl-3,6-dimethoxypyrazine (15.7 g) is first hydrolysed in the presence of 1N hydrochloric acid solution (176 ml) and then the product (R_{f} (W)=0.59) obtained after working-up and purification by FC (1.0 kg of silica gel, eluant V) is reacted with di-tert-butyl dicarbonate in the presence of Hunig base. Purification by FC (1.0 kg of silica gel, hexane-ethyl acetate 6:1) yields the title compound (13.4 g) in the form of a pale-yellow oil: R_{f} (C)=0.43.

i) (2S,2′R,5R)-2-[2′-(benzyloxymethyl)-3′-methylbutyl]-2,5-dihydro-5-isopropyl-3,6-dimethoxypyrazine: A solution of (2R)-2,5-dihydro-2-isopropyl-3,6-dimethoxypyrazine (13.0 g) in absolute tetrahydrofuran (230 ml) is reacted in a manner analogous to that described in Example 1k) first with a 1.6M n-butyllithium solution in hexane (44 ml) and then with 2(R)-(benzyloxymethyl)-3-methylbutyl bromide (12.8 g) in tetrahydrofuran (60 ml). After working up the reaction mixture, the oily crude product is purified by FC (1.0 kg of silica gel, ethyl acetate-hexane 1:20). There is obtained, in addition to the (2R,2′R,5R)-diastereoisomer (3.49 g; yellowish oil, R_{f} (ethyl acetate-hexane 1:6)=0.50), the stereoisomerically pure title compound (12.9 g) in the form of a yellowish oil: R_{f} (ethyl acetate-hexane 1:6)=0.62.

### Example 9

In a manner analogous to that described in Example 8), the following compounds are prepared:

a) From 100 mg of (2R,4′S,5′S,2″R)-3-[4′-(2″-aminomethyl-3″-methylbutyl)-3′-(tert-butoxycarbonyl)-2″,2′-dimethyl-1,3-oxazolidin-5′-yl]-2-methylpropionic acid N-(butyl)amide and 99 mg of 1-(2-methoxyethyl)-1H-indole-3-carboxylic acid, (2R,4′S,5′S,2″R)-1-(2-methoxyethyl)-1H-indole-3-carboxylic acid N-{2-[5′-(2″-butylcarbamoylpropyl)-2′,2′-dimethyl-1,3-oxazolidin-4′-ylmethyl]-3-methylbutyl}-amide, R_{f} (L)=0.68, in the form of a yellowish oil.

b) From 46 mg of (2R,4′S,5′S,2″R)-3-[4′-(2″-aminomethyl-3″-methylbutyl)-3′-(tert-butoxycarbonyl)-2′,2′-dimethyl-1,3-oxazolidin-5′-yl]-2-methylpropionic acid N-(butyl)amide and 53 mg of 1-pyridin-2-yl-1H-indole-3-carboxylic acid, (2S,4′S,5′S,2″R)-1-pyridin-2yl-1H-indole-3-carboxylic acid N-{2-[5′-(2″-butylcarbamoylpropyl)-2′,2′-dimethyl-oxazolidin-4′-ylmethyl]-3-methylbutyl}-amide, R_{f} (L)=0.81, in the form of a yellowish oil.

c) From 46 mg of (2R,4′S,5′S,2″R)-3-[4′-(2″-aminomethyl-3″-methylbutyl)-3′-(tert-butoxycarbonyl)-2′,2′-dimethyl-1,3-oxazolidin-5′-yl]-2-methylpropionic acid N-(butyl)amide and 59 mg of 1-(2-methoxybenzyl)-1H-indole-3-carboxylic acid, (2R,4′S,5′S,2″R)-1-(2-methoxybenzyl)-1H-indole-3-carboxylic acid N-{2-[5′-(2″-butylcarbamoylpropyl)-2′,2′-dimethyl-oxazolidin-4′-ylmethyl]-3-methylbutyl}-amide, R_{f} (L)=0.73, in the form of a colourless oil.

The 1-(2-methoxyethyl)-1H-indole-3-carboxylic acid used as starting material is prepared as follows:

a) 1-(2-Methoxyethyl)-1H-indole-3-carboxylic acid: 1N sodium hydroxide solution (5.1 ml) is added to a solution of 1-(2-methoxyethyl)-1H-indole-3-carboxylic acid ethyl ester (1.26 g) in ethanol (10 ml) and water (5 ml), and the mixture is heated at 50° C. for one hour, with stirring. Further 1N sodium hydroxide solution (7.1 ml) is added, and stirring is continued for a further 5 hours at 80° C. The mixture is concentrated in a rotary evaporator with removal of the ethanol, the aqueous phase is acidified by the addition of 1M potassium hydrogen sulfate solution, and extraction is carried out with dichloromethane. The organic phase is washed with saturated sodium chloride solution, dried over sodium sulfate and concentrated. The title compound (0.96 g), R_{f} (hexane-ethyl acetate-glacial acetic acid 67:33:1)=0.15, is obtained in the form of a yellowish solid.

b) 1-(2-Methoxyethyl)-1H-indole-3-carboxylic acid ethyl ester: Sodium hydride in the form of an 80% dispersion in oil (0.27 g) is added to a solution of 1H-indole-3-carboxylic acid ethyl ester (1.0 g) in N,N-dimethylformamide (25 ml), and the mixture is stirred at room temperature for 30 minutes. 3-Methoxyethyl iodide (1.5 g) is then added and the reaction mixture is stirred first for one hour at 50° C. and then overnight at 80° C. The mixture is poured into ice-water (50 ml), the aqueous phase is extracted with dichloromethane, and the combined organic phases are washed with saturated sodium chloride solution, dried over sodium sulfate and concentrated. The crude product is purified by FC (100 g of silica gel, eluant gradient from C to D). The title compound (1.26 g), R_{f} (C, double track)=0.44, is obtained in the form of an oil.

### Example 10

In a manner analogous to that described in Example 3), the following compounds are prepared:

a) From 126 mg of (2R,4′S,5′S,2″R)-1-benzyl-1H-indole-3-carboxylic acid N-{2-[5′-(2″-butylcarbamoylpropyl)-2′,2′-dimethyl-1,3-oxazolidin-4′-ylmethyl]-3-methylbutyl}-amide, (2R,4S,5S,7R)-1-benzyl-1H-indole-3-carboxylic acid N-[4-(tert-butoxycarbonyl)amino-7-butylcarbamoyl-5-hydroxy-2-isopropyl-octyl]-amide, R_{f} (L)=0.55, in the form of a white foam.

b) From 146 mg of (2R,4′S,5′S,2″R)-1-(2-methoxyethyl)-1H-indole-3-carboxylic acid N-{2-[5′-(2″-butylcarbamoylpropyl)-2′,2′-dimethyl-1,3-oxazolidin-4′-ylmethyl]-3-methylbutyl}-amide, (2R,4S,5S,7R)-1-(2-methoxyethyl)-1H-indole-3-carboxylic acid N-[4-(tert-butoxycarbonyl)-amino-7-butylcarbamoyl-5-hydroxy-2-isopropyl-octyl]-amide, R_{f} (L)=0.52, in the form of a colourless oil.

c) From 77 mg of (2R,4′S,5′S,2″R)-1-pyridin-2-yl-1H-indole-3-carboxylic acid N-{2-[5′-(2″-butylcarbamoylpropyl)-2′,2′-dimethyl-1,3-oxazolidin-4′-ylmethyl]-3-methylbutyl}-amide, with purification by FC on 10 g of silica gel (eluant S), (2R,4S,5S,7R)-1-pyridin-2-yl-1H-indole-3-carboxylic acid N-[4-(tert-butoxycarbonyl)amino-7-butylcarbamoyl-5-hydroxy-2isopropyl-octyl]-amide, R_{f} (W)=0.54, in the form of a colourless foam.

d) From 67 mg of (2R,4′S,5′S,2″R)-1-(2-methoxybenzyl)-1H-indole-3-carboxylic acid N-{2-[5′-(2″-butylcarbamoylpropyl)-2′,2′-dimethyl-1,3-oxazolidin-4′-ylmethyl]-3-methylbutyl}-amide, (2R,4S,5S,7R)-1-(2-methoxybenzyl)-1H-indole-3-carboxylic acid N-[4-(tert-butoxycarbonyl)amino-7-butylcarbamoyl-5-hydroxy-2-isopropyl-octyl]-amide, R_{f} (L)=0.61, in the form of a colourless foam.

### Example 11

In a manner analogous to that described in Example 5), the following compounds am prepared by de-Bocylation:

a) From 93 mg of (2R,4S,5S,7R)-1-benzyl-1H-indole-3-carboxylic acid N-[4-(tert-butoxycarbonyl)amino-7-butylcarbamoyl-5-hydroxy-2-isopropyl-octyl]-amide, (2R,4S,5S,7R)-1-benzyl-1H-indole-3-carboxylic acid N-(4-amino-7-butylcarbamoyl-5-hydroxy-2-isopropyl-octyl)-amide hydrochloride: R_{f} (W)=0.32. HPLC Rₜ=13.8 min. MS(FAB) m/e 535 (M⁺+1).

b) From 89 mg of (2R,4S,5S,7R)-1-(2-methoxyethyl)-1H-indole-3-carboxylic acid N-[4-(tert-butoxycarbonyl)amino-7-butylcarbamoyl-5-hydroxy-2-isopropyl-octyl]-amide, (2R,4S,5S,7R)-1-(2-methoxyethyl)-1H-indole-3-carboxylic acid N-(4-amino-7-butylcarbamoyl-5-hydroxy-2-isopropyl-octyl)-amide hydrochloride: R_{f} (W)=0.29. HPLC Rₜ=11.7 min. MS(FAB) m/e 503 (M⁺+1).

c) From 60 mg of (2R,4S,5S,7R)-1-pyridin-2-yl-1H-indole-3-carboxylic acid N-[4-(tert-butoxycarbonyl)amino-7-butylcarbamoyl-5-hydroxy-2-isopropyl-octyl]-amide, (2R,4S,5S,7R)-1-pyridin-2-yl-1H-indole-3-carboxylic acid N-(4-amino-7-butylcarbamoyl-5-hydroxy-2-isopropyl-octyl)-amide hydrochloride: R_{f} (W)=0.24. HPLC Rₜ=9.94 min. MS(FAB) m/e 536 (M⁺+4).

d) From 50 mg of (2R,4S,5S,7R)-1-(2-methoxybenzyl)-1H-indole-3-carboxylic acid N-[4-(tert-butoxycarbonyl)amino-7butylcarbamoyl-5-hydroxy-2-isopropyl-octyl]-amide, (2R,4S,5S,7R)-1H-indole-3-carboxylic acid N-(4-amino-7-butylcarbamoyl-5-hydroxy-2-isopropyl-octyl)-amide hydrochloride: R_{f} (W)=0.32. HPLC Rₜ=14.0 min. MS(FAB) m/e 565 (M⁺+1).

### Example 12

In a manner analogous to that described in Example 5), (2R,4S,5S,7R)-N-(4-amino-7-butylcarbamoyl-5-hydroxy-2-isopropyl-octyl)-2-(3-methoxypropoxy)-benzamide hydrochloride, R_{f} (W)=0.28, HPLC Rₜ=11.9 min, MS(FAB) m/e 494 (M⁺+1), is obtained from (2R,4S,5S,7R)-N-[4-(tert-butoxycarbonyl)amino-7-butylcarbamoyl-5-hydroxy-2-isopropyl-octyl]-2-(3-methoxypropoxy)-benzamide (93 mg).

The (2R,4S,5S,7R)-N-[4-(tert-butoxycarbonyl)amino-7-butyl carbamoyl-5-hydroxy-2-isopropyl-octyl]-2-(3-methoxypropoxy)-benzamide used as starting material is prepared as follows:

a) In accordance with the process described in Example 3), (2R,4S,5S,7R)-N-[4-(tert-butoxycarbonyl)amino-7-butylcarbamoyl-5-hydroxy-2-isopropyl-octyl]-2-(3-methoxypropoxy)-benzamide, R_{f} (N)=0.28, is obtained in the form of a colourless oil from 135 mg of (2R,4′S,5′S,2″R)-N-{2-[5′-(2″-butylcarbamoylpropyl)-3′-(tert-butoxycarbonyl)-2′,2′-dimethyl-1,3-oxazolidin-4′-ylmethyl]-3-methylbutyl}-2-(3-methoxypropoxy)-benzamide.

b) (2R,4′S,5′S,2″R)-N-{2-[5′-(2″-Butylcarbamoylpropyl)-3′-(tert-butoxycarbonyl)-2′,2′-dimethyl-1,3-oxazolidin-4′-ylmethyl]-3-methylbutyl}-2-(3-methoxypropoxy)-benzamide: In accordance with the process described in Example 1), the title compound, R_{f} (M)=0.57, is obtained in the form of a pale-yellow oil from 100 mg of (2R,4′S,5′S,2″R)-3-[4′-(2″-aminomethyl-3″-methylbutyl)-3′-(tert-butoxycarbonyl)-2′,2′-dimethyl-1,3-oxazolidin-5′-yl]-2-methylpropionic acid N-(butyl)amide and 95 mg of 2-(3-methoxypropoxy)-benzoic acid.

### Example 13

Hydrogenolysis of (2R,4′S,5′S,2″R)-3-[4′-(3″-azido-2″-methylpropyl)-3′-(tert-butoxycarbonyl)-2′,2′-dimethyl-1,3-oxazolidin-5′-yl]-2-methylpropionic acid N-(butyl)amide (0.67 g) in a manner analogous to that described in Example 1a) yields (2R,4′S,5′S,2″R)-3-[4′-(3″-amino-2″-methylpropyl)-3′-(tert-butoxycarbonyl)-2′,2′-dimethyl-1,3-oxazolidin-5′-yl]-2-methylpropionic acid N-(butyl)amide in the form of a pale-yellow oil: R₁ (W)=0.33.

The (2R,4′S,5′-S,2″R)-3-[4′-(3″-azido-2″-methylpropyl)-3′-(tert-butoxycarbonyl)-2′,2′-dimethyl-1,3-oxazolidin-5′-yl]-2-methylpropionic acid N-(butyl)amide used as starting material is prepared as follows:

a) (2R,4′S,5′S,2″R)-3-[4′-(3″-Azido-2″-methylpropyl)-3′-(tert-butoxycarbonyl)-2′,2′-dimethyl-1,3-oxazolidin-5′-yl]-2-methylpropionic acid N-(butyl)amide: The title compound (0.86 g), R_{f} (E)=0.71, is obtained in the form of a p ale-yellow oil from (2R,4′S,5′S,2″R)-3-[3′-(tert-butoxycarbonyl)-4′-(3″-hydroxy-2″-methylpropyl)-2′,2′-dimethyl-1,3-oxazolidin-5′-yl]-2-methylpropionic acid N-(butyl)amide (0.85 g) in a manner analogous to that described in Examples 1c) and 1b) and after purification by FC (100 g of silica gel, ethyl acetate-hexane 3:1).

b) (2R,4′S,5′S,2″R)-3-[3′-(tert-Butoxycarbonyl)-4′-(3″-hydroxy-2″-methylpropyl)-2′,2′-dimethyl-1,3-oxazolidin-5′-yl]-2-methylpropionic acid N-(butyl)amide: In a manner analogous to that described in Examples 1d), 1e) and 1f), the stereoisomerically pure title compound: colorless oil (1.46 g; purification of the crude alcohol by FC on 50 g of silica gel, eluant gradient from D to hexane-ethyl acetate 1:3), R_{f} (hexane-ethyl acetate 1:3)=0.29, is obtained by (1) stereoselective hydrogenation of (2S,3S,5R)-2-[6-(benzyloxymethyl)-3-(tert-butoxycarbonyl)amino-2-hydroxy-5-methylhexyl]-N-(butyl)acrylamide (A) (3.17 g) in absolute methanol in the presence of [Ru₂Cl₄[(S)-BINAD]₂]NEt₃ (0.057 g), followed by (2) N,O-acetalisation by reaction with 2′,2′-dimethoxypropane in the presence of para-toluenesulfonic acid and chromatographic separation of the resulting approximately 9:1 diastereoisomeric mixture of (2R,4′S,5′S,2″R)-3-[4′-(3″-benzyloxy-2″-methylpropyl)-3′-(tert-butoxycarbonyl)-2′,2′-dimethyl-1,3-oxazolidin-5′-yl]-2-methylpropionic acid N-(butyl)amide (R_{f} (E)=0.64) and (2S,4′S,5′S,2″R)-3-[4′-(3″-benzyloxy-2″-methylpropyl)-3′-(tert-butoxycarbonyl)-2′,2′-dimethyl-1,3-oxazolidin-5′-yl]-2-methylpropionic acid N-(butyl)amide (R_{f} (E)=0.54) by FC on 100 g of silica gel (eluant gradient from hexane-ethyl acetate 5:1 to ethyl acetate), and then (3) hydrogenolysis of the resulting (2R,4′S,5′S,2″R)-3-[4′-(3″-benzyloxy-2″-methylpropyl)-3′-(tert-butoxycarbonyl)-2′,2′-dimethyl-1,3-oxazolidin-5′-yl]-2-methylpropionic acid N-(butyl)amide on 10% palladium-on-carbon.

c) (2S,3S,5R)-2-[6-Benzyloxy-3-(tert-butoxycarbonyl)amino-2-hydroxy-5-methylhexyl]-N-(butyl)acrylamide (A) and (2R,3S,5R)-2-[6-benzyloxy-3-(tert-butoxycarbonyl)amino-2-hydroxy-5-methylhexyl]-N-(butyl)acrylamide (B): In a manner analogous to that described in Example 1h), there is obtained first, by reduction of (2S,4R)-5-benzyloxy-2-(tert-butoxycarbonyl)amino-4-methylpentanoic acid methyl ester (9.51 g) in the presence of a 1.2M diisobutylaluminium hydride solution in toluene, (2S,4R)-5-benzyloxy-2-(tert-butoxycarbonyl)amino-4-methyl-pentanal (8.2 g of crude product) in the form of a pale-yellow oil. The crude aldehyde is reacted in a manner analogous to that described in Example 1g) without being purified further. The crude product (17.1 g) so obtained comprises an approximately 1:1.45 mixture of the two diastereoisomers A and B. Purification by column chromatography with separation of the two stereoisomers on silica gel (0.6 kg, eluant gradient from C to E) yields title compound A (2.05 g) in the form of a waxy solid, R_{f} (E)=0.43, MS(FAB) m/e 463 (M⁺+1), and title compound B (3.01 g) in the form of a light-yellow oil, R_{f} (E)=0.35, MS(FAB) m/e 463 (M⁺+1)

d) (2S,4R)-5-benzyloxy-2-(tert-butoxycarbonyl)amino-4-methylpentanoic acid methyl ester: In a manner analogous to that described in Examples 1i and 1j), (2S,2′R,5R)-2-(3′-benzyloxy-2′-methylpropyl)-2,5-dihydro-5-isopropyl-3,6-dimethoxypyrazine (18.4 g) is first hydrolysed in the presence of 1N hydrochloric acid solution (215 ml) and then reacted with di-tert-butyl dicarbonate in the presence of Hunig base. Purification by FC (0.9 kg of silica gel, eluant A) yields the title compound (16.0 g) in the form of a colorless oil: R_{f} (C)=0.54.

e) (2S,2R,5R)-2-(3-benzyloxy-2-methylpropyl)-2,5-dihydro-5-isopropyl-3,6-dimethoxypyrazine: A solution of (2R)-2,5-dihydro-2-isopropyl-3,6-dimethoxypyrazine (14.7 ml) in absolute tetrahydrofuran (230 ml) is reacted in a manner analogous to that described in Example 1k) first with a 1.6M n-butyllithium solution in hexane (47.4 ml) and then with 2(S)-3-benzyloxy-2-methylpropyl bromide (20.0 g) in tetrahydrofuran (115 ml). After working up the reaction mixture, the oily crude product is purified by FC (0.9 kg of silica gel, ethyl acetate-hexane 5:95). There is obtained in addition to the (2R,2′R,5R)-diastereoisomer (1.1 g; yellowish oil, R_{f} (ethyl acetate-hexane 1:6)=0.27), the stereoisomerically pure title compound (18.4 g) in the form of a yellowish oil: R_{f} (ethyl acetate-hexane 1:6)=0.33.

### Example 14

In a manner analogous to that described in Example 5), (2R,4S,5S,7R)-N-(4-amino-7-butylcarbamoyl-5-hydroxy-2-methyl-octyl)-2-(3-methoxypropoxy)-benzamide hydrochloride, R_{f} (W)=0.25, HPLC Rₜ=9.0 min, MS(FAB) m/e 466 (M⁺+1), is obtained from (2R,4S,5S,7R)-N-[4-(tert-butoxycarbonyl)amino-7-butylcarbamoyl-5-hydroxyl-2-methyloctyl]-2-(3-methoxypropoxy)-benzamide (82 mg).

The (2R,4S,5S,7R)-N-[4-(tert-butoxycarbonyl)amino-7-butylcarbamoyl-5-hydroxy-2-methyloctyl]-2-(3-methoxypropoxy)-benzamide used as starting material is prepared as follows:

a) In accordance with the process described in Example 3), (2R,4S,5S,7R)-N-[4-(tert-butoxycarbonyl)amino-7-butylcarbamoyl-5-hydroxy-2-methyloctyl]-2-(3-methoxypropoxy)-benzamide, R_{f} (L)=0.36, is obtained in the form of a yellowish oil from 134 mg of (2R,4′S,5′S,2″R)-N-{3-[5′-(2″-butylcarbamoylpropyl)-3′-(tert-butoxycarbonyl)-2′,2′-dimethyl-1,3-oxazolidin-4′-yl]-2-methylpropyl}-2-(3-methoxypropoxy)-benzamide.

b) (2R,4′S,5′S,2″R)-N-{3-[5′-(2″-Butylcarbamoylpropyl)-3′-(tert-butoxycarbonyl)-2′,2′-dimethyl-1,3-oxazolidin-4′-yl]-2-methylbutyl}-2-(3-methoxypropoxy)-benzamide: In accordance with the process described in Example 1), the title compound, R_{f} (L)=0.43, is obtained in the form of a pale-yellow oil from 100 mg of (2R,4′S,5′S,2″R)-3-[4′-(3″-amino-2″-methylpropyl)-3′-(tert-butoxycarbonyl)-2′,2′-dimethyl-1,3-oxazolidin-5′-yl]-2-methylpropionic acid N-(butyl)amide and 102 mg of 2-(3-methoxypropoxy)-benzoic acid.

### Example 15

A mixture of 2-(3-methoxypropoxy)-benzoic acid (1.7 g), biS(2-oxo-3-oxazolidinyl)phosphinic acid chloride (1.90 g) and triethylamine (2.81 ml) in dichloromethane (40 ml) is stirred at room temperature for 60 minutes. Then a solution of (3S,5S,1S′,3′S)-5-[3′-aminomethyl-1′-(tert-butoxycarbonyl)amino-4′-methylpentyl]-3-isopropyl-dihydrofuran-2-one (1.80 g) in 40 ml of dichloromethane and 4-dimethylaminopyridine (380 mg) are added. The reaction mixture is stirred overnight. After the addition of dichloromethane (200 ml), the organic phase is washed in succession with dilute sodium hydroxide solution (pH 9), dilute aqueous hydrochloric acid and saturated sodium chloride solution, dried over magnesium sulfate and concentrated. FC on silica gel (eluant R) yields (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(3-methoxypropoxy)-benzamide in the form of a pale-yellow oil (2.70 g): R_{f} (E)=0.30. MS(FAB) m/e 563 (M⁺+1)

The (3S,5S,1S′,3′S)-5-[3′-aminomethyl-1′-(tert-butoxycarbonyl)amino-4′-methylpentyl]-3-isopropyl-dihydrofuran-2-one used as starting material is prepared as follows:

a) (3S,5S,1-S,3′S)-5-[3′-Azidomethyl-1′-(tert-butoxycarbonyl)amino-4′-methylpentyl]-3-isopropyl-dihydrofuran-2-one (12.4 g), dissolved in ethyl acetate (500 ml), is hydrogenated for 3 hours at room temperature and under normal pressure in the presence of 10% Pd/C (2.5 g). Filtration over Hyflo® and removal of the solvent yield the title compound in the form of a white solid (11.3 g): R_{f} (W)=0.34. M.p. 136°-138° C. (recrystallised from dichloromethane-hexane).

b) (3S,5S,1′S,3′S)-5-[3′-Azidomethyl-1′-(tert-butoxycarbonyl)amino-4′-methylpentyl]-3-isopropyl-dihydrofuran-2-one: A mixture of (2S,2′S,2″S,4″S)-methanesulfonic acid N-(tert-butoxycarbonyl)-2-[2′-amino-2′-(4″-isopropyl-5″-oxo-tetrahydrofura n-2″-yl)-ethyl]-3-methylbutyl ester (30.2 g) and sodium azide (22.5 g) in 1,3-dimethyl-3,4,5,6-tetrahydro-2(1H)-pyrimidinone (290 ml) is stirred overnight at 50° C. After cooling the reaction mixture, dichloromethane (650 ml) is added and the organic phase is washed with water at pH 8 (140 ml) and with saturated sodium chloride solution, dried over magnesium sulfate and concentrated. FC of the crude product on silica gel (2 kg, eluant B) yields the title compound in the form of a white solid (23.6 g): R_{f} (C)=0.36. M.p. 78°-81° C. MS(FAB) m/e 383 (M⁺+1).

c) (2S,2′S,2″S,4″S)-Methanesulfonic acid N-(tert-butoxycarbonyl)-2-[2′-amino-2′-(4″-isopropyl-5″-oxo-tetrahydrofuran-2″-yl)-ethyl]-3-methylbutyl ester: To a solution of (3S,5S,1′S,3′S)—N-(tert-butoxycarbonyl)-5-(1′-amino-3′-hydroxymethyl-4′-methylpentyl)-3-isopropyl-dihydrofuran-2-one (24.8 g) in dichloromethane (750 ml) there are added with stirring at −10° C. first triethylamine (14.5 ml) and then, over a period of 10 minutes, methanesulfonyl chloride (5.64 ml). After stirring for a further 30 minutes at −10° C., the reaction mixture is poured carefully onto ethyl acetate (1 liter). The organic phase is washed in succession with 0.5M aqueous H₃PO₄ solution, saturated sodium hydrogen carbonate solution and saturated sodium chloride solution, dried over magnesium sulfate and concentrated. The title compound is obtained in the form of a crude product (31.1 g, colourless oil), which slowly crystallises out when left to stand. R_{f} (E)=0.63.

d) (3S,5S,1′S,3′S)-N-(tert-butoxycarbonyl)-5-(1′-amino-3′-hydroxymethyl-4′-methylpentyl)-3-isopropyl-dihydrofuran-2-one: A solution of (3S,5S,1′S,3′S)-5-(1′-azido-3′-hydroxymethyl-4′-methylpentyl)-3-isopropyl-dihydrofuran-2-one (24.8 g) in ethyl acetate (250 ml) is hydrogenated for 24 hours at room temperature and under normal pressure in the presence of 10% Pd/C (8.68 g). Filtration is carried out over Hyflo®, followed by repeated washing with ethyl acetate and concentration. The resulting crude (3S,5S,1′S,3′S)-5-(1′-amino-3′-hydroxymethyl-4′-methylpentyl)-3-isopropyl-dihydrofuran-2-one (23.0 g; colourless oil, R_{f} (W)=0.67) is dissolved in ethyl acetate (500 ml) and there are added thereto at 0°-5°, with stirring, first N-ethyldiisopropylamine (23.7 ml) and then, dropwise, a solution of di-tert-butyl dicarbonate (21.0 g) in ethyl acetate (100 ml). After warming to room temperature, stirring is continued overnight. The reaction mixture is concentrated and the oily residue is purified by FC (250 g of silica gel, eluant D). The title compound (24.9 g) is obtained in the form of a white solid: R_{f} (dichloromethane-methanol 1:1)=0.64. M.p. 126°-128° C. (diethyl ether). MS(FAB) m/e 358 (M⁺+1).

e) (3S,5S,1′S,3′S)-5-(1′-Azido-3′-hydroxymethyl-4′-methylpentyl)-3-isopropyl-dihydrofuran-2-one: Triethylamine (5.62 ml) and chloroformic acid methyl ester (2.59 ml) are added dropwise in succession, at −10° C., to a solution of (2S,2′S,2″S,4″S-[2′-azido-2′-(4″-isopropyl-5″-oxo-tetrahydrofuran-2″-yl)-ethyl]-3-methyl-butyric acid (8.0 g) in anhydrous tetrahydrofuran (180 ml). The white suspension is then stirred first at −10° C. for one hour and then at 0° for 2 hours. The mixture is diluted with ethyl ace (100 ml) and the organic phase is washed in succession with ice-cold 0.5N hydrochloric acid, saturated sodium hydrogen carbonate solution and water, dried over sodium sulfate and concentrated. The pale-yellow oily residue is taken up in tetrahydrofuran (160 ml), and sodium borohydride (1.12 g) is added in portions at −20°, with stirring. Then methanol (1.5 ml) is added dropwise over a period of 10 minutes (slightly exothermic reaction). The slightly cloudy mixture is allowed to warm slowly to 0°-5° and is stirred overnight at that temperature, and then 1N hydrochloric acid (39 ml) is added dropwise and the aqueous phase is extracted with ethyl acetate (100 ml). The organic phase is washed until neutral with ice-cold 1N sodium carbonate solution (70 ml) and then with saturated sodium chloride solution, dried over magnesium sulfate and concentrated. Drying under a high vacuum yields the title compound in the form of a pale-yellow oil (7.18 g). Analytically pure product (pale-yellow oil) is obtained after flash chromatography on silica gel (eluant gradient hexane-ethyl acetate from 5:1 to 3:1): R_{f} (hexane-ethyl acetate 1:1)=0.50.

### Example 16

A mixture of 2-(4-methoxybutoxy)-benzoic acid (1.7 g), biS(2-oxo-3-oxazolidinyl)phosphinic acid chloride (1.90 g) and triethylamine (2.81 ml) in dichloromethane (40 ml) is stirred at room temperature for 60 minutes. Then a solution of (3S,5S,1S′,3′S)-5-[3′-aminomethyl-1′-(tert-butoxycarbonyl)amino-4′-methylpentyl]-3-isopropyl-dihydrofuran-2-one (1.80 g) in dichloromethane (40 ml) and 4-dimethylaminopyridine (380 mg) are added, and the reaction mixture is stirred overnight. After the addition of dichloromethane (200 ml), the organic phase is washed in succession with dilute sodium hydroxide solution (pH 9), dilute aqueous hydrochloric acid and saturated sodium chloride solution, dried over magnesium sulfate and concentrated. FC on silica gel (eluant R) yields (2S,2′S,2″S,4″S)-N-{2-[(2′-(tert-butoxycarbonyl)amino-2′-(4″-isopropyl-5″-oxo-tetrahydrofuran-2″-yl)-ethyl]-3-methylbutyl}-2-(4-methoxybutoxy)-benzamide in the form of a pale-yellow oil (2.70 g). R_{f} (E)=0.30. MS(FAB) m/e 563 (M⁺+1).

The 2-(4-methoxybutoxy)-benzoic acid used as starting material is prepared as follows:

a) In a manner analogous to that described in Example 1), 2-(4-methoxybutoxy)-benzoic acid ethyl ester (4.35 g) is hydrolysed with 1N sodium hydroxide solution (17.3 ml) in a 2:1 mixture of ethanol and water (30 ml). When the reaction is complete, dichloromethane (30 ml) is added and the aqueous phase is acidified by the addition of a 1M potassium hydrogen sulfate solution and extracted with dichloromethane (3×40 ml). The organic phase is washed with saturated sodium chloride solution, dried over magnesium sulfate and concentrated. The title compound, R_{f} (E)=0.39, is obtained in the form of a colourless oil, which crystallises out when left to stand.

b) 2-(4-Methoxybutoxy)-benzoic acid ethyl ester: A solution of 4-methoxybutyl bromide (4.5 g) in acetonitrile (15 ml) is added dropwise under reflux to a mixture of salicylic acid ethyl ester (2.63 ml), powdered potassium carbonate (3.10 g) and potassium iodide (10 mg) in acetonitrile (50 ml), and the reaction mixture is then stirred overnight. After cooling, filtration is carried out, the filtrate is concentrated and the residue is added under a high vacuum. The title compound (4.4 g), R_{f} (C)=0.28, is obtained in the form of a pale-yellow oil.

### Example 17

In a manner analogous to that described in Example 15) and with subsequent purification by FC on silica gel (eluant C or D), unless otherwise described in greater detail below, the following compounds are prepared:

a) From 80 mg of (3S,5S,1′S,3′S)-5-[3′-aminomethyl-1′-(tert-butoxycarbonyl)amino-4′-methylpentyl]-3-isopropyl-dihydrofuran-2-one and 81 mg of 2-propoxybenzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-propoxy-benzamide, R_{f} (E)=0.39, in the form of a yellowish oil.

b) From 100 mg of (3S,5S,1′S,3′S)-5-[3′-aminomethyl-1′-(tert-butoxycarbonyl)amino-4′-methylpentyl]-3-isopropyl-dihydrofuran-2-one and 110 mg of 2-(2-methoxyethoxy)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(2-methoxyethoxy)-benzamide, R_{f} (E)=0.28, in the form of a colorless oil.

c) From 100 mg of (3S,5S,1′S,3′S)-5-[3′-aminomethyl-1′-(tert-butoxycarbonyl)amino-4′-methylpentyl]-3-isopropyl-dihydrofuran-2-one and 102 mg of 2-(methoxymethoxy)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(methoxymethoxy)-benzamide, R_{f} (E)=0.40, in the form of a yellowish solid.

d) From 100 mg of (3S,5S,1′S,3′S)-5-[3′-aminomethyl-1′-(tert-butoxycarbonyl)amino-4′-methylpentyl]-3-isopropyl-dihydrofuran-2-one and 135 mg of 2-[2-(2-methoxyethoxy)-ethoxy]-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(2-methoxyethoxy)-ethoxy]-benzamide, R_{f} (E)=0.20, in the form of a yellowish oil.

e) From 100 mg of (3S,5S,1′S,3′S)-5-[3′-aminomethyl-1′-(tert-butoxycarbonyl)amino-4′-methylpentyl]-3-isopropyl-dihydrofuran-2-one and 135 mg of 4-methoxy-2-(3-methoxypropoxy)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-4-methoxy-2-(3-methoxypropoxy)-benzamide, R_{f} (L)=0.80, in the form of a yellowish oil.

f) From 100 mg of (3S,5S,1′S,3′S)-5-[3′-aminomethyl-1′-(tert-butoxycarbonyl)amino-4′-methylpentyl]-3-isopropyl-dihydrofuran-2-one and 135 mg of 4-methoxy-3-(3-methoxypropoxy)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-4-methoxy-3-(3-methoxypropoxy)-benzamide, R_{f} (L)=0.71, in the form of a pale-yellow oil.

g) From 100 mg of (3S,5S,1′S,3′S)-5-[3′-aminomethyl-1′-(tert-butoxycarbonyl)amino-4′-methylpentyl]-3-isopropyl-dihydrofuran-2-one and 109 mg of 2-(propoxymethyl)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(propoxymethyl)-benzamide, R_{f} (E)=0.46, in the form of a yellowish oil.

h) From 80 mg of (3S,5S,1S′,3′S)-5-[3′-aminomethyl-1′-(tert-butoxycarbonyl)amino-4′-methylpentyl]-3-isopropyl-dihydrofuran-2-one and 101 mg of 2-[2-(methoxymethoxy)-ethoxy]-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(methoxymethoxy)-ethoxy]-benzamide, R_{f} (E)=0.38, in the form of a pale-yellow oil.

i) From 50 mg of (3S,5S,1S′,3′S)-5-[3′-aminomethyl-1′-(tert-butoxycarbonyl)amino-4′-methylpentyl]-3-isopropyl-dihydrofuran-2-one and 75 mg of 2-acetamido-benzoic acid (reaction at room temperature for 48 hours and then at 50° C. for 12 hours), with subsequent purification of the crude product by FC on 20 g of silica gel (eluant D), (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-acetamido-benzamide, R_{f} (E)=0.25, in the form of a yellowish oil.

j) From 120 mg of (3S,5S,1S′,3′S)-5-[3′-aminomethyl-1′-(tert-butoxycarbonyl)amino-4′-methylpentyl]-3-isopropyl-dihydrofuran-2-one and 143 mg of 2-(3-methoxypropoxy)-nicotinic acid, with subsequent purification of the crude product by FC on 30 g of silica gel (eluant S), (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(3-methoxypropoxy)-nicotinamide, R_{f} (W)=0.77, in the form of a yellow oil.

k) From 120 mg of (3S,5S,1S′,3′S)-5-[3′-aminomethyl-1′-(tert-butoxycarbonyl)amino-4′-methylpentyl]-3-isopropyl-dihydrofuran-2-one and 151 mg of 3-(4-methoxybutoxy)-pyridine-2-carboxylic acid, with subsequent purification of the crude product by FC on 30 g of silica gel (eluant T), (2S,2′S,2′S,4″S)-N-{2-[2′-(tert-butoxycarbonyl)amino-2′-(4″-isopropyl-5″-oxo-tetrahydrofuran-2″-yl)-ethyl]-3-methylbutyl}-3-(4-methoxybutoxy)-pyridine-2-carboxylic acid amide, R_{f} (W)=0.70, in the form of a yellow oil.

l) From 50 mg of (3S,5S,1′S,3′S)-5-[3′-aminomethyl-1′-(tert-butoxycarbonyl)amino-4′-pentyl]-3-isopropyl-dihydrofuran-2-one and 63 mg of 2-[2-(acetamido)-ethoxy]-benzoic acid, with subsequent purification of the crude product on 10 g of silica gel (eluant T), (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(acetamido)-ethoxy]-benzamide, R_{f} (W)=0.65, in the form of an oil.

m) From 100 mg of (3S,5S,1′S,3′S)-5-[3′-aminomethyl-1′-(tert-butoxycarbonyl)amino-4′-methylpentyl]-3-isopropyl-dihydrofuran-2-one and 125 mg of 2-(4-methoxybutyl-2-enoxy)-benzoic acid, with subsequent purification of the crude product on 10 g of silica gel (eluant S), (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutyl-2-enoxy)-benzamide, R_{f} (W)=0.79, in the form of an oil.

n) From 100 mg of (3S,5S,1′S,3′S)-5-[3′-aminomethyl-1′-(tert-butoxycarbonyl)amino-4′-methylpentyl]-3-isopropyl-dihydrofuran-2-one and 137 mg of 2-(4-methoxybutoxy)-4-methyl-benzoic acid, with subsequent purification of the crude product on 25 g of silica gel (eluant S), (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-methyl-benzamide, R_{f} (W)=0.81, in the form of a pale-yellow oil.

o) From 10 mg of (3S,5S,1′S,3′S)-5-[3′-aminomethyl-1′-(tert-butoxycarbonyl)amino-4′-methylpentyl]-3-isopropyl-dihydrofuran-2-one and 134 mg of 2-(5-methoxypentoxy)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(5-methoxypentoxy)-benzamide, R_{f} (L)=0.79, in the form of a pale-yellow oil.

Unless otherwise described in greater detail below, the benzoic acid derivatives used as starting materials are prepared from corresponding precursors in a manner analogous to that described in Examples 1), 2), 16) and 50) or are obtained in accordance with standard general procedures.

A) 2-[2-(2-(Methoxyethoxy)-ethoxy]-benzoic acid ethyl ester: 2-[2-Methoxyethoxy]-ethyl bromide (2.25 g) and a catalytic amount of potassium iodide (20 mg) are added at 85° C. to a suspension of salicylic acid ethyl ester (1.86 g) and potassium carbonate powder (1.85 g) in anhydrous N,N-dimethylformamide (50 ml). The mixture is stirred overnight at 85° C. and, after cooling, is filtered and concentrated. Purification by FC (100 g of silica gel, eluant C) yields a pale-yellow oil (2.89 g): R_{f} (D)=0.29.

B) 2-Propoxymethyl-benzoic acid: The title compound is obtained in the form of a pale-yellow solid, R_{f} (hexane-ethyl acetate-glacial acetic acid 50:50:1)=0.63; MS(EI) m/e 194 (M.sup.+), from 2-propoxymethyl-benzoic acid propyl ester, by alkaline hydrolysis.

The 2-propoxymethyl-benzoic acid propyl ester used as starting material is prepared as follows:

a) Sodium hydride in the form of an 80% dispersion in oil (0.38 g) is added at room temperature, with stirring, to a solution of potassium 2-(hydroxymethyl)-benzoate (3.0 g), prepared in accordance with the procedure described in J. Am. Chem. Soc. (1989), 111, 1465-1473, in anhydrous N,N-dimethylformamide (20 ml). After stirring for 30 minutes, propyliodide (8.05 g) is added dropwise, the mixture is heated to 80° C., and stirring is continued for 20 hours. After cooling to room temperature, the reaction mixture is poured onto ice-water (50 ml) and the aqueous phase is extracted with diethyl ether (3×40 ml). The organic phase is dried over magnesium sulfate and concentrated. Purification by FC (80 g of silica gel, eluant B) yields 2-propoxymethyl-benzoic acid propyl ester (0.69 g), R_{f} (D)=0.30, in the form of a yellow oil.

C) 2-[2-(Methoxymethoxy)-ethoxy]-benzoic acid ethyl ester: 2-(2-Methoxymethoxy)-ethyl chloride (5.62 g), dissolved in acetone (30 ml), and potassium iodide (4.5 g) are added to a mixture of salicylic acid ethyl ester (4.43 ml) and potassium carbonate powder (4.99 g) in anhydrous acetone (50 ml) and anhydrous dimethyl sulfoxide (100 ml). The mixture is stirred at 70° C. for two days. After cooling, the suspension is filtered, the filtrate is concentrated, and the residue is purified by FC (400 g of silica gel, hexane-ethyl acetate 5:1). The title compound is obtained in the form of a yellowish oil (3.8 g), R_{f} (C)=0.35, which contains small amounts of an unidentified secondary product.

D) 2-(4-Methoxybut-2-enoxy)-benzoic acid methyl ester: A 30% methanolic sodium methoxide solution (8.83 ml) is added dropwise at 60° C. over a period of 30 minutes to 2-(4-bromo-but-2-enoxy)-benzoic acid methyl ester (12.1 g) in absolute methanol (70 ml), and the mixture is stirred for 5 hours. Customary working-up and purification by FC (hexane-ethyl acetate 8:1) yield the title compound in the form of a pale-yellow oil (6.77 g): R_{f} (C)=0.36.

The 2-(4-bromo-but-2-enoxy)-benzoic acid methyl ester used as starting material is prepared as follows: 1,4-Dibromobutene (28.1 g) is added to a mixture of salicylic acid methyl ester (20.0 g) and anhydrous potassium carbonate (27.3 g) in acetonitrile (350 ml). The mixture is stirred under reflux for 4 hours and is filtered, and the filtrate is concentrated. FC (400 g of silica gel, eluant C) yields the title compound, R_{f} (C)=0.34, in the form of an oil.

E) 2-(4-Methoxybutoxy)-4-methyl-benzoic acid: Alkaline hydrolysis of 2-(4-methoxybutoxy)-4-methyl-benzoic acid methyl ester yields the title compound, R_{f} (hexane-ethyl acetate-glacial acetic acid 50:50:1)=0.38, in the form of a pale-yellow oil.

The 2-(4-metholxybutoxy)-4-methyl-benzoic acid methyl ester used as starting material is prepared as follows: In a manner analogous to that described in Example 50f, the title compound, R_{f} (C)=0.31, is obtained in the form of an oil from 2-(4-bromobutoxy)-4-methyl-benzoic acid methyl ester (R_{f} (C)=0.47).

The 2-(3-methoxypropoxy)-nicotinic acid used above as starting material is prepared as follows:

a) Alkaline hydrolysis in a manner analogous to that described in Example 1) yields the title compound, R_{f} (hexane-ethyl acetate-glacial acetic acid 50:25:3)=0.30, in the form of a yellow oil from 2-(3-methoxypropoxy)-nicotinic acid ethyl ester.

b) 2-(3-methoxypropoxy)-nicotinic acid ethyl ester: 2-Hydroxy-nicotinic acid ethyl ester (1.67 g), 3-methoxypropyl bromide (2.3 g) and silver carbonate (1.38 g) in toluene (80 ml) are reacted in accordance with the procedure described by Labaudiniere et al. (J. Med. Chem. 1992, 35, 4315-4324). Purification of the crude product by FC (dichloromethane-methanol-conc. ammonia 95:5:1) yields 1-(3-methoxypropoxy)-3-carbomethoxy-2(1H)-pyridinone (1.17 g), R_{f} (dichloromethane-methanol-conc. ammonia 95:5:1)=0.59, in the form of a pale-yellow oil, as well as the title compound (0.93 g), R_{f} (dichloromethane-methanol-conc. ammonia 95:5:1)=0.79, in the form of a yellowish oil.

The 3-(4-methoxybutoxy)-picolinic acid used above as starting material is prepared as follows:

a) Alkaline hydrolysis as described in Example 1) yields the title compound in the form of a solid from 3-(4-methoxybutoxy)-picolinic acid ethyl ester.

b) 3-(4-Methoxybutoxy)-picolinic acid ethyl ester: Analogously to the procedure of Labaudiniere et al. (J. Med. Chem. 1992, 35, 4315-4324), the title compound (0.98 g), R_{f} (hexane-ethyl acetate-glacial acetic acid 1:1:0.01)=0.21, is obtained in the form of a yellow oil from 3-hydroxy-picolinic acid ethyl ester (2.0 g) and 4-methoxybutyl bromide (2.99 g), with subsequent purification by FC (dichloromethane-methanol-conc. ammonia 95:5:1).

### Example 18

A solution of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(3-methoxypropoxy)-benzamide (60 mg) in n-butylamine (2 ml) is stirred at 50° C. for 40 hours. The mixture is concentrated and the oily residue is purified by FC (10 g of silica gel, eluant gradient from E to hexane-ethyl acetate 1:3). (2S,4S,5S,7S)-N-[4-(tert-butoxycarbonyl)amino-7-butylcarbamoyl-5-hydroxy-2-isopropyl-8-methyl-nonyl]-2-(3-methoxypropoxy)-benzamide, R_{f} (E) 0.07; HPLC R_{f}=18.4 min; MS(FAB) m/e 622 (M⁺+1), is obtained in the form of a yellowish oil.

### Example 19

In a manner analogous to that described in Example 18), reaction of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-benzamide (71 mg) in n-butylamine (2 ml) at 50° C. for 48 hours and subsequent purification of the crude product by FC on 10 g of silica gel (eluant gradient from E to hexane-ethyl acetate 1:4) yield (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-benzamide, R_{f} (E)=0.14, in the form of a foamy solid.

### Example 20

In a manner analogous to that described in Example 18), the following compounds are obtained by lactone opening with n-butylamine:

a) From 116 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-propoxy-benzamide, (2S,4S,5S,7S)-N-[4-(tert-butoxycarbonyl)amino-7-butylcarbamoyl-5-hydroxy-2-isopropyl-8-methyl-nonyl]-2-propoxy-benzamide, R_{f} (E)=0.19, in the form of an oil.

b) From 75 mg of (2S,2′S,2″S,4″S)-N-{2-[2′-(tert-butoxycarbonyl)amino-2′-(4″-isopropyl-5″-oxo-tetrahydrofuran-2″yl)-ethyl]-3-methylbutyl}-2-(2-methoxyethoxy)-benzamide, (2S,4S,5S,7S)-N-[4-(tert-butoxycarbonyl)amino-7-butylcarbamoyl-5-hydroxy-2-isopropyl-8-methyl-nonyl]-2-(2-methoxyethoxy)-benzamide, R_{f} (E)=0.06, in the form of a foamy solid.

c) From 88 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(methoxymethoxy)-benzamide, (2S,4S,5S,7S)-N-[4-(tert-butoxycarbonyl)amino-7-butylcarbamoyl-5-hydroxy-2-isopropyl-8-methyl-nonyl]-2-(2-methoxymethoxy)-benzamide, R_{f} (E)=0.09, in the form of a foamy solid.

d) From 50 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(2-methoxyethoxy)-ethoxy]-benzamide, (2S,4S,5S,7S)-N-[4-(tert-butoxycarbonyl)amino-7-butylcarbamoyl-5-hydroxy-2-isopropyl-8-methyl-nonyl]-2-[2-(2-methoxyethoxy)-ethoxy]-benzamide, R_{f} (F)=0.11, in the form of an oil.

e) From 107 mg of (2S,2′S,2″S,4″)-N-{2-[2′-(tert-butoxycarbonyl)amino-2′-(4″-isopropyl-5″-oxo-tetrahydrofuran-2″-yl)-ethyl]-3-methylbutyl}-4-methoxyl2-(3-methoxypropoxy)-benzamide, with purification by FC on 25 g of silica gel (eluant gradient from R to P), (2S,4S,5S,7S)-N-[4-(tert-butoxycarbonyl)amino-7-butylcarbamoyl-5-hydroxy-2-isopropyl-8-methyl-nonyl]-4-methoxy-2-(3-methoxypropoxy)-benzamide, R_{f} (L)=0.63, in the form of a yellow oil.

f) From 96 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-4-methoxy-3-(3-methoxypropoxy)-benzamide, with purification by FC as described in Example 20e), (2S,4S,5S,7S)-N-[4-(tert-butoxycarbonyl)amino-7-butylcarbamoyl-5-hydroxy-2-isopropyl-8-methyl-nonyl]-4-methoxy-3-(3-methoxypropoxy)-benzamide, R_{f} (L)=0.53, in the form of a foamy solid.

g) From 70 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-propoxymethyl-benzamide, with purification by FC (25 g of silica gel, eluant R), (2S,4S,5S,7S)-N-[4-(tert-butoxycarbonyl)amino-7-butylcarbamoyl-5-hydroxy-2-isopropyl-8-methyl-nonyl]-2-propoxymethyl-benzamide, R_{f} (L)=0.56, in the form of a foamy solid.

h) From 60 mg of (2S,2′S,2″S,4″S)-N-{2-[2′-(tert-butoxycarbonyl)amino-2′-(4″-isopropyl-5″-oxo-tetrahydrofuran-2′-yl)-ethyl]-3-methylbutyl}-2-[2-(methoxymethoxy)-ethoxy]-benzamide, (2S,4S,5S,7S)-N-[4-(tert-butoxycarbonyl)amino-7-butylcarbamoyl-5-hydroxy-2-isopropyl-8-methyl-nonyl]-2-[2-(methoxymethoxy)-ethoxy)-benzamide, R_{f} (L)=0.56, in the form of a yellowish oil.

i) From 50 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-acetamido-benzamide, (2S,4S,5S,7S)-N-[4-(tert-butoxycarbonyl)amino-7-butylcarbamoyl-5-hydroxy-2isopropyl-8-methyl-nonyl]-2-acetamido-benzamide R_{f} (L)=0.64, in the form of an oil.

j) From 60 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(3-methoxypropoxy)-nicotinamide, with purification of the crude product by FC on 25 g of silica gel (eluant gradient from O to P), (2S,4S,5S,7S)-N-[4-(tert-butoxycarbonyl)amino-7-butylcarbamoyl-5-hydroxy-2-isopropyl-8-methyl-nonyl]-2-(3-methoxypropoxy)-nicotinamide, R_{f} (L)=0.56, in the form of a colourless oil.

k) From 65 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-3-(4-methoxybutoxy)-pyridine-2-carboxylic acid amide, with purification of the crude product by FC on 25 g of silica gel (eluant V), (2S,4S,5S,7S)-N-[4-(tert-butoxycarbonyl)amino-7-butylcarbamoyl-5-hydroxy-2-isopropyl-8-methyl-nonyl]-3-(4-methoxybutoxy)-pyridine-2-carboxylic acid amide, R_{f} (W)=0.56, in the form of a yellow oil.

l) From 75 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(acetamide)-ethoxy]-benzamide, with purification of the crude product by FC on 25 g of silica gel (eluant gradient from T to V), (2S,4S,5S,7S)-N-(4-(tert-butoxycarbonyl)amino-7-butylcarbamoyl-5-hydroxy-2-isopropyl-8-methyl-nonyl]-2-[2-(acetamido)-ethoxy]-benzamide, R_{f} (W)=0.41, in the form of an oil.

m) From 75 mg of (2S,2′S,2″S,4″S)—N-{2-[2′-(tert-butoxycarbonyl)amino-2′-(4″-isopropyl-5″-oxo-tetrahydrofuran-2″-yl)-ethyl]-3-methylbutyl}-2-(4-methoxybut-2-enoxy)-benzamide, with purification of the crude product by FC on 25 g of silica gel (eluant gradient from S to V), (2S,4S,5S,7S)-N-[4-(tert-butoxycarbonyl)amino-7-butylcarbamoyl-5-hydroxy-2-isopropyl-8-methyl-nonyl]-2-(4-methoxybut-2-enoxy)-benzamide, R_{f} (W)=0.57, in the form of a solid.

n) From 75 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-methyl-benzamide, (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-methyl-benzamide, R_{f} (W)=0.63, in the form of a solid.

### Example 21

A 4N hydrochloric acid solution in dioxane (2 ml) is added at 0° C. to (2S,4S,5S,7S)-N-[4-(tert-butoxycarbonyl)amino-7-butylcarbamoyl-5-hydroxy-2-isopropyl-8-methyl-nonyl]-2-(3-methoxypropoxy)-benzamide (50 mg). The reaction mixture is stirred at 0° C. for 2 hours (TLC monitoring) and then the solvent is immediately concentrated under a high vacuum with vigorous stirring until frozen and is subsequently removed by lyophilisation. After drying under a high vacuum, (2S,4S,5S,7S)-N-(4-amino-7-butylcarbamoyl-5-hydroxy-2-isopropyl-8-methyl-nonyl)-2-(3-methoxypropoxy)-benzamide hydrochloride is obtained in the form of a foamy solid: R_{f} (W)=0.31. HPLC Rₜ=12.8 min. MS(FAB) m/e 522 (M⁺+1).

### Example 22

In a manner analogous to that described in Example 21), reaction of (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-benzamide (61 mg) yields (2S,4S,5S,7S)-N-(4-amino-7-butylcarbamoyl-5-hydroxy-2-isopropyl-8-methyl-nonyl)-2-(4-methoxybutoxy)-benzamide hydrochloride: R_{f} (W)=0.29. HPLC Rₜ=13.3 min. MS(FAB) m/e 536 (M⁺+1).

### Example 23

In a manner analogous to that described in Example 21), the following compounds are prepared by de-Bocylation:

a) From 100 mg of (2S,4S,5S,7S)-N-[4-(tert-butoxycarbonyl)amino-7-butylcarbamoyl-5-hydroxy-2-isopropyl-8-methyl-nonyl]-2-propoxy-benzamide, (2S,4S,5S,7S)-N-(4-amino-7-butylcarbamoyl-5-hydroxy-2-isopropyl-8-methyl-nonyl)-2-propoxy-benzamide hydrochloride: R_{f} (W)=0.37. HPLC Rₜ=13.95 min. MS(FAB) m/e 492 (M⁺+1).

b) From 60 mg of (2S,4S,5S,7S)-N-[4-(tert-butoxycarbonyl)amino-7-butylcarbamoyl-5-hydroxy-2-isopropyl-8-methyl-nonyl]-2-(2-methoxyethoxy)-benzamide, (2S,4S,5S,7S)-N-(4-amino-7-butylcarbamoyl-5-hydroxy-2-isopropyl-8-methyl-nonyl)-2-(2-methoxyethoxy)-benzamide hydrochloride: R_{f} (W)=0.38. HPLC Rₜ=12.6 min. MS(FAB) m/e 508 (M⁺+1).

c) From 38 mg of (2S,4S,5S,7S)-N-[4-(tert-butoxycarbonyl)amino-7-butylcarbamoyl-5-hydroxy-2-isopropyl-8-methyl-nonyl]-2-[2-(2-methoxyethoxy)-ethoxy]-benzamide, (2S,4S,5S,7S)-N-(4-amino-7-butylcarbamoyl-5-hydroxy-2-isopropyl-8-methyl-nonyl)-2-[2-(2-methoxyethoxy)-ethoxy]-benzamide hydrochloride: R_{f} (W)=0.19. HPLC Rₜ=12.4 min. MS(FAB) m/e 552 (M⁺+1).

d) From 93 mg of (2S,4S,5S,7S)-N-[4-(tert-butoxycarbonyl)amino-7-butylcarbamoyl-5-hydroxy-2-isopropyl-8-methyl-nonyl]-4-methoxy-2-(3-methoxypropoxy)-benzamide, (2S,4S,5S,7S)-N-(4-amino-7-butylcarbamoyl-5-hydroxy-2-isopropyl-8-methyl-nonyl)-4-methoxy-2-(3-methoxypropoxy)-benzamide hydrochloride: R_{f} (W)=0.25. HPLC Rₜ=13.4 min. MS(FAB) m/e 552 (M⁺+1).

e) From 76 mg of (2S,4S,5S,7S)-N-[4-(tert-butoxycarbonyl)amino-7-butylcarbamoyl-5-hydroxy-2-isopropyl-8-methyl-nonyl]-4-methoxy-3-(3-methoxypropoxy)-benzamide, (2S,4S,5S,7S)-N-(4-amino-7-butylcarbamoyl-5-hydroxy-2-isopropyl-8-methyl-nonyl)-4-methoxy-3-(3-methoxypropoxy)-benzamide hydrochloride: R_{f} (W)=0.28. HPLC Rₜ=12.1 min. MS(FAB) m/e 552 (M⁺+1).

f) From 58 mg of (2S,4S,5S,7S)-N-[4-(tert-butoxycarbonyl)amino-7-butylcarbamoyl-5-hydroxy-2-isopropyl-8methyl-nonyl]-2-oxymethyl-benzamide, (24S,5S,7S)-N-(4-amino-7-butylcarbamoyl-5-hydroxy-2-isopropyl-8-methyl-nonyl)-2-(propoxymethyl)-benzamide hydrochloride: R_{f} (W)=0.25. HPLC Rₜ=13.4 min. MS(FAB) m/e 506 (M⁺+1).

g) From 40 mg of (2S,4S,5S,7S)-N-[4-(tert-butoxycarbonyl)amino-7-butylcarbamoyl-5-hydroxy-2-isopropyl-8-methyl-nonyl]-2-acetamido-benzamide, with purification of the crude product by FC on 10 g of silica gel (eluant M), (2S,4S,5S,7S)-N-(4-amino-7-butylcarbamoyl-5-hydroxy-2-isopropyl-8-methyl-nonyl)-2-acetamido-benzamide hydrochloride: R_{f} (W)=0.45. HPLC Rₜ=10.0 min. MS(FAB) m/e 473 [(M⁺+1)-H₂O).

h) From 62 mg of (2S,4S,5S,7S)-N-[4-(tert-butoxycarbonyl)amino-7-butylcarbamoyl-5-hydroxy-2-isopropyl-8-methyl-nonyl]-2-[2-(acetamido)-ethoxy]-benzamide, (2S,4S,5S,7S)-N-(4-amino-7-butylcarbamoyl-5-hydroxy-2-isopropyl-8-methyl-nonyl)-2-[2-(acetamido)-ethoxy]-benzamide hydrochloride: R_{f} (W)=0.25. HPLC Rₜ=10.4 min. MS(FAB) m/e 535 (M⁺+1).

i) From 54 mg of (2S,4S,5S,7S)-N-[4-(tert-butoxycarbonyl)amino-7-butylcarbamoyl-5-hydroxy-2-isopropyl-8-methyl-nonyl]-2-(4-methoxybut-2-enoxy)-benzamide, (2S,4S,5S,7S)-N-(4-amino-7-butylcarbamoyl-5-hydroxy-2-isopropyl-8-methyl-nonyl)-2-(4-methoxybut-2-enoxy)-benzamide hydrochloride: R_{f} (W)=0.38. HPLC Rₜ=12.6 min. MS(FAB) m/e 534 (M⁺+1).

j) From 59 mg of (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-methyl-benzamide, (2S,4S,5S,7S)-N-(4-amino-7-butylcarbamoyl-5-hydroxy-2-isopropyl-8-methyl-nonyl)-2-(4-methoxybutoxy)-4-methyl-benzamide hydrochloride: R_{f} (W)=0.33. HPLC Rₜ=14.2 min. MS(FAB) m/e 550 (M⁺+1).

k) From 54 mg of (2S,4S,5S,7S)-N-[4-(tert-butoxycarbonyl)amino-7-butylcarbamoyl-5-hydroxy-2-isopropyl-8-methyl-nonyl]-2-(3-methoxypropoxy)-nicotinamide, with subsequent purification of the crude product by FC (eluant gradient from V to U), (2S,4S,5S,7S)-N-[4-amino-7-butylcarbamoyl-5-hydroxy-2isopropyl-8-methyl-nonyl]-2-(3-methoxypropoxy)-nicotinamide hydrochloride: R_{f} (W)=0.50. HPLC Rₜ=12.4 min. MS(FAB) m/e 523 (M⁺+1).

l) From 45 mg of (2S,4S,5S,7S)-N-[4-(tert-butoxycarbonyl) amino-7-butylcarbamoyl-5-hydroxy-2-isopropyl-8-methyl-nonyl]-3-(4-methoxybutoxy)-pyridine-2-carboxylic acid amide, (2S,4S,5S,7S)-N-[4-amino-7-butylcarbamoyl-5-hydroxy-2-isopropyl-8-methyl-nonyl]-3-(4-methoxybutoxy)-pyridine-2-carboxylic acid amide hydrochloride: R_{f} (W)=0.33. HPLC Rₜ=10.2 min. MS(FAB) m/e 537 (M⁺+1)

### Example 24

Trifluoroacetic acid (0.5 ml) is added at 0° C., with stirring, to a solution of (2S,4S,5S,7S)-N-[4-(tert-butoxycarbonyl)amino-7-butylcarbamoyl-5-hydroxy-2-isopropyl-8-methyl-nonyl]-2-(methoxymethoxy)-benzamide (46 mg) in dichloromethane (2 ml). When the reaction is complete (after approximately 30 minutes), toluene (2 ml) is added and the reaction mixture is concentrated. The crude product obtained after briefly drying under a high vacuum is purified by FC on 6 g of silica gel (eluant Q), and (2S,4S,5S,7S)-N-(4-amino-7-butylcarbamoyl-5-hydroxy-2-isopropyl-8-methyl-n onyl)-2-hydroxy-benzamide trifluoroacetate is obtained: R_{f} (W)=0.34. HPLC Rₜ=12.1 min. MS(FAB) m/e 450 (M⁺+1).

### Example 25

In a manner analogous to that described in Example 24), (2S,4S,5S,7S)-N-(4-amino-7-butylcarbamoyl-5-hydroxy-2-isopropyl-8-methyl-nonyl)-2-[2-(methoxymethoxy)-ethoxy]-benzamide trifluoroacetate, R_{f} (W)=0.29; HPLC Rₜ=12.3 min; MS(FAB) m/e 538 (M⁺+1), is obtained by de-Bocylation from 44 mg of (2S,4S,5S,7S)-N-[4-(tert-butoxycarbonyl)amino-7-butylcarbamoyl-5-hydroxy-2-isopropyl-8-methyl-nonyl]-2-[2-(methoxymethoxy)-ethoxy]-benzamide.

### Example 26

In a manner analogous to that described in Example 27), and with subsequent purification of the crude product by FC on 10 to 25 g of silica gel in each case (eluant system: dichloromethane-methanol-conc. ammonia), the following compounds are obtained by lactone opening with N-(2-aminoethyl)-morpholine (0.5 ml) at 80° C. overnight:

a) From 75 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-benzamide, (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(4-methoxybutoxy)-benzamide, R_{f} (W)=0.35, in the form of a colorless oil.

b) From 68 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(2-methoxyethoxy)-benzamide, (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(2-methoxyethoxy)-benzamide, R_{f} (W)=0.24, in the form of a yellowish oil.

c) From 60 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(3-methoxypropoxy)-nicotinamide, with purification of the crude product by FC on 25 g of silica gel (eluant gradient from P to O), (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(3-methoxypropoxy)-nicotinamide, R_{f} (L)=0.35, in the form of a yellow oil.

d) From 65 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-3-(4-methoxybutoxy)-pyridine-2-carboxylic acid amide, with purification of the crude product by FC on 25 g of silica gel (eluant V), (2S,4S,5S,7S)-N-[4-(tert-butoxycarbonyl)amino-5-hydroxy-2-isopropyl-8-methyl-7-(2morpholin-4-ylethylcarbamoyl)-nonyl]-3-(4-methoxybutoxy)-pyridine-2-Carboxylic acid amide, R_{f} (W)=0.38, in the form of a foamy solid.

e) From 45 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybut-2-enoxy)-benzamide, (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(4-methoxybut-2-enoxy)-benzamide, R_{f} (W)=0.50, in the form of an oil.

f) From 75 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-methyl-benzamide, with purification of the crude product on 25 g of silica gel (eluant gradient from Q to M), (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(4-methoxybutoxy)-4-methyl-benzamide, R_{f} (L)=0.38, in the form of an oil.

g) From 90 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(5-methoxypentoxy)-benzamide (Example 17o), (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(5-methoxypentoxy)-benzamide, R_{f} (L)=0.55, in the form of a colorless oil.

### Example 27

A mixture of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-benzamide (150 mg) and N-(3-aminopropyl)-morpholine (0.5 ml) is stirred overnight at 80° C. After cooling to room temperature, the reaction mixture is immediately chromatographed on 25 g of silica gel (eluant V). (2S,4S,5S,7S)-N-[4-(tert-butoxycarbonyl)amino-5-hydroxy-2-isopropyl-8-methyl-7-(3-morpholin-4ylpropylcarbamoyl)-nonyl]-2-(4-methoxybutoxy)-benzamide, R_{f} (W)=0.43, is obtained in the form of an oil.

### Example 28

(2S,4S,5S,7S)-N-[4-(tert-Butoxycarbonyl)amino-5-hydroxy-2-isopropyl-8-methyl-7-(2-morpholin-4-ylethylcarbamoyl)-nonyl]-2-(3-methoxypropoxy)-benzamide (64 mg) is stirred in 2 ml of a 4N hydrochloric acid solution in dioxane at 0° C. for one hour, in a manner analogous to that described in Example 21). Removal of the solvent and drying under a high vacuum yield (2S,4S,5S,7S)-N-[4-amino-5-hydroxy-2-isopropyl-8-methyl-7-(2-morpholin-4-ylethylcarbamoyl)-nonyl]-2-(3-methoxypropoxy)-benzamide dihydrochloride: R_{f} (W)=0.13. HPLC Rₜ=9.59 min. MS(FAB) m/e 579 (M⁺+1).

### Example 29

In a manner analogous to that described in Example 21), the following compounds are obtained by de-Bocylation:

a) From 80 mg of (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(4-methoxybutoxy)-benzamide, (2S,4S,5S,7S)-N-[4-amino-5-hydroxy-2-isopropyl-8-methyl-7-(2-morpholin-4-ylethylcarbamoyl)-nonyl]-2-(4-methoxybutoxy)-benzamide dihydrochloride: R_{f} (W)=0.26. HPLC Rₜ=9.9 min. MS(FAB) m/e 593 (M⁺+1).

b) From 72 mg of (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(2-methoxyethoxy)-benzamide, (2S,4S,5S,7S)-N-[4-amino-5-hydroxy-2-isopropyl-8-methyl-7-(2-morpholin-4-ylethylcarbamoyl)-nonyl]-2-(2-methoxyethoxy)-benzamide dihydrochloride: R_{f} (W)=0.40. HPLC Rₜ=8.9 min. MS(FAB) m/e 565 (M⁺+1).

c) From 63 mg of (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(3-methoxypropoxy)-nicotinamide, (2S,4S,5S,7S)-N-[4-amino-5-hydroxy-2-isopropyl-8-methyl-7-(2-morpholin-4-yl-ethylcarbamoyl)-nonyl]-2-(3-methoxypropoxy)-nicotinamide dihydrochloride: R_{f} (W)=0.27. HPLC Rₜ=8.4 min. MS(FAB) m/e 580 (M⁺+1).

d) From 46 mg of (2S,4S,5S,7S)-N-[4-(tert-butoxycarbonyl)amino-5-hydroxy-2-isopropyl-8-methyl-7-(2-morpholin-4-ylethylcarbamoyl)-nonyl]-3-(4-methoxybutoxy)-pyridine-2-carboxylic acid amide, (2S,4S,5S,7S)-N-[4-amino-5-hydroxy-2-isopropyl-8-methyl-7-(2-morpholin-4-ylethylcarbamoyl)-nonyl]-3-(4-methoxybutoxy)-pyridine-2-carboxylic acid amide dihydrochloride: R_{f} (W)=0.16. MS(FAB) m/e 593 (M⁺+1).

e) From 50 mg of (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(4-methoxybut-2-enoxy)-benzamide, (2S,4S,5S,7S)-N-[4-amino-5-hydroxy-2-isopropyl-8-methyl-7-(2-morpholin-4-ylethylcarbamoyl)-nonyl]-2-(4-methoxybut-2-enoxy)-benzamide dihydrochloride: R_{f} (W)=0.17. HPLC Rₜ=9.15 min. MS(FAB) m/e 591 (M⁺+1).

f) From 75 mg of (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(4-methoxybutoxy)-4-methyl-benzamide, (2S,4S,5S,7S)-N-[4-amino-5-hydroxy-2-isopropyl-8-methyl-7-(2-morpholin-4-ylethylcarbamoyl)-nonyl]-2-(4-methoxybutoxy)-4-methyl-benzamide dihydrochloride: R_{f} (W)=0.28. HPLC Rₜ=10.6 min. MS(FAB) m/e 607 (M⁺+1)

g) From 75 mg of (2S,4S,5S,7S)-N-[4-(tert-butoxycarbonyl)amino-5-hydroxy-2-isopropyl-8-methyl-7-(2-morpholin-4-ylethylcarbamoyl)-nonyl]-2-(5-methoxypentoxy)-benzamide, (2S,4S,5S,7S)-N-[4-amino-5-hydroxy-2-isopropyl-8-methyl-7-(2-morpholin-4-ylethylcarbamoyl)-methyl-nonyl]-2-(5-methoxypentoxy)-benzamide dihydrochloride: R_{f} (W)=0.29. HPLC Rₜ=10.2 min. MS(FAB) m/e 607 (M⁺+1).

h) From 96 mg of (2S,4S,5S,7S)-N-[4-(tert-butoxycarbonyl)amino-5-hydroxy-2-isopropyl-8-methyl-7-(3-morpholin-4-ylpropylcarbamoyl)-nonyl]-2-(4-methoxybutoxy]benzamide, (2S,4S,5S,7S)-N-[4-amino-5-hydroxy-2-isopropyl-8-methyl-7-(3-morpholin-4-ylpropylcarbamoyl)-nonyl]-2-(methoxybutoxy)-benzamide dihydrochloride: R_{f} (W)=0.14. HPLC Rₜ=10.0 min. MS(FAB) m/e 607 (M⁺+1).

### Example 30

In a manner analogous to that described in Example 1), the following compounds are prepared:

a) From 100 mg of (3S,5S,1′S,3′S)-5-[3′-aminomethyl-1′-(tert-butoxycarbonyl)amino-4′-methylpentyl]-3-isopropyl-dihydrofuran-2-one and 172 mg of 2-(4-methoxybutoxy)-4-(morpholin-4-ylmethyl)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl)-2-(4-methoxybutoxy)-4-(morpholin-4-ylmethyl)-benzamide, R_{f} (W)=0.55, in the form of a yellow oil.

b) From 100 mg of (3S,5S,1′S,3′S)-5-[3′-aminomethyl-1′-(tert-butoxycarbonyl)amino-4′-methylpentyl]-3-isopropyl-dihydrofuran-2-one and 198 mg of 2-(4-methoxybutoxy)-4-(2-morpholin-4-ylethoxy)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-(2-morpholin-4-ylethoxy)-benzamide, R_{f} (W)=0.65, in the form of a yellow oil.

c) From 50 mg of (3S,5S,1′S,3′S)-5-[3′-aminomethyl-1′-(tert-butoxycarbonyl)amino-4′-methylpentyl]-3-isopropyl-dihydrofuran-2-one and 91 mg of 4-[3-(dimethylamino)-propoxy]-2-(4-methoxybutoxy)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-4-[3-(dimethylamino)propoxy]-2-(4-methoxybutoxy)-benzamide, R_{f} (W)=0.44, in the form of an oil.

d) From 50 mg of (3S,5S,1′S,3′S)-5-[3′-aminomethyl-1′-(tert-butoxycarbonyl)amino-4′-methylpentyl]-3-isopropyl-dihydrofuran-2-one and 90 mg of 2-(4-methoxybutoxy)-4-(piperidin-1-yl)methyl-benzoic acid, (2S,2′S,2″S,4″S)-N-{2-[2′(tert-butoxycarbonyl)amino-2′-(4″-isopropyl-5″-oxo-tetrahydrofuran-2″-yl)-ethyl]-3-methylbutyl}-2-(4-methoxybutoxy)-4-(piperidin-1-yl)methyl-benzamide, R_{f} (W)=0.60, in the form of an oil.

e) From 50 mg of (3S,5S,1′S,3′S)-5-[3′-aminomethyl-1′-(tert-butoxycarbonyl)amino-4′-methylpentyl]-3-isopropyl-dihydrofuran-2-one and 86 mg of 2-(4-methoxybutoxy)-4-(pyrrolidin-1-yl)methyl-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-(pyrrolidin-1-yl)methyl-benzamide, R_{f} (W)=0.56, in the form of an oil.

f) From 125 mg of (3S,5S,1′S,3′S)-5-[3′-aminomethyl-1′-(tert-butoxycarbonyl)amino-4′-methylpentyl]-3-isopropyl-dihydrofuran-2-one and 246 mg of 2-(4-methoxybutoxy)-4-(2-piperidin-1-ylethoxy)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-(2-piperidin-1-ylethoxy)-benzamide, R_{f} (W)=0.58, in the form of an oil.

g) From 80 mg of (3S,5S,1′S,3′S)-5-[3′-aminomethyl-1′-(tert-butoxycarbonyl)amino-4′-methylpentyl]-3-isopropyl-dihydrofuran-2-one and 252 mg of 4-dimethylaminomethyl-2-(4-methoxybutoxy)-benzoic acid, with subsequent purification by FC (eluant N), (2S,2′S,2″S,4″S)-N-{2-[2′-(tert-butoxycarbonyl)amino-2′-(4″-isopropyl-5″-oxo-tetrahydrofuran-2′-yl)-ethyl]-3-methylbutyl}-4-dimethylaminomethyl-2-(4-methoxybutoxy)-benzamide, R_{f} (L)=0.41, in the form of an oil.

h) From 80 mg of (3S,5S,1′S,3′S)-5-[3′-aminomethyl-1′-(tert-butoxycarbonyl)amino-4′-methylpentyl]-3-isopropyl-dihydrofuran-2-one and 274 mg of 2-(4-methoxybutoxy)-4-(4-methylpiperazin-1-yl)methyl-benzoic acid, with subsequent purification by FC (eluant gradient from p to N), (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-(4-methylpiperazin-1-yl)methyl-benzamide, R_{f} (W)=0.28, in the form of a yellowish solid.

i) From 80 mg of (3S,5S,1′S,3′S)-5-[3′-aminomethyl-1′-(tert-butoxycarbonyl)amino-4′-methylpentyl]-3-isopropyl-dihydrofuran-2-one and 255 mg of 4-(4-acetylpiperazin-1-yl)methyl-2-(4-methoxybutoxy)-benzoic acid, with subsequent purification by FC (eluant O), (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-4-(4-acetylpiperazin-1-yl)-methyl-2-(4-methoxybutoxy)benzamide, R_{f} (W)=0.55, in the form of a yellowish solid.

The benzoic acids used as starting materials are prepared as described below:

A) 2-(4-Methoxybutoxy)-4-(morpholin-4-ylmethyl)-benzoic acid: Hydrolysis of 2-(4-methoxybutoxy)-4-(morpholin-4-ylmethyl)-benzoic acid methyl ester (1.08 g) with 1N sodium hydroxide solution (4.75 ml) in a 2:1 mixture of ethanol and water (15 ml) at 50° C. and customary working-up yield the title compound in the form of a yellow oil: R_{f} (L)=0.36.

The 2-(4-methoxybutoxy)-4-(morpholin-4-ylmethyl)-benzoic acid methyl ester that is used is prepared as follows: A mixture of 2-(4-methoxybutoxy)-4-methyl-benzoic acid methyl ester (1.0 g), N-bromosuccinimide (0.70 g), 2′,2″-azoisobutyronitrile (23 mg) and dibenzoyl peroxide (34 mg) in carbon tetrachloride (10 ml) is stirred under reflux for 5 hours. After cooling to room temperature, the precipitate is filtered off, morpholine (1.03 ml) is added to the filtrate, and stirring is carried out for a further 2 hours at room temperature. The crude product obtained after filtration and concentration is purified by FC on silica gel (40 g, eluant gradient from C to F). The title compound, R_{f} (F)=0.14, is obtained in the form of a yellowish oil.

B) 2-(4-methoxybutoxy)-4-(2-morpholin-4-ylethoxy)-benzoic acid: A mixture of 2-(4-methoxybutoxy)-4-(2-morpholin-4-ylethoxy)-benzoic acid methyl ester (2.95 g) and 1N sodium hydroxide solution (8.83 ml) in ethanol (10 ml) and water (5 ml) is stirred overnight at 50° C. After customary working-up, the title compound (2.50 g), R_{f} (L)=0.51, is obtained in the form of a yellowish oil.

The 2-(4-methoxybutoxy)-4-(2-morpholin-4-ylethoxy)-benzoic acid methyl ester used as starting material is prepared as follows:

a) A suspension of 4-hydroxy-2-(4-methoxybutoxy)-benzoic acid methyl ester (2.0 g), 2-chloroethylmorpholine (11.8 g) and caesium carbonate (12.8 g) in acetone (30 ml) is stirred under reflux for 2 hours. Filtration and purification by FC (80 g of silica gel, eluant F and ethyl acetate-conc. ammonia 10:0.1) yield 2-(4-methoxy butoxy)-4-(2-morpholin-4-ylethoxy)-benzoic acid methyl ester (2.96 g), R_{f} (N)=0.73, in the form of a pale-yellow oil.

b) 4-Hydroxy-2-(4-methoxybutoxy)-benzoic acid methyl ester: A solution of 4-benzyloxy-2-(4-methoxybutoxy)-benzoic acid methyl ester (13.8 g) in ethyl acetate (130 ml) is hydrogenated for 2 hours at room temperature in the presence of 10% Pd/C (1.37 g). The title compound (10.1 g), R_{f} (C)=0.09; m.p. 62°-63° C., is obtained in the form of a white solid.

C) 2-(4-methoxybutoxy)-4-(3-dimethylaminopropoxy)-benzoic acid: 2-(4-metholoxybutoxy)-4-(3-dimethylaminopropoxy)-benzoic acid methyl ester (2.12 g), dissolved in a mixture of ethanol (10 ml) and water (5 ml), is hydrolysed in the presence of 1N sodium hydroxide solution (6.87 ml). When the reaction is complete, dichloro methane (100 ml) is added and the aqueous phase is adjusted to pH 6 by the addition of 1M potassium hydrogen sulfate solution. The aqueous phase is again extracted with dichloromethane, and the combined organic phases are washed with brine (20 ml), dried over magnesium sulfate and concentrated. The title compound is obtained in admixture with inorganic salts in the form of a yellowish oil, which is reacted further without additional purification.

The 2-(4-methoxybutoxy)-4-(3-dimethylaminopropoxy)-benzoic acid methyl ester used as starting material is obtained in the form of an oil in a manner analogous to that described in Example 30Aa) from 2-(4-methoxybutoxy)-4-methyl-benzoic acid methyl ester (2.0 g) and dimethylaminopropyl chloride (2.4 g), with subsequent purification by FC on 40 g of silica gel (eluant F and ethyl acetate-conc. ammonia 99:1).

D) 2-(4-methoxybutoxy)-4-(piperidin-1-ylmethyl)-benzoic acid: 1N sodium hydroxide solution (4.8 ml) is added to 2-(4-methoxybutoxy)-4-(piperidin-1-ylmethyl)-benzoic acid methyl ester (1.35 g), dissolved in ethanol (20 ml) and water (10 ml), and the mixture is stirred overnight at room temperature. The reaction mixture is adjusted to pH 6 by the addition of 1M potassium hydrogen sulfate solution and is largely concentrated. The residue is taken up in dioxane (30 ml) and the solution is frozen in a dry-ice-bath and lyophilised under a high vacuum. The title compound is obtained in admixture with inorganic salts in the form of a light-brown solid (1.60 g), which is reacted without being purified further: R_{f} (L)=0.05.

The 2-(4-methoxybutoxy)-4-(piperidin-1-ylmethyl)-benzoic acid methyl ester that is used is prepared in a manner analogous to that described in Example 30 An) from 2-(4-methoxybutoxy)-4-methyl-benzoic acid methyl ester and piperidine, with subsequent purification of the crude product by FC (eluants C and M): brown oil, R_{f} (N)=0.34.

E) 2-(4-Methoxybutoxy)-4-(pyrrolidin-1-ylmethyl)-benzoic acid: The title compound is obtained in admixture with inorganic salts in a manner analogous to that described in Example 30D) from 2(4-methoxybutoxy)-4-(pyrrolidin-1-ylmethyl)-benzoic acid methyl ester.

The 2-(4-methoxybutoxy)-4-(pyrrolidin-1-ylmethyl)-benzoic acid methyl ester that is used is prepared in a manner analogous to that described in Example 30Aa) from 2-(4-methoxybutoxy)-4-methyl-benzoic acid methyl ester and pyrrolidine, with subsequent purification of the crude product by FC (eluants C and M): brown-black oil, R_{f} (N)=0.22.

F) 2-(4-Methoxybutoxy)-4-(piperidin-1-ylethoxy)-benzoic acid: Alkaline hydrolysis of 2-(4-methoxybutoxy)-4-(piperidin-1-ylethoxy)-benzoic acid methyl ester, in a manner analogous to that described in Example 30D), and subsequent purification by FC (eluant gradient from N to dichloromethane-methanol 8:2) yield the title compound in the form of a yellowish oil, which slowly crystallises out when left to stand: R_{f} (dichloromethane-methanol 8:2)=0.50; m.p. 91°-94° C.

The 2-(4-methoxybutoxy)-4-(piperidin-1-ylethoxy)-benzoic acid methyl ester used as starting material is prepared as follows:

a) A solution of 2-(4-methoxybutoxy)-4-(piperidin-1-ylcarbamoylmethoxy)-benzoic acid methyl ester (2.29 g) in tetrahydrofuran (10 ml) is added dropwise at 0°-5° C. over a period of 15 minutes to a 1M borane THF complex solution in tetrahydrofuran (10.0 ml). The mixture is then heated to reflux temperature and stirred for 4 hours. A further 2.0 ml of a 1M borane THF complex solution are added. After one hour under reflux, the mixture is allowed to cool, the solvent is removed, and anhydrous methanol (0.97 ml) and 3.75N hydrochloric acid solution in diethyl ether (1.61 ml) are added to the residue. After stirring overnight at room temperature, the mixture is concentrated and the crude product is purified by FC (eluant gradient from P to N). 2-(4-Methoxybutoxy)-4-(piperidin-1ylethoxy)-benzoic acid methyl ester, R_{f} (L)=0.38, is obtained in the form of a brown oil (1.39 g).

b) 2-(4-Methoxybutoxy)-4-(piperidin-1-ylcarbamoylmethoxy)-benzoic acid methyl ester: Cyanophosphonic acid diethyl ester (0.88 ml), piperidine (0.57 ml) and triethylamine (0.73 ml) are added at 0° C. to a suspension of 4-carboxymethoxy-2-(4-methoxybutoxy)-benzoic acid methyl ester (1.64 g) in anhydrous dichloromethane (20 ml). The reaction mixture is stirred for 4 hours at 0° C. and for one hour at room temperature. Further cyanophosphonic acid diethyl ester (0.40 ml) and piperidine (0.25 ml) are added, and the mixture is stirred for a further 45 minutes at room temperature and is then diluted with dichloromethane (50 ml). The organic phase is washed with a 1M potassium hydrogen sulfate solution, saturated sodium hydrogen carbonate solution and saturated sodium chloride solution, dried over magnesium sulfate and concentrated. Purification by FC (eluant gradient from E to hexane-ethyl acetate 1:3) yields the title compound (1.95 g) in the form of a pale-yellow solid: R_{f} (E)=0.13; MS(EI) m/e 365 (M⁺).

c) 4-Carboxymethoxy-2-(4-methoxybutoxy)-benzoic acid methyl ester: 4-tert-Butoxycarbonylmethoxy-2-(4-methoxybutoxy)-benzoic acid methyl ester (3.0 g), which is obtained in the form of a pale-yellow oil (R_{f} (C)=0.13) in a manner analogous to that described in Example 30Ba) from 4-hydroxy-2-(4-methoxybutoxy)-benzoic acid methyl ester and tert-butyl bromoacetate, is dissolved in 4N hydrochloric acid solution in dioxane (25 ml) and the mixture is stirred overnight at room temperature. The solvent is then removed under a high vacuum and the solid residue is dissolved in hot ethyl acetate (10 ml). Hexane (approximately 20 ml) is added until the mixture begins to turn cloudy. After cooling to room temperature, the white precipitate is filtered off, washed with hexane and dried. The title compound (1.64 g) is obtained in the form of a white solid: R_{f} (hexane-ethyl acetate-glacial acetic acid 50:50:1)=0.12.

G) 2-(4-Methoxybutoxy)-4-[(4-methyl-piperazin-1-yl)methyl]-benzoic acid methyl ester: The title compound is prepared in a manner analogous to that described in Example 30Aa) from 2-(4-methoxybutoxy)-4-methyl-benzoic acid methyl ester and N-methylpiperidine, with subsequent purification of the crude product by FC (eluant T): yellow oil, R_{f} (V)=0.37.

H) 4-[(4-Acetyl-piperazin-1-yl)methyl]-2-(4-methoxybutoxy)-benzoic acid methyl ester: The title compound is prepared in a manner analogous to that described in Example 30Aa) from 2-(4-methoxybutoxy)-4-methyl-benzoic acid methyl ester and N-acetylpiperazine, with subsequent purification by FC (eluant T): yellow oil, R_{f} (V)=0.30.

### Example 31

In a manner analogous to that described in Example 18), with subsequent purification of the crude product by FC on 25 to 50 g of silica gel in each case (eluant system: dichloromethane-methanol-conc. ammonia), the following compounds are prepared by lactone opening:

a) From 202 mg of (2S,2′S,2″S,4″S)-N-(2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-(morpholin-4-ylmethyl)-benzamide, (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(morpholin-4-ylmethyl)-benzamide, R_{f} (w)=0.60, in the form of a foamy solid.

b) From 185 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-[2-(morpholin-4-yl)ethoxy]-benzamide, (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-[2-(morpholin-4-yl)-ethoxy]-benzamide, R_{f} (W)=0.54, in the form of an oil.

c) From 70 mg of (2S,2′S,2″S,4″S)-N-{2-[2′-(tert-butoxycarbonyl)amino-2′-(4′-isopropyl-5″-oxo-tetrahydrofuran-2″-yl)-ethyl]-3-methylbutyl}-4-[3-(dimethylamino)-propoxy]-2-(4-methoxybutoxy)-benzamide, (2S,4S,5S,7S)-N-[4-(tert-butoxycarbonyl)amino-7-butylcarbamoyl-5-hydroxy-2-isopropyl-8-methyl-nonyl]-4-[3-(dimethylamino)-propoxy]-2-(4-methoxybutoxy)-benzamide, R_{f} (W)=0.29, in the form of an oil.

d) From 84 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-(piperidin-1-yl)-methyl-benzamide, (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(piperidin-1-yl)methyl-benzamide, R_{f} (W)=0.66; MS(FAB) m/e 733 (M⁺+1), in the form of an oil.

e) From 60 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-(pyrrolidin-1-yl)-methyl-benzamide, (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(pyrrolidin-1-yl)methyl-benzamide, R_{f} (W)=0.54; MS(FAB) m/e 719 (M⁺+1), in the form of an oil.

f) From 68 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-(2-piperidin-1-ylethoxy)-benzamide, (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(2-piperidin-1-ylethoxy)-benzamide, R_{f} (W)=0.54, in the form of a colorless solid.

g) From 100 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-4-dimethylaminomethyl-2-(4-methoxybutoxy)-benzamide, (2S,4S,5S,7S)-N-[4-(tert-butoxycarbonyl)amino-7-butylcarbamoyl-5-hydroxy-2-isopropyl-8methyl-nonyl]-4-dimethylaminomethyl-2-(4-methoxybutoxy)-benzamide, R_{f} (W)=0.48, in the form of a yellowish solid.

h) From 151 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-(4-methylpiperazin-1-yl)methyl-benzamide, with purification by FC (25 g of silica gel, eluant V), (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(4-methylpiperazin-1-yl)methyl-benzamide, R_{f} (W)=0.37; HPLC Rₜ=13.8 min, in the form of an oil.

i) From 130 mg of (2S,2′S,2″S,4″S)—N-{2-[2′-(tert-butoxycarbonyl)amino-2′-(4″-isopropyl-5″-oxo-tetrahydrofuran-2″-yl)-ethyl]-3-methylbutyl}-4-(4-acetylpiperazin-1-yl)methyl-2-(4-methoxybutoxy)-benzamide, with purification by FC (eluant T), (2S,4S,5S,7S)-N-[4-(tert-butoxycarbonyl)amino-7-butylcarbamoyl-5-hydroxy-2-isopropyl-8-methyl-nonyl]-4-(4-acetylpiperazin-1-yl)methyl-2-(4-methoxybut oxy)-benzamide, R_{f} (W)=0.46; HPLC Rₜ=17.9 min, in the form of a yellowish solid.

### Example 32

In a manner analogous to that described in Example 21), the following compounds am prepared by de-Bocylation:

a) From 144 mg of (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(morpholin-4-ylmethyl)-benzamide, (2S,4S,5S,7S)-N-(4-amino-7-butylcarbamoyl-5-hydroxy-2-isopropyl-8-methyl-nonyl)-2-(4-methoxybutoxy)-4-(morpholin-4-ylmethyl)-benzamide dihydrochloride: R_{f} (W)=0.25. HPLC Rₜ=9.6 min. MS(FAB) m/e 635 (M⁺+1).

b) From 155 mg of (2S,4S,5S,7S)-N-[4-(tert-butoxycarbonyl)amino-7-butylcarbamoyl-5-hydroxy-2-isopropyl-8-methyl-nonyl]-2-(4-methoxybutoxy]-4-[2-(morpholin-4-yl)-ethoxy]-benzamide, (2S,4S,5S,7S)-N-(4-amino-7-butylcarbamoyl-5-hydroxy-2-isopropyl-8-methylnonyl)-2-(4-methoxybutoxy)-4-[2-(morpholin-4-yl)-ethoxy]-benzamide dihydrochloride, R_{f}=0.18. HPLC Rₜ=10.0 min. MS(FAB) m/e 665 (M⁺+1).

c) From 36 mg of (2S,4S,5S,7S)-N-[4-(tert-butoxycarbonyl)amino-7-butylcarbamoyl-5-hydroxy-2-isopropyl-8-methyl-nonyl]-4-[3-(dimethylamino)-propoxy]-2-(4-methoxybutoxy)-benzamide, (2S,4S,5S,7S)-N-(4-amino-7-butylcarbamoyl-5-hydroxy-2-isopropyl-8-methylnonyl)-4-[3-(dimethylamino)-propoxy]-2-(4-methoxybutoxy)-benzamide dihydrochloride: R_{f} (W)=0.11. HPLC Rₜ=9.3 min. MS(FAB) m/e 637 (M⁺+1).

d) From 50 mg of (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(piperidin-1-yl)methyl-benzamide, (2S,4S,5S,7S)-N-(4-amino-7-butylcarbamoyl-5-hydroxy-2-isopropyl-8-methyl-nonyl)-2-(4-methoxybutoxy)-4-(piperidin-1-yl)methyl-benzamide dihydrochloride: R_{f} (W)=0.41. HPLC Rₜ=10.5 min. MS(FAB) m/e 633 (M⁺+1).

e) From 48 mg of (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(pyrrolidin-1-yl)methyl-benzamide, (2S,4S,5S,7S)-N-(4-amino-7-butylcarbamoyl-5-hydroxy-2-isopropyl-8-methyl-nonyl)-2-(4-methoxybutoxy)-4-(pyrrolidin-1-yl)methyl-benzamide dihydrochloride: R_{f} (W)=0.32. HPLC Rₜ=10.2 min. MS(FAB) m/e 619 (M⁺+1).

f) From 53 mg of (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(2-piperidin-1-ylethoxy)-benzamide, (2S,4S,5S,7S)-N-(4-amino-7-butylcarbamoyl-5-hydroxy-2-isopropyl-8-methyl-nonyl)-2-(4-methoxybutoxy)-4-(2-piperidin-1-ylethoxy)-benzamide dihydrochloride: R_{f} (W)=0.16. HPLC Rₜ=9.98 min. MS(FAB) m/e 663 (M⁺+1).

g) From 79 mg of (2S,4S,5S,7S)-N-[4-(tert-butoxycarbonyl)amino-7-butylcarbamoyl-5-hydroxy-2-isopropyl-8-methyl-nonyl]-4-dimethylaminomethyl-2-(4-methoxybutoxy)-benzamide, (2S,4S,5S,7S)-N-(4-amino-7-butylcarbamoyl-5-hydroxy-2-isopropyl-8-methyl-nonyl)-4-dimethylaminomethyl-2-(4methoxybutoxy)-benzamide dihydrochloride: R_{f} (W)=0.21. HPLC Rₜ=9.57 min. MS(FAB) m/e 593 (M⁺+1).

h) From 124 mg of (2S,4S,5S,7S)-N-[4-(tert-butoxycarbonyl)amino-7-butyl carbamoyl-5-hydroxy-2-isopropyl-8-methyl-nonyl]-2-(4-methoxybutoxy)-4-(4-methylpiperazin-1-yl)methylbenzamide, (2S,4S,5S,7S)-N-(4-amino-7-butylcarbamoyl-5-hydroxy-2-isopropyl-8-methylnonyl)-2-(4-methoxybutoxy)-4-(4-methylpiperazin-1-yl)methyl-benzamide trihydrochloride: R_{f} (W)=0.21. HPLC Rₜ=10.2 min. MS(FAB) m/e 648 (M⁺+1).

i) From 83 mg of (2S,4S,5S,7S)-N-[4-(tert-butoxycarbonyl)amino-7-butylcarbamoyl-5-hydroxy-2-isopropyl-8-methyl-nonyl]-4-(4-acetylpiperazin-1-yl)methyl-2-(4-methoxybutoxy)-benzamide, (2S,4S,5S,7S)-N-(4-amino-7-butylcarbamoyl-5-hydroxy-2-isopropyl-8-methylnonyl)-4-(4-acetylpiperazin-1-yl)methyl-2-(4-methoxybutoxy)-benzamide dihydrochloride: R_{f} (W)=0.29. HPLC Rₜ=10.6 min. HRMS(FAB) m/e 676.5017 (M⁺+1).

### Example 33

In a manner analogous to that described in Example 1), the following compounds are prepared:

a) From 100 mg of (3S,5S,1′S,3′S)-5-[3′-aminomethyl-1′-(tert-butoxycarbonyl)amino-4′-methylpentyl]-3-isopropyl-dihydrofuran-2-one and 124 mg of 2-(3-azidopropoxy)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(3-azidopropoxy)-benzamide, R_{f} (hexane-diethyl ether 1:4)=0.46; HPLC Rₜ=19.2 min.

b) From 100 mg of (3S,5S,1′S,3′S)-5-[3′-aminomethyl-1′-(tert-butoxycarbonyl)amino-4′-methylpentyl]-3-isopropyl-dihydrofuran-2-one and 116 mg of 2-(2-azidoethoxy)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(2-azidoethoxy)-benzamide, R_{f} (hexane-diethyl ether 1:4)=0.41; HPLC Rₜ=18.6 min.

c) From 150 mg of (3S,5S,1′S,3′S)-5-[3′-aminomethyl-1′-(tert-butoxycarbonyl)amino-4′-methylpentyl]-3-isopropyl-dihydrofuran-2-one and 246 mg of 2-[2-(4-acetylpiperazin-1-yl)-ethoxy]-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(4-acetylpiperazin-1-yl)-ethoxy]-benzamide, R_{f} (J)=0.45; HPLC Rₜ=13.6 min.

d) From 100 mg of (3S,5S,1′S,3′S)-5-[3′-aminomethyl-1′-(tert-butoxycarbonyl)amino-4′-methylpentyl]-3-isopropyl-dihydrofuran-2-one and 132 mg of 2-[2-(morpholin-4-yl)-ethyl]-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(morpholin-4-yl)-ethyl]-benzamide, R_{f} (L)=0.50; HPLC Rₜ=15.8 min.

e) From 100 mg of (3S,5S,1′S,3′S)-5-[3′-aminomethyl-1′-(tert-butoxycarbonyl)amino-4′-methylpentyl]-3-isopropyl-dihydrofuran-2-one and 125 mg of 2-(3-dimethylaminopropoxy)-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(3-dimethylaminopropoxy)-benzamide, R_{f} (J)=0.70; HPLC Rₜ=14.6 min.

f) From 50 mg of (3S,5S,1′S,3′S)-5-[3′-aminomethyl-1′-(tert-butoxycarbonyl)amino-4′-methylpentyl]-3-isopropyl-dihydrofuran-2-one and 72 mg of 2-[3-(morpholin-4-yl)-propoxy]-benzoic acid, (2S,2′S,2″S,4″S)-N-{2-[2′-(tert-butoxycarbonyl)amino-2′-(4″-isopropyl-5′-oxo-tetrahydrofuran-2″-yl)-ethyl]-3-methylbutyl}-2-[3-(morpholin-4-yl)-propoxy]-benzamide, R_{f} (H)=0.25; HPLC Rₜ=14.4 min.

g) From 50 mg of (3S,5S,1′S,3′S)-5-[3′-aminomethyl-1′-(tert-butoxycarbonyl)amino-4′-methylpentyl]-3-isopropyl-dihydrofuran-2-one and 70 mg of 2-[2-(morpholin-4-yl)-ethoxy]-benzoic acid, (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(morpholin-4-yl)-ethoxy]-benzamide, R_{f} (H)=0.43; HPLC Rₜ=14.2 min.

The benzoic acid derivatives used as starting materials are prepared in accordance with customary methods from the literature, unless otherwise described in greater detail below:

A) 2-(3-Azidopropoxy)-benzoic acid: 1N sodium hydroxide solution (3 ml) is added to a solution of 2-(3-azidopropoxy)-benzoic acid methyl ester (0.5 g) in methanol (7 ml), and the reaction mixture is stirred at reflux for 15 minutes. The mixture is concentrated by evaporation, water (25 ml) is added and the pH is adjusted to 6 at 0° C. with 1N hydrochloric acid. The aqueous solution is extracted with dichloromethane (2×100 ml), and the organic phase is dried over sodium sulfate and concentrated by evaporation. The title compound, R_{f} (L)=0.62, is obtained.

a) 2-(3-Azidopropoxy)-benzoic acid methyl ester: Sodium azide (0.45 g) is added to a solution of 2-(3-bromopropoxy)-benzoic acid methyl ester (1.5 g; prepared in accordance with the procedure described by Smith et al. in J. Chem. Soc. Perkin Trans I (1988) 77) in N,N-dimethylformamide (10 ml), and the suspension is heated at 50° C. for 15 hours. The reaction mixture is concentrated by evaporation and partitioned between water (50 ml) and dichloromethane (100 ml). The organic phase is washed with water (2×50 ml), dried over sodium sulfate and concentrated by evaporation. The evaporation residue is purified by FC (200 g of silica gel, eluant A). The title compound, R_{f} (B)=0.25, is obtained.

B) 2-(2-Azidoethoxy)-benzoic acid: In a manner analogous to that described in Example 33A), from 1.3 g of 2-(2-azidoethoxy)-benzoic acid methyl ester, in the form of an oil, R_{f} (L)=0.59.

a) 2-(2-Azidoethoxy)-benzoic acid methyl ester: Sodium azide (0.6 g) is added to a solution of 2-(3-bromoethoxy)-benzoic acid methyl ester (2.0 g; prepared in accordance with the procedure described by W. A. Jacobs and M. Heidelberger in J. Biol. Chem. (1915) 21, 448) in 1,3-dimethyl-3,4,5,6tetrahydro-2(1H)-pyrimidinone (20 ml), and the suspension is heated at 50° C. for 5 hours. The reaction mixture is concentrated by evaporation and partitioned between water (100 ml) and diethyl ether (200 ml). The organic phase is washed with water (50 ml), dried over sodium sulfate and concentrated by evaporation. The evaporation residue is purified by FC (360 g of silica gel, eluant E). The title compound, R_{f} (E)=0.43, is obtained.

C) 2-[2-(4-Acetylpiperazin-1-yl)-ethoxy]-benzoic acid: In an analogous manner from 2-[2-(4-acetylpiperazin-1-yl)-ethoxy]-benzoic acid methyl ester (2.5 g), in the form of an oil: R_{f} (dichloromethane-methanol 4:1)=0.20.

a) 2-[2-(4-Acetylpiperazin-1-yl)-ethoxy]-benzoic acid methyl ester: N-Acetylpiperazine (3.0 g) is added to a solution of 2-(2-bromopropoxy)-benzoic acid methyl ester (2.0 g) in acetonitrile (50 ml), and the reaction mixture is heated at 50° C. When the reaction is complete, the mixture is concentrated by evaporation and the crude product is purified by FC (150 g of silica gel, dichloromethane-methanol 4:1). The title compound, R_{f} (dichloromethane-methanol 4:1)=0.76, is obtained.

D) 2-[2-(Morpholin-4-yl)-ethyl]-benzoic acid: In an analogous manner from 2-[2-(morpholin-4-yl)-ethyl]-benzoic acid ethyl ester (1.0 g), in the form of an oil: R_{f} (ethyl acetate-methanol 9:1)=0.05.

a) 2-[2-(Morpholin-4-yl)-ethyl]-benzoic acid ethyl ester: The title compound is obtained from 2-(2-bromoethyl)-benzoic acid ethyl ester (1.0 g) in a manner analogous to that described in Example 33Ca): R_{f} (ethyl acetate-methanol 9:1)=0.55.

b) 2-(2-Bromoethyl)-benzoic acid ethyl ester: Phosphorus tribromide (5.58 ml) and bromine (3.33 ml) are added at 15° C. to a solution of 1-oxo-isochroman (8.0 g) in carbon tetrachloride (80 ml). The mixture is stirred at room temperature overnight and then at 60° C. for 3 hours. Ethanol (16 ml) is added at room temperature, and the mixture is stirred for one hour. Finally, the reaction mixture is partitioned between dichloromethane (500 ml) and water (50 ml) and the organic phase is washed with water (50 ml), dried over sodium sulfate and concentrated by evaporation. The residue is purified by FC (eluant E). The title compound, R_{f} (E)=0.70, is obtained.

E) In a manner analogous to that described in Examples 33A-D), the following compounds are obtained:

a) From 2-[3-(morpholin-4-yl)-propoxy]-benzoic acid methyl ester, 2-[3-(morpholin-4-yl)-propoxy]-benzoic acid, R_{f} (dichloromethane-methanol 7:3)=0.46, in the form of an oil.

b) From 2-[2-(morpholin-4-yl)-ethoxy]-benzoic acid methyl ester, 2-[2-(morpholin-4-yl)-ethoxy]-benzoic acid, R_{f} (dichloromethane-methanol 7:3)=0.47, in the form of an oil.

c) From 2-[2-(4-methoxypiperidin-1-yl)-ethyl]-benzoic acid ethyl ester (R_{f} (ethyl acetate-methanol 9:1)=0.22), 2-[2-(4-methoxypiperidin-1-yl)-ethyl]-benzoic acid, R_{f} (dichloromethane-methanol 7:3)=0.60, in the form of an oil.

d) From 2-[2-(4-acetylpiperazin-1-yl)-ethyl]-benzoic acid ethyl ester (R_{f} (acetic acid-methanol 9:1)=0.22), 2-[2-(4-acetylpiperazin-1-yl)-ethyl]-benzoic acid, R_{f} (dichloromethane-methanol 7:3)=0.60, in the form of an oil.

### Example 34

A mixture of (3S,5S,1′S,3′S)-5-[3′-aminomethyl-1′-(tert-butoxycarbonyl)amino-41′-methylpentyl]-3-isopropyl-dihydrofuran-2-one (100 mg), 2-[2-(4-methoxypiperidin-1-yl)-ethyl]-benzoic acid (89 mg), o-benzotriazol-1-yl-N,N,N′,N′-tetramethyl-uronium hexafluorophosphate (128 mg) and triethylamine (59 ml) in acetonitrile (5 ml) is stirred at room temperature for 24 hours. The mixture is concentrated by evaporation and the residue is partitioned between dichloromethane and water. The organic phase is dried over sodium sulfate and concentrated by evaporation. FC on silica gel (50 g, eluant I) yields (2S,2′S,2″S,4″S)-N-{2-[2′(tert-butoxycarbonyl)amino-2′-(4″-isopropyl-5″-oxo-tetrahydrofuran-2″-yl)-ethyl]-3-methylbutyl}-2-[2-(4-metholoxypiperidin-1-yl)-ethyl]-benzamide, R_{f} (L)=0.44; HPLC Rₜ=16.1 min.

### Example 35

In a manner analogous to that described in Example 34), (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(4-acetylpiperazin-1-yl)-ethyl]-benzamide, R_{f} (ethyl acetate-methanol 9:1)=0.26; HPLC Rₜ=14.4 min, is obtained from (3S,5S,1′S,3′S)-5-[3′-aminomethyl-1′-tert-butoxycarbonyl)amino-4′-methylpentyl]-3-isopropyl-dihydrofuran-2-one (100 mg), 2-[2-(4-acetylpiperazin-1-yl)-ethyl]-benzoic acid (93 mg), O-benzotriazol-1-yl-N,N,N′,N′-tetramethyl-uronium hexafluorophosphate (128 mg) and triethylamine (59 ml) in acetonitrile (5 ml), with purification of the crude product by FC (50 g of silica gel, ethyl acetate-methanol 9:1).

### Example 36

In a manner analogous to that described in Example 31), the following compounds are prepared by lactone opening with n-butylamine:

a) From 110 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(3-azidopropoxy)-benzamide, (2S,4S,5S,7S)-N-[4-(tert-butoxycarbonyl)amino-7-butylcarbamoyl-5-hydroxy-2-isopropyl-8-methyl-nonyl]-2-(3-azidopropoxy)-benzamide, R_{f} (E)=0.14; HPLC Rₜ=18.8 min.

b) From 100 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(2-azidoethoxy)-benzamide, (2S,4S,5S,7S)-N-[4-(tert-butoxycarbonyl)amino-7-butylcarbamoyl-5-hydroxy-2-isopropyl-8-methyl-nonyl]-2-(2-azidoethoxy)-benzamide, R_{f} (ethyl acetate)=0.11; HPLC Rₜ=18.0 min.

c) From 200 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(4-acetylpiperazin-1-yl)-ethoxy]-benzamide, (2S,4S,5S,7S)-N-[4-(tert-butoxycarbonyl)amino-7-butylcarbamoyl-5-hydroxy-2-isopropyl-8-methyl-nonyl]-2-[2-(4-acetylpiperazin-1-yl)-ethoxy]-benzamide, R_{f} (dichloromethane-methanol 4:1)=0.70; HPLC Rₜ=13.4 min.

d) From 54 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(morpholin-4-yl)-ethyl]-benzamide, (2S,4S,5S,7S)-N-[4-(tert-butoxycarbonyl)amino-7-butylcarbamoyl-5-hydroxy-2-isopropyl-8-methyl-nonyl]-2-[2-(morpholin-4-yl)-ethyl]-benzamide, R_{f} (L)=0.38; HPLC Rₜ=15.7 min.

e) From 60 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(3-dimethylaminopropoxy)-benzamide, (2S,4S,5S,7S)-N-[4-(tert-butoxycarbonyl)amino-7-butylcarbamoyl-5-hydroxy-2-isopropyl-8-methyl-nonyl]-2-(3-dimethylaminopropoxy)-benzamide, R_{f} (J)=0.65; HPLC Rₜ=14.3 min.

f) From 49 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[3-(morpholin-4-yl)-propoxy]-benzamide, (2S,4S,5S,7S)-N-[4-(tert-butoxycarbonyl)amino-7-butylcarbamoyl-5-hydroxy-2-isopropyl-8-methyl-nonyl]-2-[3-(morpholin-4-yl)-propoxy]-benzamide, R_{f} (W)=0.70; HPLC Rₜ=14.2 min.

g) From 65 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl)}-2-[2-(morpholin-4-yl)-ethoxy]-benzamide, (2S,4S,5S,7S)-N-[4-(tert-butoxycarbonyl)amino-7-butylcarbamoyl-5-hydroxy-2-isopropyl-8-methyl-nonyl]-2-[2-(morpholin-4-yl)-ethoxy]-benzamide, R_{f} (H)=0.16; HPLC Rₜ=14.0 min.

h) From 135 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(4-methoxypiperidin-1-yl)-ethyl]-benzamide, (2S,4S,5S,7S)-N-[4-(tert-butoxycarbonyl)amino-7-butylcarbamoyl-5-hydroxy-2-isopropyl-8-methyl-nonyl]-2-[2-(4-methoxypiperidin-1-yl)-ethyl]-benzamide, R_{f} (ethyl acetate-methanol-conc. ammonia 90:15:5)=0.72; HPLC Rₜ=15.3 min.

i) From 165 mg of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-[2-(4-acetylpiperazin-1-yl)-ethyl]-benzamide, (2S,4S,5S,7S)-N-[4-(tert-butoxycarbonyl)amino-7-butyl carbamoyl-5-hydroxy-2-isopropyl-8-methyl-nonyl]-2-[2-(4-acetylpiperazin-1-yl)-ethyl]-benzamide, R_{f} (L)=0.45; HPLC Rₜ=13.9 min.

### Example 37

In a manner analogous to that described in Example 1a), the following compounds are prepared:

a) From 75 mg of (2S,4S,5S,7S)-N-[4-(tert-butoxycarbonyl)amino-7-butylcarbamoyl-5-hydroxy-2-isopropyl-8-methyl-nonyl]-2-(3-azidopropoxy)-benzamide, (2S,4S,5S,7S)-N-[4-(tert-butoxycarbonyl)amino-7-butylcarbamoyl-5-hydroxy-2-isopropyl-8-methyl-nonyl]-2-(3-aminopropoxy)-benzamide, R_{f} (J)=0.18; HPLC Rₜ=13.8 min.

b) From 47 mg of (2S,4S,5S,7S)-N-[4-(tert-butoxycarbonyl)amino-7-butylcarbamoyl-5-hydroxy-2-isopropyl-8-methyl-nonyl]-2-(2-azidoethoxy)-benzamide, (2S,4S,5S,7S)-N-(4-(tert-butoxycarbonyl)amino-7-butylcarbamoyl-5-hydroxy-2-isopropyl-8-methyl-nonyl]-2-(2-aminoethoxy)-benzamide, R_{f} (J)=0.25; HPLC Rₜ=13.4 min.

### Example 38

In a manner analogous to that described in Example 21), the following compounds are prepared by de-Bocylation:

a) From 58 mg of (2S,4S,5S,7S)-N-[4-(tert-butoxycarbonyl)amino-7-butylcarbamoyl-5-hydroxy-2-isopropyl-8-methyl-nonyl]-2-(3-aminopropoxy)-benzamide, (2S,4S,5S,7S)-N-(4-amino-7-butylcarbamoyl-5-hydroxy-2-isopropyl-8-methyl-nonyl)-2-(3-aminopropoxy)-benzamide dihydrochloride: R_{f} (ethyl acetate-methanol-conc. ammonia 50:45:5)=0.15. HPLC Rₜ=8.8 min. MS(FAB) m/e 507 (M⁺+1).

b) From 22 mg of (2S,4S,5S,7S)-N-[4-(tert-butoxycarbonyl)amino-7-butylcarbamoyl-5-hydroxy-2-isopropyl-8-methyl-nonyl]-2-(2-aminoethoxy)-benzamide, (2S_{,}4S,5S,7S)-N-(4-amino-7-butylcarbamoyl-5-hydroxy-2-isopropyl-8-methyl-nonyl)-2-(2-aminoethoxy)-benzamide dihydrochloride: R_{f} (ethyl acetate-methanol-conc. ammonia 50:45:5)=0.14. HPLC Rₜ=8.4 min. MS(FAB) m/e 493 (M⁺+1).

c) From 185 mg of (2S,4S,5S,7S)-N-[4-(tert-butoxycarbonyl)amino-7-butylcarbamoyl-5-hydroxy-2-isopropyl-8-methyl-nonyl]-2-[2-(4-acetylpiperazin-1-yl)-ethoxy]-benzamide, (2S,4S,5S,7S)-N-(4-amino-7-butylcarbamoyl-5-hydroxy-2-isopropyl-8-methyl-nonyl)-2-[2-(4-acetylpiperazin-1-yl)-ethoxy]-benzamide dihydrochloride: R_{f} (J)=0.35. HPLC Rₜ=10.0 min. MS(FAB) m/e 604 (M⁺+1).

d) From 48 mg of (2S,4S,5S,7S)-N-[4-(tert-butoxycarbonyl)amino-7-butylcarbamoyl-5-hydroxy-2-isopropyl-8-methyl-nonyl]-2-[2-(morpholin-4-yl)-ethyl]-benzamide, (2S,4S,5S,7S)-N-(4-amino-7-butylcarbamoyl-5-hydroxy-2-isopropyl-8-methyl-nonyl)-2-[2-(morpholin-4-yl)-ethyl]-benzamide dihydrochloride: R_{f} (J)=0.54. HPLC Rₜ=10.7 min. MS(FAB) m/e 547 (M⁺+1).

e) From 32 mg of (2S,4S,5S,7S)-N-[4-(tert-butoxycarbonyl)amino-7-butylcarbamoyl-5-hydroxy-2-isopropyl-8-methyl-nonyl]-2-(3-dimethylaminopropoxy)-benzamide, (2S,4S,5S,7S)-N-(4-amino-7-butylcarbamoyl-5-hydroxy-2-isopropyl-8-methyl-nonyl)-2-(3-dimethylaminopropoxy)-benzamide dihydrochloride: R_{f} (J)=0.31. HPLC Rₜ=9.2 min. MS(FAB) m/e 535 (M⁺+1).

f) From 40 mg of (2S,4S,5S,7S)-N-[4-(tert-butoxycarbonyl)amino-7-butylcarbamoyl-5-hydroxy-2-isopropyl-8-methyl-nonyl]-2-[3-(morpholin-4-yl)-propoxy]-benzamide, (2S,4S,5S,7S)-N-(4-amino-7-butylcarbamoyl-5-hydroxy-2-isopropyl-8-methyl-nonyl)-2-[3-(morpholin-4-yl)-propoxy]-benzamide dihydrochloride: R_{f} (W)=0.20. HPLC Rₜ=9.4 min. MS(FAB) m/e 577 (M⁺+1).

g) From 53 mg of (2S,4S,5S,7S)-N-[4-(tert-butoxycarbonyl)amino-7-butylcarbamoyl-5-hydroxy-2-isopropyl-8-methyl-nonyl]-2-[2-(morpholin-4-yl)-ethoxy]-benzamide, (2S,4S,5S,7S)-N-(4-amino-7-butylcarbamoyl-5-hydroxy-2-isopropyl-8-methyl-nonyl)-2-[2-(morpholin-4-yl)-ethoxy]-benzamide dihydrochloride: R_{f} (J)=0.54. HPLC Rₜ=9.3 min. MS(FAB) m/e 563 (M⁺+1).

h) From 32 mg of (2S,4S,5S,7S)-N-[4-(tert-butoxycarbonyl)amino-7-butylcarbamoyl-5-hydroxy-2-isopropyl-8-methyl-nonyl]-2-[2-(4-methoxypiperidin-1-yl)-ethyl]-benzamide, (2S,4S,5S,7S)-N-(4-amino-7-butylcarbamoyl-5-hydroxy-2-isopropy-8-methyl-nonyl)-2-[2-methoxypiperidin-1-yl)-ethyl]-benzamide dihydrochloride: R_{f} (ethylacetate-methanol-conc. ammonia 90:15:5)=0.29. HPLC Rₜ=10.4 min. MS(FAB) m/e 575 (M⁺+1).

i) From 137 mg of (2S,4S,5S,7S)-N-[4-(tert-butoxycarbonyl)amino-7-butylcarbamoyl-5-hydroxy-2-isopropyl-8-methyl-nonyl]-2-[2-(4-acetylpiperazin-1-yl)-ethyl]-benzamide, (2S,4S,5S,7S)-N-(4-amino-7-butylcarbamoyl-5-hydroxy-2-isopropyl-8-methyl-nonyl)-2-[2-(4-acetylpiperazin-1-yl)-ethyl]-benzamide dihydrochloride: R_{f} (ethylacetate-methanol-conc. ammonia 90:15:5)=0.19. HPLC Rₜ=9.5 min. MS(FAB) m/e 588 (M⁺+1).

### Example 39

Triethylamine (117 .mu.l) and cyanophosphonic acid diethyl ester (137 .mu.l) are added dropwise in succession at 0° C., with stirring, to a solution of (2S,2′S,2″S,4″S)-2-[2-azido-2′-(4″-isopropyl-5″-oxo-tetrahydrofuran-2″-yl)-ethyl]-3-methylbutyric acid (208 mg) (Case 4-19919/P1) in dichloromethane (17 ml). The reaction mixture is stirred a further 10 minutes at 0° C. and then a solution of 2-(3-methoxypropoxy)-benzylamine (164 mg) in dichloromethane (2 ml) is added dropwise. The mixture is stirred for a further 16 hours at room temperature and is then diluted with dichloromethane (100 ml), and the organic phase is washed with 10% citric acid solution (50 ml), saturated sodium hydrogen carbonate solution (50 ml) and saturated sodium chloride solution (50 ml). The aqueous phases are each back-extracted with dichloromethane (2×50 ml). The combined organic phases are dried over magnesium sulfate and concentrated by evaporation and the residue is purified by FC (18 g of silica gel, eluant E), yielding (2S,2′S,2″S,4″S)-2-[2′-azido-2′-(4″-isopropyl-5″-oxo-tetrahydrofuran-2″-yl)-ethyl]-N-[2-(3-methoxypropoxy)-benzyl]-3-methylbutyric acid amide, R_{f} (E)=0.32; HPLC Rₜ=18.0 min; MS(FAB) m/e 475 (M⁺+1), in the form of a light-yellow oil.

### Example 40

In a manner analogous to that described in Example 39), the following compounds are prepared:

a) From 208 mg of (2S,2′S,2″S,4″S)-2-[2′-azido-2′-(4″-isopropyl-5″-oxo-tetrahydrofuran-2″-yl)-ethyl]-3-methylbutyric acid and 164 mg of 3-(3-methoxypropoxy)-benzylamine, (2S,2″S,2″S,4″S)-2-[2′-azido-2′-(4″-isopropyl-5″-oxo-tetrahydrofuran-2″-yl)-ethyl]-N-[3-(3-methoxypropoxy)-benzyl]-3-methylbutyric acid amide, R_{f} (hexane-ethyl acetate-glacial acetic acid 66:33:1)=0.17; MS(FAB) m/e 475 (M⁺+1), in the form of a yellow oil.

b) From 210 mg of (2S,2′S,2″S,4″S)-2-[2′-azido-2′-(4″-isopropyl-5″-oxo-tetrahydrofuran-2″-yl)-ethyl]-3-methylbutyric acid and 177 mg of 2-(4-methoxybutoxy)-benzylamine, (2S,2′S,2″S,4″S)-2-[2′-azido-2′-(4″-isopropyl-5″-oxo-tetrahydrofuran-2″-yl)-ethyl-N-[2-(4-methoxybutoxy)-benzyl]-3-m ethylbutyric acid amide, R_{f} (hexane-ethyl acetate-glacial acetic acid 66:33:1)=0.2; HPLC Rₜ=18.3 min; MS(FAB) m/e 489 (M⁺+1), in the form of a light-yellow oil.

c) From 194 mg of (2S,2′S,2″S,4″S)-2-[2′-azido-2′-(4″-isopropyl-5″-oxo-tetrahydrofuran-2″-yl)-ethyl]-3-methylbutyric acid and 175 mg of 2-(5-methoxypentoxy)-benzylamine, (2S,2′S,2″S,4″S)-2-[2′-azido-2′-(4″-isopropyl-5″-oxo-tetrahydrofuran-2″-yl)-ethyl]-N-[2-(5-methoxypentoxy)-benzyl]-3-methylbutyric acid amide, R_{f} (hexane-ethyl acetate-glacial acetic acid 66:33:1)=0.38; HPLC Rₜ=19.1 min; MS(FAB) m/e 503 (M⁺+1), in the form of a colourless oil.

### Example 41

A solution of (2S,2′S,2″S,4″S)-2-[2′-azido-2′-(4″-isopropyl-5″-oxo-tetrahydrofuran-2″-yl)-ethyl]-N-[2-(3-methoxypropoxy)-benzyl]-3-methylbutyric acid amide (100 mg) in n-butylamine (0.5 ml) is stirred at 50°-55° C. for 16 hours and is then concentrated to dryness by evaporation. Purification of the residue by FC (5.5 g of silica gel, eluant F) yields (2S,4S,5S,7S)-N-[4-azido-5-hydroxy-2,7-diisopropyl-octanedioic acid 8-butylamide 1-[2-(3-methoxypropoxy)-benzyl]amide, R_{f} (F)=0.14; MS(FAB) m/e 548 (M⁺+1), in the form of a colourless oil.

### Example 42

In a manner analogous to that described in Example 41), the following compounds are prepared:

a) From 150 mg of (2S,2′S,2″S,4S)-2-[2′-azido-2′-(4″-isopropyl-5″-oxo-tetrahydrofuran-2″-yl)-ethyl]-N-[3-(3-methoxypropoxy)-benzyl]-3-methylbutyric acid amide, (2S,4S,5S,7S)-N-[4-azido-5-hydroxy-2,7-diisopropyl-octanedioic acid 8-butylamide 1-[3-(3-methoxypropoxy)-benzyl]amide, R_{f} (F)=0.28; MS(FAB) m/e 548 (M⁺+1), in the form of a yellow oil.

b) From 282 mg of (2S,2′S,2″S,4S)-2-[2′-azido-2′-(4″-isopropyl-5″-oxo-tetrahydrofuran-2″-yl)-ethyl]-N-[2-(4-methoxybutoxy)-benzyl]-3-methylbutyric acid amide, (2S,4S,5S,7S)-N-[4-azido-5-hydroxy-2,7-diisopropyl-octanedioic acid 8-butylamide 1-[2-(4-methoxybutoxy)-benzyl]amide, R_{f} (F)=0.21; HPLC Rₜ=17.6 min; MS(FAB) m/e 562 (M⁺+1), in the form of a light-yellow foam.

c) From 274 mg of (2S,2′S,2″S,4S)-2-[2′-azido-2′-(4″-isopropyl-5″-oxo-tetrahydrofuran-2″-yl)-ethyl]-N-[2-(5-methoxypentyloxy)-benzyl]-3-methylbutyric acid amide, (2S,4S,5S,7S)-N-[4-azido-5-hydroxy-2,7-diisopropyl-octanedioic acid 8-butylamide 1-[2-(5-methoxypentyloxy)-benzyl]amide, R_{f} (F)=0.5; HPLC Rₜ=18.3 min; MS(FAB) m/e 576 (M⁺+1), in the form of a light-yellow oil.

### Example 43

A solution of (2S,4S,5S,7S)-N-[4-azido-5-hydroxy-2,7-diisopropyl-octanedioic acid 8-butylamide 1-[2-(3-methoxypropoxy)-benzyl]amide (38 mg) in methanol (8 ml) is hydrogenated for 4 hours at room temperature and under normal pressure in the presence of 10% Pd/C (20 mg), the pH being kept at a constant value of 6 by the addition of 0.1N methanolic hydrochloric acid solution. The reaction mixture is then filtered over kieselguhr and concentrated by evaporation. The residue is dissolved twice in a small amount of toluene and is again concentrated by evaporation; one drop of 4N hydrochloric acid solution in dioxane is then added and concentration by evaporation is carried out again under a high vacuum. Purification of the crude product by FC (1.4 g of silica gel, eluant L) yields (2S,4S,5S,7S)-4-amino-5-hydroxy-2,7-diisopropyl-octanedioic acid 8-butylamide 1-[2-(3-methoxypropoxy)-benzyl]amide hydrochloride in the form of a yellowish amorphous powder: R_{f} (dichloromethane-methanol 8:2)=0.37; HPLC Rₜ=12.8 min; MS(FAB) m/e 522 (M⁺+1)

### Example 44

In a manner analogous to that described in Example 43), the following compounds are prepared:

a) From 135 mg of (2S,4S,5S,7S)-N-[4-azido-5-hydroxy-2,7-diisopropyl-octanedioic acid 8-butyliamide 1-[3-(3-methoxypropoxy)-benzyl]amide, (2S,4S,5S,7S)-4-amino-5-hydroxy-2,7-diisopropyl-octanedioic acid 8-butylamide 1-[3-(3-methoxypropoxy)-benzyl]amide hydrochloride in the form of a yellowish amorphous solid: R_{f} (dichloromethane-methanol 8:2)=0.62; HPLC Rₜ=12.4 min; MS(FAB) m/e 522 (M⁺+1).

b) From 234 mg of (2S,4S,5S,7S)-N-[4-azido-5-hydroxy-2,7-diisopropyl-octanedioic acid 8-butylamide 1-[2-(4-methoxybutoxy)-benzyl]amide, (2S,4S,5S,7S)-4-amino-5-hydroxy-2,7-diisopropyl-octanedioic acid 8-butylamide 1-[2-(4-methoxy butoxy)-benzyl]amide hydrochloride in the form of a colorless amorphous powder: R_{f} (L)=0.27; HPLC Rₜ=13.2 min; MS(FAB) m/e 536 (M⁺+1).

c) From 228 mg of (2S,4S,5S,7S)-N-[4-azido-5-hydroxy-2,7-diisopropyl-octanedioic acid 8-butylamide 1-[2-(5-methoxypentyloxy)-benzyl]amide, (2S,4S,5S,7S)-4-amino-5-hydroxy-2,7-diisopropyl-octanedioic acid 8-butylamide 1-[2-(5-methoxypentyloxy)-benzyl]amide hydrochloride in the form of a yellowish amorphous powder: R_{f} (L)=0.33; HPLC Rₜ=13.2 min; MS(FAB) m/e 550 (M⁺+1).

### Example 45

Reaction of (3S,5S,1′S,3′S)-5-[3′-aminomethyl-11′-(tert-butoxycarbonyl)amino-4′-methylpentyl]-3-isopropyl-dihydrofuran-2-one (100 mg) with 3-(4-methoxybutoxy)-terephthalic acid N-(methyl)amide (119 mg) in a manner analogous to that described in Example 1) yields (2S,2′S,2″S,4″S)-N1-{2-[2′-(tert-butoxycarbonyl)amino-2′-(4″-isopropyl-5″-oxo-tetrahydrofuran-2″-yl)-ethyl]-3-methylbutyl}-N-4-methyl-2-(4-methoxybutoxy)-terephthalamide, R_{f} (L)=0.59; MS(FAB) m/e 620 (M⁺+1), in the form of an amorphous white powder.

### Example 46

In a manner analogous to that described in Example 45), the following compounds are prepared:

a) From 400 mg of (3S,5S,1′S,3′S)-5-[3′-aminomethyl-1′-(tert-butoxycarbonyl) amino-4′-methylpentyl]-3-isopropyl-dihydrofuran-2-one and 546 mg of 3-(4-methoxybutoxy)-terephthalic acid (tert-butyl) ester, (2S,2′S,2″S,4″S)-N-{2-[2′-(tert-butoxycarbonyl) amino-2′-(4″-isopropyl-5″-oxo-tetrahydrofuran-2″-yl)-ethyl]-3-methylbutyl}-3-(4-methoxybutoxy)-terephthalamic acid (tert-butyl) ester, R_{f} (E)=0.48; MS(FAB) m/e 664 (M⁺+1), in the form of a slightly yellowish oil.

b) From 100 mg of (3S,5S,1′S,3′S)-5-[3′-aminomethyl-1′-(tert-butoxycarbonyl) amino-4′-methylpentyl]-3-isopropyl-dihydrofuran-2-one and 160 mg of 3-(4-methoxybutoxy)-terephthalic acid N-[2-morpholin-4-yl)-ethyl]amide, (2S,2′S,2″S,4″S)-N1-{2-[2′-(tert-butoxycarbonyl)amino-2′-(4″-isopropyl-5″-oxo-tetrahydrofuran-2″-yl)-ethyl]-3-methylbutyl}-N4-[2-morpholin-4-yl)-ethyl]-2-(4-methoxybutoxy)-terephthaldiamide, R_{f} (L)=0.63; MS (FAB) m/e 719 (M⁺+1), in the form of a yellowish oil.

c) From 120 mg of (3S,5S,1′S,3′S)-5-[3′-aminomethyl-1′-(tert-butoxycarbonyl) amino-4′-methylpentyl]-3-isopropyl-dihydrofuran-2-one and 135 mg of 3-(4-methoxybutoxy)-terephthalic acid monoamide, (2S,2′S,2″S,4″S)-N1-{2-[2′-(tert-butoxycarbonyl) amino-2′-(4″-isopropyl-5″-oxo-tetrahydrofuran-2″-yl)-ethyl]-3-methylbutyl}-2-(4-methoxybutoxy)-terephthaldiamide, R_{f} (L)=0.58; MS(FAB) m/e 606 (M⁺+1), in the form of a yellow amorphous powder.

The terephthalic acid derivatives used as starting materials are prepared in accordance with customary methods from the literature, unless described otherwise in greater detail below:

A) 2-(4-Methoxybutoxy)-N-methyl-terephthalamic acid: Alkaline hydrolysis of 300 mg of 2-(4-methoxybutoxy)-N-methyl-terephthalamic acid methyl ester in the manner described in Example 30) yields the title compound, R_{f} (L)=0.15, in the form of a white powder.

a) 2-(4-Methoxybutoxy)-N-methyl-terephthalamic acid methyl ester: 2-(4-Methoxybutoxy)-terephthalic acid 1-methyl ester (564 mg) and thionyl chloride (3 ml) are stirred under reflux for one hour. The acid chloride obtained after concentration by evaporation is dissolved in tetrahydrofuran (5 ml) and added in metered amounts at −10° C. to a 40% aqueous methylamine solution (5 ml). When the addition is complete, the solvent is concentrated by evaporation and the residue is partitioned between ethyl acetate and a 2N aqueous hydrochloric acid solution. The organic phase is separated off, washed with water, dried over magnesium sulfate and concentrated by evaporation. Purification by FC (eluant Q) yields the title compound, R_{f} (N)=0.55, in the form of a yellowish amorphous powder.

b) 2-(4-Methoxybutoxy)-terephthalic acid 1-methyl ester: Alkaline hydrolysis of 2-(4-methoxybutoxy)-terephthalic acid dimethyl ester (5 g) in the manner described in Example 16a) yields the title compound, R_{f} (L)=0.32, in the form of a white powder.

c) 2-(4-Methoxybutoxy)-terephthalic acid dimethyl ester: Alkylation of 2-hydroxy-terephthalic acid dimethyl ester (10 g) with 4-methoxybutyl bromide in anhydrous acetone in the presence of dried potassium carbonate and potassium iodide in a manner analogous to that described in Example 16a) yields the title compound, R_{f} (B)=0.20, in the form of a slightly yellowish oil.

B) 2-(4-Methoxybutoxy)-terephthalic acid 4-tert-butyl ester 1-methyl ester: 1,1′-Carbonyldiimidazole (1.65 g) is added to a solution of 2-(4-methoxybutoxy)-terephthalic acid 1-methyl ester (2.8 g) in N,N-dimethylformamide (10 ml), and the mixture is stirred at 40° C. for one hour. After the addition of tert-butanol (1.48 g) and 1,8-diazabicyclo[5.4.0]undec-7-ene (1.52 g), stirring is continued at 40° C. for 24 hours and the reaction mixture is then concentrated by evaporation. The residue is partitioned between ethyl acetate and water and the organic phase is separated off, dried over magnesium sulfate and concentrated by evaporation. Purification by FC (eluant B) yields the title compound, R_{f} (B)=0.3, in the form of a yellowish oil.

C) 2-(4-Methoxybutoxy)-N-(2-morpholin-4-ylethyl)-terephthalamic acid: Alkaline hydrolysis of 300 mg of 2-(4-methoxybutoxy)-N-(2-morpholin-4-ylethyl)-terephthalamic acid methyl ester in the manner described in Example 16a) yields the title compound, R_{f} (L)=0.33, in the form of a white powder.

a) 2-(4-Methoxybutoxy)-N-(2-morpholin-4-ylethyl)-terephthalamic acid methyl ester: Reaction of 500 mg of 2-(4-methoxybutoxy)-terephthalic acid 1-methyl ester with thionyl chloride and then reaction with a solution of 4-(2-aminoethyl)-morpholine and triethylamine in dichlormethane, in a manner analogous to that described in Example 46Aa), yield the title compound, R_{f} (L)=0.6, in the form of a white amorphous powder.

D) 2-(4-Methoxybutoxy)-terephthalamic acid: Alkaline hydrolysis of 2-(4-methoxybutoxy)-terephthalamic acid methyl ester (300 mg) in the manner described in Example 16a) yields the title compound, R_{f} (dichloromethane-methanol-acetic acid-water 90:10:0.5:1)=0.33, in the form of a white powder.

a) 2-(4-Methoxybutoxy)-terephthalamic acid methyl ester: Reaction of 2-(4-methoxybutoxy)-terephthalic acid 1-methyl ester (500 mg) with thionyl chloride and then reaction with 25% aqueous ammonia, in a manner analogous to that described in Example 46Aa), yield the title compound, R_{f} (N)=0.38, in the form of a white amorphous powder.

### Example 47

In a manner analogous to that described in Example 18), the following compounds are prepared by lactone opening:

a) From 100 mg of (2S,2′S,2″S,4″S)-N1-{2-[2′-(tert-butoxycarbonyl)amino-2′-(4″-isopropyl-5″-oxo-tetrahydrofuran-2″-yl)-ethyl]-3-methylbutyl}-N-4-methyl-2-(4-methoxybutoxy)-terephthaldiamide, (2S,4S,5S,7S)-N1-(4-(tert-butoxycarbonyl)amino-7-butylcarbamoyl-5-hydroxy-2-isopropyl-8-methyl-nonyl]-N-4-methyl-2-(4-methoxybutoxy)-terephthaldiamide, R_{f} (L)=0.34; MS(FAB) m/e 693 (M⁺+1), in the form of a white amorphous powder.

b) From 200 mg of (2S,2′S,2″S,4″S)-N1-{2-[2′-(tert-butoxycarbonyl)amino-2′-(4″-isopropyl-5″-oxo-tetrahydrofuran-2″-yl)-ethyl]-3-methylbutyl}-3-(4-methoxybutoxy)-terephthalamic acid (tert-butyl) ester, (2S,4S,5S,7S)-N1-(4-(tert-butoxycarbonyl) amino-7-butylcarbamoyl-5-hydroxy-2-isopropyl-8-methyl-nonyl]-3-(4-methoxybutoxy)-terephthalamic acid (tert-butyl) ester, R_{f} (E)=0.20; MS(FAB) m/e 737 (M⁺+1), in the form of a yellow oil.

c) From 170 mg of (2S,2′S,2″S,4″S)-N1-{2-[2′-(tert-butoxycarbonyl)amino-2′-(4″-isopropyl-5″-oxo-tetrahydrofuran-2″-yl)-ethyl]-3-methylbutyl}-N-4-[(2-morpholin-4-yl)-ethyl]-2-(4-methoxybutoxy)-terephthaldiamide, (2S,4S,5S,7S)-N-(4-(tert-butoxycarbonyl)amino-7-butylcarbamoyl-5-hydroxy-2-isopropyl-8-methyl-nonyl]-N-4-[2-morpholin-4-yl)-ethyl]-2-(4-methoxybutoxy)-terephthaldiamide, R_{f} (L)=0.43; MS(FAB) m/e 792 (M⁺+1), in the form of a yellow amorphous powder.

d) From 200 mg of (2S,2′S,2″S,4″S)-N1-{2-[2′-(tert-butoxycarbonyl)amino-2′-(4″isopropyl-5″-oxo-tetrahydrofuran-2″-yl)-ethyl]-3-methylbutyl}-2-(4-methoxybutoxy)-terephthaldiamide, (2S,4S,5S,7S)-N1-[4-(tert-butoxycarbonyl)amino-7-butylcarbamoyl-5-hydroxy-2-isopropyl-8-methyl-nonyl]-2-(4-methoxybutoxy)-terephthaldiamide, R_{f} (L)=0.38; MS(FAB) m/e 679 (M⁺+1).

### Example 48

In a manner analogous to that described in Example 21), the following compounds are obtained by de-Bocylation:

a) From 100 mg of (2S,4S,5S,7S)-N1-[4-(tert-butoxycarbonyl)amino-7-butylcarbamoyl-5-hydroxy-2-isopropyl-8-methyl-nonyl]-N-4-methyl-2-(4-methoxybutoxy)-terephthaldiamide, (2S,4S,5S,7S)-N1-(4-amino-7-butylcarbamoyl-5-hydroxy-2-isopropyl-8-methyl-nonyl)-N-4-methyl-2-(4-methoxybutoxy)-terephthaldiamide hydrochloride: R_{f} (L)=0.13. HPLC Rₜ=11.6 min. MS(FAB) m/e 593 (M⁺+1).

b) From 170 mg of (2S,4S,5S,7S)-N1-(4-(tert-butoxycarbonyl)amino-7-butylcarbamoyl-5-hydroxy-2-isopropyl-8-methyl-nonyl]-N-4-[(2-morpholin-4-yl)-ethyl]-2-(4-methoxybutoxy)-terephthaldiamide, (2S,4S,5S,7S)-N1-(4-amino-7-butylcarbamoyl-5-hydroxy-2-isopropyl-8-methyl-nonyl)-N-4-[2-morpholin-4-yl)-ethyl]-2-(4-methoxybutoxy)-terephthaldiamide dihydrochloride: HPLC Rₜ=9.73 min. MS(FAB) m/e 692 (M⁺+1).

c) From 175 mg of (2S,4S,5S,7S)-N1-(4-(tert-butoxycarbonyl)amino-7-butylcarbamoyl-5-hydroxy-2-isopropyl-8-methyl-nonyl]-2-(4-methoxybutoxy)-terephthalic acid diamide, (2S,4S,5S,7S)-N1-(4-amino-7-butylcarbamoyl-5-hydroxy-2-isopropyl-8-methyl-nonyl)-2-(4-methoxybutoxy)-terephthalic acid diamide hydrochloride: R_{f} (L)=0.1. HPLC Rₜ=11.0 min. MS(FAB) m/e 579 (M⁺+1)

### Example 49

Reaction of (2S,4S,5S,7S)-N1-[4-(tert-butoxycarbonyl)amino-7-butylcarbamoyl-5-hydroxy-2-isopropy-8-methylnonyl]-3-(4-methoxybutoxy)-terephthalamic acid (tert-butyl) ester (205 mg) in 3 ml of a 1:1 mixture of dichloromethane and trifluoroacetic acid at 0° C. yields (2S,4S,5S,7S)-N-4-(4-amino-7-butylcarbamoyl-5-hydroxy-2-isopropyl-8-methyl-nonyl)-3-(4-methoxybutoxy)-terephthalamic acid trifluoroacetate: HPLC Rₜ=11.9 min MS(FAB) m/e 580 (M⁺+1).

### Example 50

In a manner analogous to that described in Example 21), reaction of (2S,4S,5S,7S)-N-[4-(tert-butoxycarbonyl)amino-7-butylcarbamoyl-5-hydroxy-2-isopropyl-8-methyl-nonyl]-4-butylcarbamoylmethoxy-2-(4-methoxybutoxy)-benzamide (80 mg) in 4N hydrochloric acid solution in dioxane at 0° C. for one hour, rapid concentration of the solvent under a high vacuum and lyophilisation yield (2S,4S,5S,7S)-N-(4-amino-7-butylcarbamoyl-5-hydroxy-2-isopropyl-8-methyl-nonyl)-4-butylcarbamoylmethoxy-2-(4-methoxybutoxy)-benzamide hydrochloride: R_{f} (W)=0.23. HPLC Rₜ=13.8 min. MS(FAB) m/e 665 (M⁺+1).

The (2S,4S,5S,7S)-N-[4-(tert-butoxycarbonyl)amino-7-butyl carbamoyl-5-hydroxy-2-isopropyl-8-methyl-nonyl]-4-butylcarbamoylmethoxy-2-(4-methoxybutoxy)-benzamide used as starting material is prepared as follows:

a) A solution of (2S,2′S,2″S,4″S)-4-{2-[2′-(tert-butoxycarbonyl)amino-2′-(4″-isopropyl-5″-oxo-tetrahydrofuran-2″-yl)-ethyl]-3-methylbutylcarbamoyl}-3-(4-methoxybutoxy)-phenoxy)-acetic acid (tert-butyl) ester (239 mg) in n-butylamine (3 ml) is stirred at 50° C. for 18 hours. The reaction mixture is concentrated and the residue is chromatographed (FC on 50 g of silica gel, eluant T). There are obtained (2S,4S,5S,7S)-N-(4-(tert-butoxycarbonyl)amino-7-butylcarbamoyl-5-hydroxy-2-isopropyl-8-methyl-nonyl]-4-butylcarbamoylmethoxy-2-(4-methoxybutoxy)-benzamide (83 mg; R_{f} (W)=0.46; HPLC Rₜ=18.7 min) and 94 mg of (2S,4S,5S,7S)-{4-[4-(tert-butoxycarbonyl)amino-7-butylcarbamoyl-5-hydroxy-2-isopropyl-8-methyl-nonylcarbamoyl]-3-(4-methoxybutoxy)-phenoxy}-acetic acid (tert-butyl) ester (R_{f} (W)=0.50; HPLC Rₜ=20.1 min), as well as 26 mg of a mixed fraction of the two products.

b) (2S,2′S,2″S,4″S)-4-{2-[2′-(tert-Butoxycarbonyl)amino-2′-(4″-isopropyl-5″-oxo-tetrahydrofuran-2″-yl)-ethyl]-3-methylbutylcarbamoyl}-3-(4-methoxybutoxy)-phenoxy)-acetic acid (tert-butyl) ester: tert-Butyl bromoacetate (76 μl) and cesium carbonate (169 mg) are added at room temperature to a solution of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-4-hydroxy-2-(4-methoxybutoxy)-benzamide (200 mg) in acetone (10 ml). The white suspension is stirred under reflux for 2 hours, cooled and then filtered, and the filtrate is concentrated. Drying under a high vacuum yields the title compound in the form of a yellow oil (255 mg). R_{f} (L)=0.73. HPLC Rₜ=19.9 min.

c) (2S,2′S,2″S,4″S)-N-{2-[2′-(tert-Butoxycarbonyl)amino-2′-(4″-isopropyl-5″-oxo-tetrahydrofuran-2″-yl)-ethyl]-3-methylbutyl}-4-hydroxy-2-(4-methoxybutoxy)-benzamide: (2S,2′S,2″S,4″S)-N-{2-[2′-(tert-Butoxycarbonyl)amino-2′-(4″-isopropyl-5″-oxo-tetrahydrofuran-2″-yl)-ethyl]-3-methylbutyl}-4-benzyloxy-2-(4-methoxybut oxy)-benzamide (2.46 g), dissolved in ethyl acetate (60 ml), is hydrogenated for 15 hours at room temperature in the presence of 5% Pd/C (Degussa) (250 mg). Filtration over Celite 545 and concentration of the filtrate yield the title compound (2.05 g). R_{f} (L)=0.47. HPLC Rₜ=16.0 min.

d) (2S,2′S,2″S,4″S)-N-{2-[2′-(tert-Butoxycarbonyl)amino-2′-(4″-isopropyl-5″-oxo-tetrahydrofuran-2″-yl)-ethyl]-3-methylbutyl}-4-benzyloxy-2-(4-methoxybutoxy)-benzamide: Reaction of (3S,5S,1′S,3′S)-5-[3′-aminomethyl-1′-(tert-butoxycarbonyl)amino-4′-methylpentyl]-3 isopropyldihydrofuran-2-one (3.33 g) with 4-benzyloxy-2-(4-methoxybutoxy)-benzoic acid (3.70 g) in a manner analogous to that described in Example 1) and then purification by FC (eluant Q) yield the title compound, R_{f} (L)=0.79, in the form of an oil.

The 4-benzyloxy-2-(4-methoxybutoxy)-benzoic acid used as starting material is prepared as follows:

a) 4-Benzyloxy-2-(4-methoxybutoxy)-benzoic acid: The title compound is obtained in the form of a pale-yellow oil, R_{f} (G)=0.38, by alkaline hydrolysis of 4-benzyloxy-2-(4-methoxybutoxy)-benzoic acid methyl ester.

b) 4-Benzyloxy-2-(4-methoxybutoxy)-benzoic acid methyl ester: A 30% methanolic sodium methoxide solution (21 ml) is added dropwise under reflux over a period of 30 minutes to a solution of 4-benzyloxy-2-(4-bromobutoxy)-benzoic acid methyl ester (29.6 g) in anhydrous methanol (250 ml), and the mixture is stirred overnight. After cooling, the mixture is concentrated to half its volume, water (50 ml) is added, and the pH is adjusted to 2 by the addition of 1M potassium hydrogen sulfate solution. Extraction with dichloromethane and purification of the crude product by FC (2 kg of silica gel, hexane-ethyl acetate 7:1) yield the title compound in the form of a solid (18.8 g): R_{f} (C)=0.24. M.P. 72°-74° C.

c) 4-Benzyloxy-2-(4-bromobutoxy)-benzoic acid methyl ester: 4-Benzyloxy-2-hydroxybenzoic acid methyl ester (20.0 g) (prepared in accordance with the procedure described in J. Med. Chem. (1985), 28, 717-727) and 1,4-dibromobutane (91.2 ml), dissolved in acetone (200 ml), are stirred under reflux for 30 hours in the presence of anhydrous powdered potassium carbonate (16.0 g). After filtration and concentration, the crude product is purified by FC (400 g of silica gel, eluant A). The title compound is obtained in the form of a yellowish solid (29.7 g): R_{f} (C)=0.35.

### Example 51

After reaction of (2S,4S,5S,7S)-{4-[4-(tert-butoxycarbonyl)amino-7-butylcarbamoyl-5-hydroxy-2-isopropyl-8-methyl-nonylcarbamoyl]-3-(4-methoxybutoxy)-phenoxy}-acetic acid (tert-butyl) ester (93 mg) in a 4N hydrochloric acid solution in dioxane (2 ml) at 0° C. for 45 minutes and then at room temperature for 13 hours, the solvent is rapidly concentrated under a high vacuum with vigorous stirring until frozen and is subsequently removed by lyophilisation. (2S,4S,5S,7S)-[4-(4-amino-7-butylcarbamoyl-5-hydroxy-2-isopropyl-8-methyl-nonylcarbamoyl)-3-(4-methoxybutoxy)-phenoxy]-acetic acid is obtained: HPLC Rₜ=11.7 min. MS(FAB) m/e 610 (M⁺+1)

### Example 52

In a manner analogous to that described in Example 21), 85 mg of (2S,4S,5S,7S)-N-{4-(tert-butoxycarbonyl)amino-7-[2-(morpholin-4-yl)-ethylcarbamoyl]-5-hydroxy-2-isopropyl-8-methyl-nonyl}-2-(4-methoxybutoxy)-4-[2-(morpholin-4-yl)-ethylcarbamoylmethoxy]-benzamide yield (2S,4S,5S,7S)-N-{4-amino-5-hydroxy-2-isopropyl-8-methyl-7-[2-(morpholin-4-yl)-ethylcarbamoyl]-nonyl}-2-(4-methoxybutoxy)-4-[2-(morpholin-4-yl)-ethylcarbamoylmethoxy]-benzamide trihydrochloride: R_{f} (W)=0.07. HPLC Rₜ=7.69 min. HRMS(FAB) m/e 779.5264.

The (2S,4S,5S,7S)-N-{4-(tert-butoxycarbonyl)amino-7-[2-(morpholin-4-yl)-ethylcarbamoyl]-5-hydroxy-2-isopropyl-8-methyl-nonyl}-2-(4-methoxybutoxy)-4-[2-(morpholin-4-yl)-ethylcarbamoylmethoxy]-benzamide used as starting material is prepared as follows:

a) A solution of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-carbamoylmethoxy]benzamide (98 mg) in N-(2-aminoethyl)morpholine (0.5 ml) is stirred overnight at 80° C. The reaction mixture is then immediately chromatographed on 25 g of silica gel (eluant gradient from P to L). The title compound, R_{f} (W)=0.41; HPLC Rₜ=9.85 min; MS(FAB) m/e 880 (M⁺+1), is obtained in the form of a yellowish foam.

b) (2S,2′S,2″S, 4″S)-N-{2-[2′-(tert-Butoxycarbonyl)amino-2′-(4″-isopropyl-5″-oxo-tetrahydrofuran-2″-yl)-ethyl]-3-methylbutyl}-2-(4-methoxybutoxy)-4-carbamoylmethoxy]benzamide: A mixture of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-4-hydroxy-2-(4-methoxybutoxy)-benzamide (200 mg), 2-bromoacetamide (72 mg) and caesium carbonate (169 mg) in anhydrous acetone (10 ml) is stirred under reflux for 2 hours. After cooling, filtration is carried out, the filtrate is concentrated and the residue is dried under a high vacuum. 169 mg of the title compound, R_{f} (L)=0.59; MS(FAB m/e 636 (M⁺+1), are obtained in the form of a white solid.

### Example 53

In a manner analogous to that described in Example 21), reaction of (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(1H-tetrazol-5-ylmethoxy)-benzamide (88 mg) in a 4N hydrochloric acid solution in dioxane at 0° C. for 6 hours, rapid concentration of the solvent under a high vacuum and lyophilisation yield (2S,4S,5S,7S)-N-(4-amino-7-butylcarbamoyl-5-hydroxy-2-isopropyl-8-methyl-nonyl)-2-(4-methoxybutoxy)-4-(1H-tetrazol-5-ylmethoxy)-benzamide hydrochloride: R_{f} (W)=0.06. HPLC Rₜ=11.5 min. MS(FAB) m/e 634 (M⁺+1).

The (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(1H-tetrazol-5-ylmethoxy)-benzamide used as starting material is prepared as follows:

a) From (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-(1H-tetrazol-5-ylmethoxy)-benzamide (104 mg) in a manner analogous to that described in Example 18), by reaction in n-butylamine at 50° C. for 20 hours. The reaction mixture is concentrated and the residue is taken up in dichloromethane (50 ml). The organic phase is washed with ice-water (pH 4), and the aqueous phase is back-extracted twice with dichloromethane. The combined organic phases are washed with saturated sodium chloride solution and dried over magnesium sulfate and the solvent is removed in vacuo. Drying under a high vacuum yields (2S,4S,5S,7S)-N-[4-(tert-butoxycarbonyl)amino-7-butylcarbamoyl-5-hydroxy-2-isopropyl-8-methyl-nonyl]-2-(4-methoxybutoxy)-4-(1H-tetrazol-5-ylmethoxy)-benzamide, R_{f} (dichloromethane-methanol-glacial acetic acid 9:1:0.1)=0.31, in the form of a yellowish solid.

b) (2S,2″S,2″S,4″S)-N-{2-[2′-(tert-Butoxycarbonyl)amino-2′-(4″-isopropyl-5″-oxo-tetrahydrofuran-2″-yl)-ethyl]-3-methylbutyl}-2-(4-methoxybutoxy)-4-(1H-tetrazol-5-ylmethoxy)-benzamide: The title compound, R_{f} (W)=0.32, is obtained in the form of a solid from (3S,5S,1′S,3′S)-5-[3′-aminomethyl-1′-(tert-butoxycarbonyl)amino-4′-methylpentyl]-3-isopropyl-dihydrofuran-2-one (80 mg) and 2-(4-methoxybutoxy)-4-(1H-tetrazol-5-ylmethoxy)-benzoic acid (145 mg) after purification of the crude product by FC on 25 g of silica gel (eluant: dichloromethane-methanol-glacial acetic acid 95:5:1; mixed fractions are chromatographed repeatedly under the same conditions or with dichloromethane-methanol-conc. ammonia 95:5:1).

The 2-(4-methoxybutoxy)-4-(1H-tetrazol-5-ylmethoxy)-benzoic acid used as starting material is prepared as follows:

a) 2-(4-Methoxybutoxy)-4-(1H-tetrazol-5-ylmethoxy)-benzoic acid methyl ester (1.0 g) is hydrolysed with 1N sodium hydroxide solution (3.6 ml) in methanol (10 ml) and water (5 ml). The reaction mixture is diluted with dichloromethane (50 ml) and the aqueous phase is adjusted to pH 2 with a 1M potassium hydrogen sulfate solution. Extraction is carried out repeatedly with dichloromethane, the combined organic phases are washed with saturated sodium chloride solution and dried over magnesium sulfate, and the solvent is removed. The title compound (780 g), R_{f} (dichloromethane-methanol-glacial acetic acid 40:10:1)=0.61, is obtained in the form of a white powder.

b) 2-(4-Methoxybutoxy)-4-(1H-tetrazol-5-ylmethoxy)-benzoic acid methyl ester: A mixture of 2-(4-methoxybutoxy)-4-(cyanomethoxy)-benzoic acid methyl ester (1.0 g), sodium azide (1.02 g) and ammonium chloride (0.84 g) in absolute N,N-dimethylformamide (30 ml) is stirred overnight at 135° C. The brown suspension is concentrated and the residue is purified by FC (80 g of silica gel, eluant: dichloromethane-methanol-conc. ammonia 40:10:1). The title compound, R_{f} (dichloromethane-methanol-glacial acetic acid 40:10:1)=0.71; R_{f} (dichloromethane-methanol-conc. ammonia 40:10:1)=0.29, is obtained in the form of a brown oil.

c) 2-(4-Methoxybutoxy)-4-(cyanomethoxy)-benzoic acid methyl ester: The title compound is obtained in the form of an oil, R_{f} (E)=0.38, from 4-hydroxy-2-(4-methoxybutoxy)-benzoic acid methyl ester (3.0 g), chloroacetonitrile (1.9 ml) and caesium carbonate (5.8 g) in a manner analogous to that described in Example 30Ba).

### Example 54

Cyanophosphonic acid diethyl ester (26 μl) and 2-aminopropionic acid N,N-(dimethyl)amide (26 mg) are added at 0° C., with stirring, to a solution of (2S,4S,5S,7S)-5-(tert-butoxycarbonyl)amino-4-(tert-butyl)dimethylsilyloxy-2-isopropyl-7-{[2-(4-methoxybutoxy)-benzoylamino]-methyl}-8-methyl-nonanoic acid (100 mg) and triethylamine (48 μl) in N,N-dimethylformamide (4 ml). After 30 minutes, the mixture is allowed to warm to room temperature and stirring is continued overnight. The mixture is concentrated and the residue is taken up in ethyl acetate. After washing the organic phase with 10% citric acid solution, saturated sodium hydrogen carbonate solution and saturated sodium chloride solution, drying over magnesium sulfate and concentration are carried out. Purification by FC (25 g of silica gel, eluant V) yields (2S,4S,5S,7S)-N-(4-(tert-butoxycarbonyl)amino-5-(tert-butyl)-dimethylsilyloxy-7-[2-(dimethylaminocarbamoyl)-ethylcarbamoyl]-2-isopropyl-8-methyl-nonyl)-2-(4-methoxybutoxy)-benzamide, R_{f} (dichloromethane-methanol-conc. ammonia 95:5:1)=0.21; MS(FAB) m/e 794 (M⁺+1), in the form of a yellow oil.

The (2S,4S,5S,7S)-5-(tert-butoxycarbonyl)amino-4-(tert-butyl)dimethylsilyloxy-2-isopropyl-7-{[2-(4-methoxybutoxy)-benzoylamino]-methyl}-8-methyl-nonanoic acid used as starting material is prepared as follows:

a) (2S,4S,5S,7S)-5-(tert-Butoxycarbonyl)amino-4-(tert-butyl)dimethylsilyloxy-2-isopropyl-7-{[2-(4-methoxybutoxy)-benzoylamino]-methyl}-8-methyl-nonanoic acid: A solution of (2S,4S,5S,7S)-5-(tert-butoxycarbonyl)amino-4-hydroxy-2-isopropyl-7-{[2-(4-methoxybutoxy)-benzoylamino]-methyl}-8-methyl-nonanoic acid (2.96 g), tert-butyldimethylsilyl chloride (1.69 g) and imidazole (1.46 g) in N,N-dimethylformamide (30 ml) is stirred at room temperature for 3 days. Then the reaction mixture is concentrated and the residue is partitioned between ice-water and ethyl acetate. After extraction of the aqueous phase with ethyl acetate, the ice-cold organic phase is washed with 10% citric acid solution, saturated sodium hydrogen carbonate solution and water, dried over magnestrated. The crude concentrated. The crude silyl ester (3.81 g, yellow oil) is stirred overnight at room temperature in a mixture of tetrahydrofuran (15 ml), water (6 ml) and glacial acetic acid (15 ml). Concentration of the reaction mixture and customary working-up by extraction with ethyl acetate yield a yellow oil, from which the title compound (2.01 g) is obtained in the form of a foamy solid, R_{f} (E)=0.32; MS(FAB) m/e 695 (M⁺+1), after FC (400 g of silica gel, elution first with eluant gradient from D to F, then complete elution of the product with eluant L).

b) (2S,4S,5S,7S)-5-(tert-Butoxycarbonyl)amino-4-hydroxy-2-isopropyl-7-{[2-(4-methoxybutoxy)-benzoylamino]-methyl}-8-methyl-nonanoic acid: 21.6 ml of a 1M lithium hydroxide solution are added to a solution of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-benzamide (3.04 g) in a 2:1 mixture of 1,2-dimethoxyethane-water (150 ml), and the reaction mixture is stirred at room temperature for one hour. After removal of the ether in a rotary evaporator (bath temperature 85° C.), the mixture is acidified with ice-cold 10% citric acid solution (45 ml) and extraction is carried out with dichloromethane. The organic phase is dried over magnesium sulfate and concentrated in vacuo at room temperature. 2.96 g of the title compound, R_{f} (L)=0.26, are obtained in the form of a pale-yellow foamy solid.

### Example 55

In a manner analogous to that described in Example 54), the following compounds are prepared:

a) From 100 mg of (2S,4S,5S,7S)-5-(tert-butoxycarbonyl)amino-4-(tert-butyl)dimethylsilyloxy-2-isopropyl-7-{[2-(4-methoxybutoxy)-benzoylamino]-methyl}-8-methyl-nonanoic acid and 24 mg of 3-aminobutyric acid amide, with subsequent purification by FC (eluant gradient from U to dichloromethane-methanol-conc. ammonia 95:5:1), (2S,4S,5S,7S)-N-(4-(tert-butoxycarbonyl)amino-5-(tert-butyl)dimethylsiyloxy-7-(3-carbamoylpropylcarbamoyl)-2-isopropyl-8-methyl-nonyl)-2-(4-methoxybutoxy)-benzamide, R_{f} (W)=0.48, in the form of a yellow oil.

b) From 100 mg of (2S,4S,5S,7S)-5-(tert-butoxycarbonyl)amino-4-(tert-butyl)dimethylsilyloxy-2-isopropyl-7-{[2-(4-methoxybutoxy)-benzoylamino]-methyl}-8-methyl-nonanoic acid and 26.3 mg of 3-amino-2,2-dimethylpropionic acid amide, (2S,4S,5S,7S)-N-(4-(tert-butoxycarbonyl)amino-5-(tert-butyl)dimethylsilyloxy-7-(2-carbamoyl-2-methylpropylcarbamoyl)-2-isopropyl-8-methyl-nonyl)-2-(4-methoxybutoxy)-benzamide, R_{f} (W)=0.59, in the form of a colorless oil.

c) From 100 mg of (2S,4S,5S,7S)-5-(tert-butoxycarbonyl)amino-4-(tert-butyl)dimethylsilyloxy-2-isopropyl-7-{[2-(4-methoxybutoxy)-benzoylamino]-methyl}-8-methyl-nonanoic acid and 48 mg of 3-aminopropionic acid N-(morpholine)amide hydrochloride, (2S,4S,5S,7S)-N-{4-(tert-butoxycarbonyl)amino-5-(tert-butyl)dimethylsilyloxy-2-isopropyl-8-methyl-7-[3-(morpholin-4-yl)-3-oxo-propylcarbamoyl]-nonyl}-2-(4-methoxybutoxy)-benzamide, R_{f} (W)=0.62, in the form of a colourless oil.

d) From 100 mg of (2S,4S,5S,7S)-5-(tert-butoxycarbonyl)amino-4-(tert-butyl)dimethylsilyloxy-2-isopropyl-7-{[2-(4-methoxybutoxy)-benzoyliamino]-methyl}-8-methyl-nonanoic acid and 41 mg of 1-[4-(2-aminoethyl)-piperidin-1-yl]-ethanone, (2S,4S,5S,7S)-N-{7-[2-(4-acetylpiperidin-1-yl)-ethylcarbamoyl]-4-(tert-butoxycarbonyl)amino-5-(tert-butyl)dimethylsilyloxy-2-isopropyl-8-methyl-nonyl}-2-(4-methoxybutoxy)-benzamide, R_{f} (W)=0.58, in the form of an oil.

e) From 100 mg of (2S,4S,5S,7S)-5-(tert-butoxycarbonyl)amino-4-(tert-butyl)dimethylsilyloxy-2-isopropyl-7-{[2-(4-methoxybutoxy)-benzoylamino]-methyl}-8-methyl-nonanoic acid and 31 mg of 2-aminoethyl-thiomorpholine, with purification by FC (25 g of silica gel, eluant T), (2S,4S,5S,7S)-N-[7-(2-thiomorpholin-4-ylethylcarbamoyl)-4-(tert-butoxycarbonyl)amino-5-(tert-butyl)dimethylsilyloxy-2-isopropyl-8-methyl-nonyl]-2-(4-methoxybutoxy]-benzamide, R_{f} (dichloromethane-methanol-conc. ammonia 95:5:1)=0.36, in the form of an oil.

### Example 56

In a manner analogous to that described in Example 54), (2S,4S,5S,7S)-5-(tert-butoxycarbonyl)amino-4-(tert-butyl)dimethylsilyloxy-2-isopropyl-7-{[2-(4-methoxybutoxy)-4-[2-(morpholin-4-yl)-ethoxy)-benzoylamino]-methyl}-8-methyl-nonanoic acid (258 mg), cyanophosphonic acid diethyl ester (0.138 ml), 3-amino-2,2-dimethylpropionic acid amide hydrochloride (139 mg) and triethylamine (0.20 ml) in anhydrous N,N-dimethy]formamide (10 ml) are reacted. Purification by FC (eluant V) yields (2S,4S,5S,7S)-N-(4-(tert-butoxycarbonyl)amino-5-(tert-butyl)dimethylsilyloxy-7-(2-carbamoyl-2-methylpropylcarbamoyl)-2-isopropyl-8-methyl-nonyl)-2-(4-methoxybutoxy)-4-(2-morpholin-4-ylethoxy)-benzamide, R_{f} (W)=0.50, in the form of a white solid.

The (2S,4S,5S,7S)-5-(tert-butoxycarbonyl)amino-4-(tert-butyl)dimethylsilyloxy-2-isopropyl-7-{[2-(4-methoxybutoxy)-4-(2-morpholin-4-ylethoxy)-benzoylamino]-methyl}-8-methyl-nonanoic acid used as starting material is prepared as follows:

a) (2S,4S,5S,7S)-5-(tert-Butoxycarbonyl)amino-4-(tert-butyl)dimethylsilyloxy-2-isopropyl-7-{[2-(4-methoxybutoxy]-4-(2-morpholin-4-ylethoxy)-benzoylamino]-methyl}-8-methyl-nonanoic acid: In a manner analogous to that described in Example 54a), (2S,4S,5S,7S)-5-(tert-butoxycarbonyl)amino-4-hydroxy-2-isopropyl-7-{[2-(4-methoxybutoxy)-4-(2-morpholin-4-ylethoxy)-benzoylamino]-methyl)-8-methyl-nonanoic acid (629 mg), tert-butyldimethylsilyl chloride (294 mg) and imidazole (253 mg) in anhydrous N,N-dimethylformamide (5 ml) are stirred at room temperature for 5 days. Working-up yields 611 mg of a yellowish oil, which is dissolved in a 2:1 mixture of tetrahydrofuran-water (4 ml) and glacial acetic acid (3 ml) and is stirred overnight at room temperature. Concentration of the reaction mixture and working-up by extraction with ethyl acetate yield 920 mg of the title compound, R_{f} (W)=0.26, in the form of a yellow oil.

b) (2S,4S,5S,7S)-5-(tert-Butoxycarbonyl)amino-4-hydroxy-2-isopropyl-7-{[2-(4-methoxybutoxy)-4-(2-morpholin-4-ylethoxy)-benzoylamino]-methyl}-8-methyl-nonanoic acid: In a manner analogous to that described in Example 54b), (2S,2′S,2″S,4″S)-N-(2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-4-(2-morpholin-4-ylethoxy)-benzamide (622 mg) (Example 30b), dissolved in 30 ml of a 2:1 mixture of 1,2-dimethoxyethane-water, is reacted with a 1M lithium hydroxide solution (3.6 ml). Working-up yields the title compound (630 mg) in the form of a pale-yellow foamy solid, R_{f} (L)=0.29, which is immediately reacted further.

### Example 57

Reaction of (2S,4S,5S,7S)-5-(tert-butoxycarbonyl)amino-7-aminomethyl-4-(tert-butyl)dimethylsilyloxy-2-isopropyl-8-methyl-nonanoic acid N-(2-carbamoyl-2-methylpropyl)amide (100 mg) and 2-(4-methoxybutoxy)-4-(morpholin-4-ylmethyl)-benzoic acid (116 mg) in a manner analogous to that described in Example 15) and purification by FC (30 g of silica gel, eluant T) yield (2S,4S,5S,7S)-N-(4-(tert-butoxycarbonyl)amino-5-(tert-butyl)dimethylsilyloxy-7-(2-carbamoyl-2-methylpropylcarbamoyl)-2-isopropyl-8-methyl-nonyl)-2-(4-methoxybutoxy)-4-(morpholin-4-ylmethyl)-benzamide, R_{f} (W)=0.57, in the form of a white solid.

The (2S,4S,5S,7S)-5-(tert-butoxycarbonyl)amino-7-aminomethyl-4-(tert-butyl)dimethylsilyloxy-2-isopropyl-8-methyl-nonanoic acid N-(2-carbamoyl-2-methylpropyl)amide used as starting material is prepared as follows:

a) After hydrogenation of (2S,4S,5S,7S)-5-(tert-butoxycarbonyl)amino-7-azidomethyl-4-(tert-butyl)dimethylsilyloxy-2-isopropyl-8-methyl-nonanoic acid N-(2-carbamoyl-2-methylpropyl)amide (534 mg), dissolved in ethyl acetate (30 ml), for 5 hours at room temperature in the presence of 10% Pd/C (106 mg), filtration is carried out over Celite 545, the filtrate is concentrated and the crude product so obtained is hydrogenated again for 24 hours in the presence of fresh catalyst (106 mg of 10% Pd/C). Purification by FC (25 g of silica gel, eluant gradient dichloromethane-methanol-conc. ammonia from 94:6:1 to T) yields (2S,4S,5S,7S)-5-(tert-butoxycarbonyl)amino-7-aminomethyl-4-(tert-butyl)dimethylsilyloxy-2-isopropyl-8-methyl-nonanoic acid N-(2-carbamoyl-2-methylpropyl)amide, R_{f} (W)=0.28, in the form of a white solid.

b) (2S,4S,5S,7S)-5-(tert-Butoxycarbonyl)amino-7-azidomethyl-4-(tert-butyl)dimethylsilyloxy-2-isopropyl-8-methyl-nonanoic acid N-(2-carbamoyl-2-methylpropyl)amide: Reaction of (2S,4S,5S,7S)-5-(tert-butoxycarbonyl)amino-7-azidomethyl-4-(tert-butyl)dimethylsilyloxy-2-isopropyl-8-methyl-nonanoic acid (490 mg) with 3-amino-2,2-dimethylpropionic acid amide hydrochloride (290 mg) in a manner analogous to that described in Example 56) and purification by FC (50 g of silica gel, eluant T) yield the title compound, R_{f} (W)=0.67; MS(FAB) m/e 613 (M⁺+1), in the form of a white solid.

c) (2S,4S,5S,7S)-5-(tert-Butoxycarbonyl)amino-7-azidomethyl-4-(tert-butyl)dimethylsilyloxy-2-isopropyl-8-methyl-nonanoic acid is obtained in a manner analogous to that described in Examples 54a) and 54b) from 500 mg of (3S,5S,1′S,3′S)-5-[3′-azidomethyl-1′-(tert-butoxycarbonyl)amino-4′-methylpentyl]-3-isopropyl-dihydrofuran-2-one (Example 15b) via (2S,4S,5S,7S)-5-(tert-butoxycarbonyl)amino-7-azidomethyl-4-hydroxy-2-isopropyl-8-methyl-nonanoic acid (R_{f} (L)=0.43) after purification by FC (50 g of silica gel, eluant Q), in the form of a white solid (which contains a small amount of starting material on account of re-lactonisation): R_{f} (L)=0.64.

### Example 58

Reaction of (2S,4S,5S,7S)-5-(tert-butoxycarbonyl)amino-7-aminomethyl-4-(tert-butyl)dimethylsilyloxy-2-isopropyl-8-methyl-nonanoic acid N-(2-carbamoyl-2-methylpropyl)amide (100 mg) and 2-(2-morpholin-4-ylethoxy)-benzoic acid (89 mg) in a manner analogous to that described in Example 54) and purification by FC yield (2S,4S,5S,7S)-N-[4-(tert-butoxycarbonyl)amino-5-(tert-butyl)dimethylsilyloxy-7-(2-carbamoyl-2-methylpropylcarbamoyl)-2-isopropyl-8-methyl-nonyl]-2-(2-morpholin-4-ylethoxy)-benzamide in the form of an oil: R_{f} (L)=0.40; HPLC Rₜ=17.3 min.

### Example 59

Tetrabutylammonium fluoride trihydrate (31 mg) is added to a solution of (2S,4S,5S,7S)-N-(4-(tert-butoxycarbonyl)amino-5-(tert-butyl)dimethylsilyloxy-7-[2-dimethylaminocarbamoyl)-ethylcarbamoyl]-2-isopropyl-8-methyl-nonyl)-2-(4-methoxybutoxy)-benzamide (71 mg) in anhydrous N,N-dimethylformamide (5 ml), and the mixture is stirred overnight at room temperature. The reaction mixture is concentrated and the residue is partitioned between saturated sodium hydrogen carbonate solution (20 ml) and ethyl acetate (30 ml). The aqueous phase is separated off and extracted with ethyl acetate, and the combined organic phases are washed with saturated sodium chloride solution, dried over magnesium sulfate and concentrated. FC (25 g of silica gel, eluant 0) yields (2S,4S,5S,7S)-N-[4-(tert-butoxycarbonyl)amino-7-[2-(dimethylaminocarbamoyl)-ethylcarbamoyl]-5-hydroxy-2isopropyl-8-methyl-nonyl)-2-(4-methoxybutoxy)-benzamide, R_{f} (N)=0.32, in the form of a yellowish oil.

### Example 60

In a manner analogous to that described in Example 59), the following compounds are prepared by removal of the silyloxy-protecting group:

a) From 76 mg of (2S,4S,5S,7S)-N-(4-(tert-butoxycarbonyl)amino-5-(tert-butyl)dimethylsilyloxy-7-(3-carbamoylpropylcarbamoyl)-2-isopropyl-8-methyl-nonyl)-2-(4-methoxybutoxy)-benzamide, with purification by FC (25 g of silica gel, eluant gradient from N to L), (2S,4S,5S,7S)-N-[4-(tert-butoxycarbonyl)amino-7-(3-carbamoylpropylcarbamoyl)-5-hydroxy-2-isopropyl-8-methyl-nonyl)-2-(4-methoxybutoxy)-benzamide, R_{f} (L)=0.45, in the form of an oil.

b) From 66 mg of (2S,4S,5S,7S)-N-(4-(tert-butoxycarbonyl)amino-5-(tert-butyl)dimethylsilyloxy-7-(2-carbamoyl-2-methylpropylcarbamoyl)-2-isopropyl-8-methyl-nonyl)-2-(4-methoxybutoxy)-benzamide, (2S,4S,5S,7S)-N-[4-(tert-butoxycarbonyl)amino-7-(2-carbamoyl-2-methylpropylcarbamoyl)-5-hydroxy-2-isopropyl-8-methyl-nonyl)-2-(4-methoxybutoxy)-benzamide, R_{f} (L)=0.48, in the form of an oil.

c) From 101 mg of (2S,4S,5S,7S)-N-{4-(tert-butoxycarbonyl)amino-5-(tert-butyl)dimethylsilyloxy-2-isopropyl-8-methyl-7-[3-(morphoin-4-yl)-3-oxo-propylcarbamoyl]-nonyl}-2-(4-methoxybutoxy)-benzamide, (2S,4S,5S,7S)-N-{4-(tert-butoxycarbonyl)amino-5-hydroxy-2-isopropyl-8-methyl-7-[3-(morpholin-4-yl)-3-oxo-propylcarbamoyl]-nonyl}-2-(4-methoxybutoxy)-benzamide, R_{f} (W)=0.54; HPLC Rₜ=15.7 min, in the form of an oil.

d) From 134 mg of (2S,4S,5S,7S)-N-{7-[2-(4-acetylpiperidin-1-yl)-ethylcarbamoyl]-4-tert-butoxycarbonyl)amino-5-(tert-butyl)dimethylsilyloxy-2-isopropyl-8-methyl-nonyl}-2-(4-methoxybutoxy)-benzamide, (2S,4S,5S,7S)-N-{7-[2-(4-acetylpiperidin-1-yl)-ethylcarbamoyl]-4-(tert-butoxycarbonyl)amino-5-hydroxy-2-isopropyl-8-methyl-nonyl}-2-(4-methoxybutoxy)-benzamide, R_{f} (W)=0.46; HPLC Rₜ=17.1 min, in the form of a white solid.

e) From 76 mg of (2S,4S,5S,7S)-N-[4-(tert-butoxycarbonyl)amino-S-(tert-butyl)dimethylsilyloxy-2-isopropyl-8-methyl-7-(2-thiomorpholin-4-ylethylcarbamoyl)-nonyl]-2-(4-methoxybutoxy)-benzamide, (2S,4S,5S,7S)-N-[4-(tert-butoxycarbonyl)amino-5-hydroxy-2-isopropyl-8-methyl-7-(2-thiomorpholin-4-ylethylcarbamoyl)-nonyl]-2-(4-methoxybutoxy)-benzamide, R_{f} (N)=0.28; HPLC Rₜ=14.7 min, in the form of an oil.

f) From 150 mg of (2S,4S,5S,7S)-N-(4-(tert-butoxycarbonyl)amino-5-(tert-butyl)dimethylsilyloxy-7-(2-carbamoyl-2-methylpropylcarbamoyl)-2-isopropyl-8-methyl-nonyl)-2-(4-methoxybutoxy)-4-(2-morpholin-4-ylethoxy)-benzamide, (2S,4S,5S,7S)-N-[4-(tert-butoxycarbonyl)amino-7-(2-carbamoyl-2-methylpropylcarbamoyl)-5-hydroxy-2-isopropyl-8-methyl-nonyl)-2-(4-methoxybutoxy)-4-(2-morpholin-4-ylethoxy)-benzamide, R_{f} (W)=0.40, in the form of an oil.

g) From 116 mg of (2S,4S,5S,7S)-N-(4-(tert-butoxycarbonyl)amino-5-(tert-butyl)dimethylsilyloxy-7-(2-carbamoyl-2-methylpropylcarbamoyl)-2-isopropyl-8-methyl-nonyl)-2-(4-methoxybutoxy)-4-(morpholin-4-ylmethyl)-benzamide, (2S,4S,5S,7S)-N-[4-(tert-butoxycarbonyl)amino-7-(2-carbamoyl-2-methylpropylcarbamoyl)-5-hydroxy-2-isopropyl-8-methyl-nonyl)-2-(4-methoxybutoxy)-4-(morpholin-4-ylmethyl)-benzamide, R_{f} (W)=0.33; HPLC Rₜ=11.5 min, in the form of an oil.

h) From 96 mg of (2S,4S,5S,7S)-N-[4-(tert-butoxycarbonyl)amino-5-(tert-butyl)dimethylsilyloxy-7-(2-carbamoyl-2-methylpropylcarbamoyl)-2-isopropyl-8-methyl-nonyl]-2-(4-methoxybutoxy)-4-(2-morpholin-4-ylethoxy)-benzamide, (2S,4S,5S,7S)-N-[4-(tert-butoxycarbonyl)-amino-7-(2-carbamoyl-2-methylpropylcarbamoyl)-5-hydroxy-2-isopropyl-8-methyl-nonyl]-2-(2-morpholin-4-ylethoxy)-benzamide, R_{f} (L)=0.35; HPLC Rₜ=11.6 min, in the form of an oil.

### Example 61

In a manner analogous to that described in Example 21), 71 mg of (2S,4S,5S,7S,2′R)-N-[4-(tert-butoxycarbonyl)amino-7-(2′-methylcarbamoyl-propylcarbamoyl)-5-hydroxy-2-isopropyl-8-methyl-nonyl]-2-(4-methoxybutoxy)-benzamide yield (2S,4S,5S,7S,2′R)-N-[4-amino-7-(2′-methylcarbamoyl-propylcarbamoyl)-5-hydroxy-2-isopropyl-8-methyl-nonyl]-2-(4-methoxybutoxy)-benzamide hydrochloride: R_{f} (W)=0.33. HPLC Rₜ=12.7 min. MS(FAB) m/e 579 (M⁺+1)

The (2S,4S,5S,7S,2′R)-N-[4-(tert-butoxycarbonyl)amino-7-(2′-methylcarbamoyl-propylcarbamoyl)-5-hydroxy-2-isopropyl-8-methyl-nonyl]-2-(4-methoxybutoxy)-benzamide used as starting material is prepared as follows:

a) In a manner analogous to that described in Example 59), (2S,4S,5S,7S,2′R)-N-[4-(tert-butoxycarbonyl)amino-(tert-butyl)dimethylsilyloxy-7-(2′-methylcarbamoyl-propylcarbamoyl)-2-isopropyl-8-methyl-nonyl]-2-(4-methoxybutoxy)-benzamide (132 mg) and tetrabutyl-ammonium fluoride trihydrate (52 mg) in N,N-dimethylformamide (5 ml) are stirred at room temperature for 20 hours. Aqueous working-up and purification by FC (25 g of silica gel, eluant V) yield (2S,4S,5S,7S,2′R)-N-[4-(tert-butoxycarbonyl)amino-7-(2′-methylcarbamoylpropylcarbamoyl)-5-hydroxy-2-isopropyl-8-methyl-nonyl]-2-(4-methoxybutoxy)-benzamide, R_{f} (W)=0.51; HPLC Rₜ=17.6 min, in the form of a colourless foam.

b) (2S,4S,5S,7S,2′R)-N-[4-(tert-Butoxycarbonyl)amino-(tert-butyl)dimethylsilyloxy-7-(2′-methylcarbamoyl-propylcarbamoyl)-2-isopropyl-8-methyl-nonyl]-2-(4-methoxybutoxy)-benzamide: A solution of (2R,2′S,4′S,5″S,7″S)-3-{5′-(tert-butoxycarbonyl)amino-4′-(tert-butyl)-dimethylsilyloxy-2′-isopropyl-7′-[2-(4-methoxybutoxy)-benzylcarbamoyl]-8-methyl-nonanoyl-amino}-2-methylpropionic acid methyl ester (150 mg) in a 33% ethanolic methylamine solution (6 ml) is stirred at 40° C. for 40 hours. After concentration of the reaction mixture, the residue is purified by FC (eluant W). The title compound, R_{f} (W)=0.54, is obtained in the form of a yellow oil.

c) (2R,2′S,4′S,5′S,7′S)-3-{5′-(tert-Butoxycarbonyl)amino-4′-(tert-butyl)dimethylsilyloxy-2′-isopropyl-7′-[2-(4-methoxybutoxy)-benzylcarbamoyl]-8-methyl-nonanoylamino}-2-methylpropionic acid methyl ester: In a manner analogous to that described in Example 54), (2S,4S,5S,7S)-5-(tert-butoxycarbonyl)amino-4-(tert-butyl)dimethylsilyloxy-2-isopropyl-7-{[2-(4-methoxybutoxy)-benzoylamino]-methyl}-8-methyl-nonanoic acid (800 mg), (2R)-3-amino-2-methyl-propionic acid methyl ester hydrochloride (112 mg), cyanophosphonic acid diethyl ester (110 μl) and triethylamine (204 μl) in N,N-dimethylformamide (10 ml) are reacted. The reaction mixture is concentrated and the residue is taken up in ethyl acetate. The organic phase is washed with 1M citric acid solution, saturated sodium hydrogen carbonate solution and saturated sodium chloride solution, dried over magnesium sulfate and concentrated. Purification of the crude product by FC (80 g of silica gel, eluant S) yields the title compound, R_{f} (W)=0.82, in the form of an oil.

### Example 62

In a manner analogous to that described in Example 21), the following compounds are prepared by de-Bocylation:

a) From 35 mg of (2S,4S,5S,7S)-N-[4-(tert-butoxycarbonyl)amino-7-[2-(dimethylaminocarbamoyl)-ethylcarbamoyl]-5-hydroxy-2-isopropyl-8-methyl-nonyl)-2-(4-methoxybutoxy)-benzamide, (2S,4S,5S,7S)-N-(4-amino-7-[2-(dimethylaminocarbamoyl)-ethylcarbamoyl]-5-hydroxy-2-isopropyl-8-methyl-nonyl)-2-(4-methoxybutoxy)-benzamide hydrochloride: R_{f} (W)=0.26. HPLC Rₜ=11.5 min. MS(FAB) m/e 579 (M⁺+1).

b) From 45 mg of (2S,4S,5S,7S)-N-[4-(tert-butoxycarbonyl)amino-7-(3-carbamoylpropylcarbamoyl)-5-hydroxy-2-isopropyl-8-methyl-nonyl]-2-(4-methoxybutoxy)-benzamide, (2S,4S,5S,7S)-N-[4-amino-7-(3-carbamoylpropylcarbamoyl)-5-hydroxy-2-isopropyl-8-methyl-nonyl]-2-(4-methoxybutoxy)-benzamide hydrochloride: R_{f} (W)=0.21. HPLC Rₜ=10.3 min. MS(FAB) m/e 565 (M⁺+1).

c) From 46 mg of (2S,4S,5S,7S)-N-[4-(tert-butoxycarbonyl)amino-7-(2-carbamoyl-2-methylpropylcarbamoyl)-5-hydroxy-2-isopropyl-8-methyl-nonyl)-2-(4-methoxybutoxy)-benzamide, (2S,4S,5S,7S)-N-[4-amino-7-(2-carbamoyl-2-methylpropylcarbamoyl)-5-hydroxy-2-isopropyl-8-methyl-nonyl]-2-(4-methoxybutoxy)-benzamide hydrochloride: R_{f} (W)=0.27. HPLC Rₜ=11.1 min. MS(FAB) m/e 579 (M⁺+1).

d) From 65 mg of (2S,4S,5S,7S)-N-{4-(ted-butoxycarbonyl)amino-5-hydroxy-2-isopropyl-8-methyl-7-[3-(morpholin-4-yl)-3-oxo-propylcarbamoyl]-nonyl}-2-(4-methoxybutoxy)-benzamide, (2S,4S,5S,7S)-N-{4-amino-5-hydroxy-2-isopropyl-8-methyl-7-[3-(morpholin-4-yl)-3-oxo-propylcarbamoyl]-nonyl}-2-(4-methoxybutoxy)-benzamide hydrochloride: R_{f} (W)=0.25. HPLC Rₜ=11.3 min. MS(FAB) m/e 621 (M⁺+1).

e) From 71 mg of (2S,4S,5S,7S)-N-{7-(2-(4-acetylpiperidin-1-yl)-ethylcarbamoyl]-4-(tert-butoxycarbonyl)amino-5-hydroxy-2-isopropyl-8-methyl-nonyl}-2-(4-methoxybutoxy)-benzamide, (2S,4S,5S,7S)-N-{7-[2-(4-acetylpiperidin-1-yl)-ethylcarbamoyl]-4-amino-5-hydroxy-2-isopropyl-8-methyl-nonyl}-2-(4-methoxybutoxy)-benzamide hydrochloride: R_{f} (W)=0.29. HPLC Rₜ=12.7 min. MS(FAB) m/e 633 (M⁺+1).

f) From 40 mg of (2S,4S,5S,7S)-N-[4-(tert-butoxycarbonyl)amino-5-hydroxy-2-isopropyl-8-methyl-7-(2-thiomorpholin-4-ylethylcarbamoyl)-nonyl]-2-(4-methoxybutoxy)-benzamide, (2S,4S,5S,7S)-N-[4-amino-5-hydroxy-2-isopropyl-8-methyl-7-(2-thiomorpholin-4-ylethylcarbamoyl)-methyl-nonyl]-2-(4-methoxybutoxy)-benzamide dihydrochloride: R_{f} (W)=0.43. HPLC Rₜ=10.7 min. MS(FAB) m/e 609 (M⁺+1).

g) From 102 mg of (2S,4S,5S,7S)-N-[4-(tert-butoxycarbonyl)amino-7-(2-carbamoyl-2-methylpropylcarbamoyl)-5-hydroxy-2-isopropyl-8-methyl-nonyl)-2-(4-methoxybutoxy)-4-(2-morpholin-4-ylethoxy)-benzamide, (2S,4S,5S,7S)-N-(4-amino-7-(2-carbamoyl-2-methylpropylcarbamoyl)-5-hydroxy-2-isopropyl-8methyl-nonyl)-2-(4-methoxybutoxy)-4-(2-morpholin-4-ylethoxy-benzamide dihydrochloride: R_{f} (W)=0.18. HPLC Rₜ=8.48 min. MS(FAB) m/e 708 (M⁺+1).

h) From 78 mg of (2S,4S,5S,7S)-N-(4-(tert-butoxycarbonyl)amino-7-(2-carbamoyl-2-2-methylpropylcarbamoyl)-5-hydroxy-2-isopropy-8-methyl-nonyl)-2-(4-methoxybutoxy)-4-(morpholin-4-ylmethyl)-benzamide, (2S,4S,5S,7S)-N-(4-amino-7-(2-carbamoyl-2-methylpropylcarbamoyl)-5-hydroxy-2-isopropyl-8-methyl-nonyl)-2-(4-methoxybutoxy)-4-(morpholin-4-ylmethyl)-benzamide dihydrochloride: R_{f} (W)=0.17. HPLC Rₜ=7.83 min. MS(FAB) m/e 678 (M⁺+1).

i) From 62 mg of (2S,4S,5S,7S)-N-[4-(tert-butoxycarbonyl)amino-7-(2-carbamoyl-2-methylpropylcarbamoyl)-5-hydroxy-2-isopropyl-8-methyl-nonyl]-2-(2-morpholin-4-ylethoxy)-benzamide, (2S,4S,5S,7S)-N-[4-amino-7-(2-carbamoyl-2-methylpropylcarbamoyl)-5-hydroxy-2-isopropyl-8-methyl-nonyl]-2-(2-morpholin-4-ylethoxy)-benzamide dihydrochloride: R_{f} (J)=0.20. HPLC Rₜ=7.0 min. MS(FAB) m/e 606 (M⁺+1).

j) From 490 mg of (2S,4S,5S,7S)-N-{4-(tert-butoxycarbonyl)amino-5-hydroxy-2-isopropyl-7-[2-(4-methoxycarbonylpiperidin-1-yl)-ethylcarbamoyl]-8-methyl-nonyl}-2-(4-methoxybutoxy)-benzamide (Example 66), (2S,4S,5S,7S)-N-{4-amino-5-hydroxy-2-isopropyl-7-[2-(4-methoxycarbonylpiperidin-1-yl)-ethylcarbamoyl]-8-methyl-nonyl}-2-(4-methoxybutoxy)-benzamide hydrochloride: R_{f} (W)=0.30. HPLC Rₜ=11.8 min. MS(FAB) m/e 649 (M⁺+1).

### Example 63

A solution of (2S,4S,5S,7R)-N-{4-(tert-butoxycarbonyl)amino-5-hydroxy-2-isopropyl-7-[(2-morpholin-4-ylethyl)-carbamoyl]-octyl}-2-(3-methoxypropoxy)-benzamide (105 mg) in 4N hydrochloric acid solution in dioxane (4 ml) is stirred at 0° C. for one hour. The reaction mixture is then lyophilised. (2S,4S,5S,7R)-N-[4-Amino-5-hydroxy-2-methyl-7-[(2-morpholin-4-ylethyl)-carbamoyl]-octyl)-2-(3-methoxypropoxy)-benzamide dihydrochloride is obtained in the form of a beige powder: R_{f} (dichloromethane-methanol 8:2)=0.28. HPLC Rₜ=7.73 min. MS(FAB) m/e 551 (M⁺+1).

The (2S,4S,5S,7R)-N-{4-(tert-butoxycarbonyl)amino-5-hydroxy-2-isopropyl-7-[(2-morpholin-4-ylethyl)-carbamoyl]-octyl}-2-(3-methoxypropoxy)-benzamide used as starting material is prepared as follows:

a) (2S,4S,5S,7R)-N-{4-(tert-Butoxycarbonyl)amino-5-hydroxy-2-isopropyl-7-[(2-morpholin-4-ylethyl)-carbamoyl]-octyl}-2-(3-methoxypropoxy)-benzamide: A mixture of (2S,2S′,2″S,4″R)-N-{2-(2′-(tert-butoxycarbonyl)amino-2′-(4″-isopropyl-5″-oxo-tetrahydrofuran-2″-yl)-ethyl]-3-methylbutyl}-2-(3-methoxypropoxy)-benzamide (104 mg) and 4-(2-aminoethyl)-morpholine (2 ml) is stirred at 80° C. for 2 hours. The excess 4-(2-aminoethyl)-morpholine is then distilled off and the evaporation residue is purified by FC (100 g of silica gel, eluant L). The title compound (110 mg) is obtained in the form of a white foam: R_{f} (L)=0.36; HPLC Rₜ=12.1 min.

b) (2S,2″S,2″S,4″R)-N-{2-[2′-(tert-Butoxycarbonyl)amino-2′-(4″-isopropyl-5″-oxo-tetrahydrofuran-2″-yl)-ethyl]-3-methylbutyl}-2-(3-methoxypropoxy)-benzamide: p-Toluenesulfonic acid monohydrate (134 mg) is added at 0° C. to a solution of (2S,4S,5S,7R)-N-[4-(tert-butoxycarbonyl)amino-7-butylcarbamoyl-5-hydroxy-2-isopropyl-octyl]-2-(3-methoxypropoxy)-benzamide (380 mg) in chloroform (20 ml). The mixture is stirred at room temperature for 20 hours. The solvent is evaporated off and the residue is purified by FC (60 g of silica gel, eluant E). The title compound is obtained in the form of a colorless oil: R_{f} (F)=0.36; HPLC Rₜ=17.1 min; MS(FAB) m/e 521 (M⁺+1)

### Example 64

In a manner analogous to that described in Example 21), (2S,4S,5S,7S)-N-{4-amino-5-hydroxy-2-isopropyl-8-methyl-7-[2-(morpholin-4-yl)-ethylcarbamoyl]-nonyl}-4-carbamoylmethoxy-2-(4-methoxybutoxy)-benzamide dihydrochloride, R_{f} (W)=0.23; HPLC Rₜ=9.81 min; MS(FAB) m/e 666 (M⁺+1), is prepared from (2S,4S,5S,7S)-N-{4-(tert-butoxycarbonyl)amino-5-hydroxy-2-isopropyl-8-methyl-7-[2-(morpholin-4-yl)-ethylcarbamoyl]-nonyl}-4-carbamoylmethoxy-2-(4-methoxybutoxy)-benzamide (103 mg).

The (2S,4S,5S,7S)-N-{4-(tert-butoxycarbonyl)amino-5-hydroxy-2-isopropyl-8-methyl-7-[2-(morpholin-4-yl)-ethylcarbamoyl]-nonyl}-4-carbamoylmethoxy-2-(4-methoxybutoxy)-benzamide used as starting material is prepared as follows:

a) A mixture of (2S,4S,5S,7S)-N-{4-(tert-butoxycarbonyl)amino-5-hydroxy-2-isopropyl-8-methyl-7-[2-(morpholin-4-yl)-ethylcarbamoyl]-nonyl}-4-hydroxy-2-(4-methoxybutoxy)-benzamide (100 mg), 2-bromoacetamide (24 mg) and cesium carbonate (69 mg) in anhydrous acetone (5 ml) is stirred under reflux for 2 hours. The crude product obtained after filtration and concentration of the solvent is purified by FC on 25 g of silica gel (eluant gradient from V to dichloromethane-methanol-conc. ammonia 95:5:1). The title compound, R_{f} (dichloromethane-methanol-conc. ammonia 95:5:1; double track)=0.24; HPLC Rₜ=13.7 min; MS(FAB) m/e 766 (M⁺+1), is obtained in the form of a white solid.

b) (2S,4S,5S,7S)-N-{4-(tert-Butoxycarbonyl)amino-5-hydroxy-2-isopropyl-8-methyl-7-[2-(morpholin-4-yl)-ethylcarbamoyl]-nonyl}-4-hydroxy-2-(4-methoxybutoxy)-benzamide: A solution of (2S,4S,5S,7S)-N-{4-(tert-butoxycarbonyl)amino-5-hydroxy-2-isopropyl-8-methyl-7-[2-(morphoin-4-yl)-ethylcarbamoyl]-nonyl)-4-benzyloxy-2-(4-methoxybutoxy)-benzamide (0.86 g) in ethyl acetate (30 ml) is hydrogenated for 6 hours at room temperature in the presence of 5% Pd/C (Degussa) (170 mg). The crude product is purified by FC (eluant gradient from dichloromethane-methanol-conc. ammonia 96:4:1 to W), yielding the title compound (0.76 g) in the form of a white foamy solid: R_{f} (L)=0.27.

c) (2S,4S,5S,7S)-N-{4-(tert-Butoxycarbonyl)amino-5-hydroxy-2-isopropyl-8-methyl-7-[2-(morpholin-4-yl)-ethylcarbamoyl]-nonyl}-4-benzyloxy-2-(4-methoxybutoxy)-benzamide: In a manner analogous to that described in Example 52a), the title compound, R_{f} (W)=0.50, is prepared by reaction of (2S,2′S,2″S,4″S)-N-(2-[2′-(tert-butoxycarbonyl)amino-2′-(41′-isopropyl-5′-oxo-tetrahydrofuran-2″-yl)-ethyl]-3-methylbutyl}-4-benzyloxy-2-(4-methoxybutoxy)-benzamide (0.79 g) in N-(2-aminoethyl)-morpholine (5 ml) and then purification by FC (eluant V).

### Example 65

A mixture of (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-benzamide (1.0 g), 1-[4-(2-aminoethyl)-piperidin-1-yl]-ethanone (0.91 g) and 2-hydroxypyridine (169 mg) in triethylamine (7.5 ml) is stirred at 80° C. for 16 hours (two phases). The upper phase is concentrated to approximately 25% of its volume, and the reaction mixture is stirred at 80° C. for a further 3 hours. After cooling, the mixture is diluted with dichloromethane and the organic phase is washed with saturated sodium hydrogen carbonate solution, dried over magnesium sulfate and concentrated. The crude product is purified by FC on silica gel (eluant V). (2S,4S,5S,7S)-N-{7-[2-(4-acetylpiperidin-1-yl)-ethylcarbamoyl]-4-(tert-butoxycarbonyl)amino-5-hydroxy-2-isopropyl-8-methyl-nonyl}-2-(4-methoxybutoxy)-benzamide is obtained after lyophilisation from a solution in dioxane, in the form of a white powder: R_{f} (W)=0.46.

### Example 66

In a manner analogous to that described in Example 65), the following compound is prepared:

a) From (2S,2′S,2″S,4″S)-N-{2-[2′-(tert-butoxycarbonyl)amino-2′-(4″-isopropyl-5″-oxo-tetrahydrofuran-2″-yl)-ethyl]-3-methylbutyl}-2-(4-methoxybutoxy)-benzamide (400 mg), 4-aminoethyl-1-methoxycarboxypiperidine (397 mg) and 2-hydroxypyridine (68 mg) in triethylamine (5 ml), (2S,4S,5S,7S)-N-{4-(tert-butoxycarbonyl)amino-5-hydroxy-2-isopropyl-7-[2-(4-methoxycarbonylpiperidin-1-yl)-ethylcarbamoyl]-8-methyl-nonyl}-2-(4-methoxybutoxy)-benzamide in the form of an oil: R_{f} (L)=0.50; HPLC Rₜ=18.0 min.

### Example 66

In accordance with the processes described in Examples 1 to 65, the following compounds are prepared analogously:

a) (2R,4S,5S,7R)-1-(2-methoxypropyl)-1H-indole-3-carboxylic acid N-(4-amino-7-butylcarbamoyl-5-hydroxy-2-isopropyl-octyl)-amide hydrochloride

b) (2R,4S,5S,7R)-1-(2-ethoxypropyl)-1H-indole-3-carboxylic acid N-(4-amino-7-butylcarbamoyl-5-hydroxy-2-isopropyl-octyl)-amide hydrochloride

c) (2R,4S,5S,7R)-N-(4-amino-7-butylcarbamoyl-5-hydroxy-2-isopropyl-8-methyl-nonyl)-2-benzyloxy-benzamide hydrochloride

d) (2S,4S,5S,7S)-N-(4-amino-7-butylcarbamoyl-5-hydroxy-2-isopropyl-8-methyl-nonyl)-2-[2-methoxyethoxy)-ethyl]-benzamide

e) (2S,4S,5S,7S)-N-(4-amino-7-butylcarbamoyl-5-hydroxy-2-isopropyl-8-methyl-nonyl)-2-[2-ethoxyethyl)-benzamide hydrochloride

f) (2R,4S,5S,7R,2′S)-N-{4-amino-5-hydroxy-2-isopropyl-8-methyl-7-[(5′-oxo-pyrrolidin-2′-ylmethyl)-carbamoyl]-nonyl}-2-(4-methoxybutoxy)-benzamide hydrochloride

g) (2R,4S,5S,7R,2′R)-N-{4-amino-5-hydroxy-2-isopropyl-8-methyl-7-[(5′-oxo-pyrrolidin-2′-ylmethyl)-carbamoyl]-nonyl}-2-(4-methoxybutoxy)-benzamide hydrochloride

h) (2R,4S,5S,7R)-N-{4-amino-5-hydroxy-2-isopropyl-8-methyl-7-[2-methyl-2-(morpholin-4-yl)-propylcarbamoyl]-nonyl}-2-(4-methoxybutoxy)-benzamide dihydrochloride

i) (2R,4S,5S,7R,1′R)-N-[4-amino-5-hydroxy-2-isopropyl-8-methyl-7-(1′-methyl-2-methylcarbamoyl-ethylcarbamoyl)-nonyl]-2-(4-methoxybutoxy)-benzamide hydrochloride

j) (2R,4S,5S,7R,1′S)-N-[4-amino-5-hydroxy-2-isopropyl-8-methyl-7-(1′-methyl-2-methylcarbamoyl-ethylcarbamoyl)-nonyl]-2-(4-methoxybutoxy)-benzamide hydrochloride

k) (2R,4S,5S,7R,2′S)-N-[4-amino-5-hydroxy-2-isopropyl-7-(2′-carbamoyl-propylcarbamoyl)-8-methyl-nonyl]-2-(4-methoxybutoxy)-benzamide hydrochloride

l) (2R,4S,5S,7R,2′R)-N-[4-amino-5-hydroxy-2-isopropyl-7-(2′-carbamoyl-propylcarbamoyl)-8-methyl-nonyl]-2-(4-methoxybutoxy)-benzamide hydrochloride

m) (2R,4S,5S,7R)-N-[4-amino-5-hydroxy-2-isopropyl-7-(dimethylcarbamoylmethylcarbamoyl)-8-methyl-nonyl]-2-(4-methoxybutoxy)-benzamide hydrochloride

n) (2S,4S,5S,7S,1′S)-N-{4-amino-7-[1-methyl-2′-(morpholin-4-yl)-2′-oxo-ethylcarbamoyl]-5-hydroxy-2-isopropyl-8-methyl-nonyl}-2-(4-methoxybutoxy)-benzamide hydrochloride

o) (2S,4S,5S,7S,2R′)-N-[4-amino-7-(2′-methyl-2′-methylcarbamoyl-propylcarbamoyl)-5-hydroxy-2-isopropyl-8-methyl-nonyl]-2-(4-methoxybutoxy)-benzamide hydrochloride

p) (2S,4S,5S,7S)-3-{5-amino-4-hydroxy-7-[2-(4-methoxybutoxy)-benzoylamino)-methyl]-2-isopropyl-8-methyl-nonanoylamino)-2,2-dimethyl-propionic acid

q) (2S,4S,5S,7S)-N-[4-amino-7-(3-methoxypropylcarbamoyl)-5-hydroxy-2-isopropyl-8-methyl-nonyl]-2-(4-methoxybutoxy)-benzamide hydrochloride

r) (2S,4S,5S,7S,1′S,2′S)-N-[4-amino-7-(1′-carbamoyl-2′-methyl-butylcarbamoyl)-5-hydroxy-2-isopropyl-8-methyl-nonyl]-2-(4-methoxybutoxy)-benzamide hydrochloride

s) (2S,4S,5S,7S)-N-[4-amino-7-(3-methylcarbamoyl-propylcarbamoyl)-5-hydroxy-2-isopropyl-8-methyl-nonyl]-2-(4-methoxybutoxy)-benzamide hydrochloride

t) (2R,4S,5S,7R,1′R)-N-[4-amino-5-hydroxy-2-isopropyl-8-methyl-7-(1′-methyl-2′-carbamoylethylcarbamoyl)-nonyl]-2-(4-methoxybutoxy)-benzamide hydrochloride

u) (2R,4S,5S,7R,1′S)-N-[4-amino-5-hydroxy-2-isopropyl-8-methyl-7-(1′-methyl-2′-carbamoyl-ethylcarbamoyl)-nonyl]-2-(4-methoxybutoxy)-benzamide hydrochloride

v) (2S,4S,5S,7S,1′R)-N-[4-amino-7-(1′-isopropyl-2′-carbamoyl-ethylcarbamoyl)-5-hydroxy-2-isopropyl-8-methyl-nonyl]-2-(4-methoxybutoxy)-benzamide hydrochloride

w) (2S,4S,5S,7S)-N-[4-amino-7-(3-dimethylcarbamoyl-propylcarbamoyl)-5-hydroxy-2-isopropyl-8-methyl-nonyl]-2-(4-methoxybutoxy)-benzamide hydrochloride

x) (2S,4S,5S,7S,2S,)-N-[4-amino-7-(2′-methylcarbamoyl-propylcarbamoyl)-5-hydroxy-2-isopropyl-8-methyl-nonyl]-2-(4-methoxybutoxy)-benzamide hydrochloride.

### Example 67

Gelatin Solution:

A sterile-filtered aqueous solution, comprising 20% cyclodextrins as solubilizer, of one of the compounds of formula I mentioned in the above Examples as active ingredient is mixed under aseptic conditions, with heating, with a sterile gelatin solution, which comprises phenol as preservative, in such a manner that 1.0 ml of the solution has the following composition:

| | | |
|---|---|---|
| active ingredient | 3 | mg |
| gelatin | 150.0 | mg |
| phenol | 4.7 | mg |
| distilled water comprising 20% cyclodextrins | 1.0 | ml |
| as solubilizer | | |

### Example 68

Sterile Dry Substance for Injection:

5 mg of one of the compounds of formula I mentioned in the above Examples as active ingredient are dissolved in 1 ml of an aqueous solution comprising 20 mg of mannitol and 20% cyclodextrins as solubiliser. The solution is sterile-filtered, introduced under aseptic conditions into a 2 ml ampoule, deep-frozen and lyophilised. Prior to use, the lyophilisate is dissolved in 1 ml of distilled water or 1 ml of physiological saline. The solution is administered intramuscularly or intravenously. This formulation can also be introduced into double-chamber injection ampoules.

### Example 69

Nasal Spray:

500 mg of finely ground (<5.0 μm) powder of one of the compounds of formula I mentioned in the above Examples as active ingredient am suspended in a mixture of 3.5 ml of “Myglyol 812®” and 0.08 g of benzyl alcohol. The suspension is introduced into a container having a metering valve. 5.0 g of “Freon 12®” are introduced under pressure into the container through the valve. The “Freon®” is dissolved in the Myglyol-®-benzyl alcohol mixture by shaking. The spray container contains approximately 100 single doses, which can be administered individually.

### Example 70

Film-Coated Tablets:

For the preparation of 10 000 tablets each comprising 100 mg of active ingredient, the following constituents are processed:

| | |
|---|---|
| active ingredient | 1000 g |
| corn starch | 680 g |
| colloidal silica | 200 g |
| magnesium stearate | 20 g |
| stearic acid | 50 g |
| sodium carboxymethyl starch | 250 g |
| water | quantum satis |

A mixture of one of the compounds of formula I mentioned in the above Examples as active ingredient, 50 g of corn starch and the colloidal silica is processed with a starch paste comprising 250 g of corn starch and 2.2 kg of demineralised water to form a moist mass. The mass is pressed through a sieve having a mesh size of 3 mm and is dried at 45° for 30 minutes in a fluidized bed dryer. The dried granules are pressed through a sieve having a mesh size of 1 mm, are mixed with a previously sieved mixture (1 mm sieve) of 330 g of corn starch, the magnesium stearate, the stearic acid and the sodium carboxymethyl starch, and the mixture is compressed to form slightly biconvex tablets.

1. A method according to claim 5, wherein the disease is Alzheimer's disease.

2. A method of treating Alzheimer's disease in a subject in need of such treatment comprising administering to the subject a compound disclosed in claim 1, or a pharmaceutically acceptable salt thereof.

3. A method of treating Alzheimer's disease by modulating the activity of beta amyloid converting enzyme, comprising administering to a subject in need of such treatment a compound disclosed in claim 1, or a pharmaceutically acceptable salt thereof.

4. The method according to claim 1, further comprising the administration of a P-gp inhibitor, or a pharmaceutically acceptable salt thereof.

5. A method of treating a subject who has, or in preventing a subject from getting, a disease or condition selected from the group consisting of Alzheimer's disease, for helping prevent or delay the onset of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which includes administration of a therapeutically effective amount of a compound of formula (I), or a pharmaceutically acceptable salt thereof: 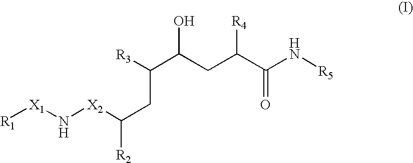
wherein R₁ is a 2-R_{A}-3-R_{B}-phenyl radical, a 2-R_{A}-4-R_{C}-phenyl radical, a 2-R_{A}-pyridin-3-yl radical a 3-R_{A}-pyridin-2-yl radical or a 1-R_{D}-indol-3-yl radical,
wherein one of the radicals R_{A} and R_{B} is an aliphatic or heterecycloaliphatic-aliphatic radical or free or aliphatically, araliphatically or heteroaraliphatically etherified hydroxy and the other is hydrogen, an aliphatic radical or free or esterified or amidated carboxy,
R_{C} is hydrogen, an aliphatic radical, free or aliphatically, araliphatically, heterearaliphatically or heterearylaliphatically etherified hydroxy or an unsubstituted or heteroaliphatically substituted amino group, and
R_{D} is an aliphatic, araliphatic or heteroaliphatic radical,
one of the radicals X₁ and X₂ is carbonyl and the other is methylene,
R₂ is an aliphatic radical,
R₃ is unsubstituted or aliphatically substituted amino,
R₄ is an aliphatic or araliphatic radical, and
R₅ is an aliphatic or cycloaliphatic-aliphatic radical or an optionally hydrogenated and/or oxo-substituted heteroaryl radical or an optionally hydrogenated and/or oxo-substituted heteroaryl or heteroaliphatyl radical bonded via a carbon atom.

6. The method according to claim 5 wherein the compound of formula (I) is selected from the group consisting of:
(2S,4S,5S,7R)-N-(4-Amino-7-butylcarbamoyl-5-hydroxy-2-isopropyl-octyl)-2-(3-methoxypropoxy)-benzamide;
(2S,4S,5S,7R)-N-(4-Amino-7-butylcarbamoyl-5-hydroxy-2-isopropyl-octyl)-3-methoxy-2-(3-methoxypropoxy)-benzamide;
(2S,4S,5S,7R)-N-(4-Amino-7-butylcarbamoyl-5-hydroxy-2-isopropyl-octyl)-4-methoxy-2-(3-methoxypropoxy)-benzamide;
(2S,4S,5S,7R)-N-(4-Amino-7-butylcarbamoyl-5-hydroxy-2-isopropyl-octyl)-3-(3-methoxypropoxy)-benzamide;
(2S,4S,5S,7R)-N-(7-Butylcarbamoyl-4-formylamino-5-hydroxy-2-isopropyl-octyl)-3-methoxy-2-(3-methoxypropoxy)-benzamide;
(2R,4S,5S,7R)-1-Benzyl-1H-indole-3-carboxylic acid N-(4-amino-7-butylcarbamoyl-5-hydroxy-2-isopropyl-octyl)-amide;
(2R,4S,5S,7R)-1-(2-Methoxyethyl)-1H-indole-3-carboxylic acid N-(4-amino-7-butylcarbamoyl-5-hydroxy-2-isopropyl-octyl)-amide;
(2R,4S,5S,7R)-1-Pyridin-2-yl-1H-indole-3-carboxylic acid N-(4-amino-7-butylcarbamoyl-5-hydroxy-2-isopropyl-octyl)-amide;
(2R,4S,5S,7R)-1-(2-Methoxybenzyl)-1H-indole-3-carboxylic acid N-(4-amino-7-butylcarbamoyl-5-hydroxy-2-isopropyl-octyl)-amide;
(2R,4S,5S,7R)-N-(4-Amino-7-butylcarbamoyl-5-hydroxy-2-isopropyl-octyl)-2-(3-methoxypropoxy)-benzamide;
(2R,4S,5S,7R)-N-(4-Amino-7-butylcarbamoyl-5-hydroxy-2-methyl-octyl)-2-(3-methoxypropoxy)-benzamide;
(2R,4S,5S,7R)-N-(4-Amino-7-butylcarbamoyl-5-hydroxy-2-methyl-octyl)-2-(3-methoxypropoxy)-benzamide;
(2S,4S,5S,7S)-N-(4-Amino-7-butylcarbamoyl-5-hydroxy-2-isopropyl-8-methyl-nonyl)-2-(3-methoxypropoxy)-benzamide;
(2S,4S,5S,7S)-N-(4-Amino-7-butylcarbamoyl-5-hydroxy-2-isopropyl-8-methyl-nonyl)-2-(4-methoxybutoxy)-benzamide;
(2S,4S,5S,7S)-N-(4-Amino-7-butylcarbamoyl-5-hydroxy-2-isopropyl-8-methyl-nonyl)-2-propoxy-benzamide;
(2S,4S,5S,7S)-N-(4-amino-7-butylcarbamoyl-5-hydroxy-2-isopropyl-8-methyl-nonyl)-2-(2-methoxyethoxy)-benzamide;
(2S,4S,5S,7S)-N-(4-Amino-7-butylcarbamoyl-5-hydroxy-2-isopropyl-8-methyl-nonyl)-2-[2-(2-methoxyethoxy)-ethoxy]-benzamide;
(2S,4S,5S,7S)-N-(4-Amino-7-butylcarbamoyl-5-hydroxy-2-isopropyl-8-methyl-nonyl)-4-methoxy-2-(3-methoxypropoxy)-benzamide;
(2S,4S,5S,7S)-N-(4-Amino-7-butylcarbamoyl-5-hydroxy-2-isopropyl-8-methyl-nonyl)-4-methoxy-3-(3-methoxypropoxy)-benzamide;
4S,5S,7S)-N-(4-amino-7-butylcarbamoyl-5-hydroxy-2-isopropyl-8-methyl-nonyl)-2-(propoxymethyl)-benzamide;
4S,5S,7S)-N-(4-amino-7-butylcarbamoyl-5-hydroxy-2-isopropyl-8-methyl-nonyl)-2-acetamido-benzamide;
(2S,4S,5S,7S)-N-(4-Amino-7-butylcarbamoyl-5-hydroxy-2-isopropyl-8-methyl-nonyl)-2-[2-(acetamido)-ethoxy]-benzamide;
(2S,4S,5S,7S)-N-(4-Amino-7-butylcarbamoyl-5-hydroxy-2-isopropyl-8-methyl-nonyl)-2-(4-methoxybut-2-enoxy)-benzamide;
(2S,4S,5S,7S)-N-(4-Amino-7-butylcarbamoyl-5-hydroxy-2-isopropyl-8-methyl-nonyl)-2-(4-methoxybutoxy)-4-methyl-benzamide;
(2S,4S,5S,7S)-N-[4-Amino-7-butylcarbamoyl-5-hydroxy-2-isopropyl-8-methyl-nonyl]-2-(3-methoxypropoxy)-nicotinamide;
(2S,4S,5S,7S)-N-[4-Amino-7-butylcarbamoyl-5-hydroxy-2-isopropyl-8-methyl-nonyl]-3-(4-methoxybutoxy)-pyridine-2-carboxylic acid amide;
(2S,4S,5S,7S)-N-(4-Amino-7-butylcarbamoyl-5-hydroxy-2-isopropyl-8-methyl-nonyl)-2-hydroxy-benzamide;
(2S,4S,5S,7S)-N-(4-Amino-7-butylcarbamoyl-5-hydroxy-2-isopropyl-8-methyl-nonyl)-2-[2-(methoxymethoxy)-ethoxy]-benzamide;
(2S,4S,5S,7S)-N-[4-Amino-5-hydroxy-2-isopropyl-8-methyl-7-(2-morpholin-4-ylethylcarbamoyl)-nonyl]-2-(3-methoxypropoxy)-benzamide;
(2S,4S,5S,7S)-N-[4-Amino-5-hydroxy-2-isopropyl-8-methyl-7-(2-morpholin-4-ylethylcarbamoyl)-nonyl]-2-(4-methoxybutoxy)-benzamide;
(2S,4S,5S,7S)-N-[4-Amino-5-hydroxy-2-isopropyl-8-methyl-7-(2-morpholin-4-ylethylcarbamoyl)-nonyl]-2-(2-methoxyethoxy)-benzamide;
(2S,4S,5S,7S)-N-[4-Amino-5-hydroxy-2-isopropyl-8-methyl-7-(2-morpholin-4-ethylcarbamoyl)-nonyl]-2-(3-methoxypropoxy)-nicotinamide;
(2S,4S,5S,7S)-N-[4-Amino-5-hydroxy-2-isopropyl-8-methyl-7-(2-morpholin-4-ylethylcarbamoyl)-nonyl]-3-(4-methoxybutoxy)-pyridine-2-carboxylic acid amide;
(2S,4S,5S,7S)-N-[4-Amino-5-hydroxy-2-isopropyl-8-methyl-7-(2-morpholin-4-ylethylcarbamoyl)-nonyl]-2-(4-methoxybut-2-enoxy)-benzamide;
(2S,4S,5S,7S)-N-[4-Amino-5-hydroxy-2-isopropyl-8-methyl-7-(2-morpholin-4-ylethylcarbamoyl)-nonyl]-2-(4-methoxybutoxy)-4-methyl-benzamide;
(2S,4S,5S,7S)-N-[4-Amino-5-hydroxy-2-isopropyl-8-methyl-7-(2-morpholin-4-ylethylcarbamoyl)-methyl-nonyl]-2-(5-methoxypentyloxy)-benzamide;
(2S,4S,5S,7S)-N-[4-Amino-5-hydroxy-2-isopropyl-8-methyl-7-(3-morpholin-4-ylpropylcarbamoyl)-nonyl]-2-(4-methoxybutoxy)-benzamide;
(2S,4S,5S,7S)-N-(4-Amino-7-butylcarbamoyl-5-hydroxy-2-isopropyl-8-methyl-nonyl)-2-(4-methoxybutoxy)-4-(morpholin-4-ylmethyl)-benzamide;
(2S,4S,5S,7S)-N-(4-Amino-7-butylcarbamoyl-5-hydroxy-2-isopropyl-8-methyl-nonyl-2-(4-methoxybutoxy)-4-[2-(morpholin-4-yl)-ethoxy]-benzamide;
(2S,4S,5S,7S)-N-(4-Amino-7-butylcarbamoyl-5-hydroxy-2-isopropyl-8-methyl-nonyl)-4-[3-(dimethylamino)-propoxy]-2-(4-methoxybutoxy)-benzamide;
(2S,4S,5S,7S)-N-(4-Amino-7-butylcarbamoyl-5-hydroxy-2-isopropyl-8-methyl-nonyl)-2-(4-methoxybutoxy)-4-(piperidin-1-yl)methyl-benzamide;
(2S,4S,5S,7S)-N-(4-Amino-7-butylcarbamoyl-5-hydroxy-2-isopropyl-8-methyl-nonyl)-2-(4-methoxybutoxy)-4-(pyrrolidin-1-yl)methyl-benzamide;
(2S,4S,5S,7S)-N-(4-Amino-7-butylcarbamoyl-5-hydroxy-2-isopropyl-8-methyl-nonyl)-2-(4-methoxybutoxy)-4-(2-piperidin-1-ylethoxy)-benzamide;
(2S,4S,5S,7S)-N-(4-Amino-7-butylcarbamoyl-5-hydroxy-2-isopropyl-8-methyl-nonyl)-4-dimethylaminomethyl-2-(4-methoxybutoxy)-benzamide;
(2S,4S,5S,7S)-N-(4-Amino-7-butylcarbamoyl-5-hydroxy-2-isopropyl-8-methyl-nonyl)-2-(4-methoxybutoxy)-4-(4-methylpiperazin-1-yl)methyl-benzamide;
(2S,4S,5S,7S)-N-(4-Amino-7-butylcarbamoyl-5-hydroxy-2-isopropyl-8-methyl-nonyl)-4-(4-acetylpiperazin-1-yl)methyl-2-(4-methoxybutoxy)-benzamide;
(2S,4S,5S,7S)-N-(4-Amino-7-butylcarbamoyl-5-hydroxy-2-isopropyl-8-methyl-nonyl)-2-(3-aminopropoxy)-benzamide;
(2S,4S,5S,7S)-N-(4-Amino-7-butylcarbamoyl-5-hydroxy-2-isopropyl-8-methyl-nonyl)-2-(2-aminoethoxy)-benzamide;
(2S,4S,5S,7S)-N-(4-Amino-7-butylcarbamoyl-5-hydroxy-2-isopropyl-8-methyl-nonyl)-2-[2-(4-acetylpiperazin-1-yl)-ethoxy]-benzamide;
(2S,4S,5S,7S)-N-(4-Amino-7-butylcarbamoyl-5-hydroxy-2-isopropyl-8-methyl-nonyl)-2-[2-(morpholin-4-yl)-ethyl]-benzamide;
(2S,4S,5S,7S)-N-(4-Amino-7-butylcarbamoyl-5-hydroxy-2-isopropyl-8-methyl-nonyl)-2-(3-dimethylaminopropoxy)-benzamide;
(2S,4S,5S,7S)-N-(4-Amino-7-butylcarbamoyl-5-hydroxy-2-isopropyl-8-methyl-nonyl)-2-[3-(morpholin-4-yl)-propoxy]-benzamide;
(2S,4S,5S,7S)-N-(4-Amino-7-butylcarbamoyl-5-hydroxy-2-isopropyl-8-methyl-nonyl)-2-[2-(morpholin-4-yl)-ethoxy]-benzamide;
(2S,4S,5S,7S)-N-(4-Amino-7-butylcarbamoyl-5-hydroxy-2-isopropyl-8-methyl-nonyl)-2-[2(4-methoxypiperidin-1-yl)-ethyl]-benzamide;
(2S,4S,5S,7S)-N-(4-Amino-7-butylcarbamoyl-5-hydroxy-2-isopropyl-8-methyl-nonyl)-2-[2(4-acetylpiperazin-1-yl)-ethyl]-benzamide;
(2S,4S,5S,7S)-4-Amino-5-hydroxy-2,7-diisopropyl-octanedioic acid 8-butylamide 1-[2-(3-methoxypropoxy)-benzyl]amide;
(2S,4S,5S,7S)-4-Amino-5-hydroxy-2,7-diisopropyl-octanedioic acid 8-butylamide 1-[3-(3-methoxypropoxy)-benzyl]amide;
(2S,4S,5S,7S)-4-Amino-5-hydroxy-2,7-diisopropyl-octandioic acid 8-butylamide 1-[2-(4-methoxybutoxy)-benzyl]amide;
(2S,4S,5S,7S)-4-Amino-5-hydroxy-2,7-diisopropyl-octandioic acid 8-butylamide 1-[2-(5-methoxypentyloxy)-benzyl]amide;
(2S,4S,5S,7S)-N1-(4-Amino-7-butylcarbamoyl-5-hydroxy-2-isopropyl-8-methyl-nonyl)-N-4-methyl-2-(4-methoxybutoxy)-terephthaldiamide;
(2S,4S,5S,7S)-N1-(4-Amino-7-butylcarbamoyl-5-hydroxy-2-isopropyl-8-methyl-nonyl)-N-4-[(2-morpholin-4-yl)-ethyl]-2-(4-methoxybutoxy)-terephthaldiamide;
(2S,4S,5S,7S)-N1-(4-Amino-7-butylcarbamoyl-5-hydroxy-2-isopropyl-8-methyl-nonyl)-2-(4-methoxybutoxy)-terephthaldiamide;
(2S,4S,5S,7S)-N-4-(4-Amino-7-butylcarbamoyl-5-hydroxy-2-isopropyl-8-methyl-nonyl)-3-(4-methoxybutoxy)-terephthalmic acid;
(2S,4S,5S,7S)-N-(4-Amino-7-butylcarbamoyl-5-hydroxy-2-isopropyl-8-methyl-nonyl)-4-butylcarbamoylmethoxy-2-(4-methoxybutoxy)-benzamide;
(2S,4S,5S,7S)-[4-(4-Amino-7-butylcarbamoyl-5-hydroxy-2-isopropyl-8-methyl-nonylcarbamoyl)-3-(4-methoxybutoxy)-phenoxy]-acetic acid;
(2S,4S,5S,7S)-N-{4-Amino-5-hydroxy-2-isopropyl-8-methyl-7-[2-(morpholin-4-yl)-ethylcarbamoyl]-nonyl}-2-(4-methoxybutoxy)-4-[2-(morpholin-4-yl)-ethylcarbamoylmethoxy]-benzamide;
(2S,4S,5S,7S)-N-(4-Amino-7-butylcarbamoyl-5-hydroxy-2-isopropyl-8-methyl-nonyl)-2-(4-methoxybutoxy)-4-(1H-tetrazol-5-ylmethoxy)-benzamide;
(2S,4S,5S,7S,2R′)-N-[4-Amino-7-(2′-methylcarbamoyl-propylcarbamoyl)-5-hydroxy-2-isopropyl-8-methyl-nonyl]-2-(4-methoxybutoxy)-benzamide;
(2S,4S,5S,7S)-N-(4-Amino-7-[2-(dimethylaminocarbamoyl)-ethylcarbamoyl]-5-hydroxy-2-isopropyl-8-methyl-nonyl)-2-(4-methoxybutoxy)-benzamide;
(2S,4S,5S,7S)-N-[4-Amino-7-(3-carbamoylpropylcarbamoyl)-5-hydroxy-2-isopropyl-8-methyl-nonyl]-2-(4-methoxybutoxy)-benzamide;
(2S,4S,5S,7S)-N-[4-Amino-7-(2-carbamoyl-2-methylpropylcarbamoyl)-5-hydroxy-2-isopropyl-8-methyl-nonyl]-2-(4-methoxybutoxy)-benzamide;
(2S,4S,5S,7S)-N-{4-Amino-5-hydroxy-2-isopropyl-8-methyl-7-[3-(morpholin-4-yl)-3-oxopropylcarbamoyl]-nonyl}-2-(4-methoxybutoxy)-benzamide;
(2S,4S,5S,7S)-N-{7-[2-(4-Acetylpiperidin-1-yl)-ethylcarbamoyl]-4amino-5-hydroxy-2-isopropyl-8-methyl-nonyl}-2-(4-methoxybutoxy)-benzamide;
(2S,4S,5S,7S)-N-[4-Amino-5-hydroxy-2-isopropyl-8-methyl-7-(2-thiomorpholin-4-ylethylcarbamoyl)-methyl-nonyl]-2-(4-methoxybutoxy)-benzamide;
(2S,4S,5S,7S)-N-(4-Amino-7-(2-carbamoyl-2-methylpropylcarbamoyl)-5-hydroxy-2-isopropyl-8-methyl-nonyl)-2-(4-methoxybutoxy)-4-(2-morpholin-4-ylmethoxy)-benzamide;
(2S,4S,5S,7S)-N-(4-Amino-7-(2-carbamoyl-2-methylpropylcarbamoyl)-5-hydroxy-2-isopropyl-8-methy-nonyl)-2-(4-methoxybutoxy)-4-(morpholin-4-ylmethyl)-benzamide;
(2S,4S,5S,7S)-N-[4-Amino-7-(2-carbamoyl-2-methylpropylcarbamoyl)-5-hydroxy-2-isopropyl-8-methyl-nonyl]-2-(2-morpholin-4-ylethoxy)-benzamide;
(2S,4S,5S,7S)-N-{4-Amino-5-hydroxy-2-isopropyl-7-[2-(4-methoxycarbonylpiperidin-1-yl)-ethylcarbamoyl]-8-methyl-nonyl}-2-(4-methoxybutoxy)-benzamide;
(2S,4S,5S,7R)-N-[4-Amino-5-hydroxy-2-methyl-7-[(2-morpholin-4-ylethyl)-carbamoyl]-octyl}-2-(3-methoxypropoxy)-benzamide; and
(2S,4S,5S,7S)-N-{4-Amino-5-hydroxy-2-isopropyl-8-methyl-7-[2-(morpholin-4-yl)-ethyl-carbamoyl]-nonyl}-4-carbamoylmethoxy-2-(4-methoxybutoxy)-benzamide;
or pharmaceutically acceptable salts thereof.

7-8. (canceled)

9. A method for inhibiting beta-secretase activity, comprising contacting an effective amount for inhibition of a compound of formula (I): 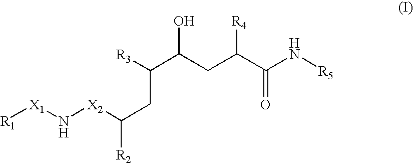
wherein R₁ is a 2-R_{A}-3-R_{B}-phenyl radical, a 2-R_{A}-4-R_{C}-phenyl radical, a 2-R_{A}-pyridin-3-yl radical a 3-R_{A}-pyridin-2-yl radical or a 1-R_{D}-indol-3-yl radical,
wherein one of the radicals R_{A} and R_{B} is an aliphatic or heterecycloaliphatic-aliphatic radical or free or aliphatically, araliphatically or heteroaraliphatically etherified hydroxy and the other is hydrogen, an aliphatic radical or free or esterified or amidated carboxy,
R_{C} is hydrogen, an aliphatic radical, free or aliphatically, araliphatically, heterearaliphatically or heterearylaliphatically etherified hydroxy or an unsubstituted or heteroaliphatically substituted amino group, and
R_{D} is an aliphatic, araliphatic or heteroaliphatic radical,
one of the radicals X₁ and X₂ is carbonyl and the other is methylene,
R₂ is an aliphatic radical,
R₃ is unsubstituted or aliphatically substituted amino,
R₄ is an aliphatic or araliphatic radical, and
R₅ is an aliphatic or cycloaliphatic-aliphatic radical or an optionally hydrogenated and/or oxo-substituted heteroaryl radical or an optionally hydrogenated and/or oxo-substituted heteroaryl or heteroaliphatyl radical bonded via a carbon atom.

10. (canceled)

11. A method for inhibiting production of amyloid beta peptide (A beta) in a cell, comprising administering to said cell an effective inhibitory amount of a compound of formula (I): 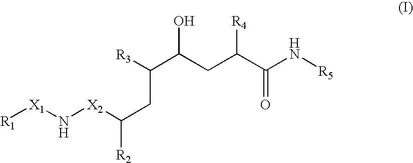
wherein R₁ is a 2-R_{A}-3-R_{B}-phenyl radical, a 2-R_{A}-4-R_{C}-phenyl radical, a 2-R_{A}-pyridin-3-yl radical a 3-R_{A}-pyridin-2-yl radical or a 1-R_{D}-indol-3-yl radical,
wherein one of the radicals R_{A} and R_{B} is an aliphatic or heterecycloaliphatic-aliphatic radical or free or aliphatically, araliphatically or heteroaraliphatically etherified hydroxy and the other is hydrogen, an aliphatic radical or free or esterified or amidated carboxy,
R_{C} is hydrogen, an aliphatic radical, free or aliphatically, araliphatically, heterearaliphatically or heterearylaliphatically etherified hydroxy or an unsubstituted or heteroaliphatically substituted amino group, and
R_{D} is an aliphatic, araliphatic or heteroaliphatic radical,
one of the radicals X₁ and X₂ is carbonyl and the other is methylene,
R₂ is an aliphatic radical,
R₃ is unsubstituted or aliphatically substituted amino,
R₄ is an aliphatic or araliphatic radical, and
R₅ is an aliphatic or cycloaliphatic-aliphatic radical or an optionally hydrogenated and/or oxo-substituted heteroaryl radical or an optionally hydrogenated and/or oxo-substituted heteroaryl or heteroaliphatyl radical bonded via a carbon atom.

12. The method of claim 11, wherein the cell is an animal cell.

13. The method of claim 12, wherein the animal cell is a mammalian cell.

14. The method of claim 13, wherein the mammalian cell is human.

15-19. (canceled)

20. A method of treatment according to claim 5, further comprising administration of one or more therapeutic agents selected from the group consisting of an antioxidant, an anti-inflammatory, a gamma secretase inhibitor, a neurotrophic agent, an acetyl cholinesterase inhibitor, a statin, an A beta peptide, and an anti-A beta peptide.

21. (canceled)

22. A method of according to claim 1 where the compound is represented by Formula (I-A) or a pharmaceutically acceptable salt thereof: 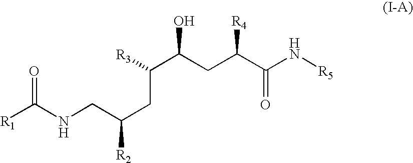
wherein R₁ is a 2-R_{A}-4-R_{C}-phenyl radical, a 2-R_{A}-pyridin-3-yl radical or a 3-R_{A}-pyridin-2-yl radical, wherein
R_{A}, is C₁-C₄ alkoxy-C₁-C₄ alkyl, such as propyloxymethyl, morpholino-C₁-C₄ alkyl, such as 2-morpholinoethyl or 3-morpholinopropyl, C₁-C₇ alkanoylpiperazino-C₁-C₄ alkyl, such as N′-acetylpiperazinomethyl, C₁-C₇ alkoxy, such as propyloxy, C₁-C₄ alkoxy-C₁-C₄ alkoxy, such as 2-methoxyethoxy, 3-methoxypropyloxy, 4-methoxybutyloxy or 5-methoxypentyloxy, C₁-C₄ alkoxy-C₁-C₄ alkenyloxy, such as 4-methoxy-but-2-enyloxy, C₁-C₄ alkoxy-C₁ C₄ alkoxy, such as 2-(methoxymethoxy)ethoxy or 2-(2-methoxyethoxy)ethoxy, amino-C₁-C₄ alkoxy, such as 2-aminoethoxy or 3-aminopropyloxy, di-C₁-C₄ alkylamino-C₁-C₄ alkoxy, such as 3-dimethylaminopropyloxy, carbamoyl-C₁-C₄ alkoxy, such as 2-carbamoylethoxy, or carbamoyl, and
R_{C} is hydrogen, di-C₁-C₄ alkylamino-C₁-C₄ alkyl, such as dimethylaminomethyl, piperidino-C₁-C₄ alkyl, such as piperidinomethyl, pyrrolidino-C₁-C₄ alkyl, such as pyrrolidinomethyl, morpholino-C₁-C₄ alkyl, such as morpholinomethyl, C₁-C₇ alkanoylpiperazino-C₁-C₄ alkyl, such as N′-acetylpiperazinomethyl, or C₁-C₄ alkylpiperazino-C₁-C₄ alkyl, such as N′-methylpiperazinomethyl, morpholino, C₁-C₄ alkoxy, such as methoxy, morpholino-C₁-C₄ alkoxy, such as 2-morpholinoethoxy or 3-morpholinopropyloxy, morpholino-C₁-C₄ alkylcarbamoyl-C₁-C₄ alkoxy, such as 2-morpholinoethylcarbamoylmethoxy, piperidino-C₁-C₄ alkoxy, such as 2-piperidinoethoxy, carboxy, carbamoyl, C₁-C₄ alkylcarbamoyl, such as methylcarbamoyl, carboxy-C₁-C₄ alkoxy, such as carboxymethoxy, di-C₁-C₄ alkylamino-C₁-C₄ alkoxy, such as 3-dimethylaminopropyloxy, C₁-C₇ alkylcarbamoyl-C₁-C₄ alkoxy, such as butylcarbamoylmethoxy, or tetrazolyl-C₁-C₄ alkoxy, such as tetrazol-5-ylmethoxy,
X₁ is carbonyl and X₂ is methylene,
R₂ and R₄ are each independently of the other C₁-C₄ alkyl, such as methyl or isopropyl,
R₃ is amino and
R₅ is C₁-C₄ alkyl, such as butyl, morpholino-C₁-C₄ alkyl, such as 2-morpholinoethyl or 3-morpholinopropyl, thiomorpholino-C₁-C₄ alkyl, such as 2-thiomorpholinoethyl, morpholinocarbonyl-C₁-C₄ alkyl, such as 2-morpholinocarbonylethyl, carbamoyl-C₁-C₄ alkyl, such as 3-carbamoylpropyl or 2-carbamoyl-2-methyl-ethyl, C₁-C₄ alkylcarbamoyl-C₁-C₄ alkyl, such as 2-methylcarbamoyl-2-methyl-ethyl, di-C₁-C₄ alkylcarbamoyl-C₁-C₄ alkyl, such as 2-dimethylcarbamoylethyl, N′-C₁-C₄ alkylpiperazino-C₁-C₄ alkyl, such as N′-methylpiperazinomethyl, N′-C₁-C₄ alkoxycarbonylpiperazino-C₁-C₄ alkyl, such as N′-methoxycarbonylpiperazinomethyl, or N′-C₁-C₇ alkanoylpiperazino-C₁-C₄ alkyl, such as N′-acetylpiperazinomethyl.

23. A method according to claim 20, wherein the compound is represented by formula (I-A), or a pharmaceutically acceptable salt thereof: 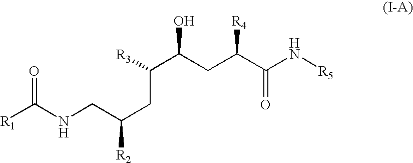
wherein R₁ is a 2-R_{A}-4-R_{C}-phenyl radical, a 2-R_{A}-pyridin-3-yl radical or a 3-R_{A}-pyridin-2-yl radical, wherein
R_{A}, is C₁-C₄ alkoxy-C₁-C₄ alkyl, such as propyloxymethyl, morpholino-C₁-C₄ alkyl, such as 2-morpholinoethyl or 3-morpholinopropyl, C₁-C₇ alkanoylpiperazino-C₁-C₄ alkyl, such as N′-acetylpiperazinomethyl, C₁-C₇ alkoxy, such as propyloxy, C₁-C₄ alkoxy-C₁-C₄ alkoxy, such as 2-methoxyethoxy, 3-methoxypropyloxy, 4-methoxybutyloxy or 5-methoxypentyloxy, C₁-C₄ alkoxy-C₁-C₄ alkenyloxy, such as 4-methoxy-but-2-enyloxy, C₁-C₄ alkoxy-C₁-C₄ alkoxy, such as 2-(methoxymethoxy)ethoxy or 2-(2-methoxyethoxy)ethoxy, amino-C₁-C₄ alkoxy, such as 2-aminoethoxy or 3-aminopropyloxy, di-C₁-C₄ alkylamino-C₁-C₄ alkoxy, such as 3-dimethylaminopropyloxy, carbamoyl-C₁-C₄ alkoxy, such as 2-carbamoylethoxy, or carbamoyl, and
R_{C} is hydrogen, di-C₁-C₄ alkylamino-C₁-C₄ alkyl, such as dimethylaminomethyl, piperidino-C₁-C₄ alkyl, such as piperidinomethyl, pyrrolidino-C₁-C₄ alkyl, such as pyrrolidinomethyl, morpholino-C₁-C₄ alkyl, such as morpholinomethyl, C₁-C₇ alkanoylpiperazino-C₁-C₄ alkyl, such as N′-acetylpiperazinomethyl, or C₁-C₄ alkylpiperazino-C₁-C₄ alkyl, such as N′-methylpiperazinomethyl, morpholino, C₁-C₄ alkoxy, such as methoxy, morpholino-C₁-C₄ alkoxy, such as 2-morpholinoethoxy or 3-morpholinopropyloxy, morpholino-C₁-C₄ alkylcarbamoyl-C₁-C₄ alkoxy, such as 2-morpholinoethylcarbamoylmethoxy, piperidino-C₁-C₄ alkoxy, such as 2-piperidinoethoxy, carboxy, carbamoyl, C₁-C₄ alkylcarbamoyl, such as methylcarbamoyl, carboxy-C₁-C₄ alkoxy, such as carboxymethoxy, di-C₁-C₄ alkylamino-C₁-C₄ alkoxy, such as 3-dimethylaminopropyloxy, C₁-C₇ alkylcarbamoyl-C₁-C₄ alkoxy, such as butylcarbamoylmethoxy, or tetrazolyl-C₁-C₄ alkoxy, such as tetrazol-5-ylmethoxy,
X₁ is carbonyl and X₂ is methylene,
R₂ and R₄ are each independently of the other C₁-C₄ alkyl, such as methyl or isopropyl,
R₃ is amino and
R₅ is C₁-C₄ alkyl, such as butyl, morpholino-C₁-C₄ alkyl, such as 2-morpholinoethyl or 3-morpholinopropyl, thiomorpholino-C₁-C₄ alkyl, such as 2-thiomorpholinoethyl, morpholinocarbonyl-C₁-C₄ alkyl, such as 2-morpholinocarbonylethyl, carbamoyl-C₁-C₄ alkyl, such as 3-carbamoylpropyl or 2-carbamoyl-2-methyl-ethyl, C₁-C₄ alkylcarbamoyl-C₁-C₄ alkyl, such as 2-methylcarbamoyl-2-methyl-ethyl, di-C₁-C₄ alkylcarbamoyl-C₁-C₄ alkyl, such as 2-dimethylcarbamoylethyl, N′-C₁-C₄ alkylpiperazino-C₁-C₄ alkyl, such as N′-methylpiperazinomethyl, N′-C₁-C₄ alkoxycarbonylpiperazino-C₁-C₄ alkyl, such as N′-methoxycarbonylpiperazinomethyl, or N′-C₁-C₇ alkanoylpiperazino-C₁-C₄ alkyl, such as N′-acetylpiperazinomethyl.

24. (canceled)

25. A method according to claim 5, wherein
R₁ is a 2-R_{A}-3-R_{B}-phenyl radical, a 2-R_{A}-4-R_{C}-phenyl radical, a 2-R_{A}-pyridin-3-yl radical, a 3-R_{A}-pyridin-2-yl radical or a 1-R_{D}-indol-3-yl radical, wherein
one of the radicals R_{A} and R_{B} is an aliphatic or heterocycloaliphatic-aliphatic radical or free or aliphatically, araliphatically or heteroaraliphatically etherified hydroxy and the other is hydrogen, an aliphatic radical or free or esterified or amidated carboxy,
R_{C} is hydrogen, an aliphatic radical, free or aliphatically, araliphatically, heteroaraliphatically or heteroarylaliphatically etherified hydroxy or an unsubstituted or heteroaliphatically substituted amino group, and
R_{D} is an aliphatic, araliphatic or heteroaliphatic radical,
one of the radicals X₁ and X₂ is carbonyl and the other is methylene,
R₂ is an aliphatic radical,
R₃ is unsubstituted or aliphatically substituted amino,
R₄ is an aliphatic or araliphatic radical, and
R₅ is an aliphatic or cycloaliphatic-aliphatic radical or an optionally hydrogenated and/or oxo-substituted heteroaryl radical or an optionally hydrogenated and/or oxo-substituted heteroaryl or heteroaliphatyl radical bonded via a carbon atom,
or a pharmaceutically acceptable salt thereof.

26. The method according to claim 25, wherein
R₁ is a 2-R_{A}-3-R_{B}-phenyl radical, a 2-R_{A}-4-R_{C}-phenyl radical, a 2-R_{A}-pyridin-3-yl radical, a 3-R_{A}-pyridin-2-yl radical or a 1-R_{D}-indol-3-yl radical,
wherein one of the radicals R_{A} and R_{B} is lower alkyl, hydroxy-lower alkyl, lower alkanoyloxy-lower alkyl, lower alkoxy-lower alkyl, lower alkoxy-lower alkoxy-lower alkyl; an amino-lower alkyl or amino-lower alkoxy radical that is unsubstituted or N-lower alkanoylated or N-mono- or N,N-di lower alkylated or N,N-disubstituted by lower alkylene, hydroxy-, lower alkoxy- or lower alkoxy-lower alkoxy-lower alkylene, by unsubstituted or N′-lower alkanoylated, lower alkoxycarbonyl- or lower alkoxy-lower alkyl-N′-substituted or N′-lower alkylated aza-lower alkylene, by oxa-lower alkylene or by optionally S-oxidised thia-lower alkylene; hydroxy, lower alkoxy, hydroxy-lower alkoxy, lower alkanoyloxy-lower alkoxy, lower alkoxy-lower alkoxy, lower alkoxy-lower alkoxy-lower alkoxy, polyhalo-lower alkoxy, cyano-lower alkoxy, unsubstituted or substituted phenyl- or pyridyl-lower alkoxy, lower alkoxy-lower alkenyloxy, optionally S-oxidised lower alkylthio-lower alkoxy, or amino-lower alkoxy that is unsubstituted or N-lower alkanoylated or N-mono- or N,N-di-lower alkylated or N,N-disubstituted by lower alkylene, hydroxy-, lower alkoxy- or lower alkoxy-lower alkoxy-lower alkylene, by unsubstituted or N′-lower alkanoylated, lower alkoxycarbonyl- or lower alkoxy-lower alkyl-N′-substituted or N′-lower alkylated aza-lower alkylene, by oxa-lower alkylene or by optionally S-oxidised thia-lower alkylene; and the other is hydrogen, lower alkyl, carbamoyl, hydroxy, lower alkoxy or polyhalo-lower alkoxy,
R_{C} is hydrogen, lower alkyl, hydroxy, lower alkoxy, hydroxy-lower alkoxy, lower alkoxy-lower alkoxy, morpholino-lower alkylcarbamoyl-lower alkoxy, lower alkoxy-lower alkoxy-lower alkyl; an amino, amino-lower alkyl or amino-lower alkoxy group that is unsubstituted or N-lower alkanoylated or N-mono- or N,N-di-lower alkylated or N,N-disubstituted by lower alkylene, hydroxy-, lower alkoxy-, lower alkoxycarbonyl- or lower alkoxy-lower alkoxy-lower alkylene, by unsubstituted or N′-lower alkanoylated, lower alkoxycarbonyl- or lower alkoxy-lower alkyl-N′-substituted or N′-lower alkylated aza-lower alkylene, by oxa-lower alkylene or by optionally S-oxidised thia-lower alkylene; or a free or amidated carboxy or carboxy-lower alkoxy group or tetrazolyl-lower alkoxy, and
R_{D} is lower alkyl, hydroxy-lower alkyl, lower alkoxy-lower alkyl, lower alkoxy-lower alkoxy-lower alkyl, hydroxy-lower alkoxy-lower alkyl, a free or amidated carboxy or carboxy-lower alkyl group or an unsubstituted or substituted phenyl- or pyridyl-lower alkyl group, one of the radicals X₁ and X₂ is carbonyl and the other is methylene,
R₂ is lower alkyl,
R₃ is unsubstituted or N-lower alkanoylated or N-mono- or N, N-di-lower alkylated amino,
R₄ is lower alkyl or phenyl-lower alkyl, and
R₅ is lower alkyl, cycloalkyl-lower alkyl, hydroxy-lower alkyl, lower alkoxy-lower alkyl, lower alkanoyloxy-lower alkyl; amino-lower alkyl that is unsubstituted or N-lower alkanoylated or N-mono- or N,N-di-lower alkylated or N,N-disubstituted by lower alkylene, hydroxy-, lower alkoxy-, lower alkoxy-lower alkyl- or lower alkanoyloxy-lower alkylene, by unsubstituted or N′-lower alkanoylated, lower alkoxycarbonyl- or lower alkoxy-lower alkyl-N′-substituted or N′-lower alkylated aza-lower alkylene, by oxa-lower alkylene or by optionally S-oxidised thia-lower alkylene; free or esterified or amidated carboxy-lower alkyl, cyano-lower alkyl, free or esterified or amidated dicarboxy-lower alkyl, free or esterified or amidated carboxy(hydroxy)-lower alkyl, free or esterified or amidated carboxycycloalkyl-lower alkyl, lower alkanesulfonyl-lower alkyl, unsubstituted or N-mono- or N,N-di-lower alkylated thio carbamoyl-lower alkyl, unsubstituted or N-mono- or N,N-di-lower alkylated sulfamoyl-lower alkyl or an optionally hydrogenated and/or oxo-substituted heteroaryl radical or lower alkyl substituted by an optionally hydrogenated and/or oxo-substituted heteroaryl radical that is bonded via a carbon atom,
or a pharmaceutically acceptable salt thereof.

27. A method according to claim 25 wherein,
R₁ is a 2-R_{A}-3-R_{B}-phenyl radical, a 2-R_{A}-4-R_{C}-phenyl radical, a 2-R_{A}-pyridin-3-yl radical, a 3-R_{A}-pyridin-2-yl radical or a 1-R_{D}-indol-3-yl radical, wherein
one of the radicals R_{A} and R_{B} is lower alkyl, hydroxy-lower alkyl, lower alkanoyloxy-lower alkyl, lower alkoxy-lower alkyl, lower alkoxy-lower alkoxy-lower alkyl, amino-lower alkyl, lower alkanoylamino-lower alkyl, lower alkylamino-lower alkyl, di-lower alkylamino-lower alkyl; piperidino- or pyrrolidino-lower alkyl that is unsubstituted or substituted by hydroxy, lower alkoxy or by lower alkoxy-lower alkyl; piperazino-lower alkyl that is unsubstituted or N′-lower alkylated, N′-lower alkanoylated or N′-substituted by lower alkoxycarbonyl or by lower alkoxy-lower alkyl; unsubstituted or lower alkylated morpholino-lower alkyl, optionally S-oxidised thiomorpholino-lower alkyl, amino-lower alkoxy, lower alkanoylamino-lower alkoxy, lower alkylamino-lower alkoxy, di-lower alkylamino-lower alkoxy; piperidino- or pyrrolidino-lower alkoxy that is unsubstituted or substituted by hydroxy, lower alkoxy or by lower alkoxy-lower alkyl; piperazino-lower alkoxy that is unsubstituted or N′-lower alkylated, N′-lower alkanoylated or N′-substituted by lower alkoxycarbonyl or by lower alkoxy-lower alkyl; unsubstituted or lower alkylated morpholino-lower alkoxy, optionally S-oxidised thiomorpholio-lower alkoxy, hydroxy, lower alkoxy, hydroxy-lower alkoxy, lower alkanoyloxy-lower alkoxy, lower alkoxy-lower alkoxy, lower alkoxy-lower alkoxy-lower alkoxy, polyhalo-lower alkoxy, cyano-lower alkoxy; phenyl- or pyridyl-lower alkoxy that is unsubstituted or substituted by lower alkyl, lower alkoxy, hydroxy, nitro, amino, lower alkylamino, di-lower alkylamino, halogen and/or by trifluoromethyl; lower alkoxy-lower alkenyloxy, lower alkylthio-lower alkoxy, lower alkanesulfinyl-lower alkoxy, lower alkanesulfonyl-lower alkoxy, amino-lower alkoxy, lower alkanoylamino-lower alkoxy, lower alkylamino-lower alkoxy, di-lower alkylamino-lower alkoxy; piperidino- or pyrrolidino-lower alkoxy that is unsubstituted or substituted by hydroxy, lower alkoxy or by lower alkoxy-lower alkyl; piperazino-lower alkoxy that is unsubstituted or N′-lower alkylated, N′-lower alkanoylated or N′-substituted by lower alkoxycarbonyl or by lower alkoxy-lower alkyl; unsubstituted or lower alkylated morpholino-lower alkoxy or optionally S-oxidised thiomorpholino-lower alkoxy, and the other is hydrogen, carbamoyl, hydroxy, lower alkoxy or polyhalo-lower alkoxy,
R_{C} is hydrogen, lower alkyl, lower alkoxy-lower alkoxy-lower alkyl, amino-lower alkyl, lower alkanoylamino-lower alkyl, lower alkylamino-lower alkyl, di-lower alkylamino-lower alkyl; piperidino- or pyrrolidino-lower alkyl that is unsubstituted or substituted by hydroxy, lower alkoxy or by lower alkoxy-lower alkyl; piperazino-lower alkyl that is unsubstituted or N′-lower alkylated, N′-lower alkanoylated or N′-substituted by lower alkoxycarbonyl or by lower alkoxy-lower alkyl; unsubstituted or lower alkylated morpholino-lower alkyl, optionally S-oxidised thiomorpholino-lower alkyl, di-lower alkylamino; a piperidino or pyrrolidino group that is unsubstituted or substituted by hydroxy, lower alkoxy or by lower alkoxy-lower alkyl; piperazino that is unsubstituted or N′-lower alkylated, N′-lower alkanoylated or N′-substituted by lower alkoxycarbonyl or by lower alkoxy-lower alkyl; unsubstituted or lower alkylated morpholino, optionally S-oxidised thiomorpholino, hydroxy, lower alkoxy, hydroxy-lower alkoxy, lower alkoxy-lower alkoxy, morpholino-lower alkylcarbamoyl-lower alkoxy, amino-lower alkoxy, lower alkanoylamino-lower alkoxy, lower alkylamino-lower alkoxy, di-lower alkylamino-lower alkoxy; piperidino- or pyrrolidino-lower alkoxy that is unsubstituted or substituted by hydroxy, lower alkoxy or by lower alkoxy-lower alkyl; piperazino-lower alkoxy that is unsubstituted or N′-lower alkylated, N′-lower alkanoylated or N′-substituted by lower alkoxycarbonyl or by lower alkoxy-lower alkyl; unsubstituted or lower alkylated morpholino-lower alkoxy, optionally S-oxidised thiomorpholino-lower alkoxy, carboxy-lower alkoxy, carbamoyl-lower alkoxy, lower alkylcarbamoyl-lower alkoxy, di-lower alkylcarbamoyl-lower alkoxy; piperidino- or pyrrolidino-carbonyl-lower alkoxy that is unsubstituted or substituted by hydroxy, lower alkoxy or by lower alkoxy-lower alkyl; piperazinocarbonyl-lower alkoxy that is unsubstituted or N′-lower alkylated, N′-lower alkanoylated or N′-substituted by lower alkoxycarbonyl or by lower alkoxy-lower alkyl; unsubstituted or lower alkylated morpholinocarbonyl-lower alkoxy, optionally S-oxidised thiomorpholinocarbonyl-lower alkoxy, tetrazolyl-lower alkoxy, carboxy, carbamoyl, lower alkylcarbamoyl or di-lower alkylcarbamoyl, and RD is lower alkyl, hydroxy-lower alkyl, lower alkoxy-lower alkyl, lower alkoxy-lower alkoxy-lower alkyl, hydroxy-lower alkoxy-lower alkyl, carboxy, lower alkoxycarbonyl, carboxy-lower alkyl, lower alkoxycarbonyl-lower alkyl, carbamoyl-lower alkyl, lower alkylcarbamoyl-lower alkyl, di-lower alkylcarbamoyl-lower alkyl; piperidino- or pyrrolidino-carbonyl-lower alkyl that is unsubstituted or substituted by hydroxy, lower alkoxy or by lower alkoxy-lower alkyl; piperazinocarbonyl-lower alkyl that is unsubstituted or N′-lower alkylated, N′-lower alkanoylated or N′-substituted by lower alkoxycarbonyl or by lower alkoxy-lower alkyl; unsubstituted or lower alkylated morpholinocarbonyl-lower alkyl, optionally S-oxidised thiomorpholinocarbonyl-carbonyl-lower alkyl, carboxy-lower alkyl, lower alkoxycarbonyl-lower alkyl or a phenyl- or pyridyl-lower alkyl group that is unsubstituted or substituted by lower alkyl, lower alkoxy, hydroxy, nitro, amino, lower alkylamino, di-lower alkylamino, halogen and/or by trifluoromethyl,
one of the radicals X₁ and X₂ is carbonyl and the other is methylene,
R₂ is lower alkyl,
R₃ is amino, lower alkanoylamino, lower alkylamino or di-lower alkylamino,
R₄ is lower alkyl or phenyl-lower alkyl and
R₅ is lower alkyl, cycloalkyl-lower alkyl, hydroxy-lower alkyl, lower alkoxy-lower alkyl, lower alkanoyloxy-lower alkyl; piperidino- or pyrrolidino-carbonyl-lower alkyl that is unsubstituted or substituted by hydroxy, lower alkoxy or by lower alkoxy-lower alkyl; piperazinocarbonyl-lower alkyl that is unsubstituted or N′-lower alkylated, N′-lower alkanoylated or N′-substituted by lower alkoxycarbonyl or by lower alkoxy-lower alkyl; unsubstituted or lower alkylated morpholinocarbonyl-lower alkyl, optionally S-oxidised thiomorpholinocarbonyl-lower alkyl, carboxy-lower alkyl, lower alkoxycarbonyl-lower alkyl, carbamoyl-lower alkyl, lower alkylcarbamoyl-lower alkyl, di-lower alkylcarbamoyl-lower alkyl; piperidino- or pyrrolidinocarbonyl-lower alkyl that is unsubstituted or substituted by hydroxy, lower alkoxy or by lower alkoxy-lower alkyl; piperazinocarbonyl-lower alkyl that is unsubstituted or N′-lower alkylated, N′-lower alkanoylated or N′-substituted by lower alkoxycarbonyl or by lower alkoxy-lower alkyl; unsubstituted or lower alkylated morpholinocarbonyl-lower alkyl, optionally S-oxidised thiomorpholinocarbonyl-lower alkyl, cyano-lower alkyl, dicarboxy-lower alkyl, lower alkoxycarbonyl(carbonyl)-lower alkyl, di-lower alkoxycarbonyl-lower alkyl, dicarbamoyl-lower alkyl, carbamoyl(carboxy)-lower alkyl, di-(lower alkylcarbamoyl)-lower alkyl, di-(di-lower alkylcarbamoyl)-lower alkyl, carboxy(hydroxy)-lower alkyl, lower alkoxycarbonyl(hydroxy)-lower alkyl, carbamoyl(hydroxy)-lower alkyl, lower alkylcarbamoyl(hydroxy)-lower alkyl or di-lower alkylcarbamoyl(hydroxy)-lower alkyl, carboxycycloalkyl-lower alkyl, lower alkoxycarbonylcycloalkyl-lower alkyl, carbamoylcycloalkyl-lower alkyl, lower alkylcarbamoylcycloalkyl-lower alkyl, di-lower alkylcarbamoylcycloalkyl-lower alkyl, lower alkanesulfonyl-lower alkyl, thiocarbamoyl-lower alkyl, N-lower alkylthiocarbamoyl-lower alkyl or N,N-di-lower alkylthiocarbamoyl-lower alkyl, sulfamoyl-lower alkyl, lower alkylsulfamoyl-lower alkyl or di-lower alkylsulfamoyl-lower alkyl, unsubstituted or oxo-substituted pyrrolidinyl, imidazolyl, benzimidazolyl, oxadiazolyl, pyridyl, oxopiperidinyl, dioxopiperidinyl, oxothiazolyl, oxo-oxazolinyl or quinolinyl, unsubstituted or oxo-substituted pyrrolidinyl-lower alkyl, imidazolyl-lower alkyl, benzimidazolyl-lower alkyl, oxadiazolyl-lower alkyl, pyridyl-lower alkyl, oxopiperidinyl-lower alkyl, dioxopiperidinyl-lower alkyl, oxothiazolyl-lower alkyl, oxo-oxazolinyl-lower alkyl or quinolinyl-lower alkyl, morpholinocarbonyl-lower alkyl or unsubstituted or N-lower alkanoylated piperidyl-lower alkyl or unsubstituted or N-lower alkanoylated piperidyl,
or a pharmaceutically acceptable salt thereof.

28. A method according to claim 25 wherein,
R₁ is a 2-R_{A}-3-R_{B}-phenyl radical, a 2-R_{A}-4-R_{C}-phenyl radical, a 2-R_{A}-pyridin-3-yl radical, a 3-R_{A}-pyridin-2-yl radical or a 1-R_{D}-indol-3-yl radical, wherein
one of the radicals R_{A} and R_{B} is C₁-C₄ alkyl, hydroxy-C₁-C₄ alkyl, C₁-C₄ alkanoyloxy-C₁-C₄ alkyl, C₁-C₄ alkoxy-C₁-C₄ alkyl, C₁-C₄ alkoxy-C₁-C₄ alkoxy-C₁-C₄ alkyl, amino-C₁-C₄ alkyl, C₁-C₄ alkanoylamino-C₁-C₄ alkyl, C₁-C₄ alkylamino-C₁-C₄ alkyl, di-C₁-C₄ alkylamino-C₁-C₄ alkyl, piperidino-C₁-C₄-alkyl, hydroxypiperidino-C₁-C₄ alkyl, C₁-C₄ alkoxypiperidino-C₁-C₄ alkyl, C₁-C₄ alkoxy-C₁-C₄-alkoxypiperidino-C₁-C₄ alkyl, C₁-C₄ alkoxycarbonylpiperidino-C₁-C₄ alkyl, pyrrolidino-C₁-C₄ alkyl, hydroxypyrrolidino-C₁-C₄ alkyl, C₁-C₄ alkoxypyrrolidino-C₁-C₄ alkyl, C₁-C₄ alkoxy-C₁-C₄ alkoxypyrrolidino-C₁-C₄ alkyl, piperazino-C₁-C₄ alkyl, N′-C₁-C₄ alkylpiperazino-C₁-C₄ alkyl, N′-C₁-C₄-alkanoylpiperazino-C₁-C₄ alkyl, N′-C₁-C₄ alkoxycarbonylpiperazino-C₁-C₄ alkyl, N′-C₁-C₄ alkoxy-C₁-C₄ alkylpiperazino-C₁-C₄ alkyl, morpholino-C₁-C₄ alkyl, C₁-C₄ alkylmorpholino-C₁-C₄ alkyl, thiomorpholino-C₁-C₄ alkyl, S-oxythiomorpholino-C₁-C₄ alkyl, S,S-dioxythiomorpholino-C₁-C₄ alkyl, C₁-C₇ alkoxy, such as propyloxy, amino-C₁-C₇ alkoxy, C₁-C₄ alkanoylamino-C₁-C₄ alkoxy, C₁-C₄ alkylamino-C₁-C₄ alkoxy, di-C₁-C₄ alkylamino-C₁-C₄ alkoxy, piperidino-C₁-C₄ alkoxy, hydroxypiperidino-C₁-C₄ alkoxy, C₁-C₄ alkoxypiperidino-C₁-C₄ alkoxy, C₁-C₄ alkoxy-C₁-C₄-alkoxypiperidino-C₁-C₄ alkoxy, pyrrolidino-C₁-C₄ alkoxy, hydroxypyrrolidino-C₁-C₄ alkoxy, C₁-C₄-alkoxypyrrolidino-C₁-C₄ alkoxy, C₁-C₄ alkoxy-C₁-C₄ alkoxypyrrolidino-C₁-C₄ alkoxy, piperazino-C₁-C₄ alkoxy, N′-C₁-C₄ alkylpiperazino-C₁-C₄ alkoxy, N′-C₁-C₄ alkanoylpiperazino-C₁-C₄ alkoxy, N′-C₁-C₄ alkoxycarbonylpiperazino-C₁-C₄ alkoxy, N′-C₁-C₄ alkoxy-C₁-C₄ alkylpiperazino-C₁-C₄ alkoxy, morpholino-C₁-C₄ alkoxy or C₁-C₄ alkylmorpholino-C₁-C₄ alkoxy, thiomorpholino-C₁-C₄ alkoxy, S-oxythiomorpholino-C₁-C₄ alkoxy, S,S-dioxythiomorpholino-C₁-C₄ alkoxy, hydroxy, hydroxy-C₁-C₄ alkoxy, C₁-C₄ alkanoyloxy-C₁-C₄ alkoxy, C₁-C₄ alkoxy-C₁-C₄ alkoxy, C₁-C₄ alkoxy-C₁-C₄ alkoxy-C₁-C₄ alkoxy, polyhalo-C₁-C₄ alkoxy, cyano-C₁-C₄ alkoxy, carbamoyl-C₁-C₄ alkoxy, such as 2-carbamoylethoxy; phenyl- or pyridyl-C₁-C₄ alkoxy that is unsubstituted or substituted by C₁-C₄ alkyl, C₁-C₄ alkoxy, hydroxy, nitro, amino, C₁-C₄ alkylamino, di-C₁-C₄ alkylamino, halogen and/or by trifluoromethyl; C₁-C₄ alkoxy-C₁-C₄ alkenyloxy, C₁-C₄ alkylthio-C₁-C₄ alkoxy, C₁-C₄ alkanesulfinyl-C₁-C₄ alkoxy, C₁-C₄ alkanesulfonyl-C₁-C₄ alkoxy, amino-C₁-C₇ alkoxy, C₁-C₄ alkanoylamino-C₁-C₄ alkoxy, C₁-C₄ alkylamino-C₁-C₄ alkoxy, di-C₁-C₄ alkylamino-C₁-C₄ alkoxy, piperidino-C₁-C₄ alkoxy, hydroxypiperidino-C₁-C₄ alkoxy, C₁-C₄ alkoxypiperidino-C₁-C₄ alkoxy, C₁-C₄ alkoxy-C₁-C₄ alkoxypiperidino-C₁-C₄ alkoxy, pyrrolidino-C₁-C₄ alkoxy, hydroxypyrrolidino-C₁-C₄ alkoxy, C₁-C₄ alkoxypyrrolidino-C₁-C₄ alkoxy, C₁-C₄ alkoxy-C₁-C₄ alkoxypyrrolidino-C₁-C₄ alkoxy, piperazino-C₁-C₄ alkoxy, N′-C₁-C₄ alkylpiperazino-C₁-C₄ alkoxy, N′-C₁-C₄ alkanoylpiperazino-C₁-C₄ alkoxy, N′-C₁-C₄ alkoxycarbonylpiperazino-C₁-C₄ alkoxy, N′-C₁-C₄ alkoxy-C₁-C₄ alkylpiperazino-C₁-C₄ alkoxy, morpholino-C₁-C₄ alkoxy or C₁-C₄ alkylmorpholino-C₁-C₄ alkoxy or thiomorpholino-C₁-C₄ alkoxy, and the other is hydrogen, carbamoyl, C₁-C₄ alkyl, hydroxy, C₁-C₄ alkoxy or trihalo-C₁-C₄ alkoxy, R_{C} is hydrogen, hydroxy, di-C₁-C₄ alkylamino, piperidino, pyrrolidino, morpholino, thiomorpholino, S-oxythiomorpholino, S,S-dioxythiomorpholino, C₁-C₄ alkoxy, hydroxy-C₁-C₄ alkoxy, C₁-C₄ alkoxy-C₁-C₄ alkoxy, morpholino-C₁-C₄ alkylcarbamoyl-C₁-C₄ alkoxy, C₁-C₄ alkoxy-C₁-C₄ alkoxy-C₁-C₄ alkyl, amino-C₁-C₄ alkyl, C₁-C₄ alkanoylamino-C₁-C₄ alkyl, C₁-C₄ alkylamino-C₁-C₄ alkyl, di-C₁-C₄ alkylamino-C₁-C₄ alkyl; piperidino- or pyrrolidino-C₁-C₄ alkyl that is unsubstituted or substituted by hydroxy, C₁-C₄ alkoxy or by C₁-C₄ alkoxy-C₁-C₄ alkyl; amino-C₁-C₄ alkyl, C₁-C₄ alkanoylamino-C₁-C₄ alkyl, C₁-C₄ alkylamino-C₁-C₄ alkyl, di-C₁-C₄ alkylamino-C₁-C₄ alkyl, piperidino-C₁-C₄ alkyl, hydroxypiperidino-C₁-C₄ alkyl, C₁-C₄ alkoxypiperidino-C₁-C₄ alkyl, C₁-C₄ alkoxy-C₁-C₄ alkoxypiperidino-C₁-C₄ alkyl, C₁-C₄ alkoxycarbonylpiperidino-C₁-C₄ alkyl, pyrrolidino-C₁-C₄ alkyl, hydroxypyrrolidino-C₁-C₄ alkyl, C₁-C₄ alkoxypyrrolidino-C₁-C₄ alkyl, C₁-C₄ alkoxy-C₁-C₄ alkoxypyrolidino-C₁-C₄ alkyl, piperazino-C₁-C₄ alkyl, N′-C₁-C₄ alkylpiperazino-C₁-C₄ alkyl, N′-C₁-C₄ alkanoylpiperazino-C₁-C₄ alkyl, N′-C₁-C₄ alkoxycarbonylpiperazino-C₁-C₄ alkyl, N′-C₁-C₄ alkoxy-C₁-C₄ alkylpiperazino-C₁-C₄ alkyl, morpholino-C₁-C₄ alkyl, C₁-C₄ alkylmorpholino-C₁-C₄ alkyl, thiomorpholino-C₁-C₄ alkyl, S-oxythiomorpholino-C₁-C₄ alkyl, S,S-dioxythiomorpholino-C₁-C₄ alkyl, amino-C₁-C₇ alkoxy, C₁-C₄ alkanoylamino-C₁-C₄ alkoxy, C₁-C₄ alkylamino-C₁-C₄ alkoxy, di-C₁-C₄ alkylamino-C₁-C₄ alkoxy, piperidino-C₁-C₄ alkoxy, hydroxypiperidino-C₁-C₄ alkoxy, C₁-C₄ alkoxypiperidino-C₁-C₄ alkoxy, C₁-C₄ alkoxy-C₁-C₄ alkoxypiperidino-C₁-C₄ alkoxy, pyrrolidino-C₁-C₄ alkoxy, hydroxypyrrolidino-C₁-C₄ alkoxy, C₁-C₄ alkoxypyrrolidino-C₁-C₄ alkoxy, C₁-C₄ alkoxy-C₁-C₄ alkoxypyrrolidino-C₁-C₄ alkoxy, piperazino-C₁-C₄ alkoxy, N′-C₁-C₄ alkylpiperazino-C₁-C₄ alkoxy, N′-C₁-C₄ alkanoylpiperazino-C₁-C₄ alkoxy, N′-C₁-C₄ alkoxycarbonylpiperazino-C₁-C₄ alkoxy, N′-C₁-C₄ alkoxy-C₁-C₄ alkylpiperazino-C₁-C₄ alkoxy, morpholino-C₁-C₄ alkoxy or C₁-C₄ alkylmorpholino-C₁-C₄ alkoxy, thiomorpholino-C₁-C₄ alkoxy, S-oxythiomorpholino-C₁-C₄ alkoxy, S,S-dioxythiomorpholino-C₁-C₄ alkoxy, carboxy-C₁-C₄ alkoxy, carbamoyl-C₁-C₄ alkoxy, C₁-C₄ alkylcarbamoyl-C₁-C₄ alkoxy, di-C₁-C₄-alkylcarbamoyl-C₁-C₄ alkoxy, di-C₁-C₄ alkylamino-C₁-C₄ alkoxy, such as 3-dimethylaminopropyloxy, piperidinocarbonyl-C₁-C₄ alkoxy, hydroxypiperidinocarbonyl-C₁-C₄ alkoxy, C₁-C₄ alkoxypiperidinocarbonyl-C₁-C₄ alkoxy, C₁-C₄ alkoxy-C₁-C₄ alkoxypiperidinocarbonyl-C₁-C₄ alkoxy, pyrrolidinocarbonyl-C₁-C₄ alkoxy, hydroxypiperidinocarbonyl-C₁-C₄ alkoxy, C₁-C₄ alkoxypyrrolidinocarbonyl-C₁-C₄ alkoxy, C₁-C₄ alkoxy-C₁-C₄ alkoxypyrrolidinocarbonyl-C₁-C₄ alkoxy, piperazinocarbonyl-C₁-C₄ alkoxy, N′-C₁-C₄ alkylpiperazinocarbonyl-C₁-C₄ alkoxy, N′-C₁-C₄ alkanoylpiperazinocarbonyl-C₁-C₄ alkoxyl, N′-C₁-C₄ alkoxycarbonylpiperazinocarbonyl or N′-C₁-C₄ alkoxy-C₁-C₄ alkylipiperazinocarbonyl-C₁-C₄ alkoxy, morpholinocarbonyl-C₁-C₄ alkoxy, C₁-C₄ alkylmorpholinocarbonyl-C₁-C₄ alkoxy, thiomorpholinocarbonyl-C₁-C₄ alkoxy, S-oxythiomorpholinocarbonyl, S,S-dioxythiomorpholinocarbonyl-C₁-C₄ alkoxy, tetrazolyl-C₁-C₄ alkoxy, carboxy, carbamoyl or C₁-C₄ alkylcarbamoyl, such as methylcarbamoyl, and
R_{D} is C₁-C₄ alkyl, hydroxy-C₁-C₄ alkyl, C₁-C₄ alkoxy-C₁-C₄ alkyl, C₁-C₄ alkoxy-C₁-C₄ alkoxy-C₁-C₄ alkyl, hydroxy-C₁-C₄ alkoxy-C₁-C₄ alkyl, carboxy, C₁-C₄ alkoxycarbonyl, carboxy-C₁-C₄ alkyl, C₁-C₄ alkoxycarbonyl-C₁-C₄ alkyl, carbamoyl-C₁-C₄ alkyl, C₁-C₄ alkylcarbamoyl-C₁-C₄ alkyl, di-C₁-C₄ alkylcarbamoyl-C₁-C₄ alkyl, piperidino-C₁-C₄ alkyl, hydroxypiperidino-C₁-C₄ alkyl, C₁-C₄ alkoxypiperidino-C₁-C₄ alkyl, C₁-C₄ alkoxy-C₁-C₄ alkoxypiperidino-C₁-C₄ alkyl, C₁-C₄ alkoxycarbonylpiperidino-C₁-C₄ alkyl, pyrrolidino-C₁-C₄ alkyl, hydroxypyrrolidino-C₁-C₄ alkyl, C₁-C₄ alkoxypyrrolidino-C₁-C₄ alkyl, C₁-C₄ alkoxy-C₁-C₄ alkoxypyrrolidino-C₁-C₄ alkyl, piperazino-C₁-C₄ alkyl, N′-C₁-C₄ alkylpiperazino-C₁-C₄ alkyl, N′-C₁-C₄ alkanoylpiperazino-C₁-C₄ alkyl, N′-C₁-C₄ alkoxycarbonylpiperazino-C₁-C₄ alkyl, N′-C₁-C₄ alkoxy-C₁-C₄ alkylpiperazino-C₁-C₄ alkyl, morpholino-C₁-C₄ alkyl, C₁-C₄ alkylmorpholino-C₁-C₄ alkyl, thiomorpholino-C₁-C₄ alkyl, S-oxythiomorpholino-C₁-C₄ alkyl, S,S-dioxythiomorpholino-C₁-C₄ alkyl, carboxy-C₁-C₄ alkyl, C₁-C₄ alkoxycarbonyl-C₁-C₄ alkyl, or is phenyl-C₁-C₄ alkyl or pyridyl-C₁-C₄ alkyl that is unsubstituted or substituted by C₁-C₄ alkyl, C₁-C₄ alkoxy, hydroxy, nitro, amino, C₁-C₄ alkylamino, di-C₁-C₄ alkylamino, halogen and/or by trifluoromethyl,
one of the radicals X₁ and X₂ is carbonyl and the other is methylene,
R₂ is C₁-C₄ alkyl,
R₃ is amino, C₁-C₄ alkanoylamino, C₁-C₄ alkylamino or di-C₁-C₄ alkylamino,
R₄ is C₁-C₄ alkyl or phenyl-C₁-C₄ alkyl, and
R₅ is C₁-C₄ alkyl, cycloalkyl-C₁-C₄ alkyl, hydroxy-C₁-C₄ alkyl, C₁-C₄ alkoxyl-C₁-C₄ alkyl, C₁-C₄ alkanoyloxy-C₁-C₄ alkyl, piperidino-C₁-C₄ alkyl, hydroxypiperidino-C₁-C₄ alkyl, C₁-C₄ alkoxypiperidino-C₁-C₄ alkyl, C₁-C₄ alkoxy-C₁-C₄ alkoxypiperidino-C₁-C₄ alkyl, C₁-C₄ alkoxycarbonylpiperidino-C₁-C₄ alkyl, pyrrolidino-C₁-C₄ alkyl, hydroxypyrrolidino-C₁-C₄ alkyl, C₁-C₄ alkoxypyrrolidino-C₁-C₄ alkyl, C₁-C₄ alkoxy-C₁-C₄ alkoxypyrrolidino-C₁-C₄ alkyl, piperazino-C₁-C₄ alkyl, N′-C₁-C₄ alkylpiperazino-C₁-C₄ alkyl, N′-C₁-C₄ alkanoylpiperazino-C₁-C₄ alkyl, N′-C₁-C₄ alkoxycarbonylpiperazino-C₁-C₄ alkyl, N′-C₁-C₄ alkoxy-C₁-C₄ alkylpiperazino-C₁-C₄ alkyl, morpholino-C₁-C₄ alkyl, C₁-C₄ alkylmorpholino-C₁-C₄ alkyl, thiomorpholino-C₁-C₄ alkyl, S-oxythiomorpholino-C₁-C₄ alkyl, S,S-dioxythiomorpholino-C₁-C₄ alkyl, carboxy-C₁-C₄ alkyl, C₁-C₄ alkoxycarbonyl-C₁-C₄ alkyl, carbamoyl-C₁-C₄ alkyl, C₁-C₄ alkylcarbamoyl-C₁-C₄ alkyl, di-C₁-C₄ alkylcarbamoyl-C₁-C₄ alkyl, piperidinocarbonyl-C₁-C₄ alkyl, hydroxypiperidinocarbonyl-C₁-C₄ alkyl, C₁-C₄ alkoxypiperidinocarbonyl-C₁-C₄ alkyl, C₁-C₄ alkoxy-C₁-C₄ alkoxypiperidinocarbonyl-C₁-C₄ alkyl, pyrrolidinocarbonyl-C₁-C₄ alkyl, hydroxypyrrolidinocarbonyl-C₁-C₄ alkyl, C₁-C₄ alkoxypyrrolidinocarbonyl-C₁-C₄ alkyl, C₁-C₄ alkoxy-C₁-C₄ alkoxypyrrolidinocarbonyl-C₁-C₄ alkyl, piperazinocarbonyl-C₁-C₄ alkyl, N′-C₁-C₄ alkylpiperazinocarbonyl-C₁-C₄ alkyl, N′-C₁-C₄ alkanoylpiperazinocarbonyl-C₁-C₄ alkyl, N′-C₁-C₄ alkoxycarbonylpiperazinocarbonyl, N′-C₁-C₄ alkoxy-C₁-C₄ alkylpiperazinocarbonyl-C₁-C₄ alkyl, morpholinocarbonyl-C₁-C₄ alkyl, C₁-C₄ alkylmorpholinocarbonyl-C₁-C₄ alkyl, thiomorpholinocarbonyl-C₁-C₄ alkyl, S-oxythiomorpholinocarbonyl-C₁-C₄ alkyl, S,S-dioxythiomorpholinocarbonyl-C₁-C₄ alkyl, carbamoyl-C₁-C₄ alkyl, C₁-C₄ alkylcarbamoyl-C₁-C₄ alkyl, di-C₁-C₄ alkylcarbamoyl-C₁-C₄ alkyl, cyano-C₁-C₄ alkyl, dicarboxy-C₁-C₄ alkyl, C₁-C₄ alkoxycarbonyl(carboxy)-C₁-C₄ alkyl, di-C₁-C₄ alkoxycarbonyl-C₁-C₄ alkyl, dicarbamoyl-C₁-C₄ alkyl, carbamoyl(carboxy)-C₁-C₄ alkyl, di-(C₁-C₄ alkylcarbamoyl)-C₁-C₄ alkyl, di-(di-C₁-C₄ alkylcarbamoyl)-C₁-C₄ alkyl, carboxy(hydroxy)-C₁-C₄ alkyl, C₁-C₄ alkoxycarbonyl(hydroxy)-C₁-C₄ alkyl, carbamoyl(hydroxy)-C₁-C₄ alkyl, C₁-C₄ alkylcarbamoyl(hydroxy)-C₁-C₄ alkyl or di-C₁-C₄ alkylcarbamoyl(hydroxy)-C₁-C₄ alkyl, carboxycycloalkyl-C₁-C₄ alkyl, C₁-C₄ alkoxycarbonylcycloalkyl-C₁-C₄ alkyl, carbamoylcycloalkyl-C₁-C₄ alkyl, C₁-C₄ alkylcarbamoylcycloalkyl-C₁-C₄ alkyl, di-C₁-C₄ alkylcarbamoylcycloalkyl-C₁-C₄ alkyl, C₁-C₄ alkanesulfonyl-C₁-C₄ alkyl, thiocarbamoyl-C₁-C₄ alkyl, N—C₁-C₄ alkylthiocarbamoyl-C₁-C₄ alkyl or N,N-di-C₁-C₄ alkylthiocarbamoyl-C₁-C₄ alkyl, sulfamoyl-C₁-C₄ alkyl, C₁-C₄ alkylsulfamoyl-C₁-C₄ alkyl or di-C₁-C₄ alkylsulfamoyl-C₁-C₄ alkyl, unsubstituted or oxo-substituted pyrrolidinyl, imidazolyl, benzimidazolyl, oxadiazolyl, pyridyl, oxopiperidinyl, dioxopiperidinyl, oxothiazolyl, oxo-oxazolinyl or quinolinyl, unsubstituted or oxo-substituted pyrrolidinyl-C₁-C₄ alkyl, imidazolyl-C₁-C₄ alkyl, benzimidazolyl-C₁-C₄ alkyl, oxadiazolyl-C₁-C₄ alkyl, pyridyl-C₁-C₄ alkyl, oxopiperidinyl-C₁-C₄ alkyl, dioxopiperidinyl-C₁-C₄ alkyl, oxothiazolyl-C₁-C₄ alkyl, oxo-oxazolinyl-C₁-C₄ alkyl or quinolinyl-C₁-C₄ alkyl, morpholinocarbonyl-C₁-C₄ alkyl or unsubstituted or N—C₁-C₄ alkanoylated piperidyl-C₁-C₄ alkyl or unsubstituted or N—C₁-C₄ alkanoylated piperidyl,
or a pharmaceutically acceptable salt thereof.

29. A method according to claim 25, wherein
R₁ is a 2-R_{A}-3-R_{B}-phenyl radical, a 2-R_{A}-4-R_{C}-phenyl radical, a 2-R_{A}-pyridin-3-yl radical, a 3-R_{A}-pyridin-2-yl radical or a 1-R_{D}-indol-3-yl radical, wherein
one of the radicals R_{A} and R_{B} is C₁-C₄ alkyl, C₁-C₄ alkoxy-C₁-C₄ alkyl, di-C₁-C₄ alkylamino-C₁-C₄ alkyl, piperidino-C₁-C₄ alkyl, C₁-C₄ alkanoylpiperidinyl-C₁-C₄ alkyl, C₁-C₄ alkoxycarbonylpiperidino-C₁-C₄ alkyl, pyrrolidino-C₁-C₄ alkyl, piperazino-C₁-C₄ alkyl, N′-C₁-C₄ alkylpiperazino-C₁-C₄ alkyl, N′-C₁-C₄ alkanoylpiperazino-C₁-C₄ alkyl, morpholino-C₁-C₄ alkyl, C₁-C₄ alkylmorpholino-C₁-C₄ alkyl, thiomorpholino-C₁-C₄ alkyl, amino-C₁-C₇ alkoxy, C₁-C₄ alkanoylamino-C₁-C₄ alkoxy, di-C₁-C₄ alkylamino-C₁-C₄ alkoxy, piperidino-C₁-C₄ alkoxy, morpholino-C₁-C₄ alkoxy, hydroxy, C₁-C₇ alkoxy, C₁-C₄ alkoxy-C₁-C₄ alkoxy, C₁-C₄ alkoxy-C₁-C₄ alkoxy-C₁-C₄ alkoxy, C₁-C₄ alkoxy-C₁-C₄ alkenyloxy, amino-C₁-C₄ alkoxy, C₁-C₄ alkanoylamino-C₁-C₄ alkoxy, di-+C₁-C₄ alkylamino-C₁-C₄ alkoxy, piperidino-C₁-C₄ alkoxy, morpholino-C₁-C₄ alkoxy, carbamoyl or carbamoyl-C₁-C₄ alkoxy, and the other is hydrogen, C₁-C₄ alkyl, such as methyl, hydroxy or C₁-C₄ alkoxy,
R_{C} is hydrogen, hydroxy, C₁-C₄ alkoxy, C₁-C₄ alkoxy-C₁-C₄ alkoxy, morpholino-C₁-C₄ alkylcarbamoyl-C₁-C₄ alkoxy, di-C₁-C₄ alkylamino-C₁-C₄ alkyl, piperidino-C₁-C₄ alkyl, C₁-C₄ alkoxycarbonylpiperidino-C₁-C₄ alkyl, pyrrolidino-C₁-C₄ alkyl, piperazinocarbonyl-C₁-C₄ alkyl, N′-C₁-C₄ alkylpiperazinocarbonyl-C₁-C₄ alkyl, N′-C₁-C₄ alkanoylpiperazinocarbonyl-C₁-C₄ alkyl, morpholino, morpholino-C₁-C₄ alkyl, thiomorpholino-C₁-C₄ alkyl, C₁-C₄ alkoxy, amino-C₁-C₁-C₄ alkoxy, C₁-C₄ alkanoylamino-C₁-C₄ alkoxy, di-C₁-C₄ alkylamino-C₁-C₄ alkoxy, piperidino-C₁-C₄ alkoxy, morpholino-C₁-C₄ alkoxy, morpholino-C₁-C₄ alkylcarbamoyl-C₁-C₄ alkoxy, carboxy, carbamoyl, C₁-C₄ alkylcarbamoyl, carboxy-C₁-C₄ alkoxy, carbamoyl-C₁-C₄ alkoxy, C₁-C₄ alkylcarbamoyl-C₁-C₄ alkoxy, di-C₁-C₄ alkylamino-C₁-C₄ alkoxy or tetrazolyl-C₁-C₄ alkoxy, and
R_{D} is C₁-C₄ alkyl, C₁-C₄ alkoxy-C₁-C₄ alkyl, carbamoyl-C₁-C₄ alkyl, C₁-C₄ alkylcarbamoyl-C₁-C₄ alkyl, di-C₁-C₄ alkylcarbamoyl-C₁-C₄ alkyl, piperidino-C₁-C₄ alkyl, or C₁-C₄ alkoxycarbonylpiperidino-C₁-C₄ alkyl,
one of the radicals X₁ and X₂ is carbonyl and the other is methylene,
R₂ is C₁-C₄ alkyl,
R₃ is amino or C₁-C₄ alkanoylamino,
R₄ is C₁-C₄ alkyl, and
R₅ is C₁-C₄ alkyl, C₁-C₄ alkoxy-C₁-C₄ alkyl, C₁-C₄ alkoxycarbonylpiperidino-C₁-C₄ alkyl, pyrrolidino-C₁-C₄ alkyl, N′-C₁-C₄ alkylpiperazino-C₁-C₄ alkyl, N′-C₁-C₄ alkoxycarbonylpiperazino-C₁-C₄ alkyl or N′-C₁-C₇ alkanoylpiperazino-C₁-C₄ alkyl, morpholino-C₁-C₄ alkyl, thiomorpholino-C₁-C₄ alkyl, morpholinocarbonyl-C₁-C₄ alkyl, carbamoyl-C₁-C₄ alkyl, C₁-C₄ alkylcarbamoyl-C₁-C₄ alkyl, di-C₁-C₄ alkylcarbamoyl-C₁-C₄ alkyl, piperidinocarbonyl-C₁-C₄ alkyl, piperazinocarbonyl-C₁-C₄ alkyl, N′-C₁-C₄ alkylpiperazinocarbonyl-C₁-C₄ alkyl, N′-C₁-C₄ alkanoylpiperazinocarbonyl-C₁-C₄ alkyl, N′-C₁-C₄ alkylpiperazinocarbonyl-C₁-C₄ alkyl, or morpholinocarbonyl-C₁-C₄ alkyl,
or a pharmaceutically acceptable salt thereof.

30. A method according to claim 23, wherein
R₁ is a 2-R_{A}-4-R_{C}-phenyl radical, a 2-R_{A}-pyridin-3-yl radical or a 3-R_{A}-pyridin-2-yl radical, wherein
R_{A} is C₁-C₄ alkoxy-C₁-C₄ alkyl, morpholino-C₁-C₄ alkyl, C₁-C₇ alkanoylpiperazino-C₁-C₄ alkyl, C₁-C₇ alkoxy, C₁-C₄ alkoxy-C₁-C₄ alkoxy, C₁-C₄ alkoxy-C₁-C₄ alkenyloxy, C₁-C₄ alkoxy-C₁-C₄ alkoxy-C₁-C₄ alkoxy, amino-C₁-C₄ alkoxy, di-C₁-C₄ alkylamino-C₁-C₄ alkoxy, carbamoyl-C₁-C₄ alkoxy or carbamoyl, and
R_{C} is hydrogen, di-C₁-C₄ alkylamino-C₁-C₄ alkyl, piperidino-C₁-C₄ alkyl, pyrrolidino-C₁-C₄ alkyl, morpholino-C₁-C₄ alkyl, C₁-C₄ alkanoylpiperazino-C₁-C₇ alkyl, or C₁-C₄ alkylpiperazino-C₁-C₄ alkyl, morpholino-C₁-C₄ alkoxy, morpholino-C₁-C₄ alkylcarbamoyl-C₁-C₄ alkoxy, piperidino-C₁-C₄ alkoxy, carboxy, carbamoyl, C₁-C₄ alkylcarbamoyl, carboxy-C₁-C₄ alkoxy, di-C₁-C₄ alkylamino-C₁-C₄ alkoxy, C₁-C₄ alkylcarbamoyl-C₁-C₄ alkoxy or tetrazolyl-C₁-C₇ alkoxy,
X₁ is carbonyl and X₂ is methylene,
R₂ and R₄ are each independently of the other C₁-C₄ alkyl,
R₃ is amino and
R₅ is C₁-C₄ alkyl, morpholino-C₁-C₄ alkyl, thiomorpholino-C₁-C₄ alkyl, morpholinocarbonyl-C₁-C₄ alkyl, carbamoyl-C₁-C₄ alkyl, C₁-C₄ alkylcarbamoyl-C₁-C₄ alkyl, di-C₁-C₄ alkylcarbamoyl-C₁-C₄ alkyl, N′-C₁-C₄ alkylpiperazino-C₁-C₄ alkyl, N′-C₁-C₄ alkoxycarbonylpiperazino-C₁-C₄ alkyl or N′-C₁-C₇ alkanoylpiperazino-C₁-C₄ alkyl,
or a pharmaceutically acceptable salt thereof.


SEQUENCE LISTING (9 sequences)

SEQ ID NO: 1 - protein, 13 aa, Artificial sequence
  synthetic peptide
SEVNLDAEFRCKK

SEQ ID NO: 2 - protein, 13 aa, Artificial sequence
  synthetic peptide
SEVKMDAEFRCKK

SEQ ID NO: 3 - protein, 22 aa, Artificial sequence
  synthetic peptide
GLNIKTEEISEISYEVEFRCKK

SEQ ID NO: 4 - protein, 34 aa, Artificial sequence
  synthetic peptide
ADRGLTTRPGSGLTNIKTEEISEVNLDAEFRCKK

SEQ ID NO: 5 - protein, 33 aa, Artificial sequence
  synthetic peptide
FVNQHLCGSHLVEALYLVCGERGFFYTPKACKK

SEQ ID NO: 6 - protein, 33 aa, Artificial sequence
  synthetic peptide
CGGADRGLTTRPGSGLTNIKTEEISEVNLDAEF

SEQ ID NO: 7 - protein, 29 aa, Artificial sequence
  synthetic peptide
CGGADRGLTTRPGSGLTNIKTEEISEVNL

SEQ ID NO: 8 - protein, 9 aa, Artificial sequence
  synthetic peptide
SEVNLDAEF

SEQ ID NO: 9 - protein, 30 aa, Artificial sequence
  synthetic peptide
ADRGLTTRPGSGLTNIKTEEISEVNLDAEF